(19) United States
(12) Patent Application Publication
(10) Pub. No.: US 2026/0217688 A1
(43) Pub. Date: Jul. 30, 2026

(54) INHIBITORS OF JAK2

(71) Applicant: Llydaw Therapeutics, Inc., Dover, DE (US)

(72) Inventors: Alexander Khvat, San Diego, CA (US); Igor Rezekin, Khimki (RU); Ruben Abagyan, La Jolla, CA (US); Nikolay Savchuk, San Diego, CA (US)

(21) Appl. No.: 19/141,044

(22) PCT Filed: Dec. 18, 2023
(86) PCT No.: PCT/US2023/084697

§ 371 Date: Jun. 18, 2025

Related U.S. Application Data
(60) Provisional application No. 63/434,412, filed on Dec. 21, 2022.

(51) Int. Cl. C07D 403/14; A61K 31/506; C07D 401/14; C07D 405/14; C07D 413/14; C07D 417/14; C07D 498/04

(52) U.S. Cl. CPC C07D 403/14; A61K 31/506; C07D 401/14; C07D 405/14; C07D 413/14; C07D 417/14; C07D 498/04

(57) ABSTRACT

The present invention is directed to the compounds of Formula (I)—inhibitors JAK2. The inhibitors described herein can be useful in the treatment of diseases or disorders associated with JAK2, such as blood diseases. In particular, the invention is concerned with compounds and pharmaceutical compositions inhibiting JAK2, methods of treating diseases or disorders associated with JAK2, and methods of synthesizing these compounds.

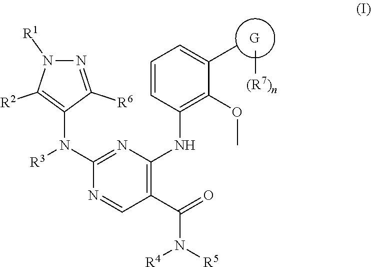

### CROSS-REFERENCE TO RELATED APPLICATIONS

This application claims priority to and benefit of U.S. Provisional Patent Application Ser. No. 63/434,412 filed Dec. 21, 2022, entitled “Inhibitors of JAK2” the disclosure of which are incorporated by reference in its entirety for all purposes.

### FIELD OF INVENTION

The present invention is directed to inhibitors of JAK2. The inhibitors described herein can be useful in the treatment of diseases or disorders associated with JAK2, such as blood diseases. In particular, the invention is concerned with compounds and pharmaceutical compositions inhibiting JAK2, methods of treating diseases or disorders associated with JAK2, and methods of synthesizing these compounds.

### BACKGROUND

Myeloproliferative disorders (MPD) are clonal disorders of hematopoietic progenitors, and include the classical MPD chronic myeloid leukemia (CML), polycythemia vera (PV), essential thrombocythemia (ET) and primary myelofibrosis (PMF), as well as chronic eosinophilic leukemia (CEL), chronic myelomonocytic leukemia (CMML), and systemic mastocytosis (SM) and others. Although each of the MPD is recognized as a distinct clinicopathological entity, these disorders share cardinal features that distinguish the MPD from other myeloid malignancies, namely myelodysplastic syndromes (MDS) and acute myeloid leukemia (AML).

In the past three decades, mutant alleles have been identified in CML, CMML, CEL and SM, and in each case the causative mutation results in constitutive activation of tyrosine kinase signaling. Perhaps most importantly from a clinical perspective, specific inhibition of these activated kinases results in dramatic clinical efficacy in the treatment of MPD. Collectively, these data indicate that tyrosine kinase activation is a common pathogenetic mechanism in MPD, and that these mutated kinases serve as validated targets for the design of molecularly targeted therapies.

In 2005, several independent groups used different experimental approaches to identify a recurrent mutation in the JAK2 tyrosine kinase in most patients with PV, ET or PMF. JAK2 is a member of the Janus family of cytoplasmic non-receptor tyrosine kinases, which also includes JAK1, JAK3 and TYK2. The mutation is a guanine-to-thymidine substitution, which results in a substitution of valine for phenylalanine at codon 617 of JAK2 (JAK2V617F). The mutation is present in hematopoietic cells but not germline DNA in patients with MPD, demonstrating that JAK2V617F is a somatic mutation that is acquired in the hematopoietic compartment. In addition, the JAK2V617F allele can occasionally be present in different hematopoietic compartments, including B and T lymphoid cells. These findings suggest that the mutation might occur in the pluripotent hematopoietic stem cell; indeed, the JAK2V617F allele has recently been identified in the hematopoietic stem cell (HSC) compartment in patients with PV.

JAKs are unique among tyrosine kinases in that they possess a pseudokinase domain, which is just upstream of the C-terminal tyrosine kinase domain. A wealth of biochemical and clinical data has established that the pseudokinase domain of JAKs is crucial for maintaining a low basal (absence of cytokine) level of tyrosine kinase activity. In particular, gain-of-function mutations in the JAK genes, most frequently, V617F in the pseudokinase domain of JAK2, have been mapped in patients with blood disorders, including myeloproliferative neoplasms and leukemias.

The tyrosine kinase domain (JH1) of JAKs is responsible for the majority, if not all, of the phosphoryl transfer activity of JAKs. In particular, JH1 catalyzes trans-phosphorylation of two tyrosine residues in the kinase activation loop (Tyr1007 and Tyr1008 in JAK2), which stabilizes the active state. Trans-phosphorylation of these two tyrosines is the key step in JAK activation. The activated kinase domain then phosphorylates specific tyrosine residues in the associated cytokine receptor and in the recruited STAT molecules, as well as other tyrosines in the JAK molecule.

Both biochemical and clinical evidence have demonstrated an important regulatory function for JH2 in JAKs, and at present, 32 different mutations in JH2 of JAK2 have been shown to cause, or are linked to, hematological diseases. The most frequent somatic mutation, V617F, results in constitutively active JAK2, and is responsible for >95% of polycythemia vera cases and ~50% of essential thrombocythemia and primary myelofibrosis cases. The mechanism(s) by which JH2 negatively regulates the tyrosine kinase activity of JH1 is currently not known, but it is likely to involve an intramolecular interaction between JH2 and JH1

Even if JAK2 inhibitor therapy results in molecular and clinical remissions in patients with PV, ET or PMF, a subset of patients will probably develop resistance to JAK2 inhibitor therapy, possibly through the emergence of resistance mutations, as has been observed in imatinib-resistant CML. Development of second-generation JAK2 inhibitors designed to inhibit specific resistance alleles will appropriate approach to overcome this resistance. This strategy has been used to design second-generation ABL inhibitors for the treatment of imatinib refractory CML.

### SUMMARY

A first aspect of the invention relates to compounds of Formula (I):

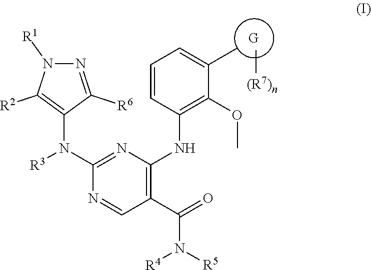

or a pharmaceutically acceptable salt, prodrug, stereoisomer, solvate, or tautomer thereof, wherein
   Ring G is 5-10 membered monocyclic or bicyclic heteroaryl, comprising 1-3 heteroatoms selected from N, O, S;
   R¹ is selected from C₁-C₆ alkyl, C₂-C₆ alkenyl, C₂-C₆ alkynyl, C₃-C₁₀ cycloalkyl, heterocycle, aryl, and heteroaryl, wherein the alkyl, alkenyl, alkynyl, cycloalkyl, heterocycle, aryl, or heteroaryl is optionally substituted with one or more substituents independently selected from halogen, CN, NO₂, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocyclyl, aryl, heteroaryl, wherein the C₃-C₁₀ cycloalkyl, heterocyclyl, aryl, or heteroaryl is optionally substituted with one or more substituents independently selected from C₁-C₆ alkyl, halogen, OH, CN;
   R² is selected from H, C₁-C₆ alkyl;
   R³ is selected from hydrogen, C₁-C₆ alkyl, C₃-C₁₀ cycloalkyl;
   R⁴ is selected from H, C₁-C₆ alkyl;
   R⁵ is selected from H, C₁-C₆ alkyl;
   or R⁴ and R⁵ together with the atoms to which they are attached and any intervening atoms, form a 3-14 membered heterocycle optionally substituted with one or more substituents independently selected from halogen, C₁-C₆ alkyl, and C₁-C₆ alkoxy;
   R⁶ is selected from H, C₁-C₆ alkyl;
   each R⁷ is independently selected from oxo, C₁-C₆ alkyl, C₃-C₁₀ cycloalkyl, heterocyclyl, —NH-heteroaryl, —(CH₂)ₘ—NH—C(O)—R⁸, —(CH₂)ₘ—C(O)NH—R⁸, —(CH₂)ₘ—C(O)NH-heteroaryl, —(CH₂)ₘ—S(O)₂N—R⁸, —(CH₂)ₘ—S(O)₂N-heterocyclyl, wherein the alkyl, cycloalkyl, heterocyclyl or heteroaryl is optionally substituted with one or more substituents independently selected from halogen, —CN, —OH, N(R⁸)₂, C₁-C₆ alkyl, C₁-C₆ alkoxy, cycloalkyl;
   each R⁸ is independently selected from H, C₁-C₆ alkyl, C₃-C₁₀ cycloalkyl;
   n is an integer selected from 0, 1, 2 and 3;
   m is an integer selected from 0, 1, and 2;
wherein,
   cycloalkyl is a mono or polycyclic saturated carbon rings containing 3-18 carbon atoms;
   aryl is a cyclic, aromatic hydrocarbon groups that have 1 to 3 aromatic rings;
   heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
   heteroaryl is a monovalent monocyclic or a polycyclic aromatic radical of 5 to 24 ring atoms, containing one or more ring heteroatoms selected from N, O, S, P, or B, the remaining ring atoms being C.

Another aspect of the invention is directed to pharmaceutical compositions comprising a compound of Formula (I), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention relates to a method of treating a disease or disorder associated with JAK2. The method comprises administering to a patient in need of a treatment for diseases or disorders associated with JAK2 an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting of JAK2. The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting of TYK2. The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for inhibiting of JAK2.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease associated with inhibiting of JAK2.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the invention is directed to a method of treating or preventing a disease or disorder disclosed herein in a subject in need thereof. The method involves administering to a patient in need of the treatment an effective amount of a compound of Formula (I), or a pharmaceutically acceptable salt, hydrate, solvate, prodrug, stereoisomer, tautomer, or pharmaceutical composition thereof.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease or disorder disclosed herein.

The present invention further provides methods of treating a disease or disorder associated with JAK2, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention provides inhibitors of JAK2 that are therapeutic agents in the treatment of diseases and disorders.

The present invention further provides compounds and compositions with an improved efficacy and safety profile relative to known inhibitors of JAK2. The present disclosure also provides agents with novel mechanisms of action toward JAK2 in the treatment of various types of diseases.

The present invention further provides methods of treating a disease or disorder associated with JAK2, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention provides inhibitors of JAK2 that are therapeutic agents in the treatment of diseases and disorders.

The present invention further provides methods of treating a disease, disorder, or condition selected from polycythemia vera, essential thrombocytosis, thrombocythemia 3, primary myelofibrosis, chronic myelomonocytic leukemia, acute myeloid leukemia, myelodysplasia, acute myeloid leukemia, Budd-Chiari syndrome comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

In some aspects, the present disclosure provides a compound obtainable by, or obtained by, a method for preparing compounds described herein (e.g., a method comprising one or more steps described in General Procedure).

In some aspects, the present disclosure provides an intermediate as described herein, being suitable for use in a method for preparing a compound as described herein (e.g., the intermediate is selected from the intermediates described in Preparative part—P1-P84).

In some aspects, the present disclosure provides a method of preparing compounds of the present disclosure.

In some aspects, the present disclosure provides a method of preparing compounds of the present disclosure, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims

### DETAILED DESCRIPTION

The present disclosure provides methods of treating, preventing, or ameliorating a disease or disorder in which associated with JAK2 by administering to a subject in need thereof a therapeutically effective amount of a compound as disclosed herein.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have one or more substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups but does not necessarily have any further functional groups. Suitable substituents used in the optional substitution of the described groups include, without limitation, halogen, oxo, —OH, —CN, —COOH, —CH₂CN, —O—(C₁-C₆)alkyl, (C₁-C₆)alkyl, (C₁-C₆)alkoxy, (C₁-C₆) haloalkyl, (C₁-C₆) haloalkoxy, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O(C₁-C₆)alkyl, —NH₂, —NH((C₁-C₆)alkyl), —N((C₁-C₆)alkyl)₂, —NHC(O)(C₁-C₆) alkyl, —C(O)NH(C₁-C₆)alkyl, —S(O)₂(C₁-C₆)alkyl, —S(O)NH(C₁-C₆)alkyl, and —S(O)N((C₁-C₆)alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

Unless otherwise specifically defined, the term “aryl” refers to cyclic, aromatic hydrocarbon groups that have 1 to 3 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C₁-C₆)alkyl, (C₁-C₆)alkyl, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O(C₁-C₆)alkyl, —NH₂, —NH((C₁-C₆)alkyl), —N((C₁-C₆)alkyl)₂, —S(O)₂—(C₁-C₆)alkyl, —S(O)NH(C₁-C₆)alkyl, and —S(O)N((C₁-C₆)alkyl)₂. The substituents can themselves be optionally substituted. Furthermore, when containing two fused rings the aryl groups herein defined may have one or more saturated or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or a polycyclic aromatic radical of 5 to 24 ring atoms, containing one or more ring heteroatoms selected from N, O, S, P, or B, the remaining ring atoms being C. A polycyclic aromatic radical includes two or more fused rings and may further include two or more spiro-fused rings, e.g., bicyclic, tricyclic, tetracyclic, and the like. Unless otherwise specifically defined, “fused” means two rings sharing two ring atoms. Unless otherwise specifically defined, “spiro-fused” means two rings sharing one ring atom. Heteroaryl as herein defined also means a bicyclic heteroaromatic group wherein the heteroatom is selected from N, O, S, P, or B. Heteroaryl as herein defined also means a tricyclic heteroaromatic group containing one or more ring heteroatoms selected from N, O, S, P, or B. Heteroaryl as herein defined also means a tetracyclic heteroaromatic group containing one or more ring heteroatoms selected from N, O, S, P, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two or more fused rings, the heteroaryl groups defined herein may have one or more saturated or partially unsaturated ring fused with one or more fully unsaturated aromatic ring. In heteroaryl ring systems containing more than two fused rings, a saturated or partially unsaturated ring may further be fused with a saturated or partially unsaturated ring described herein. Furthermore, when containing three or more fused rings, the heteroaryl groups defined herein may have one or more saturated or partially unsaturated ring spiro-fused. Any saturated or partially unsaturated ring described herein is optionally substituted with one or more oxo. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7(4H)-only, 3,4-dihydropyrazino[1,2-a]indol-1(2H)-onyl, benzo[c][1,2]oxaborol-1(3H)-olyl, 6,6a,7,8-tetrahydro-9H-pyrido[2,3-b]puyrrolo[1,2-d][1,4]oxazin-9-onyl, or 6a′,7′-dihydro-6′H,9′H-spiro[cyclopropane-1,8′-pyrido[2,3-b]pyrrolo[1,2-d][1,4]oxazin]-9′-onyl.

Halogen or “halo” refers to fluorine, chlorine, bromine, or iodine.

Alkyl refers to a straight or branched chain saturated hydrocarbon containing 1-12 carbon atoms. Examples of a (C₁-C₆)alkyl group include, but are not limited to, methyl, ethyl, propyl, butyl, pentyl, hexyl, iso-propyl, iso-butyl, sec-butyl, tert-butyl, iso-pentyl, neo-pentyl, and iso-hexyl.

“Alkoxy” refers to a straight or branched chain saturated hydrocarbon containing 1-12 carbon atoms containing a terminal “O” in the chain, i.e., —O (alkyl). Examples of alkoxy groups include without limitation, methoxy, ethoxy, propoxy, butoxy, t-butoxy, or pentoxy groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

The term “alkylene” or “alkylenyl” refers to a divalent alkyl radical. Any of the above mentioned monovalent alkyl groups may be an alkylene by abstraction of a second hydrogen atom from the alkyl. As herein defined, alkylene may also be a C₁-C₆ alkylene. An alkylene may further be a C₁-C₄ alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Cycloalkyl” means mono or polycyclic saturated or partially unsaturated carbon rings containing 3-18 carbon atoms. Polycyclic cycloalkyl may be fused bicyclic cycloalkyl, bridged bicyclic cycloalkyl, or spiro-fused bicyclic cycloalkyl. A polycyclic cycloalkyl comprises at least one non-aromatic ring. Examples of cycloalkyl groups include, without limitations, cyclopropyl, cyclobutyl, cyclopentyl, cyclohexyl, cycloheptanyl, cyclooctanyl, norbornyl, norborenyl, 1,2,3,4-tetrahydronaphthyl, 2,3-dihydro-1H-indenyl, spiro[3.5]nonyl, spiro[5.5]undecyl, bicyclo[1.1.1]pentanyl, bicyclo[2.2.2]octanyl, or bicyclo[2.2.2]octenyl.

“Heterocyclyl”, “heterocycle” or “heterocycloalkyl” mono or polycyclic rings containing 3-24 atoms which include carbon and one or more heteroatoms selected from N, O, S, P, or B and wherein the rings are not aromatic. The heterocycloalkyl ring structure may be substituted by one or more substituents. A polycyclic heterocycloalkyl comprises at least one non-aromatic ring. Polycyclic heterocycles may be bridged, fused, or spiro-fused. The substituents can themselves be optionally substituted. Examples of heterocyclyl rings include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, homotropanyl, 2-oxa-5-azabicyclo[2.2.2]octane, and 2,6-diazaspiro[3.3]heptanyl.

The term “aromatic” means a planar ring having 4n+2 electrons in a conjugated system. As used herein, “conjugated system” means a system of connected p-orbitals with delocalized electrons, and the system may include lone electron pairs.

The term “haloalkyl” as used herein refers to an alkyl group, as defined herein, which is substituted one or more halogen. Examples of haloalkyl groups include, but are not limited to, trifluoromethyl, difluoromethyl, pentafluoroethyl, trichloromethyl, etc.

The term “haloalkoxy” as used herein refers to an alkoxy group, as defined herein, which is substituted with one or more halogen. Examples of haloalkyl groups include, but are not limited to, trifluoromethoxy, difluoromethoxy, pentafluoroethoxy, trichloromethoxy, etc.

The term “cyano” as used herein means a substituent having a carbon atom joined to a nitrogen atom by a triple bond, i.e., C≡N.

“Spirocycloalkyl” or “spirocyclyl” means carbogenic bicyclic ring systems with both rings connected through a single atom. The ring can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. One or more of the carbon atoms in the spirocycle can be substituted with a heteroatom (e.g., O, N, S, or P). A (C₃-C₁₂) spirocycloalkyl is a spirocycle containing between 3 and 12 carbon atoms. One or more of the carbon atoms can be substituted with a heteroatom.

The term “spiroheterocycloalkyl”, “spiroheterocycle”, or “spiroheterocyclyl” is understood to mean a spirocycle wherein at least one of the rings is a heterocycle (e.g., at least one of the rings is furanyl, morpholinyl, or piperidinyl).

The term “solvate” refers to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

As used herein, the term “alkyl-aryl” (and variations thereof, e.g. C₁-C₆ alkyl-aryl) refers to a chemical moiety comprising an alkyl group covalently attached to an aryl group, wherein the linkage to the rest of the molecule is on the first group recited, i.e., the alkyl group. Similarly, alkyl-alkoxy refers to a chemical moiety comprising an alkyl group covalently attached to an alkoxy group wherein the linkage to the rest of the molecule is on the alkyl group. This nomenclature may also be used for, e.g. alkenyl-aryl, alkenyl-heteroaryl, alkynyl-aryl, alkynyl-heteroaryl. As non-limiting examples, C₁ alkyl-phenyl refers to

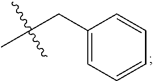 [SMILES: *Cc1ccccc1]

C₂ alkenyl-furanyl refers to

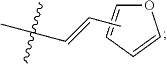

C₁ alkyl-C₂ alkoxy refers to

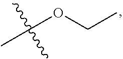 [SMILES: *OCC]

and so on.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (I) may have one or more asymmetric carbon atom and may occur as racemates, racemic mixtures and as individual enantiomers or diastereomers.

The present disclosure also contemplates isotopically-labelled compounds of Formula I (e.g., those labeled with ²H and ¹⁴C). Deuterated (i.e., ²H or D) and carbon-14 (i.e., ¹⁴C) isotopes are particularly preferred for their ease of preparation and detectability. Further, substitution with heavier isotopes such as deuterium may afford certain therapeutic advantages resulting from greater metabolic stability (e.g., increased in vivo half-life or reduced dosage requirements) and hence may be preferred in some circumstances. Isotopically labelled compounds of Formula I can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising a therapeutically effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, pantothenate, phosphate/diphosphate, picrate, polygalacturonate, propionate, p-toluenesulfonate, salicylate, stearate, subacetate, succinate, sulfate, sulfosalicylate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon, or rhesus.

An “effective amount” when used in connection with a compound is an amount effective for treating or preventing a disease in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a disclosed compound

The term “salt’ refers to pharmaceutically acceptable salts

The term “pharmaceutically acceptable salt” also refers to a salt of the compositions of the present disclosure having an acidic functional group, such as a carboxylic acid functional group, and a base.

“JAK2 inhibitor” as used herein refer to compounds of Formula I and/or compositions comprising a compound of Formula I which inhibits JAK2.

The amount of compound of composition described herein needed for achieving a therapeutic effect may be determined empirically in accordance with conventional procedures for the particular purpose. Generally, for administering therapeutic agents (e.g. compounds or compositions of Formula I (and/or additional agents) described herein) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. For example, administration of therapeutic agents to a subject suffering from cancer provides a therapeutic benefit not only when the underlying condition is eradicated or ameliorated, but also when the subject reports a decrease in the severity or duration of the symptoms associated with the disease. Therapeutic benefit also includes halting or slowing the progression of the underlying disease or disorder, regardless of whether improvement is realized.

### Compounds of the Present Disclosure

In one aspect, the present disclosure provides compounds of Formula (I) and salts, stereoisomers, solvates, prodrugs, isotopic derivatives, and tautomers thereof:

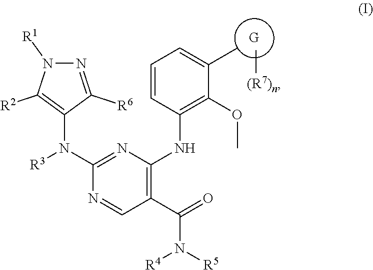

wherein R¹, R², R³, R⁴, R⁵, R⁶, R⁷, G, and n are as described herein.

It is understood that, for a compound of Formula (I), R¹, R², R³, R⁴, R⁵, R⁶, R⁷, G, and n can each be, where applicable, selected from the groups described herein, and any group described herein for any of R¹, R², R³, R⁴, R⁵, R⁶, R⁷, G, and n can be combined, where applicable, with any group described herein for one or more of the remainders of R¹, R², R³, R⁴, R⁵, R⁶, R⁷, G, and n.

In some embodiments,
Ring G is 5-10 membered monocyclic or bicyclic heteroaryl, comprising 1-3 heteroatoms selected from N, O, S;
R¹ is selected from C₁-C₆ alkyl, C₂-C₆ alkenyl, C₂-C₆ alkynyl, C₃-C₁₀ cycloalkyl, heterocycle, aryl, and heteroaryl, wherein the alkyl, alkenyl, alkynyl, cycloalkyl, heterocycle, aryl, or heteroaryl is optionally substituted with one or more substituents independently selected from halogen, CN, NO₂, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocyclyl, aryl, heteroaryl, wherein the C₃-C₁₀ cycloalkyl, heterocyclyl, aryl, or heteroaryl is optionally substituted with one or more substituents independently selected from C₁-C₆ alkyl, halogen, OH, CN;
R² is selected from H, C₁-C₆ alkyl;
R³ is selected from hydrogen, C₁-C₆ alkyl, C₃-C₁₀ cycloalkyl;
R⁴ is selected from H, C₁-C₆ alkyl;
R⁵ is selected from H, C₁-C₆ alkyl;
or R⁴ and R⁵ together with the atoms to which they are attached and any intervening atoms, form a 3-14 membered heterocycle optionally substituted with one or more substituents independently selected from halogen, C₁-C₆ alkyl, and C₁-C₆ alkoxy;
R⁶ is selected from H, C₁-C₆ alkyl;
each R⁷ is independently selected from oxo, C₁-C₆ alkyl, C₃-C₁₀ cycloalkyl, heterocyclyl, —NH-heteroaryl, —(CH₂)ₘ—NH—C(O)—R⁸, —(CH₂)ₘ—C(O)NH—R⁸, —(CH₂)ₘ—C(O)NH-heteroaryl, —(CH₂)ₘ—S(O)₂N—R⁸, —(CH₂)ₘ—S(O)₂N-heterocyclyl, wherein the alkyl, cycloalkyl, heterocyclyl or heteroaryl is optionally substituted with one or more substituents independently selected from halogen, —CN, —OH, N(R⁸)₂, C₁-C₆ alkyl, C₁-C₆ alkoxy, cycloalkyl;
each R⁸ is independently selected from H, C₁-C₆ alkyl, C₃-C₁₀ cycloalkyl;
n is an integer selected from 0, 1, 2 and 3;
m is an integer selected from 0, 1, and 2.

In some embodiments, Ring G is 5-10 membered monocyclic or bicyclic heteroaryl, comprising 1-3 heteroatoms selected from N, O, S, optionally substituted with 1-3 R⁷.

In some embodiments, Ring G is 5 membered monocyclic heteroaryl, comprising 1-3 heteroatom selected from N, O, S.

In some embodiments, Ring G is 5 membered monocyclic heteroaryl, comprising 1 heteroatom selected from N, O, S.

In some embodiments, Ring G is 5 membered monocyclic heteroaryl, comprising 2 heteroatoms independently selected from N, O, S.

In some embodiments, Ring G is 5 membered monocyclic heteroaryl, comprising 3 heteroatoms independently selected from N, O, S.

In some embodiments, Ring G is 6 membered monocyclic heteroaryl, comprising 1-3 heteroatom selected from N, O, S.

In some embodiments, Ring G is 6 membered monocyclic heteroaryl, comprising 1 heteroatom selected from N, O, S.

In some embodiments, Ring G is 6 membered monocyclic heteroaryl, comprising 2 heteroatoms independently selected from N, O, S.

In some embodiments, Ring G is 6 membered monocyclic heteroaryl, comprising 3 heteroatoms independently selected from N, O, S.

In some embodiments, Ring G is 9 membered bicyclic heteroaryl, comprising 1-3 heteroatom selected from N, O, S.

In some embodiments, Ring G is 9 membered bicyclic heteroaryl, comprising 1 heteroatom selected from N, O, S.

In some embodiments, Ring G is 9 membered bicyclic heteroaryl, comprising 2 heteroatoms independently selected from N, O, S.

In some embodiments, Ring G is 9 membered bicyclic heteroaryl, comprising 3 heteroatoms independently selected from N, O, S.

In some embodiments, Ring G is 10 membered bicyclic heteroaryl, comprising 1-3 heteroatom selected from N, O, S.

In some embodiments, Ring G is 10 membered bicyclic heteroaryl, comprising 1 heteroatom selected from N, O, S.

In some embodiments, Ring G is 10 membered bicyclic heteroaryl, comprising 2 heteroatoms independently selected from N, O, S.

In some embodiments, Ring G is 10 membered bicyclic heteroaryl, comprising 3 heteroatoms independently selected from N, O, S.

In some embodiments, Ring G is selected from

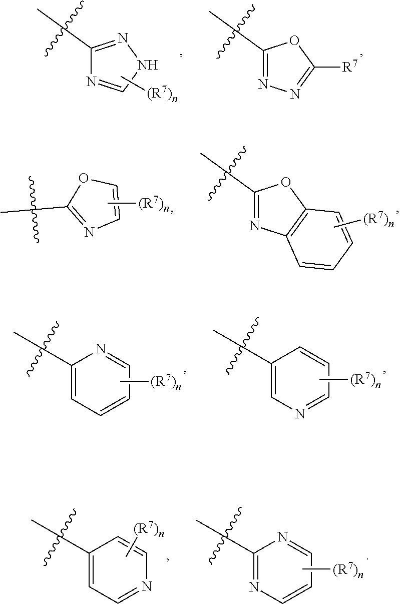

In some embodiments, Ring G is

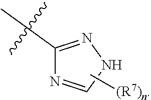

In some embodiments, Ring G is

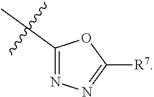

In some embodiments, Ring G is

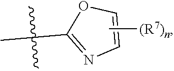

wherein n is selected from 0, 1 and 2.

In some embodiments, Ring G is

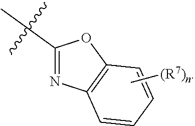

In some embodiments, Ring G is

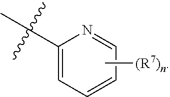

In some embodiments, Ring G is

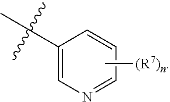

In some embodiments, Ring G is

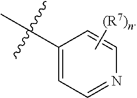

In some embodiments, Ring G is

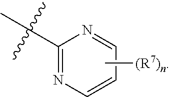

In some embodiments, Ring G is selected from

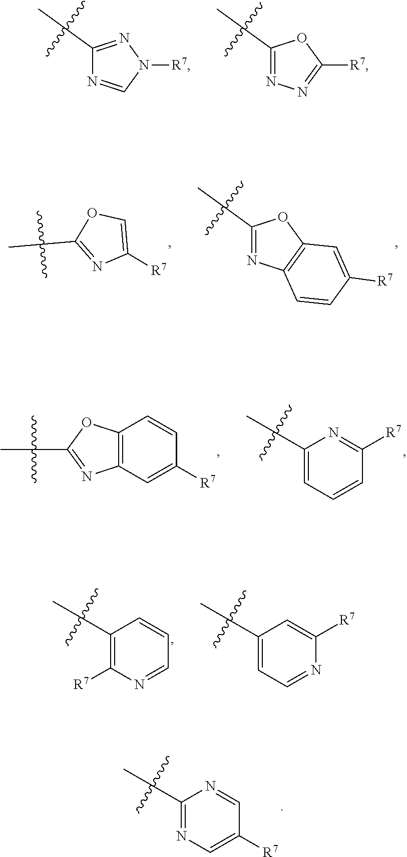

In some embodiments, Ring G is

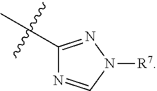

In some embodiments, Ting G is

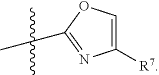

In some embodiments, Ring G is

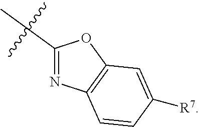

In some embodiments, Ring G is

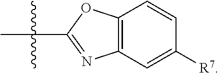

In some embodiments, Ring G is

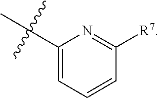

In some embodiments, Ring G is

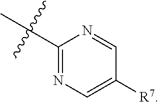

In some embodiments, Ring G is selected from

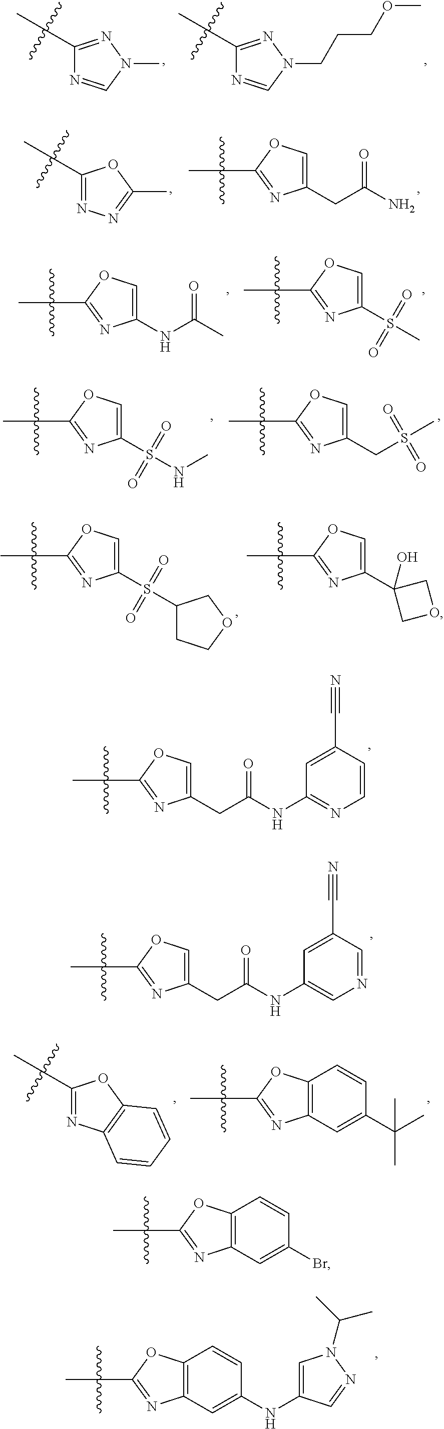

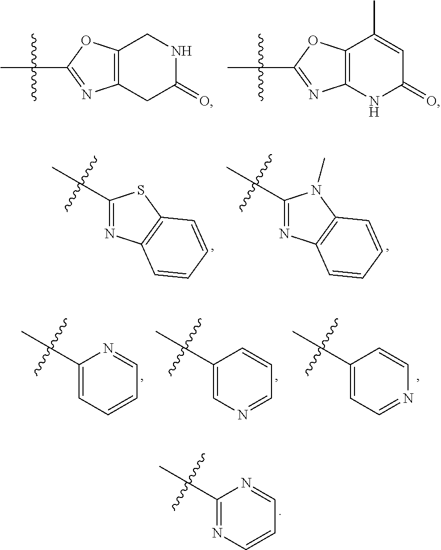

In some embodiments, Ring G is

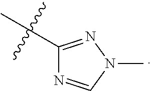 [SMILES: *c1ncn(C)n1]

In some embodiments, Ring G is

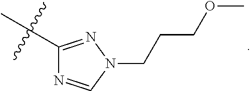 [SMILES: *c1ncn(CCCOC)n1]

In some embodiments, Ring G is

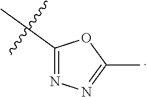 [SMILES: *c1nnc(C)o1]

In some embodiments, Ring G is

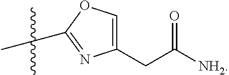 [SMILES: *c1nc(CC(N)=O)co1]

In some embodiments, Ring G is

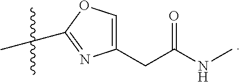 [SMILES: *c1nc(CC(=O)NC)co1]

In some embodiments, Ring G is

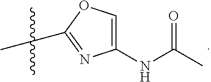 [SMILES: *c1nc(NC(C)=O)co1]

In some embodiments, Ring G is

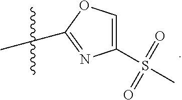 [SMILES: *c1nc(S(C)(=O)=O)co1]

In some embodiments, Ring G is

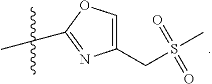 [SMILES: *c1nc(CS(C)(=O)=O)co1]

In some embodiments, Ring G is

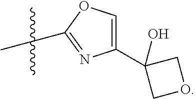 [SMILES: *c1nc(C2(O)COC2)co1]

In some embodiments, Ring G is

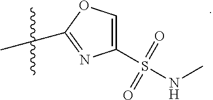 [SMILES: *c1nc(S(=O)(=O)NC)co1]

In some embodiments, Ring G is

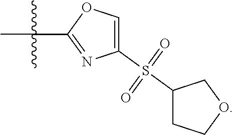 [SMILES: *c1nc(S(=O)(=O)C2CCOC2)co1]

In some embodiments, Ring G is

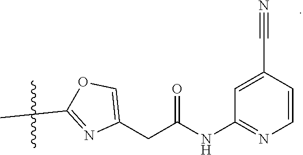 [SMILES: *c1nc(CC(=O)Nc2cc(C#N)ccn2)co1]

In some embodiments, Ring G is

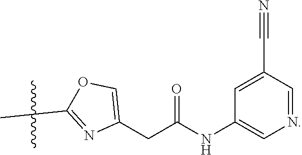 [SMILES: *c1nc(CC(=O)Nc2cncc(C#N)c2)co1]

In some embodiments, Ring G is

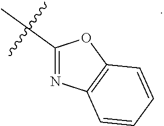 [SMILES: *c1nc2ccccc2o1]

In some embodiments, Ring G is

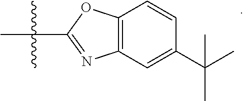 [SMILES: *c1nc2cc(C(C)(C)C)ccc2o1]

In some embodiments, Ring G is

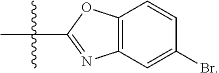 [SMILES: *c1nc2cc(Br)ccc2o1]

In some embodiments, Ring G is

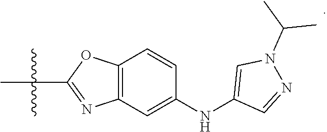 [SMILES: *c1nc2cc(Nc3cnn(C(C)C)c3)ccc2o1]

In some embodiments, Ring G is

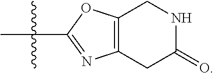 [SMILES: *c1nc2c(o1)CNC(=O)C2]

In some embodiments, Ring G is

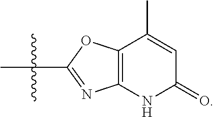 [SMILES: *c1nc2[nH]c(=O)cc(C)c2o1]

In some embodiments, Ring G is

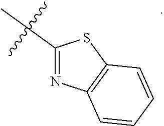 [SMILES: *c1nc2ccccc2s1]

In some embodiments, Ring G is

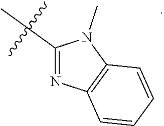 [SMILES: *c1nc2ccccc2n1C]

In some embodiments, Ring G is

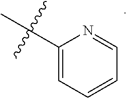 [SMILES: *c1ccccn1]

In some embodiments, Ring G is

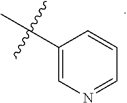 [SMILES: *c1cccnc1]

In some embodiments, Ring G is

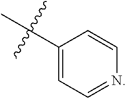 [SMILES: *c1ccncc1]

In some embodiments, Ring G is

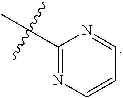 [SMILES: *c1ncccn1]

In some embodiments, R¹ is selected from C₁-C₆ alkyl, C₂-C₆ alkenyl, C₂-C₆ alkynyl, C₃-C₁₀ cycloalkyl, heterocycle, aryl, and heteroaryl, wherein the alkyl, alkenyl, alkynyl, cycloalkyl, heterocycle, aryl, or heteroaryl is optionally substituted with one or more substituents independently selected from halogen, CN, NO₂, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocyclyl, aryl, heteroaryl, wherein the C₃-C₁₀ cycloalkyl, heterocyclyl, aryl, or heteroaryl is optionally substituted with one or more substituents independently selected from C₁-C₆ alkyl, halogen, OH, CN.

In some embodiments, R¹ is C₁-C₆ alkyl optionally substituted with one or more substituents independently selected from halogen, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl, wherein the C₃-C₁₀ cycloalkyl, heterocyclyl, aryl, or heteroaryl is optionally substituted with one or more substituents independently selected from C₁-C₆ alkyl, halogen, OH, CN.

In some embodiments, R¹ is C₁-C₆ alkyl.

In some embodiments, R¹ is selected from methyl, ethyl, n-propyl, i-propyl, n-butyl, i-butyl, tert-butyl, n-pentyl, i-pentyl, n-hexyl, i-hexyl.

In some embodiments, R¹ is methyl.

In some embodiments, R¹ is ethyl.

In some embodiments, R¹ is propyl.

In some embodiments, R¹ is n-propyl.

In some embodiments, R¹ is i-propyl.

In some embodiments, R¹ is n-butyl.

In some embodiments, R¹ is i-butyl.

In some embodiments, R¹ is tert-butyl.

In some embodiments, R¹ is n-pentyl.

In some embodiments, R¹ is i-pentyl.

In some embodiments, R¹ is hexyl.

In some embodiments, R¹ is n-hexyl.

In some embodiments, R¹ is C₁-C₆ alkyl substituted with one or more N(R⁸)₂.

In some embodiments, R¹ is C₁-C₆ alkyl substituted with one N(R⁸)₂.

In some embodiments, R¹ is methyl substituted with one N(R⁸)₂.

In some embodiments, R¹ is ethyl substituted with one N(R⁸)₂.

In some embodiments, R¹ is propyl substituted with one N(R⁸)₂.

In some embodiments, R¹ is butyl substituted with one N(R⁸)₂.

In some embodiments, R¹ is pentyl substituted with one N(R⁸)₂.

In some embodiments, R¹ is hexyl substituted with one N(R⁸)₂.

In some embodiments, R¹ is

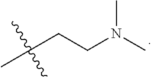 [SMILES: *CCN(C)C]

In some embodiments, R¹ is

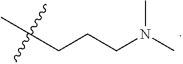 [SMILES: *CCCN(C)C]

In some embodiments, R¹ is

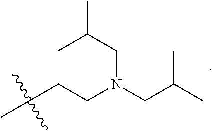 [SMILES: *CCN(CC(C)C)CC(C)C]

In some embodiments, R¹ is C₁-C₆ alkyl substituted with one or more C₁-C₆ alkoxy.

In some embodiments, R¹ is C₁-C₆ alkyl substituted with one C₁-C₆ alkoxy.

In some embodiments, R¹ is

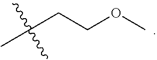 [SMILES: *CCOC]

In some embodiments, R¹ is

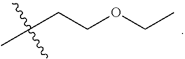 [SMILES: *CCOCC]

In some embodiments, R¹ is

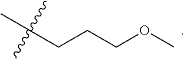 [SMILES: *CCCOC]

In some embodiments, R¹ is C₁-C₆ alkyl optionally substituted with C₁-C₆ alkyl.

In some embodiments, R¹ is C₁-C₆ alkyl substituted with C₁-C₆ alkyl.

In some embodiments, R¹ is octyl.

In some embodiments, R¹ is n-octyl.

In some embodiments, R¹ is C₁-C₆ alkyl substituted with C₃-C₁₀ cycloalkyl.

In some embodiments, R¹ is methyl substituted with C₃-C₁₀ cycloalkyl.

In some embodiments, R¹ is methyl substituted with cyclopropyl.

In some embodiments, R¹ is methyl substituted with cyclobutyl.

In some embodiments, R¹ is methyl substituted with cyclohexyl.

In some embodiments, R¹ is methyl substituted with adamantyl.

In some embodiments, R¹ is methyl substituted with 1-adamantyl.

In some embodiments, R¹ is C₁-C₆ alkyl optionally substituted with one or more halogen.

In some embodiments, R¹ is C₁-C₆ alkyl optionally substituted with one or more F.

In some embodiments, R¹ is

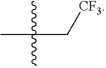 [SMILES: *CC(F)(F)F]

In some embodiments, R¹ is C₁-C₆ alkyl substituted with one heterocyclyl, wherein heterocyclyl is optionally substituted with one or more C₁-C₆ alkyl.

In some embodiments, R¹ is methyl substituted with one heterocyclyl, wherein heterocyclyl is optionally substituted with one or more C₁-C₆ alkyl.

In some embodiments, R¹ is methyl substituted with one 5 membered heterocyclyl, wherein heterocyclyl is optionally substituted with one or more C₁-C₆ alkyl.

In some embodiments, R¹ is methyl substituted with one 6 membered heterocyclyl, wherein heterocyclyl is optionally substituted with one or more C₁-C₆ alkyl.

In some embodiments, R¹ is methyl substituted with one 7 membered heterocyclyl, wherein heterocyclyl is optionally substituted with one or more C₁-C₆ alkyl.

In some embodiments, R¹ is methyl substituted with piperidinyl.

In some embodiments, R¹ is methyl substituted with 4-piperidinyl.

In some embodiments, R¹ is methyl substituted with 3-piperidinyl.

In some embodiments, R¹ is methyl substituted with 2-piperidinyl.

In some embodiments, R¹ is

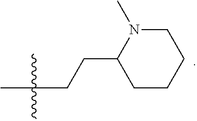 [SMILES: *CCC1CCCCN1C]

In some embodiments, R¹ is C₃-C₁₀ cycloalkyl optionally substituted with one or more substituents independently selected from halogen, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl.

In some embodiments, R¹ is C₃-C₁₀ cycloalkyl.

In some embodiments, R¹ is monocyclic C₃-C₁₀ cycloalkyl.

In some embodiments, R¹ is cyclopropyl.

In some embodiments, R¹ is cyclobutyl.

In some embodiments, R¹ is cyclopentyl.

In some embodiments, R¹ is cyclohexyl.

In some embodiments, R¹ is cycloheptyl.

In some embodiments, R¹ is cyclooctyl.

In some embodiments, R¹ is cyclononyl.

In some embodiments, R¹ is cyclodecyl.

In some embodiments, R¹ is polycyclic C₃-C₁₀ cycloalkyl.

In some embodiments, R¹ is adamantyl.

In some embodiments, R¹ is 1-adamantyl.

In some embodiments, R¹ is heterocyclyl optionally substituted with one or more substituents independently selected from halogen, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl.

In some embodiments, R¹ is 4-7 membered heterocyclyl optionally substituted with one or more substituents independently selected from halogen, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl.

In some embodiments, R¹ is 4-7 membered heterocyclyl, comprising 1-3 heteroatoms independently selected from N, O, S, wherein heterocyclyl is optionally substituted with one or more substituents independently selected from halogen, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl.

In some embodiments, R¹ is 4 membered heterocyclyl, comprising 1 heteroatom selected from N, O, S, wherein heterocycle is optionally substituted with one or more substituents independently selected from halogen, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocyclyl, aryl, heteroaryl.

In some embodiments, R¹ is 5 membered heterocyclyl, comprising 1-2 heteroatoms selected from N, O, S, wherein heterocyclyl is optionally substituted with one or more substituents independently selected from halogen, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocyclyl, aryl, heteroaryl.

In some embodiments, R¹ is 6 membered heterocyclyl, comprising 1-3 heteroatoms selected from N, O, S, wherein heterocyclyl is optionally substituted with one or more substituents independently selected from halogen, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocyclyl, aryl, heteroaryl.

In some embodiments, R¹ is 6 membered heterocyclyl, comprising N, wherein heterocyclyl is optionally substituted with one or more substituents independently selected from halogen, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocyclyl, aryl, heteroaryl.

In some embodiments, R¹ is piperidinyl.

In some embodiments, R¹ is 4-piperidinyl.

In some embodiments, R¹ is 3-piperidinyl.

In some embodiments, R¹ is 2-piperidinyl.

In some embodiments, R¹ is

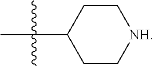 [SMILES: *C1CCNCC1]

In some embodiments, R¹ is 6 membered heterocyclyl, comprising N, wherein heterocyclyl is substituted with C₁-C₆ alkyl.

In some embodiments, R¹ is

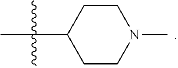 [SMILES: *C1CCN(C)CC1]

In some embodiments, R¹ is

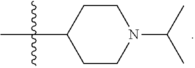 [SMILES: *C1CCN(C(C)C)CC1]

In some embodiments, R¹ is

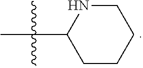 [SMILES: *C1CCCCN1]

In some embodiments, R¹ is

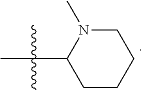 [SMILES: *C1CCCCN1C]

In some embodiments, R¹ is 6 membered heterocyclyl, comprising O, wherein heterocyclyl is optionally substituted with one or more substituents independently selected from halogen, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocyclyl, aryl, heteroaryl.

In some embodiments, R¹ is tetrahydropyran.

In some embodiments, R¹ is 4-tetrahydropyran.

In some embodiments, R¹ is 3-tetrahydropyran.

In some embodiments, R¹ is 2-tetrahydropyran.

In some embodiments, R¹ is

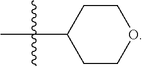 [SMILES: *C1CCOCC1]

In some embodiments, R¹ is 7 membered heterocyclyl, comprising 1-3 heteroatoms selected from N, O, S, wherein heterocyclyl is optionally substituted with one or more substituents independently selected from halogen, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocyclyl, aryl, heteroaryl.

In some embodiments, R¹ is aryl optionally substituted with one or more substituents independently selected from halogen, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl.

In some embodiments, R¹ is phenyl optionally substituted with one or more substituents independently selected from halogen, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl. In some embodiments, R¹ is phenyl.

In some embodiments, R¹ is

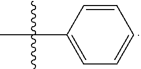 [SMILES: *c1ccccc1]

In some embodiments, R² is selected from H, C₁-C₆ alkyl.

In some embodiments, R² is H.

In some embodiments, R² is C₁-C₆ alkyl.

In some embodiments, R² is methyl.

In some embodiments, R³ is selected from hydrogen, C₁-C₆ alkyl, C₃-C₁₀ cycloalkyl.

In some embodiments, R³ is hydrogen.

In some embodiments, R³ is C₁-C₆ alkyl.

In some embodiments, R³ is C₃-C₁₀ cycloalkyl.

In some embodiments, R⁴ is selected from H, C₁-C₆ alkyl.

In some embodiments, R⁴ is H.

In some embodiments, R⁴ is C₁-C₆ alkyl.

In some embodiments, R⁴ is methyl.

In some embodiments, R⁵ is selected from H, C₁-C₆ alkyl.

In some embodiments, R⁵ is H.

In some embodiments, R⁵ is C₁-C₆ alkyl.

In some embodiments, R⁵ is methyl.

In some embodiments, R⁴ and R⁵ together with the atoms to which they are attached and any intervening atoms, form a 3-14 membered heterocycle optionally substituted with one or more substituents independently selected from halogen, C₁-C₆ alkyl, and C₁-C₆ alkoxy.

In some embodiments, R⁶ is selected from H, C₁-C₆ alkyl.

In some embodiments, R⁶ is H.

In some embodiments, R⁶ is C₁-C₆ alkyl.

In some embodiments, R⁶ is methyl.

In some embodiments, each R⁷ is independently selected from oxo, C₁-C₆ alkyl, C₃-C₁₀ cycloalkyl, heterocyclyl, —NH-heteroaryl, —(CH₂)ₘ—NH—C(O)—R⁸, —(CH₂)ₘ—C(O)NH—R⁸, —(CH₂)ₘ—C(O)NH-heteroaryl, —(CH₂)ₘ—S(O)₂N—R⁸, —(CH₂)ₘ—S(O)₂N-heterocyclyl, wherein the alkyl, cycloalkyl, heterocyclyl or heteroaryl is optionally substituted with one or more substituents independently selected from halogen, —CN, —OH, N(R⁸)₂, C₁-C₆ alkyl, C₁-C₆ alkoxy, cycloalkyl.

In some embodiments, R⁷ is C₁-C₆ alkyl optionally substituted with one or more substituents independently selected from halogen, —OH, N(R⁸)₂, C₁-C₆ alkyl, C₁-C₆ alkoxy, cycloalkyl.

In some embodiments, R⁷ is methyl.

In some embodiments, R⁷ is C₃-C₁₀ cycloalkyl optionally substituted with one or more substituents independently selected from halogen, —OH, N(R⁸)₂, C₁-C₆ alkyl, C₁-C₆ alkoxy, cycloalkyl.

In some embodiments, each R⁸ is independently selected from H, C₁-C₆ alkyl, C₃-C₁₀ cycloalkyl.

In some embodiments, each R⁸ is H.

In some embodiments, one R⁸ is H and one R⁸ is C₁-C₆ alkyl.

In some embodiments, each R⁸ is C₁-C₆ alkyl.

In some embodiments, each R⁸ is methyl.

In some embodiments, each R⁸ is ethyl.

In some embodiments, each R⁸ is propyl.

In some embodiments, each R⁸ is n-propyl.

In some embodiments, each R⁸ is i-propyl.

In some embodiments, each R⁸ is butyl.

In some embodiments, each R⁸ is n-butyl.

In some embodiments, each R⁸ is i-butyl.

In some embodiments, n is an integer selected from 0, 1, 2 and 3.

In some embodiments, n is 0.

In some embodiments, n is 1.

In some embodiments, n is 2.

In some embodiments, n is 3.

In some embodiments, m is an integer selected from 0, 1 and 2.

In some embodiments, m is 0.

In some embodiments, m is 1.

In some embodiments, m is 2.

In some embodiments, the compound is of Formula (I-ABC):

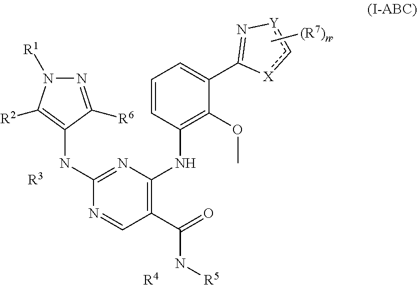

or a pharmaceutically acceptable salt, stereoisomer, solvate, prodrug, isotopic derivative, or tautomer thereof, wherein X is selected from N and O, Y is selected from N, O and CH, the bond X [symbol] is a single or double bond, the bond [symbol] Y is a single or double bond; provided that when X is N the bond X [symbol] is a double bond and the bond [symbol] Y is a single bond and when X is O the bond X [symbol] is a single bond and the bond [symbol] Y is a double bond; optionally two of R⁷ together with the atoms to which they are attached and any intervening atoms, form an aryl optionally substituted with 1-3 R⁷ and all other variables are as defined herein.

In some embodiments, the compound is of Formula (I-A):

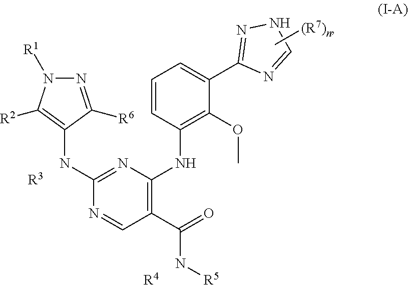

or a pharmaceutically acceptable salt, stereoisomer, solvate, prodrug, isotopic derivative, or tautomer thereof, wherein all variables are as defined herein.

In some embodiments, the compound is of Formula (I-B):

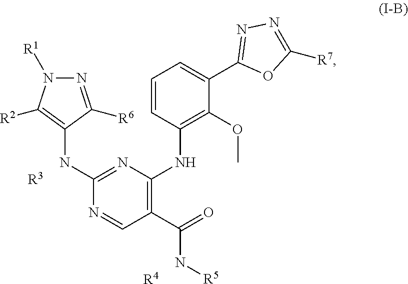

or a pharmaceutically acceptable salt, stereoisomer, solvate, prodrug, isotopic derivative, or tautomer thereof, wherein all variables are as defined herein.

In some embodiments, the compound is of Formula (I-C):

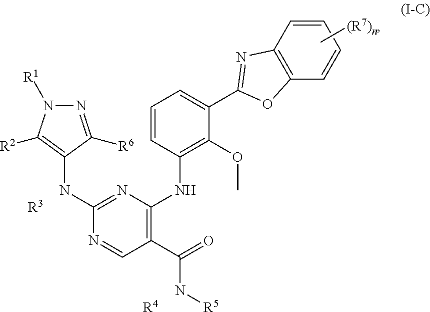

or a pharmaceutically acceptable salt, stereoisomer, solvate, prodrug, isotopic derivative, or tautomer thereof wherein all variables are as defined herein.

In some embodiments, the compound is of Formula (I-C*):

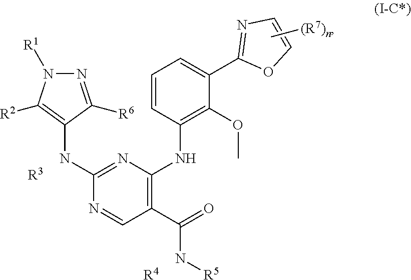

or tautomer thereof wherein all variables are as defined herein, wherein n is selected from 0, 1, and 2, and all other variables are as defined herein.

In some embodiments, the compound is of Formula (I-C′):

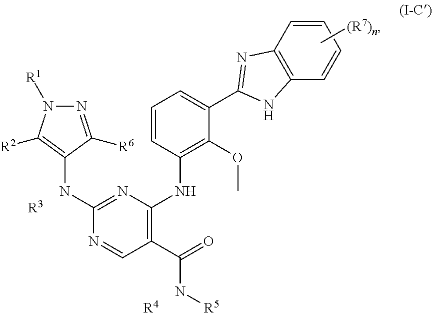

or a pharmaceutically acceptable salt, stereoisomer, solvate, prodrug, isotopic derivative, or tautomer thereof wherein all variables are as defined herein.

In some embodiments, the compound is of Formula (I-C″):

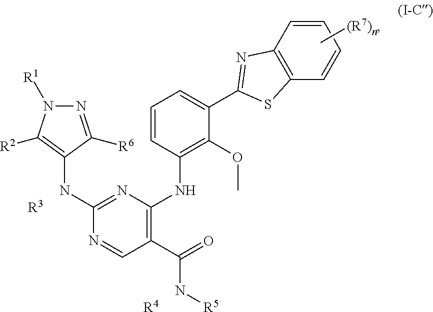

or a pharmaceutically acceptable salt, stereoisomer, solvate, prodrug, isotopic derivative, or tautomer thereof wherein all variables are as defined herein.

In some embodiments, the compound is of Formula (I-C′″):

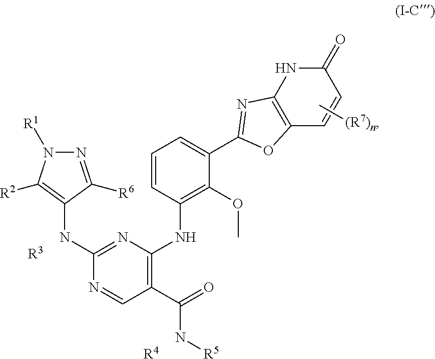

or a pharmaceutically acceptable salt, stereoisomer, solvate, prodrug, isotopic derivative, or tautomer thereof wherein all variables are as defined herein.

In some embodiments, the compound is of Formula (I-C″″):

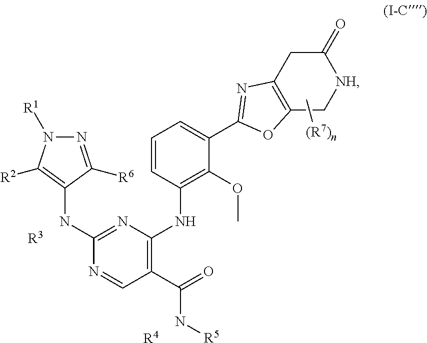

or a pharmaceutically acceptable salt, stereoisomer, solvate, prodrug, isotopic derivative, or tautomer thereof wherein all variables are as defined herein.

In some embodiments, the compound is of Formula (I-DE):

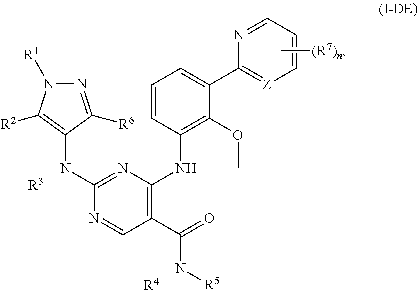

or a pharmaceutically acceptable salt, stereoisomer, solvate, prodrug, isotopic derivative, or tautomer thereof, wherein Z is selected from N and C, optionally two of R⁷ together with the atoms to which they are attached and any intervening atoms, form an aryl and all other variables are as defined herein.

In some embodiments, the compound is of Formula (I-D):

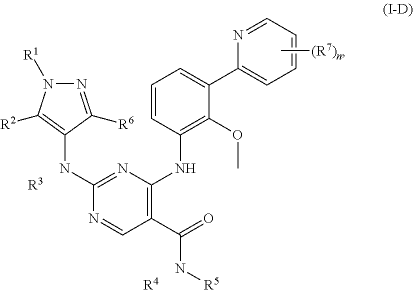

or a pharmaceutically acceptable salt, stereoisomer, solvate, prodrug, isotopic derivative, or tautomer thereof wherein all variables are as defined herein.

In some embodiments, the compound is of Formula (I-E):

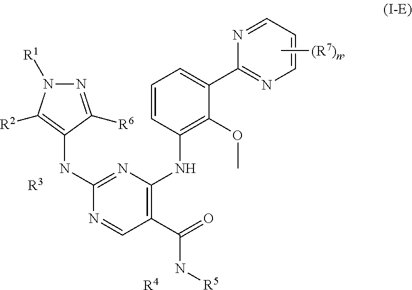

or tautomer thereof wherein all variables are as defined herein.

In some embodiments, the compound is of Formula (I-A-1):

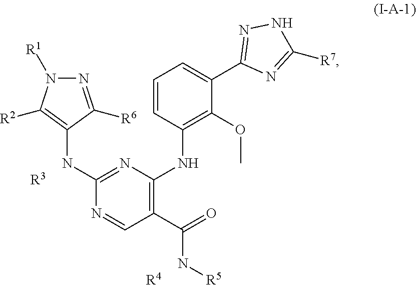

or a pharmaceutically acceptable salt, stereoisomer, solvate, prodrug, isotopic derivative, or tautomer thereof wherein all variables are as defined herein.

In some embodiments, the compound is of Formula (I-A-2):

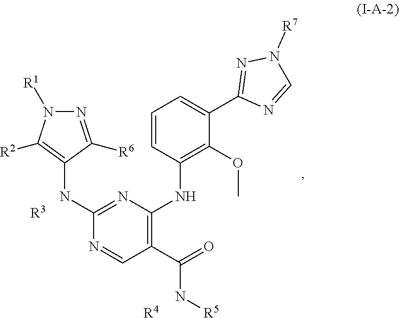

or a pharmaceutically acceptable salt, stereoisomer, solvate, prodrug, isotopic derivative, or tautomer thereof wherein all variables are as defined herein.

In some embodiments, the compound is of Formula (I-A-2-a):

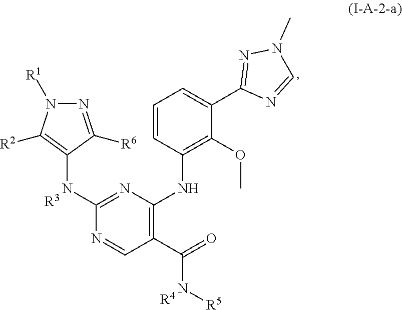

or a pharmaceutically acceptable salt, stereoisomer, solvate, prodrug, isotopic derivative, or tautomer thereof wherein all variables are as defined herein.

In some embodiments, the compound is of Formula (I-A-3):

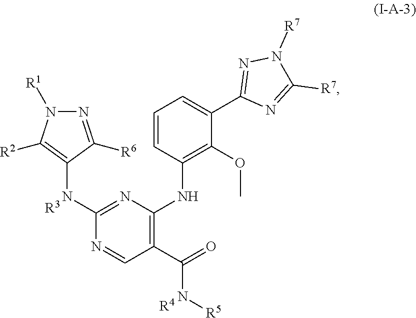

or a pharmaceutically acceptable salt, stereoisomer, solvate, prodrug, isotopic derivative, or tautomer thereof wherein all variables are as defined herein.

In some embodiments, the compound is of Formula (I-A-4):

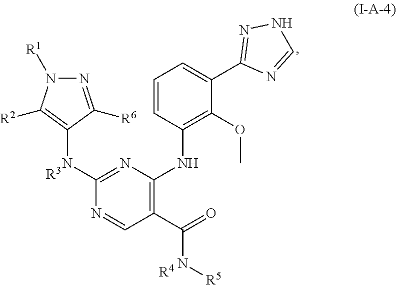

or tautomer thereof wherein all variables are as defined herein.

In some embodiments, the compound is of Formula (I-B-a):

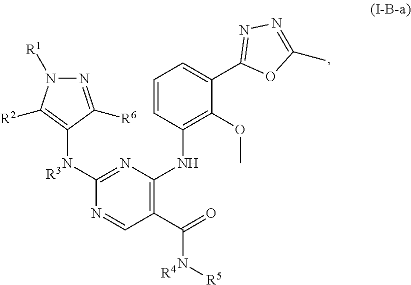

or a pharmaceutically acceptable salt, stereoisomer, solvate, prodrug, isotopic derivative, or tautomer thereof, wherein all variables are as defined herein.

In some embodiments, the compound is of Formula (I-C-1):

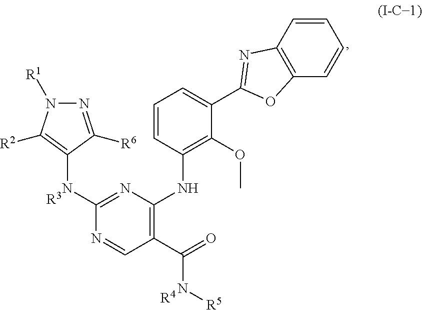

or a pharmaceutically acceptable salt, stereoisomer, solvate, prodrug, isotopic derivative, or tautomer thereof wherein all variables are as defined herein.

In some embodiments, the compound is of Formula (I-C-2):

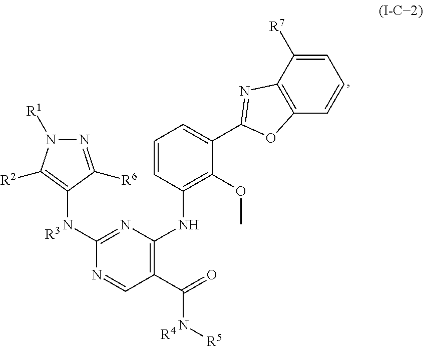

or a pharmaceutically acceptable salt, stereoisomer, solvate, prodrug, isotopic derivative, or tautomer thereof wherein all variables are as defined herein.

In some embodiments, the compound is of Formula (I-C-3):

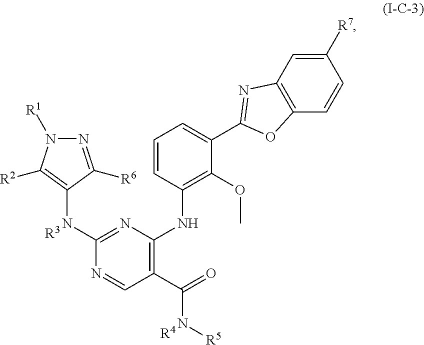

or a pharmaceutically acceptable salt, stereoisomer, solvate, prodrug, isotopic derivative, or tautomer thereof wherein all variables are as defined herein.

In some embodiments, the compound is of Formula (I-C-4):

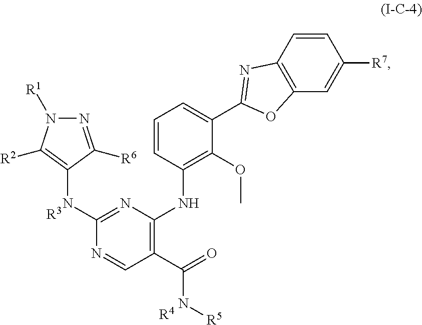

or a pharmaceutically acceptable salt, stereoisomer, solvate, prodrug, isotopic derivative, or tautomer thereof wherein all variables are as defined herein.

In some embodiments, the compound is of Formula (I-C-5):

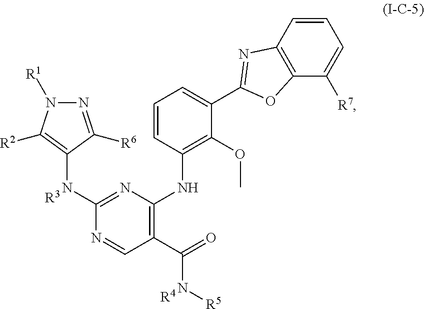

or a pharmaceutically acceptable salt, stereoisomer, solvate, prodrug, isotopic derivative, or tautomer thereof wherein all variables are as defined herein.

In some embodiments, the compound is of Formula (I-C-6):

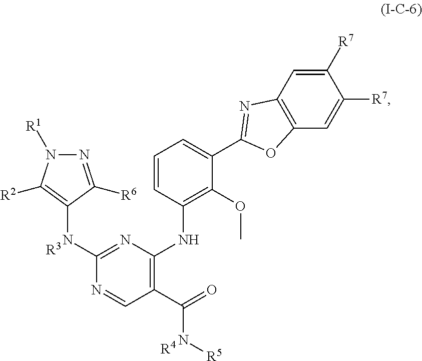

or a pharmaceutically acceptable salt, stereoisomer, solvate, prodrug, isotopic derivative, or tautomer thereof wherein all variables are as defined herein.

In some embodiments, the compound is of Formula (I-C-7):

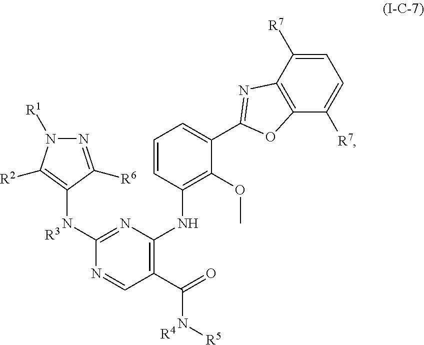

or a pharmaceutically acceptable salt, stereoisomer, solvate, prodrug, isotopic derivative, or tautomer thereof wherein all variables are as defined herein.

In some embodiments, the compound is of Formula (I-C-8):

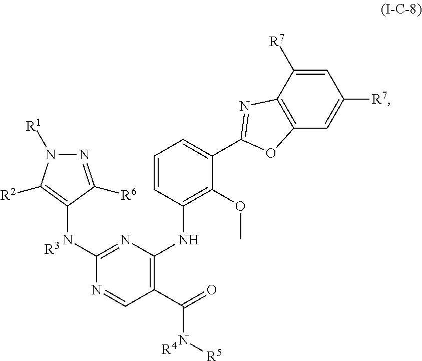

or a pharmaceutically acceptable salt, stereoisomer, solvate, prodrug, isotopic derivative, or tautomer thereof wherein all variables are as defined herein.

In some embodiments, the compound is of Formula (I-C-9):

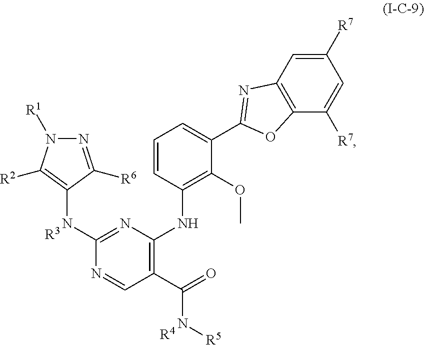

or a pharmaceutically acceptable salt, stereoisomer, solvate, prodrug, isotopic derivative, or tautomer thereof wherein all variables are as defined herein.

In some embodiments, the compound is of Formula (I-C-10):

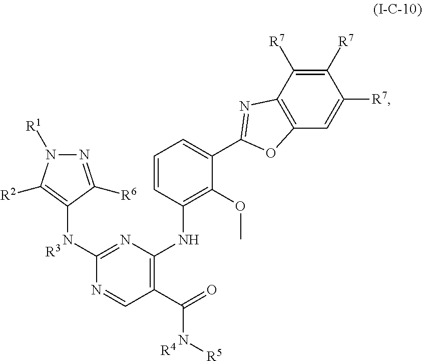

or a pharmaceutically acceptable salt, stereoisomer, solvate, prodrug, isotopic derivative, or tautomer thereof wherein all variables are as defined herein.

In some embodiments, the compound is of Formula (I-C-11):

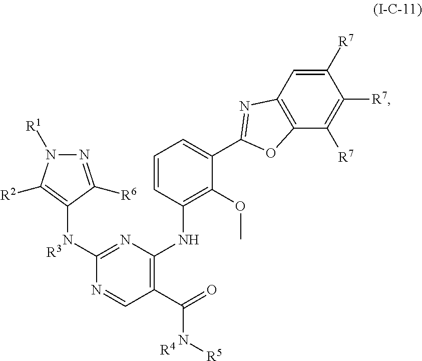

or a pharmaceutically acceptable salt, stereoisomer, solvate, prodrug, isotopic derivative, or tautomer thereof wherein all variables are as defined herein.

In some embodiments, the compound is of Formula (I-C′-1):

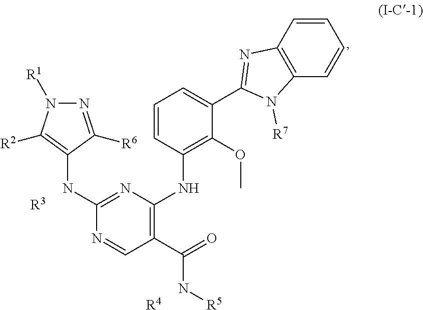

or a pharmaceutically acceptable salt, stereoisomer, solvate, prodrug, isotopic derivative, or tautomer thereof wherein all variables are as defined herein.

In some embodiments, the compound is of Formula (I-C′-2):

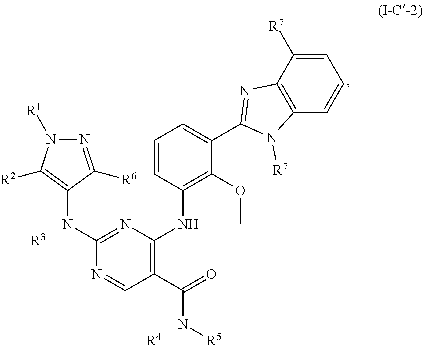

or a pharmaceutically acceptable salt, stereoisomer, solvate, prodrug, isotopic derivative, or tautomer thereof wherein all variables are as defined herein.

In some embodiments, the compound is of Formula (I-C′-3):

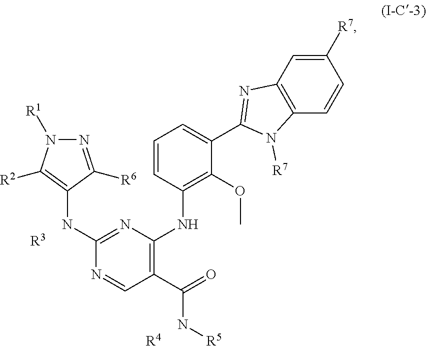

or a pharmaceutically acceptable salt, stereoisomer, solvate, prodrug, isotopic derivative, or tautomer thereof wherein all variables are as defined herein.

In some embodiments, the compound is of Formula (I-C′-4):

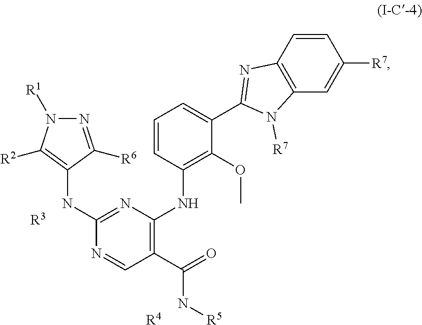

or a pharmaceutically acceptable salt, stereoisomer, solvate, prodrug, isotopic derivative, or tautomer thereof wherein all variables are as defined herein.

In some embodiments, the compound is of Formula (I-C′-5):

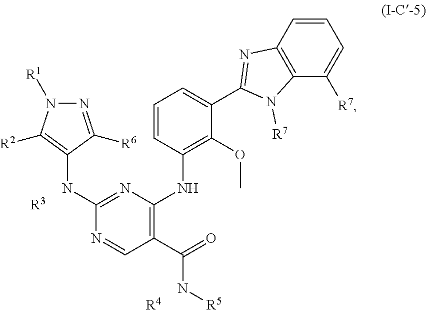

or a pharmaceutically acceptable salt, stereoisomer, solvate, prodrug, isotopic derivative, or tautomer thereof wherein all variables are as defined herein.

In some embodiments, the compound is of Formula (I-C′-6):

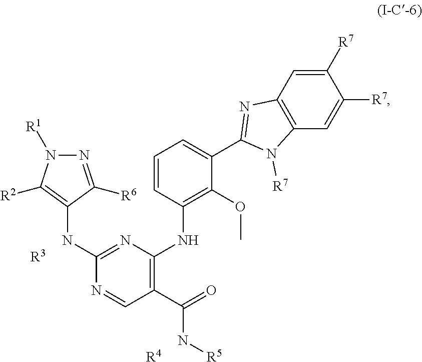

or a pharmaceutically acceptable salt, stereoisomer, solvate, prodrug, isotopic derivative, or tautomer thereof wherein all variables are as defined herein.

In some embodiments, the compound is of Formula (I-C′-7):

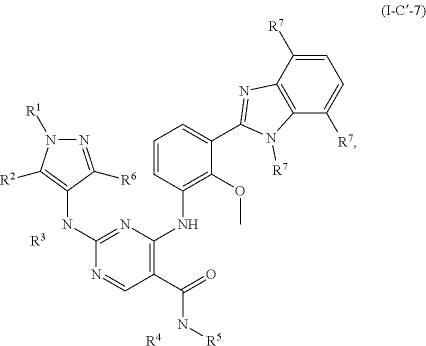

or a pharmaceutically acceptable salt, stereoisomer, solvate, prodrug, isotopic derivative, or tautomer thereof wherein all variables are as defined herein.

In some embodiments, the compound is of Formula (I-C′-8):

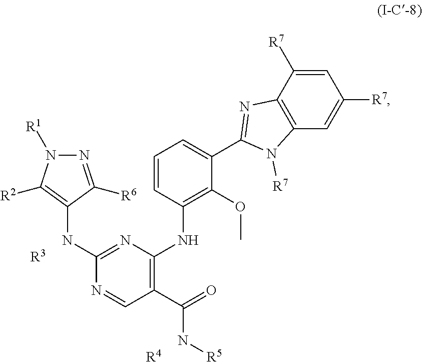

or a pharmaceutically acceptable salt, stereoisomer, solvate, prodrug, isotopic derivative, or tautomer thereof wherein all variables are as defined herein.

In some embodiments, the compound is of Formula (I-C′-9):

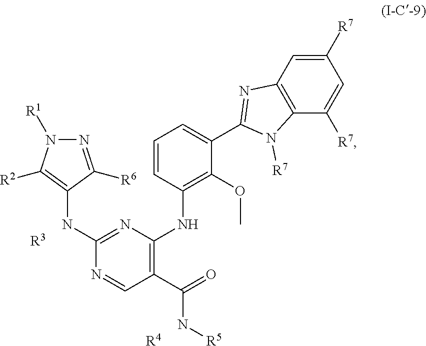

or a pharmaceutically acceptable salt, stereoisomer, solvate, prodrug, isotopic derivative, or tautomer thereof wherein all variables are as defined herein.

In some embodiments, the compound is of Formula (I-C′-10):

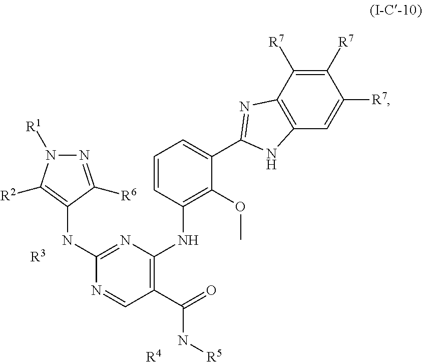

or a pharmaceutically acceptable salt, stereoisomer, solvate, prodrug, isotopic derivative, or tautomer thereof wherein all variables are as defined herein.

In some embodiments, the compound is of Formula (I-C′-11):

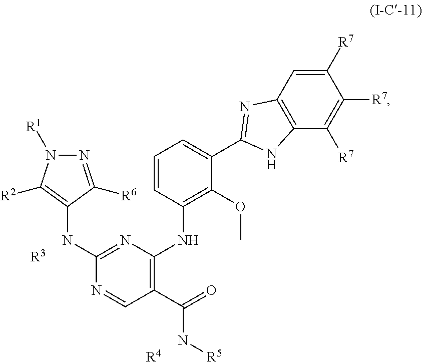

or a pharmaceutically acceptable salt, stereoisomer, solvate, prodrug, isotopic derivative, or tautomer thereof wherein all variables are as defined herein.

In some embodiments, the compound is of Formula (I-C″-1):

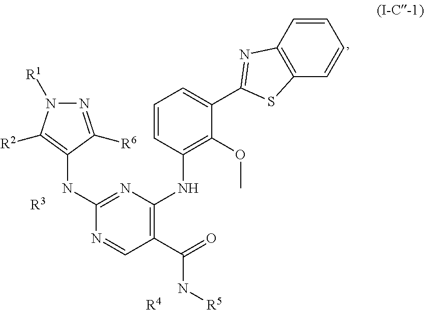

or a pharmaceutically acceptable salt, stereoisomer, solvate, prodrug, isotopic derivative, or tautomer thereof wherein all variables are as defined herein.

In some embodiments, the compound is of Formula (I-C″-2):

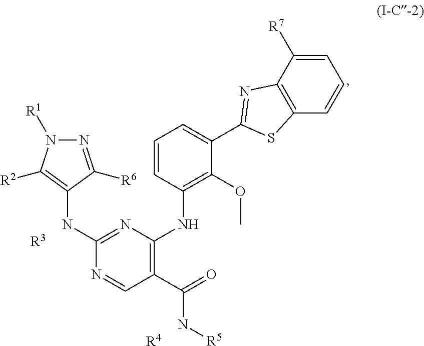

or a pharmaceutically acceptable salt, stereoisomer, solvate, prodrug, isotopic derivative, or tautomer thereof wherein all variables are as defined herein.

In some embodiments, the compound is of Formula (I-C″-3):

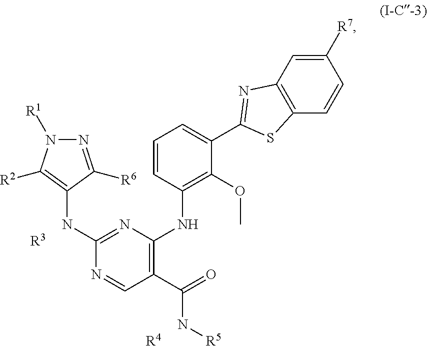

or a pharmaceutically acceptable salt, stereoisomer, solvate, prodrug, isotopic derivative, or tautomer thereof wherein all variables are as defined herein.

In some embodiments, the compound is of Formula (I-C″-4):

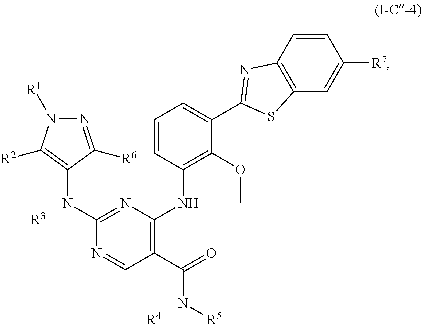

or a pharmaceutically acceptable salt, stereoisomer, solvate, prodrug, isotopic derivative, or tautomer thereof wherein all variables are as defined herein.

In some embodiments, the compound is of Formula (I-C″-5):

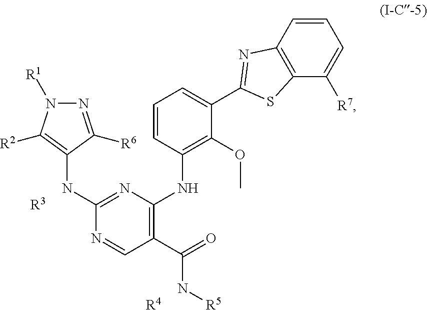

or a pharmaceutically acceptable salt, stereoisomer, solvate, prodrug, isotopic derivative, or tautomer thereof wherein all variables are as defined herein.

In some embodiments, the compound is of Formula (I-C″-6):

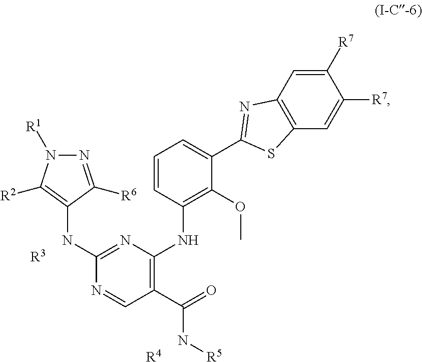

or a pharmaceutically acceptable salt, stereoisomer, solvate, prodrug, isotopic derivative, or tautomer thereof wherein all variables are as defined herein.

In some embodiments, the compound is of Formula (I-C″-7):

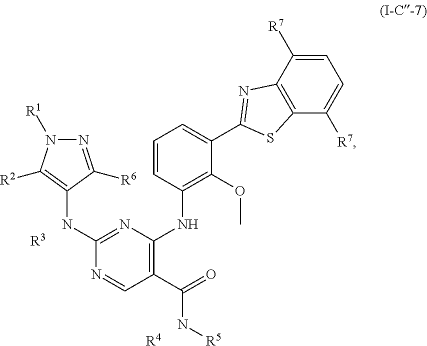

or a pharmaceutically acceptable salt, stereoisomer, solvate, prodrug, isotopic derivative, or tautomer thereof wherein all variables are as defined herein.

In some embodiments, the compound is of Formula (I-C″-8):

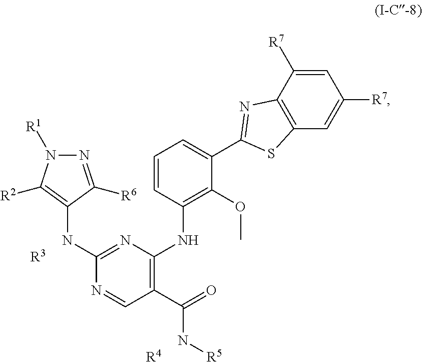

or a pharmaceutically acceptable salt, stereoisomer, solvate, prodrug, isotopic derivative, or tautomer thereof wherein all variables are as defined herein.

In some embodiments, the compound is of Formula (I-C″-9):

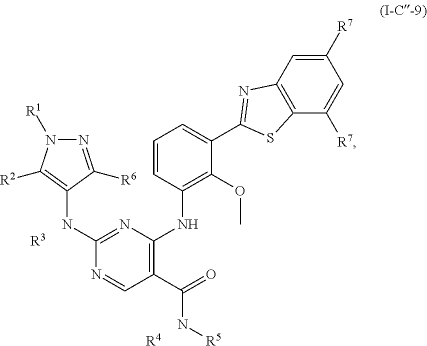

or a pharmaceutically acceptable salt, stereoisomer, solvate, prodrug, isotopic derivative, or tautomer thereof wherein all variables are as defined herein.

In some embodiments, the compound is of Formula (I-C″-10):

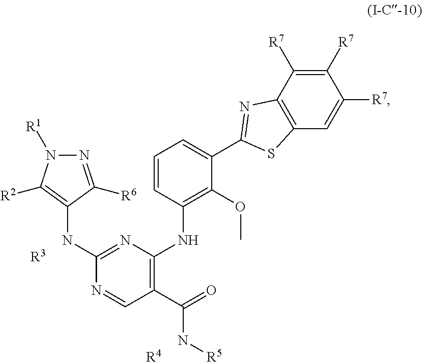

or a pharmaceutically acceptable salt, stereoisomer, solvate, prodrug, isotopic derivative, or tautomer thereof wherein all variables are as defined herein.

In some embodiments, the compound is of Formula (I-C″-11):

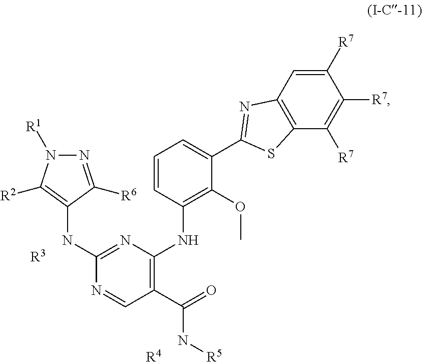

or a pharmaceutically acceptable salt, stereoisomer, solvate, prodrug, isotopic derivative, or tautomer thereof wherein all variables are as defined herein.

In some embodiments, the compound is of Formula (I-C′″-1):

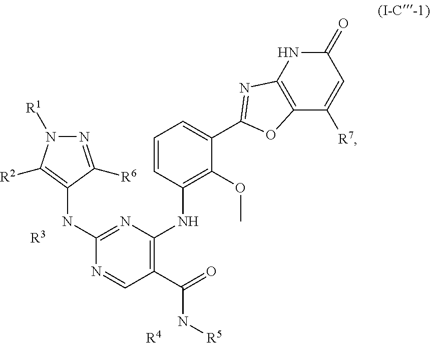

or a pharmaceutically acceptable salt, stereoisomer, solvate, prodrug, isotopic derivative, or tautomer thereof wherein all variables are as defined herein.

In some embodiments, the compound is of Formula (I-D-1):

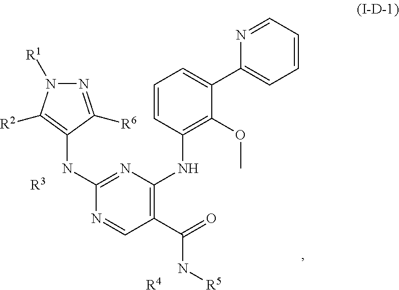

or a pharmaceutically acceptable salt, stereoisomer, solvate, prodrug, isotopic derivative, or tautomer thereof wherein all variables are as defined herein.

In some embodiments, the compound is of Formula (I-D-2):

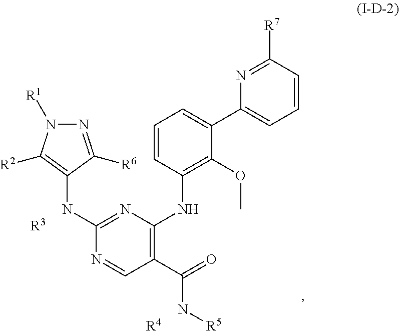

or a pharmaceutically acceptable salt, stereoisomer, solvate, prodrug, isotopic derivative, or tautomer thereof wherein all variables are as defined herein.

In some embodiments, the compound is of Formula (I-D-3):

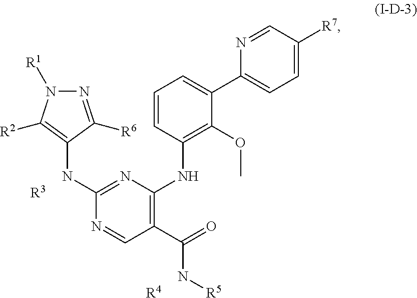

or a pharmaceutically acceptable salt, stereoisomer, solvate, prodrug, isotopic derivative, or tautomer thereof wherein all variables are as defined herein.

In some embodiments, the compound is of Formula (I-D-4):

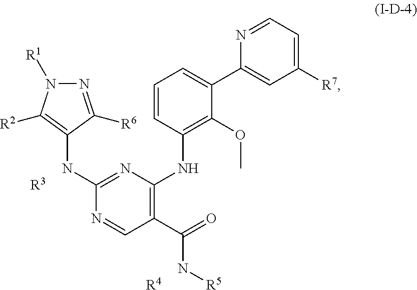

or tautomer thereof wherein all variables are as defined herein.

In some embodiments, the compound is of Formula (I-D-5):

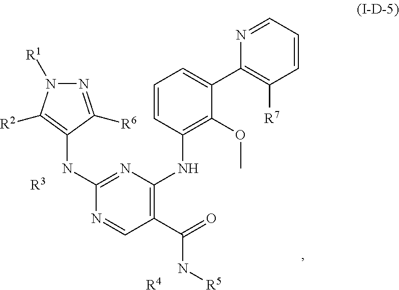

or a pharmaceutically acceptable salt, stereoisomer, solvate, prodrug, isotopic derivative, or tautomer thereof wherein all variables are as defined herein.

In some embodiments, the compound is of Formula (I-D-6):

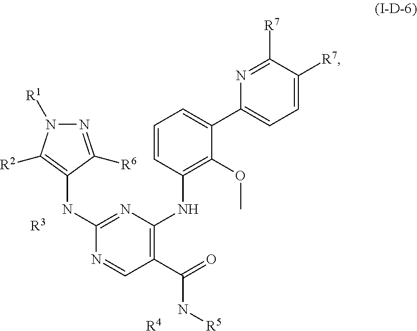

or a pharmaceutically acceptable salt, stereoisomer, solvate, prodrug, isotopic derivative, or tautomer thereof wherein all variables are as defined herein.

In some embodiments, the compound is of Formula (I-D-7):

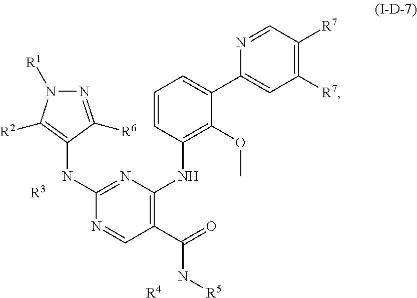

or a pharmaceutically acceptable salt, stereoisomer, solvate, prodrug, isotopic derivative, or tautomer thereof wherein all variables are as defined herein.

In some embodiments, the compound is of Formula (I-D-8):

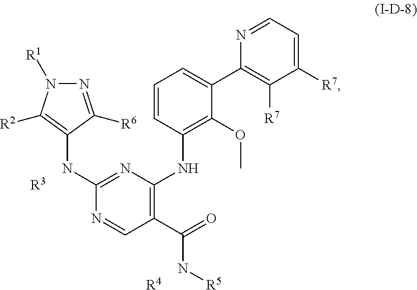

or a pharmaceutically acceptable salt, stereoisomer, solvate, prodrug, isotopic derivative, or tautomer thereof wherein all variables are as defined herein.

In some embodiments, the compound is of Formula (I-D-9):

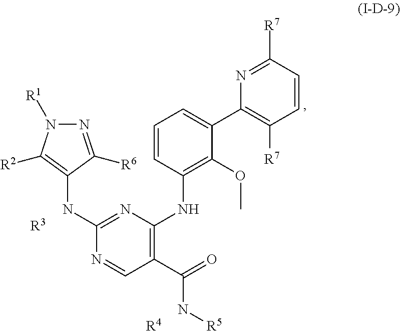

or tautomer thereof wherein all variables are as defined herein.

In some embodiments, the compound is of Formula (I-D-10):

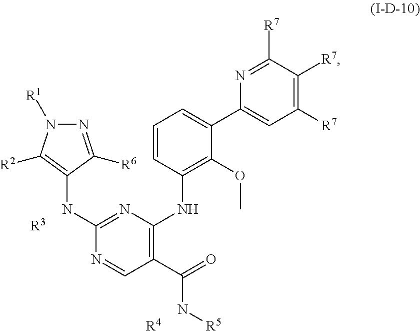

or a pharmaceutically acceptable salt, stereoisomer, solvate, prodrug, isotopic derivative, or tautomer thereof wherein all variables are as defined herein.

In some embodiments, the compound is of Formula (I-D-11):

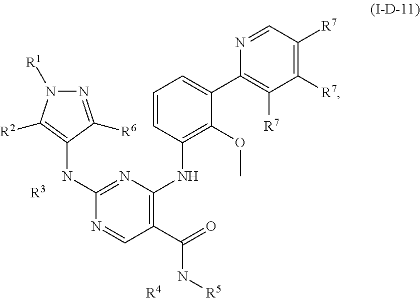

or a pharmaceutically acceptable salt, stereoisomer, solvate, prodrug, isotopic derivative, or tautomer thereof wherein all variables are as defined herein.

In some embodiments, the compound is of Formula (I-D-12):

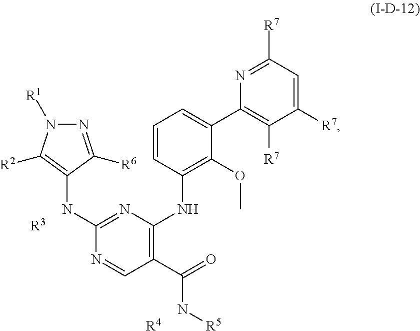

or a pharmaceutically acceptable salt, stereoisomer, solvate, prodrug, isotopic derivative, or tautomer thereof wherein all variables are as defined herein.

In some embodiments, the compound is of Formula (I-D-13):

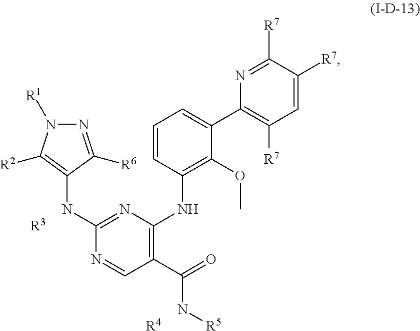

or a pharmaceutically acceptable salt, stereoisomer, solvate, prodrug, isotopic derivative, or tautomer thereof wherein all variables are as defined herein.

In some embodiments, the compound is of Formula (I-E-1):

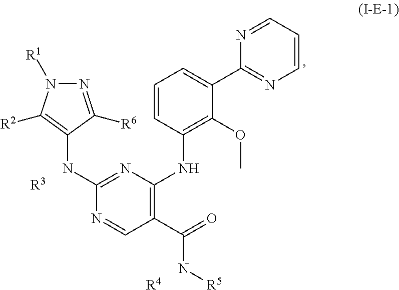

or a pharmaceutically acceptable salt, stereoisomer, solvate, prodrug, isotopic derivative, or tautomer thereof, wherein all variables are as defined herein.

In some embodiments, the compound is of Formula (I-E-2):

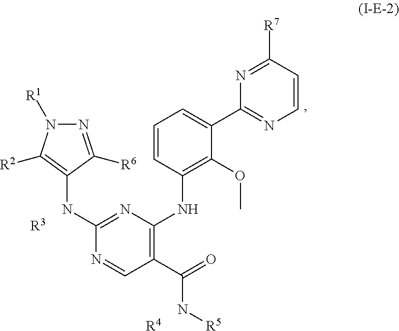

or a pharmaceutically acceptable salt, stereoisomer, solvate, prodrug, isotopic derivative, or tautomer thereof, wherein all variables are as defined herein.

In some embodiments, the compound is of Formula (I-E-3):

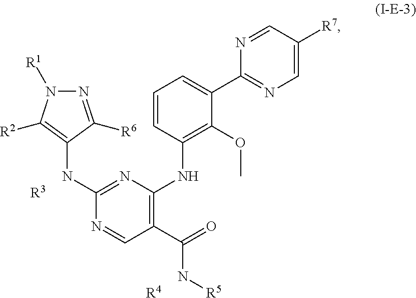

or tautomer thereof, wherein all variables are as defined herein.

In some embodiments, the compound is of Formula (I-E-4):

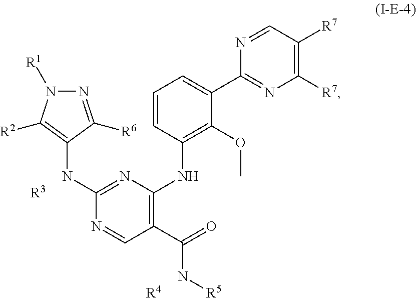

or a pharmaceutically acceptable salt, stereoisomer, solvate, prodrug, isotopic derivative, or tautomer thereof, wherein all variables are as defined herein.

In some embodiments, the compound is of Formula (I-E-5):

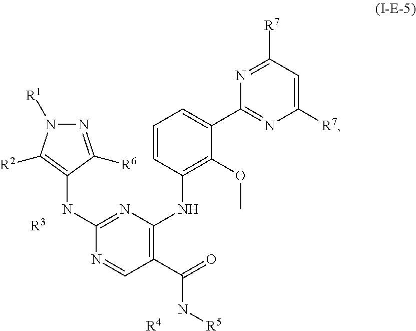

or a pharmaceutically acceptable salt, stereoisomer, solvate, prodrug, isotopic derivative, or tautomer thereof, wherein all variables are as defined herein.

In some embodiments, the compound is of Formula (I-E-6):

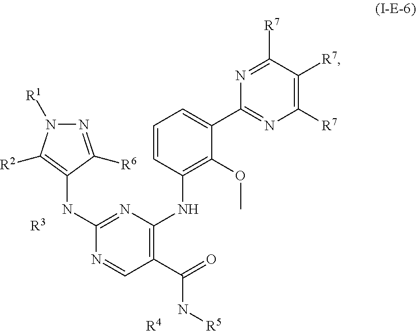

or a pharmaceutically acceptable salt, stereoisomer, solvate, prodrug, isotopic derivative, or tautomer thereof, wherein all variables are as defined herein.

In some embodiments, the compound is of Formula (I-A-2-I):

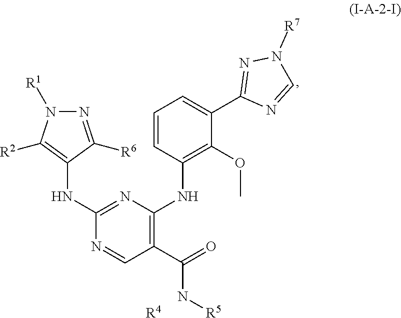

or a pharmaceutically acceptable salt, stereoisomer, solvate, prodrug, isotopic derivative, or tautomer thereof wherein all variables are as defined herein.

In some embodiments, the compound is of Formula (I-A-2-I-A):

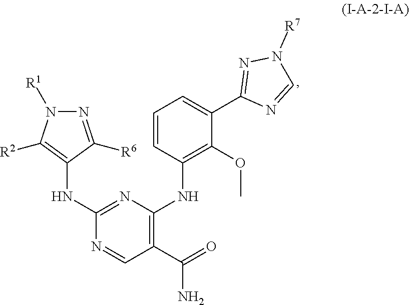

or a pharmaceutically acceptable salt, stereoisomer, solvate, prodrug, isotopic derivative, or tautomer thereof wherein all variables are as defined herein.

In some embodiments, the compound is of Formula (I-A-2-I-B):

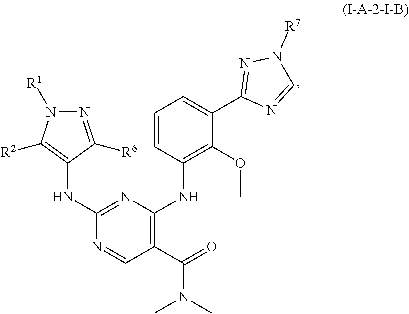

or a pharmaceutically acceptable salt, stereoisomer, solvate, prodrug, isotopic derivative, or tautomer thereof wherein all variables are as defined herein.

In some embodiments, the compound is of Formula (I-B-a-I):

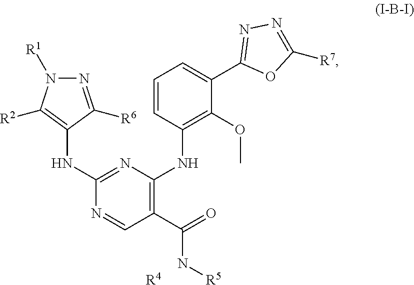

or tautomer thereof, wherein all variables are as defined herein.

In some embodiments, the compound is of Formula (I-B-I-A):

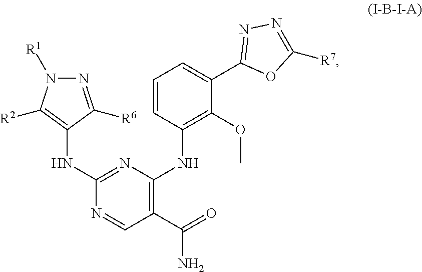

or a pharmaceutically acceptable salt, stereoisomer, solvate, prodrug, isotopic derivative, or tautomer thereof, wherein all variables are as defined herein.

In some embodiments, the compound is of Formula (I-B-I-B):

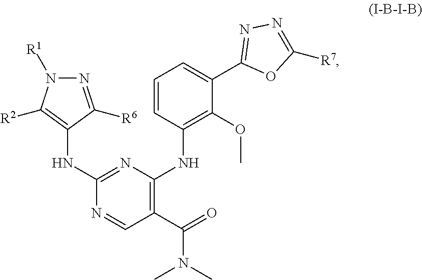

or a pharmaceutically acceptable salt, stereoisomer, solvate, prodrug, isotopic derivative, or tautomer thereof, wherein all variables are as defined herein.

In some embodiments, the compound is of Formula (I-C-1-I):

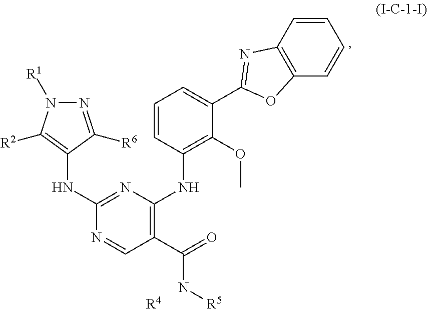

or a pharmaceutically acceptable salt, stereoisomer, solvate, prodrug, isotopic derivative, or tautomer thereof wherein all variables are as defined herein.

In some embodiments, the compound is of Formula (I-C-1-I-A):

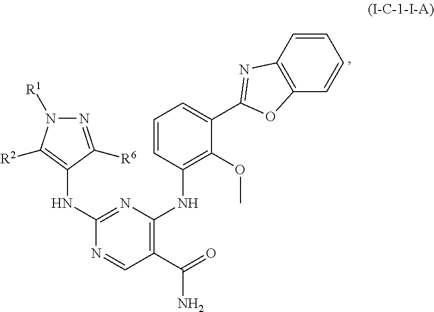

or a pharmaceutically acceptable salt, stereoisomer, solvate, prodrug, isotopic derivative, or tautomer thereof wherein all variables are as defined herein.

In some embodiments, the compound is of Formula (I-C-1-I-B):

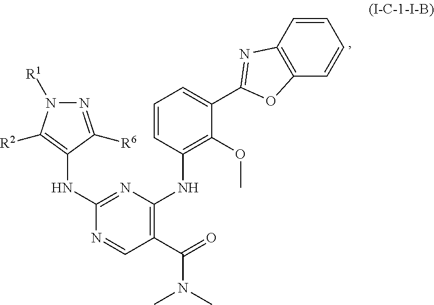

or tautomer thereof wherein all variables are as defined herein.

In some embodiments, the compound is of Formula (I-D-1-I):

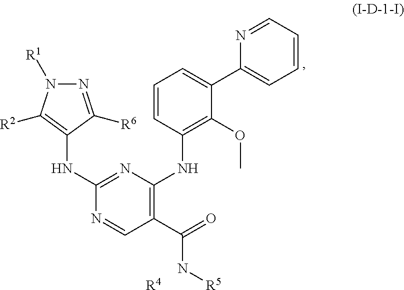

or a pharmaceutically acceptable salt, stereoisomer, solvate, prodrug, isotopic derivative, or tautomer thereof wherein all variables are as defined herein.

In some embodiments, the compound is of Formula (I-D-1-I-A):

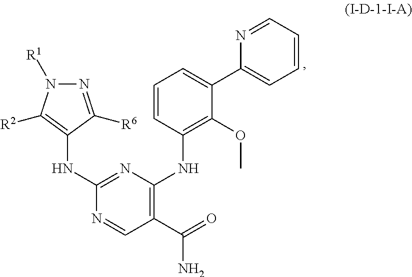

or a pharmaceutically acceptable salt, stereoisomer, solvate, prodrug, isotopic derivative, or tautomer thereof wherein all variables are as defined herein.

In some embodiments, the compound is of Formula (I-D-1-I-B):

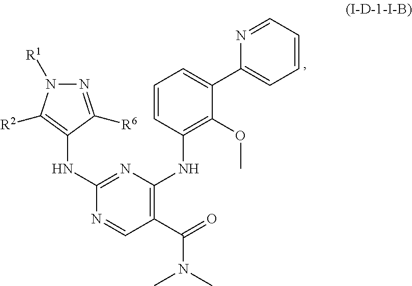

or a pharmaceutically acceptable salt, stereoisomer, solvate, prodrug, isotopic derivative, or tautomer thereof wherein all variables are as defined herein.

In some embodiments, the compound is of Formula (I-E-1-I):

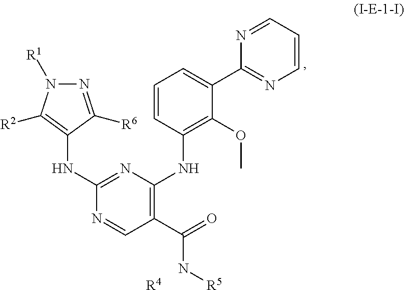

or a pharmaceutically acceptable salt, stereoisomer, solvate, prodrug, isotopic derivative, or tautomer thereof, wherein all variables are as defined herein.

In some embodiments, the compound is of Formula (I-E-1-I-A):

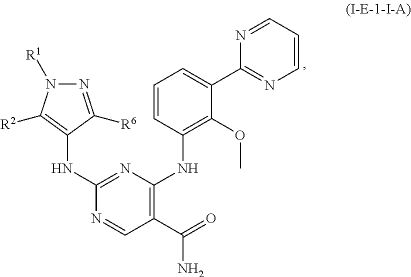

or a pharmaceutically acceptable salt, stereoisomer, solvate, prodrug, isotopic derivative, or tautomer thereof, wherein all variables are as defined herein.

In some embodiments, the compound is of Formula (I-E-1-I-B):

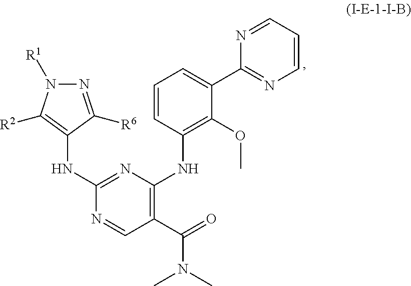

or a pharmaceutically acceptable salt, stereoisomer, solvate, prodrug, isotopic derivative, or tautomer thereof, wherein all variables are as defined herein.

In some embodiments the compound is of Formula (I-1):

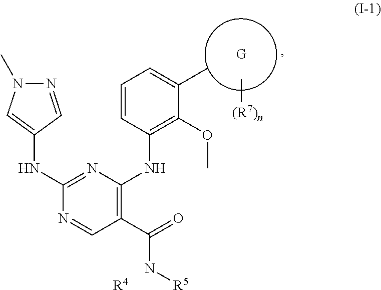

or a pharmaceutically acceptable salt, stereoisomer, solvate, prodrug, isotopic derivative, or tautomer thereof.

In some embodiments the compound is of Formula (I-2):

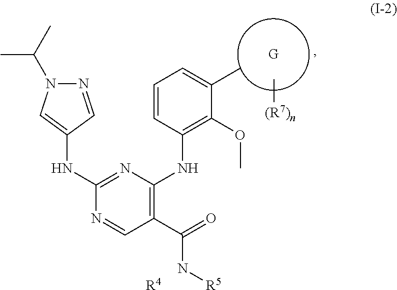

or tautomer thereof.

In some embodiments the compound is of Formula (I-3):

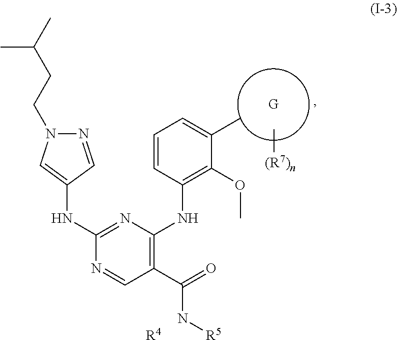

or a pharmaceutically acceptable salt, stereoisomer, solvate, prodrug, isotopic derivative, or tautomer thereof.

In some embodiments the compound is of Formula (I-4):

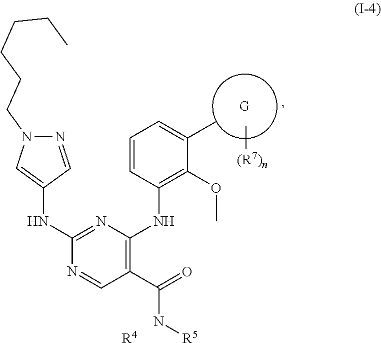

or a pharmaceutically acceptable salt, stereoisomer, solvate, prodrug, isotopic derivative, or tautomer thereof.

In some embodiments the compound is of Formula (I-5):

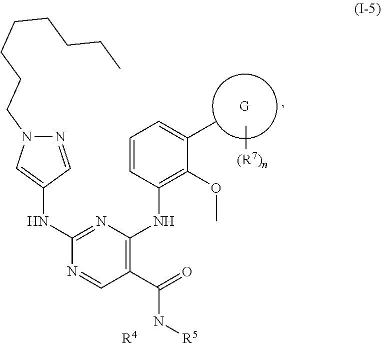

or a pharmaceutically acceptable salt, stereoisomer, solvate, prodrug, isotopic derivative, or tautomer thereof.

In some embodiments the compound is of Formula (I-6):

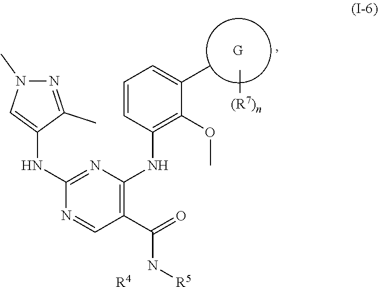

or a pharmaceutically acceptable salt, stereoisomer, solvate, prodrug, isotopic derivative, or tautomer thereof.

In some embodiments the compound is of Formula (I-7):

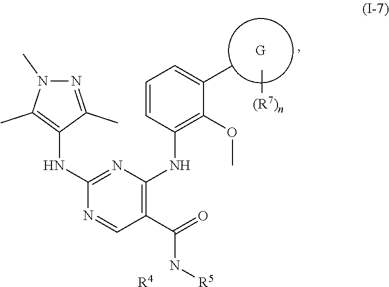

or tautomer thereof.

In some embodiments the compound is of Formula (I-8):

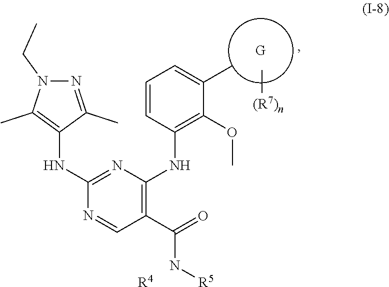

or a pharmaceutically acceptable salt, stereoisomer, solvate, prodrug, isotopic derivative, or tautomer thereof.

In some embodiments the compound is of Formula (I-9):

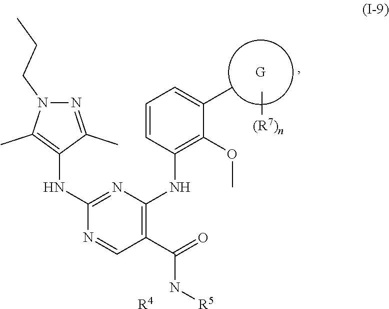

or a pharmaceutically acceptable salt, stereoisomer, solvate, prodrug, isotopic derivative, or tautomer thereof.

In some embodiments the compound is of Formula (I-10):

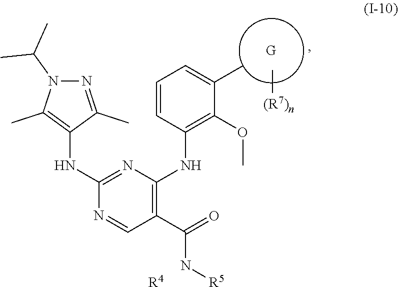

or a pharmaceutically acceptable salt, stereoisomer, solvate, prodrug, isotopic derivative, or tautomer thereof.

In some embodiments the compound is of Formula (I-11):

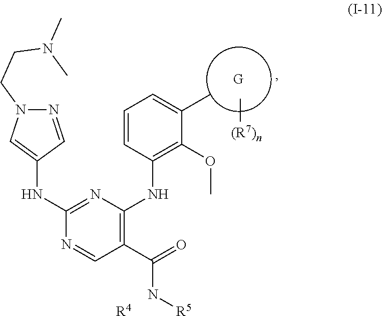

or a pharmaceutically acceptable salt, stereoisomer, solvate, prodrug, isotopic derivative, or tautomer thereof.

In some embodiments the compound is of Formula (I-12):

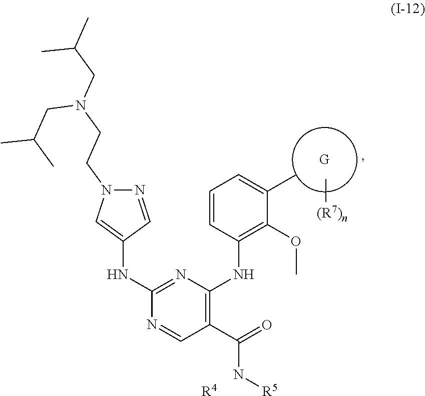

or a pharmaceutically acceptable salt, stereoisomer, solvate, prodrug, isotopic derivative, or tautomer thereof.

In some embodiments the compound is of Formula (I-13):

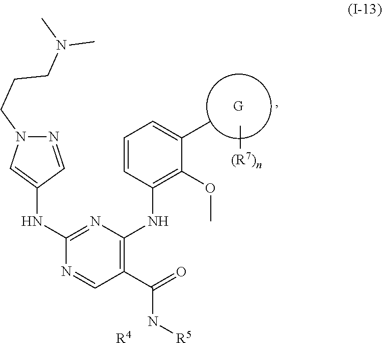

or a pharmaceutically acceptable salt, stereoisomer, solvate, prodrug, isotopic derivative, or tautomer thereof.

In some embodiments the compound is of Formula (I-14):

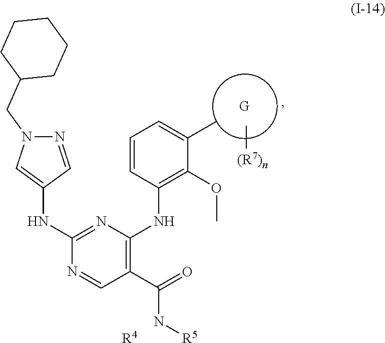

or a pharmaceutically acceptable salt, stereoisomer, solvate, prodrug, isotopic derivative, or tautomer thereof.

In some embodiments the compound is of Formula (I-15):

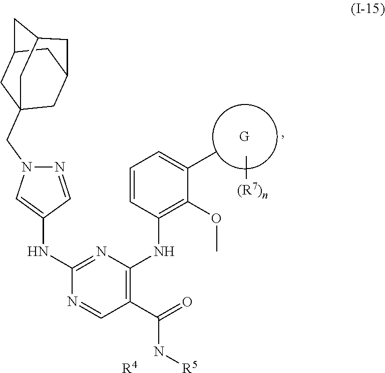

or a pharmaceutically acceptable salt, stereoisomer, solvate, prodrug, isotopic derivative, or tautomer thereof.

In some embodiments the compound is of Formula (I-16):

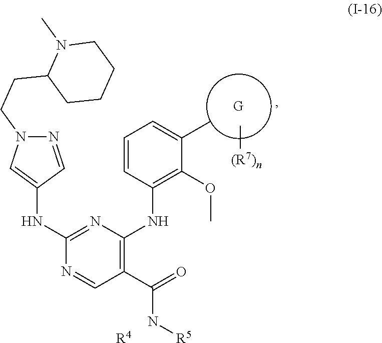

or a pharmaceutically acceptable salt, stereoisomer, solvate, prodrug, isotopic derivative, or tautomer thereof.

In some embodiments the compound is of Formula (I-17):

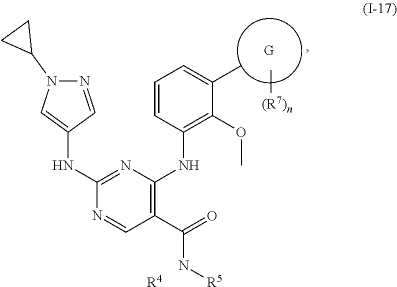

or a pharmaceutically acceptable salt, stereoisomer, solvate, prodrug, isotopic derivative, or tautomer thereof.

In some embodiments the compound is of Formula (I-18):

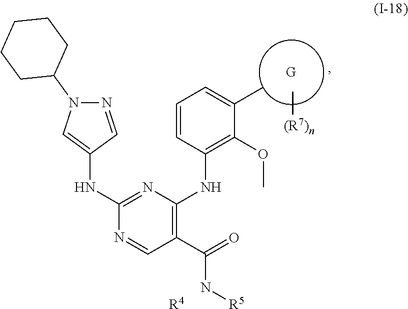

or tautomer thereof.

In some embodiments the compound is of Formula (I-19):

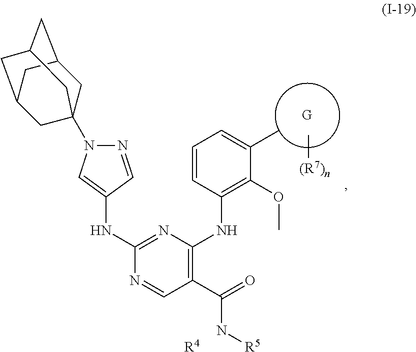

or a pharmaceutically acceptable salt, stereoisomer, solvate, prodrug, isotopic derivative, or tautomer thereof.

In some embodiments the compound is of Formula (I-20):

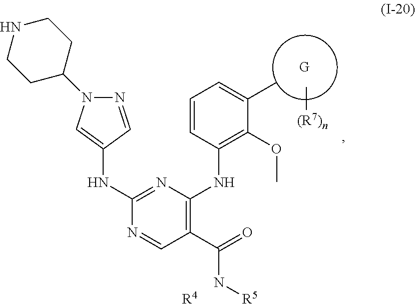

or a pharmaceutically acceptable salt, stereoisomer, solvate, prodrug, isotopic derivative, or tautomer thereof.

In some embodiments the compound is of Formula (I-21):

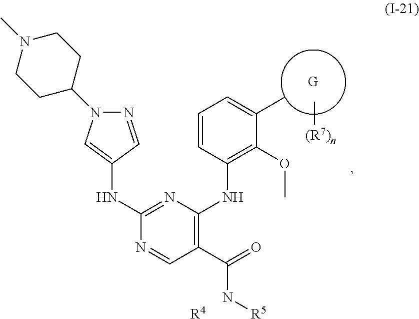

or a pharmaceutically acceptable salt, stereoisomer, solvate, prodrug, isotopic derivative, or tautomer thereof.

In some embodiments the compound is of Formula (I-22):

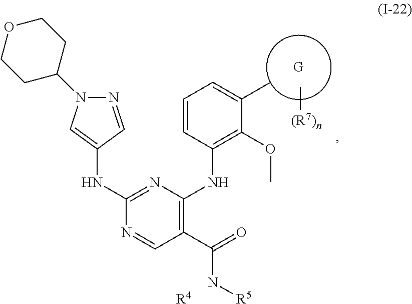

or a pharmaceutically acceptable salt, stereoisomer, solvate, prodrug, isotopic derivative, or tautomer thereof.

In some embodiments the compound is of Formula (I-23):

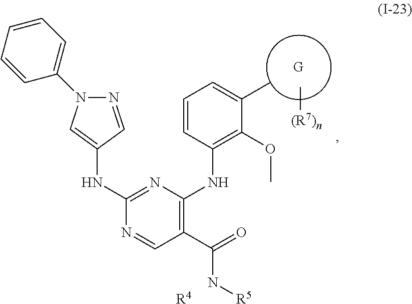

or a pharmaceutically acceptable salt, stereoisomer, solvate, prodrug, isotopic derivative, or tautomer thereof.

In some embodiments the compound is of Formula (I):

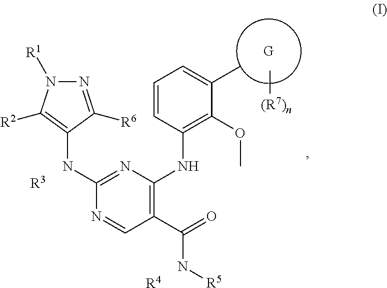

or a pharmaceutically acceptable salt, stereoisomer, solvate, prodrug, isotopic derivative, or tautomer thereof, wherein each substitutent is independently selected from the Table 1

TABLE 1
Possible combinations of substitutients

| | R¹ | | | |
|---|---|---|---|---|
| R¹ | —CH₃ | 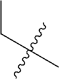 [SMILES: *CC] | 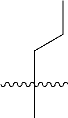 [SMILES: *CCC] | 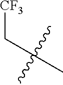 [SMILES: *CC(F)(F)F] |
| | 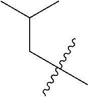 [SMILES: *CC(C)C] | 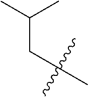 [SMILES: *CC(C)C] | 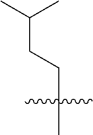 [SMILES: *CCC(C)C] | 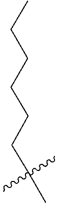 [SMILES: *CCCCCC] |
| | 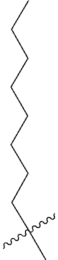 [SMILES: *CCCCCCCC] | 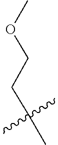 [SMILES: *CCOC] | 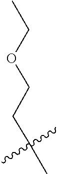 [SMILES: *CCOCC] | 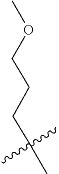 [SMILES: *CCCOC] |
| | 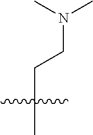 [SMILES: *CCN(C)C] | 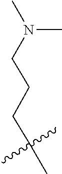 [SMILES: *CCCN(C)C] | 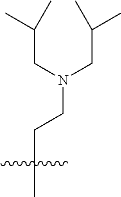 [SMILES: *CCN(CC(C)C)CC(C)C] | 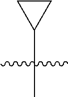 [SMILES: *C1CC1] |
| | 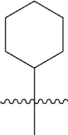 [SMILES: *C1CCCCC1] | 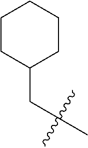 [SMILES: *CC1CCCCC1] | 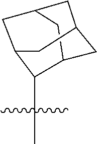 [SMILES: *C1C2CC3CC(C2)CC1C3] | 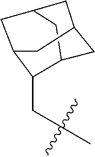 [SMILES: *CC1C2CC3CC(C2)CC1C3] |
| | 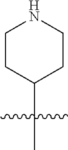 [SMILES: *C1CCNCC1] | 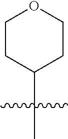 [SMILES: *C1CCOCC1] | 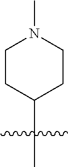 [SMILES: *C1CCN(C)CC1] | 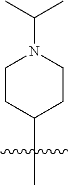 [SMILES: *C1CCN(C(C)C)CC1] |
| | 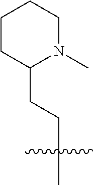 [SMILES: *CCC1CCCCN1C] | | 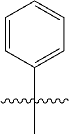 [SMILES: *c1ccccc1] | |
| | R2 | | | |
| R² | H | —CH₃ | 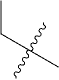 [SMILES: *CC] | 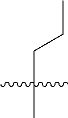 [SMILES: *CCC] |
| | 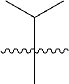 [SMILES: *C(C)C] | | 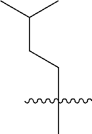 [SMILES: *CCC(C)C] | |
| | R³ | | | |
| R³ | H | —CH₃ | 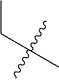 [SMILES: *CC] | 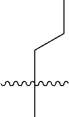 [SMILES: *CCC] |
| | 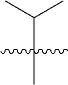 [SMILES: *C(C)C] | | 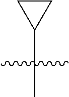 [SMILES: *C1CC1] | |
| | R⁴ | | | |
| R⁴ | H | —CH₃ | 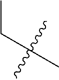 [SMILES: *CC] | |
| | R⁵ | | | |
| R⁵ | H | —CH₃ | 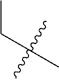 [SMILES: *CC] | |
| | R⁴, R⁵ | | | |
| R⁴, R⁵ | 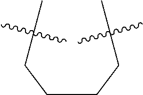 [SMILES: *CCCC*] | | 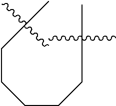 [SMILES: *CCCCC*] | |
| | R⁶ | | | |
| R⁶ | H | —CH₃ | 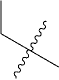 [SMILES: *CC] | 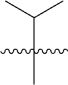 [SMILES: *C(C)C] |
| | 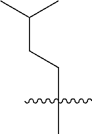 [SMILES: *CCC(C)C] | | | |
| | 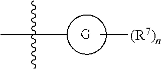 | | | |
| 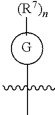 | 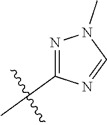 [SMILES: *c1ncn(C)n1] | 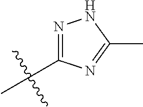 [SMILES: *c1n[nH]c(C)n1] | 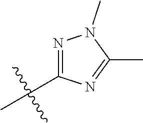 [SMILES: *c1nc(C)n(C)n1] | 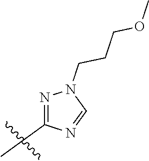 [SMILES: *c1ncn(CCCOC)n1] |
| | 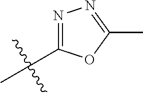 [SMILES: *c1nnc(C)o1] | 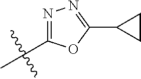 [SMILES: *c1nnc(C2CC2)o1] | 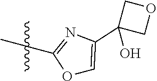 [SMILES: *c1nc(C2(O)COC2)co1] | 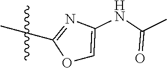 [SMILES: *c1nc(NC(C)=O)co1] |
| | 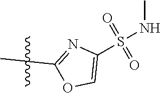 [SMILES: *c1nc(S(=O)(=O)NC)co1] | 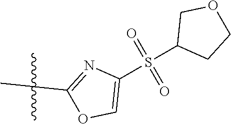 [SMILES: *c1nc(S(=O)(=O)C2CCOC2)co1] | | 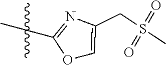 [SMILES: *c1nc(CS(C)(=O)=O)co1] |
| | 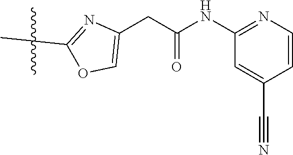 [SMILES: *c1nc(CC(=O)Nc2cc(C#N)ccn2)co1] | | 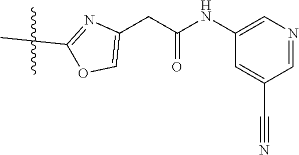 [SMILES: *c1nc(CC(=O)Nc2cncc(C#N)c2)co1] | |
| | 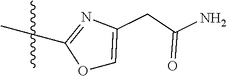 [SMILES: *c1nc(CC(N)=O)co1] | | 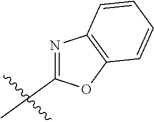 [SMILES: *c1nc2ccccc2o1] | 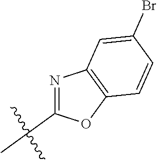 [SMILES: *c1nc2cc(Br)ccc2o1] |
| | 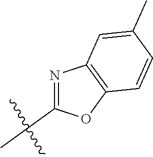 [SMILES: *c1nc2cc(C)ccc2o1] | 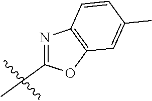 [SMILES: *c1nc2ccc(C)cc2o1] | 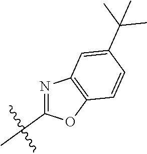 [SMILES: *c1nc2cc(C(C)(C)C)ccc2o1] | 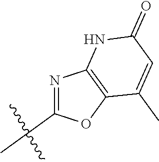 [SMILES: *c1nc2[nH]c(=O)cc(C)c2o1] |
| | 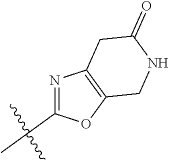 [SMILES: *c1nc2c(o1)CNC(=O)C2] | 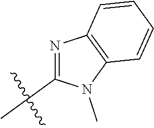 [SMILES: *c1nc2ccccc2n1C] | 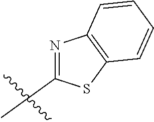 [SMILES: *c1nc2ccccc2s1] | 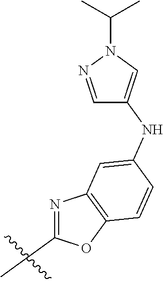 [SMILES: *c1nc2cc(Nc3cnn(C(C)C)c3)ccc2o1] |
| | 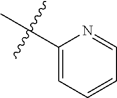 [SMILES: *c1ccccn1] | 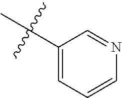 [SMILES: *c1cccnc1] | 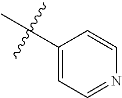 [SMILES: *c1ccncc1] | 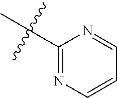 [SMILES: *c1ncccn1] |

| | | |
|---|---|---|
| 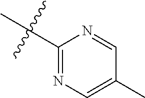 [SMILES: *c1ncc(C)cn1] | 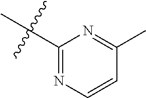 [SMILES: *c1nccc(C)n1] | 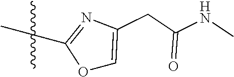 [SMILES: *c1nc(CC(=O)NC)co1] |

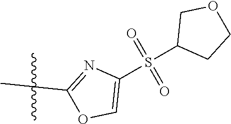 [SMILES: *c1nc(S(=O)(=O)C2CCOC2)co1]

In some embodiments, the compound is selected from the compounds described in Table 2 and pharmaceutically acceptable salts, stereoisomers, solvates, prodrugs, isotopic derivatives, or tautomers thereof.

In some embodiments, the compound is selected from the compounds described in Table 2 and pharmaceutically acceptable salts thereof.

In some embodiments, the compound is selected from the prodrugs of the compounds described in Table 2 and pharmaceutically acceptable salts thereof.

In some embodiments, the compound is selected from the compounds described in Table 2.

TABLE 2
Certain examples of the compound of Formula I

| # | Structure | IUPAC Name |
|---|---|---|
| 1 | 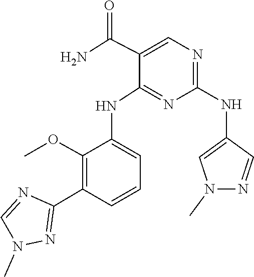 [SMILES: COc1c(Nc2nc(Nc3cnn(C)c3)ncc2C(N)=O)cccc1-c1ncn(C)n1] | 4-[2-methoxy-3-(1-methyl- 1,2,4-triazol-3-yl)anilino]-2-[(1- methylpyrazol-4- yl)amino]pyrimidine-5- carboxamide |
| 2 | 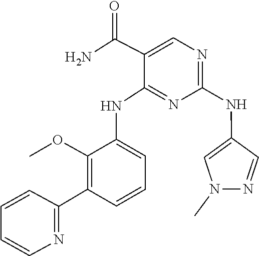 [SMILES: COc1c(Nc2nc(Nc3cnn(C)c3)ncc2C(N)=O)cccc1-c1ccccn1] | 4-[2-methoxy-3-(2- pyridyl)anilino]-2-[(1- methylpyrazol-4- yl)amino]pyrimidine-5- carboxamide |
| 3 | 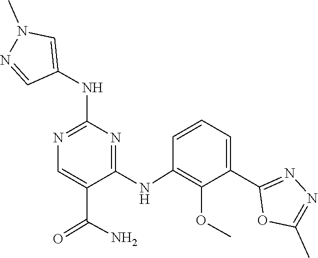 [SMILES: COc1c(Nc2nc(Nc3cnn(C)c3)ncc2C(N)=O)cccc1-c1nnc(C)o1] | 4-[2-methoxy-3-(5-methyl- 1,3,4-oxadiazol-2-yl)anilino]-2- [(1-methylpyrazol-4- yl)amino]pyrimidine-5- carboxamide |
| 4 | 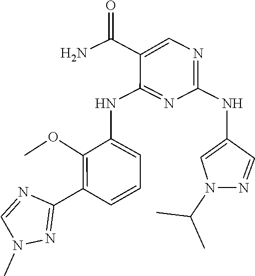 [SMILES: COc1c(Nc2nc(Nc3cnn(C(C)C)c3)ncc2C(N)=O)cccc1-c1ncn(C)n1] | 2-[(1-isopropylpyrazol-4- yl)amino]-4-[2-methoxy-3-(1- methyl-1,2,4-triazol-3- yl)anilino]pyrimidine-5- carboxamide |
| 5 | 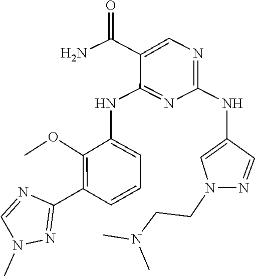 [SMILES: COc1c(Nc2nc(Nc3cnn(CCN(C)C)c3)ncc2C(N)=O)cccc1-c1ncn(C)n1] | 2-[[1-[2- (dimethylamino)ethyl]pyrazol- 4-yl]amino]-4-[2-methoxy-3-(1- methyl-1,2,4-triazol-3- yl)anilino]pyrimidine-5- carboxamide |
| 6 | 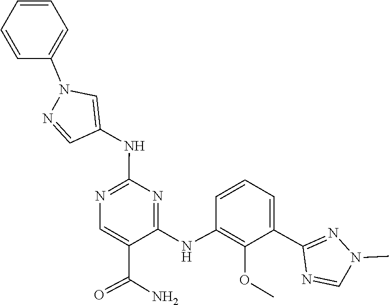 [SMILES: COc1c(Nc2nc(Nc3cnn(-c4ccccc4)c3)ncc2C(N)=O)cccc1-c1ncn(C)n1] | 4-[2-methoxy-3-(1-methyl- 1,2,4-triazol-3-yl)anilino]-2-[(1- phenylpyrazol-4- yl)amino]pyrimidine-5- carboxamide |
| 7 | 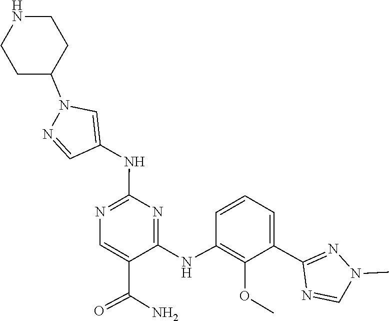 [SMILES: COc1c(Nc2nc(Nc3cnn(C4CCNCC4)c3)ncc2C(N)=O)cccc1-c1ncn(C)n1] | 4-[2-methoxy-3-(1-methyl- 1,2,4-triazol-3-yl)anilino]-2-[[1- (4-piperidyl)pyrazol-4- yl]amino]pyrimidine-5- carboxamide |
| 8 | 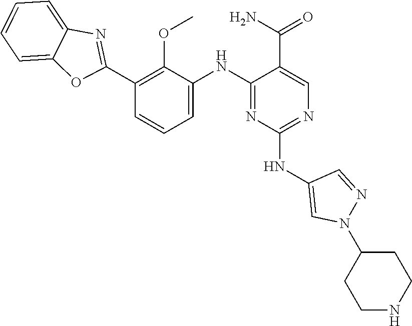 [SMILES: COc1c(Nc2nc(Nc3cnn(C4CCNCC4)c3)ncc2C(N)=O)cccc1-c1nc2ccccc2o1] | 4-[3-(1,3-benzoxazol-2-yl)-2- methoxy-anilino]-2-[[1-(4- piperidyl)pyrazol-4- yl]amino]pyrimidine-5- carboxamide |
| 9 | 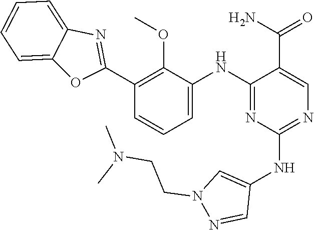 [SMILES: COc1c(Nc2nc(Nc3cnn(CCN(C)C)c3)ncc2C(N)=O)cccc1-c1nc2ccccc2o1] | 4-[3-(1,3-benzoxazol-2-yl)-2- methoxy-anilino]-2-[1-[2- (dimethylamino)ethyl]pyrazol- 4-yl]amino]pyrimidine-5- carboxamide |
| 10 | 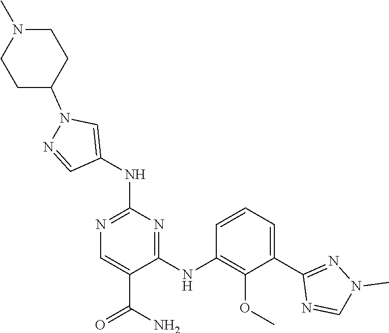 [SMILES: COc1c(Nc2nc(Nc3cnn(C4CCN(C)CC4)c3)ncc2C(N)=O)cccc1-c1ncn(C)n1] | 4-[2-methoxy-3-(1-methyl- 1,2,4-triazol-3-yl)anilino]-2-[[1- (1-methyl-4-piperidyl)pyrazol- 4-yl]amino]pyrimidine-5- carboxamide |
| 11 | 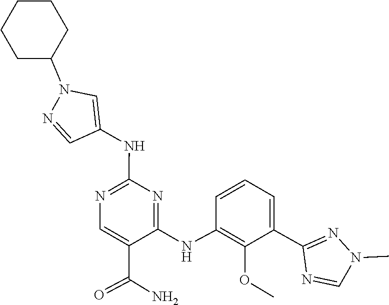 [SMILES: COc1c(Nc2nc(Nc3cnn(C4CCCCC4)c3)ncc2C(N)=O)cccc1-c1ncn(C)n1] | 2-[(1-cyclohexylpyrazol-4- yl)amino]-4-[2-methoxy-3-(1- methyl-1,2,4-triazol-3- yl)anilino]pyrimidine-5- carboxamide |
| 12 | 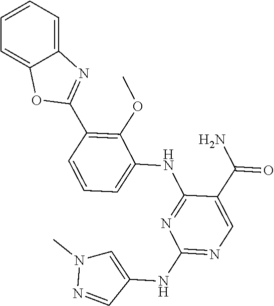 [SMILES: COc1c(Nc2nc(Nc3cnn(C)c3)ncc2C(N)=O)cccc1-c1nc2ccccc2o1] | 4-[3-(1,3-benzoxazol-2-yl)-2- methoxy-anilino]-2-[(1- methylpyrazol-4- yl)amino]pyrimidine-5- carboxamide |
| 13 | 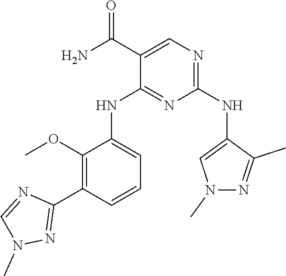 [SMILES: COc1c(Nc2nc(Nc3cn(C)nc3C)ncc2C(N)=O)cccc1-c1ncn(C)n1] | 2-[(1,3-dimethylpyrazol-4- yl)amino]-4-[2-methoxy-3-(1- methyl-1,2,4-triazol-3- yl)anilino]pyrimidine-5- carboxamide |
| 14 | 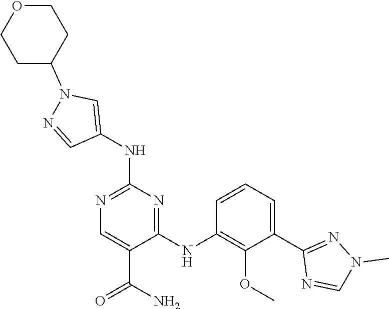 [SMILES: COc1c(Nc2nc(Nc3cnn(C4CCOCC4)c3)ncc2C(N)=O)cccc1-c1ncn(C)n1] | 4-[2-methoxy-3-(1-methyl- 1,2,4-triazol-3-yl)anilino]-2-[(1- tetrahydropyran-4-ylpyrazol-4- yl)amino]pyrimidine-5- carboxamide |
| 15 | 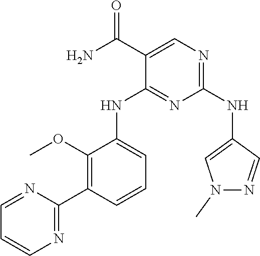 [SMILES: COc1c(Nc2nc(Nc3cnn(C)c3)ncc2C(N)=O)cccc1-c1ncccn1] | 4-(2-methoxy-3-pyrimidin-2-yl- anilino)-2-[(1-methylpyrazol-4- yl)amino]pyrimidine-5- carboxamide |
| 16 | 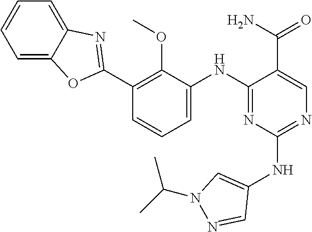 [SMILES: COc1c(Nc2nc(Nc3cnn(C(C)C)c3)ncc2C(N)=O)cccc1-c1nc2ccccc2o1] | 4-[3-(1,3-benzoxazol-2-yl)-2- methoxy-anilino]-2-[(1- isopropylpyrazol-4- yl)amino]pyrimidine-5- carboxamide |
| 17 | 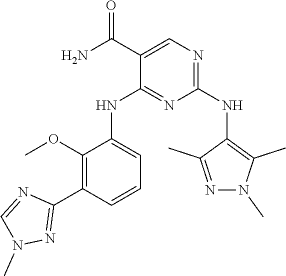 [SMILES: COc1c(Nc2nc(Nc3c(C)nn(C)c3C)ncc2C(N)=O)cccc1-c1ncn(C)n1] | 4-[2-methoxy-3-(1-methyl- 1,2,4-triazol-3-yl)anilino]-2- [(1,3,5-trimethylpyrazol-4- yl)amino]pyrimidine-5- carboxamide |
| 18 | 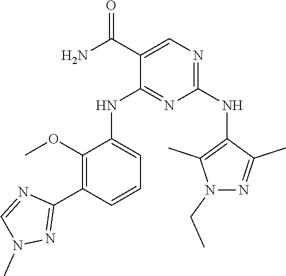 [SMILES: CCn1nc(C)c(Nc2ncc(C(N)=O)c(Nc3cccc(-c4ncn(C)n4)c3OC)n2)c1C] | 2-[(1-ethyl-3,5-dimethyl- pyrazol-4-yl)amino]-4-[2- methoxy-3-(1-methyl-1,2,4- triazol-3-yl)anilino]pyrimidine- 5-carboxamide |
| 19 | 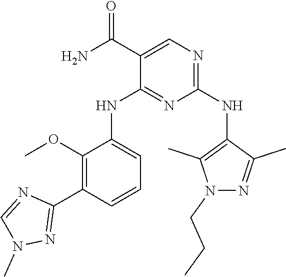 [SMILES: CCCn1nc(C)c(Nc2ncc(C(N)=O)c(Nc3cccc(-c4ncn(C)n4)c3OC)n2)c1C] | 2-[(3,5-dimethyl-1-propyl- pyrazol-4-yl)amino]-4-[2- methoxy-3-(1-methyl-1,2,4- triazol-3-yl)anilino]pyrimidine- 5-carboxamide |
| 20 | 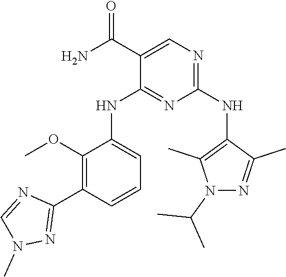 [SMILES: COc1c(Nc2nc(Nc3c(C)nn(C(C)C)c3C)ncc2C(N)=O)cccc1-c1ncn(C)n1] | 2-[(1-isopropyl-3,5-dimethyl- pyrazol-4-yl)amino]-4-[2- methoxy-3-(1-methyl-1,2,4- triazol-3-yl)anilino]pyrimidine- 5-carboxamide |
| 21 | 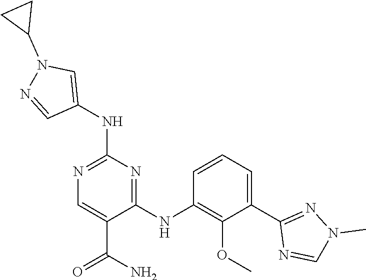 [SMILES: COc1c(Nc2nc(Nc3cnn(C4CC4)c3)ncc2C(N)=O)cccc1-c1ncn(C)n1] | 2-[(1-cyclopropylpyrazol-4- yl)amino]-4-[2-methoxy-3-(1- methyl-1,2,4-triazol-3- yl)anilino]pyrimidine-5- carboxamide |
| 22 | 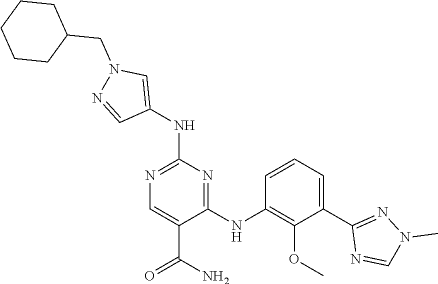 [SMILES: COc1c(Nc2nc(Nc3cnn(CC4CCCCC4)c3)ncc2C(N)=O)cccc1-c1ncn(C)n1] | 2-[[1- (cyclohexylmethyl)pyrazol-4- yl]amino]-4-[2-methoxy-3-(1- methyl-1,2,4-triazol-3- yl)anilino]pyrimidine-5- carboxamide |
| 23 | 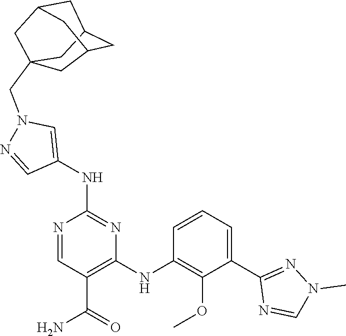 [SMILES: COc1c(Nc2nc(Nc3cnn(CC45CC6CC(CC(C6)C4)C5)c3)ncc2C(N)=O)cccc1-c1ncn(C)n1] | 2-[[1-(1- adamantylmethyl)pyrazol-4- yl]amino]-4-[2-methoxy-3-(1- methyl-1,2,4-triazol-3- yl)anilino]pyrimidine-5- carboxamide |
| 24 | 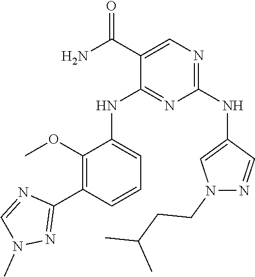 [SMILES: COc1c(Nc2nc(Nc3cnn(CCC(C)C)c3)ncc2C(N)=O)cccc1-c1ncn(C)n1] | 2-[(1-isopentylpyrazol-4- yl)amino]-4-[2-methoxy-3-(1- methyl-1,2,4-triazol-3- yl)anilino]pyrimidine-5- carboxamide |
| 25 | 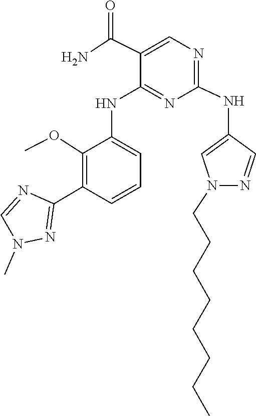 [SMILES: CCCCCCCCn1cc(Nc2ncc(C(N)=O)c(Nc3cccc(-c4ncn(C)n4)c3OC)n2)cn1] | 4-[2-methoxy-3-(1-methyl- 1,2,4-triazol-3-yl)anilino]-2-[(1- octylpyrazol-4- yl)amino]pyrimidine-5- carboxamide |
| 26 | 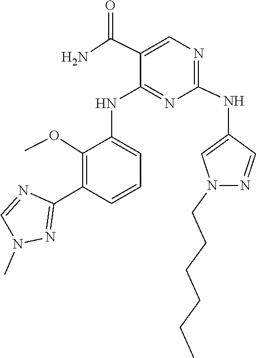 [SMILES: CCCCCCn1cc(Nc2ncc(C(N)=O)c(Nc3cccc(-c4ncn(C)n4)c3OC)n2)cn1] | 2-[(1-hexylpyrazol-4- yl)amino]-4-[2-methoxy-3-(1- methyl-1,2,4-triazol-3- yl)anilino]pyrimidine-5- carboxamide |
| 27 | 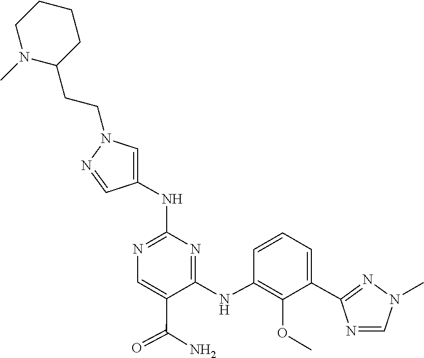 | 4-[2-methoxy-3-(1-methyl- 1,2,4-triazol-3-yl)anilino]-2-[[1- [2-(1-methyl-2- piperidyl)ethyl]pyrazol-4- yl]amino]pyrimidine-5- carboxamide |
| 28 | 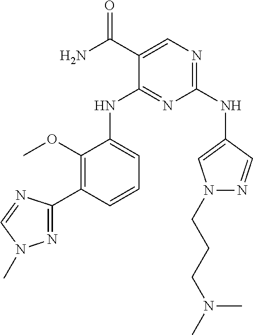 [SMILES: COc1c(Nc2nc(Nc3cnn(CCCN(C)C)c3)ncc2C(N)=O)cccc1-c1ncn(C)n1] | 2-[[1-[3- (dimethylamino)propyl]pyrazol- 4-yl]amino]-4-[2-methoxy-3- (1-methyl-1,2,4-triazol-3- yl)anilino]pyrimidine-5- carboxamide |
| 29 | 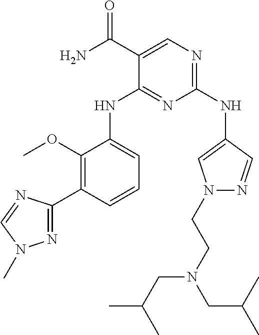 [SMILES: COc1c(Nc2nc(Nc3cnn(CCN(CC(C)C)CC(C)C)c3)ncc2C(N)=O)cccc1-c1ncn(C)n1] | 2-[[1-[2- (diisobutylamino)ethyl]pyrazol- 4-yl]amino]-4-[2-methoxy-3-(1- methyl-1,2,4-triazol-3- yl)anilino]pyrimidine-5- carboxamide |
| 30 | 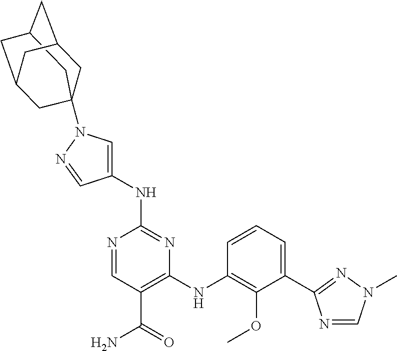 [SMILES: COc1c(Nc2nc(Nc3cnn(C45CC6CC(CC(C6)C4)C5)c3)ncc2C(N)=O)cccc1-c1ncn(C)n1] | 2-[[1-(1-adamantyl)pyrazol-4- yl]amino]-4-[2-methoxy-3-(1- methyl-1,2,4-triazol-3- yl)anilino]pyrimidine-5- carboxamide |
| 31 | 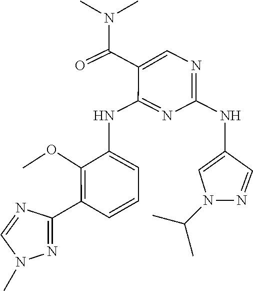 [SMILES: COc1c(Nc2nc(Nc3cnn(C(C)C)c3)ncc2C(=O)N(C)C)cccc1-c1ncn(C)n1] | 2-[(1-isopropylpyrazol-4- yl)amino]-4-[2-methoxy-3-(1- methyl-1,2,4-triazol-3- yl)anilino]-N,N-dimethyl- pyrimidine-5-carboxamide |
| 32 | 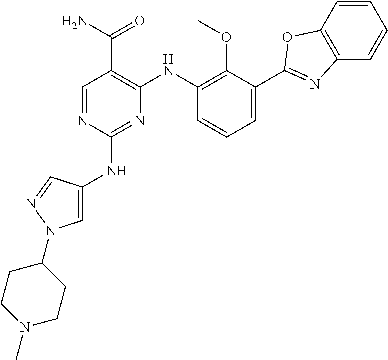 [SMILES: COc1c(Nc2nc(Nc3cnn(C4CCN(C)CC4)c3)ncc2C(N)=O)cccc1-c1nc2ccccc2o1] | 4-[3-(1,3-benzoxazol-2-yl)-2- methoxy-anilino]-2-[[1-(1- methyl-4-piperidyl)pyrazol-4- yl]amino]pyrimidine-5- carboxamide |
| 33 | 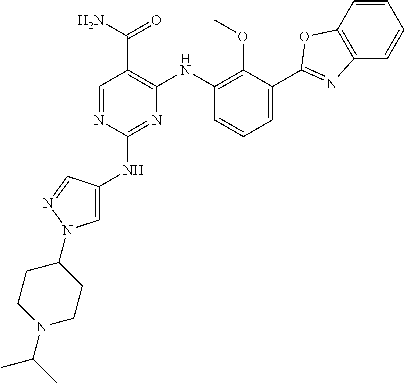 [SMILES: COc1c(Nc2nc(Nc3cnn(C4CCN(C(C)C)CC4)c3)ncc2C(N)=O)cccc1-c1nc2ccccc2o1] | 4-[3-(1,3-benzoxazol-2-yl)-2- methoxy-anilino]-2-[[1-(1- isopropyl-4-piperidyl)pyrazol- 4-yl]amino]pyrimidine-5- carboxamide |
| 34 | 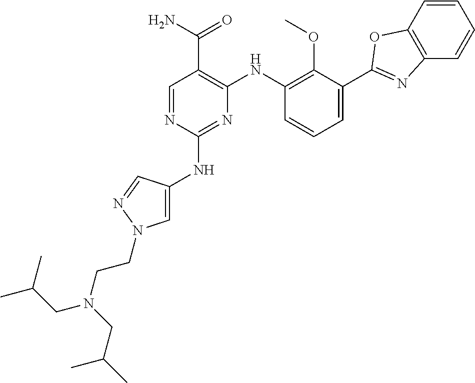 [SMILES: COc1c(Nc2nc(Nc3cnn(CCN(CC(C)C)CC(C)C)c3)ncc2C(N)=O)cccc1-c1nc2ccccc2o1] | 4-[3-(1,3-benzoxazol-2-yl)-2- methoxy-anilino]-2-[1-[2- (diisobutylamino)ethyl]pyrazol- 4-yl]amino]pyrimidine-5- carboxamide |
| 35 | 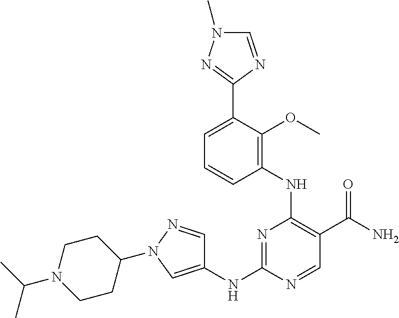 [SMILES: COc1c(Nc2nc(Nc3cnn(C4CCN(C(C)C)CC4)c3)ncc2C(N)=O)cccc1-c1ncn(C)n1] | 2-[[1-(1-isopropyl-4- piperidyl)pyrazol-4-yl]amino]- 4-[2-methoxy-3-(1-methyl- 1,2,4-triazol-3- yl)anilino]pyrimidine-5- carboxamide |
| 36 | 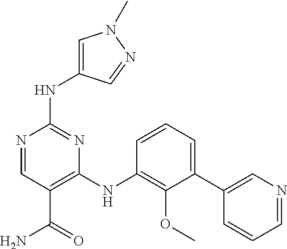 [SMILES: COc1c(Nc2nc(Nc3cnn(C)c3)ncc2C(N)=O)cccc1-c1cccnc1] | 4-[2-methoxy-3-(3- pyridyl)anilino]-2-[(1- methylpyrazol-4- yl)amino]pyrimidine-5- carboxamide |
| 37 | 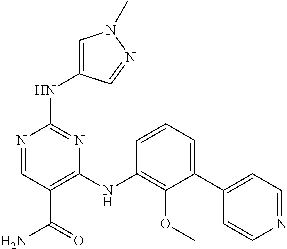 [SMILES: COc1c(Nc2nc(Nc3cnn(C)c3)ncc2C(N)=O)cccc1-c1ccncc1] | 4-[2-methoxy-3-(4- pyridyl)anilino]-2-[(1- methylpyrazol-4- yl)amino]pyrimidine-5- carboxamide |
| 38 | 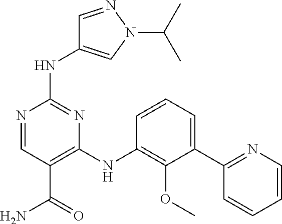 [SMILES: COc1c(Nc2nc(Nc3cnn(C(C)C)c3)ncc2C(N)=O)cccc1-c1ccccn1] | 2-[(1-isopropylpyrazol-4- yl)amino]-4-[2-methoxy-3-(2- pyridyl)anilino]pyrimidine-5- carboxamide |
| 39 | 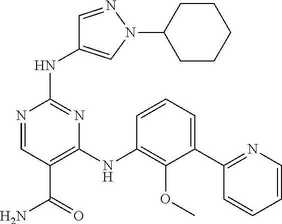 [SMILES: COc1c(Nc2nc(Nc3cnn(C4CCCCC4)c3)ncc2C(N)=O)cccc1-c1ccccn1] | 2-[(1-cyclohexylpyrazol-4- yl)amino]-4-[2-methoxy-3-(2- pyridyl)anilino]pyrimidine-5- carboxamide |
| 40 | 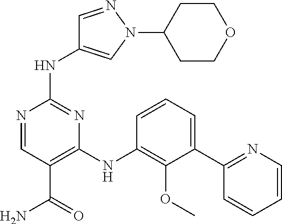 [SMILES: COc1c(Nc2nc(Nc3cnn(C4CCOCC4)c3)ncc2C(N)=O)cccc1-c1ccccn1] | 4-[2-methoxy-3-(2- pyridyl)anilino]-2-[(1- tetrahydropyran-4-ylpyrazol-4- yl)amino]pyrimidine-5- carboxamide |
| 41 | 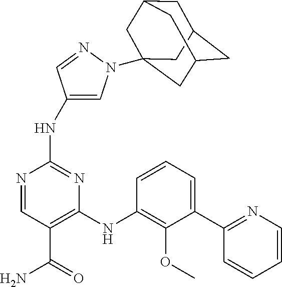 [SMILES: COc1c(Nc2nc(Nc3cnn(C45CC6CC(CC(C6)C4)C5)c3)ncc2C(N)=O)cccc1-c1ccccn1] | 2-[[1-(1-adamantyl)pyrazol-4- yl]amino]-4-[2-methoxy-3-(2- pyridyl)anilino]pyrimidine-5- carboxamide |
| 42 | 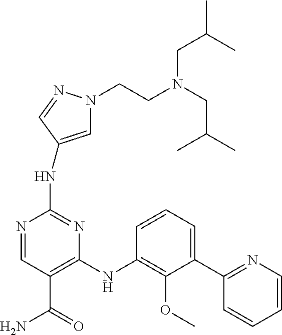 [SMILES: COc1c(Nc2nc(Nc3cnn(CCN(CC(C)C)CC(C)C)c3)ncc2C(N)=O)cccc1-c1ccccn1] | 2-[[1-[2- (diisobutylamino)ethyl]pyrazol- 4-yl]amino]-4-[2-methoxy-3-(2- pyridyl)anilino]pyrimidine-5- carboxamide |
| 43 | 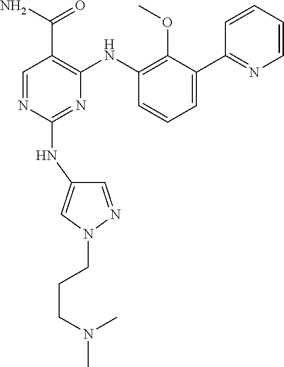 [SMILES: COc1c(Nc2nc(Nc3cnn(CCCN(C)C)c3)ncc2C(N)=O)cccc1-c1ccccn1] | 2-[[1-[3- (dimethylamino)propyl]pyrazol- 4-yl]amino]-4-[2-methoxy-3- (2-pyridyl)anilino]pyrimidine- 5-carboxamide |
| 44 | 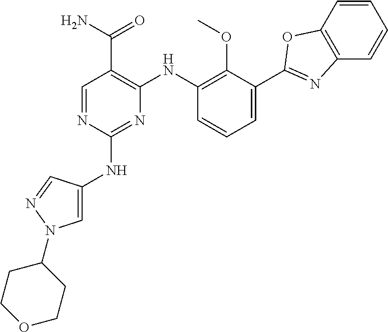 [SMILES: COc1c(Nc2nc(Nc3cnn(C4CCOCC4)c3)ncc2C(N)=O)cccc1-c1nc2ccccc2o1] | 4-[3-(1,3-benzoxazol-2-yl)-2- methoxy-anilino]-2-[(1- tetrahydropyran-4-ylpyrazol-4- yl)amino]pyrimidine-5- carboxamide |
| 45 | 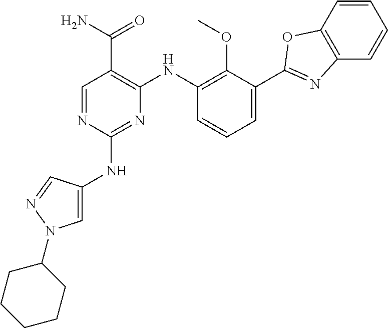 [SMILES: COc1c(Nc2nc(Nc3cnn(C4CCCCC4)c3)ncc2C(N)=O)cccc1-c1nc2ccccc2o1] | 4-[3-(1,3-benzoxazol-2-yl)-2- methoxy-anilino]-2-[(1- cyclohexylpyrazol-4- yl)amino]pyrimidine-5- carboxamide |
| 46 | 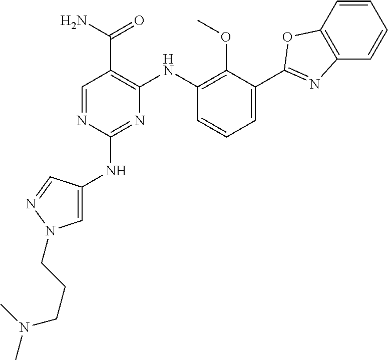 [SMILES: COc1c(Nc2nc(Nc3cnn(CCCN(C)C)c3)ncc2C(N)=O)cccc1-c1nc2ccccc2o1] | 4-[3-(1,3-benzoxazol-2-yl)-2- methoxy-anilino]-2-[[1-[3- (dimethylamino)propyl]pyrazol- 4-yl]amino]pyrimidine-5- carboxamide |
| 47 | 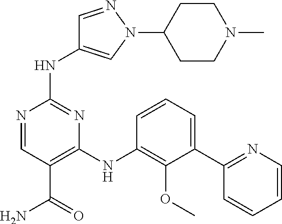 [SMILES: COc1c(Nc2nc(Nc3cnn(C4CCN(C)CC4)c3)ncc2C(N)=O)cccc1-c1ccccn1] | 4-[2-methoxy-3-(2- pyridyl)anilino]-2-[1-(1- methyl-4-piperidyl)pyrazol-4- yl]amino]pyrimidine-5- carboxamide |
| 48 | 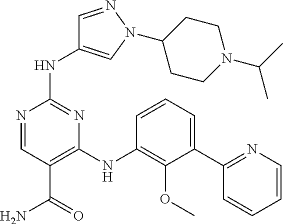 [SMILES: COc1c(Nc2nc(Nc3cnn(C4CCN(C(C)C)CC4)c3)ncc2C(N)=O)cccc1-c1ccccn1] | 2-[[1-(1-isopropyl-4- piperidyl)pyrazol-4-yl]amino]- 4-[2-methoxy-3-(2- pyridyl)anilino]pyrimidine-5- carboxamide |
| 49 | 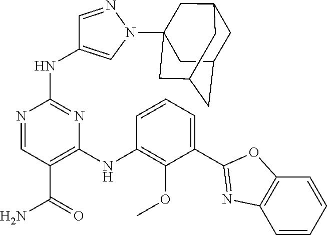 [SMILES: COc1c(Nc2nc(Nc3cnn(C45CC6CC(CC(C6)C4)C5)c3)ncc2C(N)=O)cccc1-c1nc2ccccc2o1] | 2-[[1-(1-adamantyl)pyrazol-4- yl]amino]-4-[3-(1,3- benzoxazol-2-yl)-2-methoxy- anilino]pyrimidine-5- carboxamide |
| 50 | 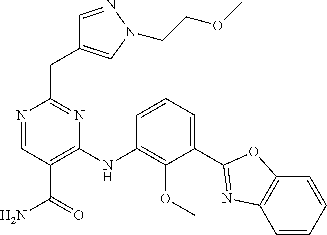 [SMILES: COCCn1cc(Cc2ncc(C(N)=O)c(Nc3cccc(-c4nc5ccccc5o4)c3OC)n2)cn1] | 4-[3-(1,3-benzoxazol-2-yl)-2- methoxy-anilino]-2-[1-(2- methoxyethyl)pyrazol-4- yl]amino]pyrimidine-5- carboxamide |
| 51 | 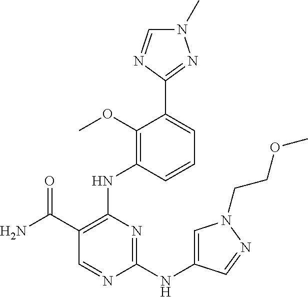 [SMILES: COCCn1cc(Nc2ncc(C(N)=O)c(Nc3cccc(-c4ncn(C)n4)c3OC)n2)cn1] | 2-[1-(2-methoxyethyl)pyrazol- 4-yl]amino]-4-[2-methoxy-3-(1- methyl-1,2,4-triazol-3- yl)anilino]pyrimidine-5- carboxamide |
| 52 | 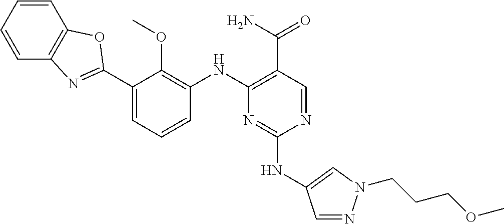 [SMILES: COCCCn1cc(Nc2ncc(C(N)=O)c(Nc3cccc(-c4nc5ccccc5o4)c3OC)n2)cn1] | 4-[3-(1,3-benzoxazol-2-yl)-2- methoxy-anilino]-2-[[1-(3- methoxypropyl)pyrazol-4- yl]amino]pyrimidine-5- carboxamide |
| 53 | 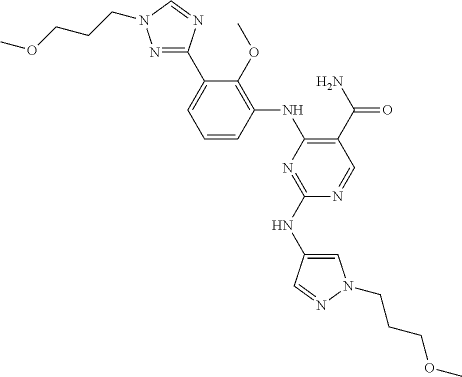 | 4-[2-methoxy-3-[1-(3- methoxypropyl)-1,2,4-triazol-3- yl]anilino]-2-[1-(3- methoxypropyl)pyrazol-4- yl]amino]pyrimidine-5- carboxamide |
| 54 | 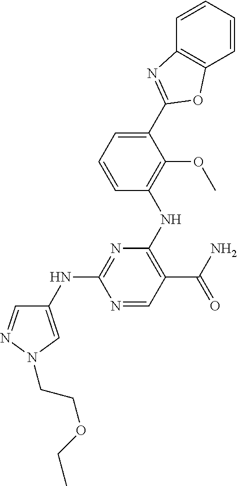 [SMILES: CCOCCn1cc(Nc2ncc(C(N)=O)c(Nc3cccc(-c4nc5ccccc5o4)c3OC)n2)cn1] | 4-[3-(1,3-benzoxazol-2-yl)-2- methoxy-anilino]-2-[1-(2- ethoxyethyl)pyrazol-4- yl]amino]pyrimidine-5- carboxamide |
| 55 | 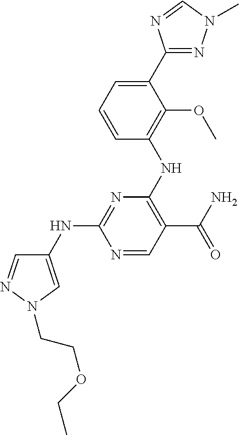 [SMILES: CCOCCn1cc(Nc2ncc(C(N)=O)c(Nc3cccc(-c4ncn(C)n4)c3OC)n2)cn1] | 2-[[1-(2-ethoxyethyl)pyrazol-4- yl]amino]-4-[2-methoxy-3-(1- methyl-1,2,4-triazol-3- yl)anilino]pyrimidine-5- carboxamide |
| 56 | 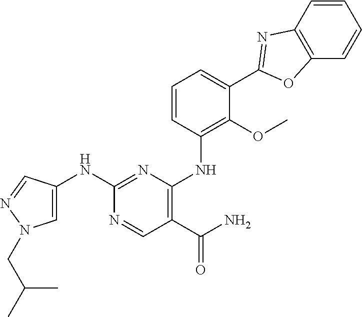 [SMILES: COc1c(Nc2nc(Nc3cnn(CC(C)C)c3)ncc2C(N)=O)cccc1-c1nc2ccccc2o1] | 4-[3-(1,3-benzoxazol-2-yl)-2- methoxy-anilino]-2-[(1- isobutylpyrazol-4- yl)amino]pyrimidine-5- carboxamide |
| 57 | 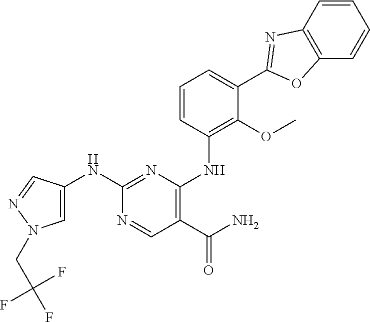 [SMILES: COc1c(Nc2nc(Nc3cnn(CC(F)(F)F)c3)ncc2C(N)=O)cccc1-c1nc2ccccc2o1] | 4-[3-(1,3-benzoxazol-2-yl)-2- methoxy-anilino]-2-[[1-(2,2,2- trifluoroethyl)pyrazol-4- yl]amino]pyrimidine-5- carboxamide |
| 58 | 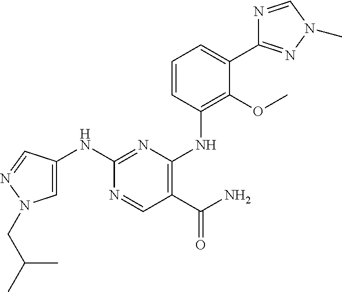 [SMILES: COc1c(Nc2nc(Nc3cnn(CC(C)C)c3)ncc2C(N)=O)cccc1-c1ncn(C)n1] | 2-[(1-isobutylpyrazol-4- yl)amino]-4-[2-methoxy-3-(1- methyl-1,2,4-triazol-3- yl)anilino]pyrimidine-5- carboxamide |
| 59 | 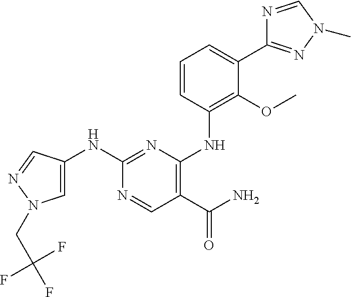 [SMILES: COc1c(Nc2nc(Nc3cnn(CC(F)(F)F)c3)ncc2C(N)=O)cccc1-c1ncn(C)n1] | 4-[2-methoxy-3-(1-methyl- 1,2,4-triazol-3-yl)anilino]-2-[1- (2,2,2-trifluoroethyl)pyrazol-4- yl]amino]pyrimidine-5- carboxamide |
| 60 | 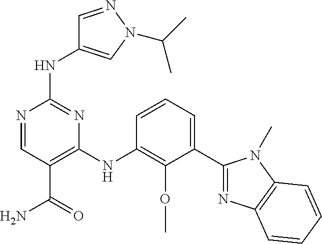 [SMILES: COc1c(Nc2nc(Nc3cnn(C(C)C)c3)ncc2C(N)=O)cccc1-c1nc2ccccc2n1C] | 2-[(1-isopropylpyrazol-4- yl)amino]-4-[2-methoxy-3-(1- methylbenzimidazol-2- yl)anilino]pyrimidine-5- carboxamide |
| 61 | 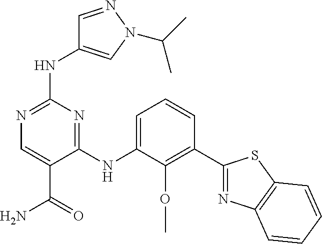 [SMILES: COc1c(Nc2nc(Nc3cnn(C(C)C)c3)ncc2C(N)=O)cccc1-c1nc2ccccc2s1] | 4-[3-(1,3-benzothiazol-2-yl)-2- methoxy-anilino]-2-[(1- isopropylpyrazol-4- yl)amino]pyrimidine-5- carboxamide |
| 62 | 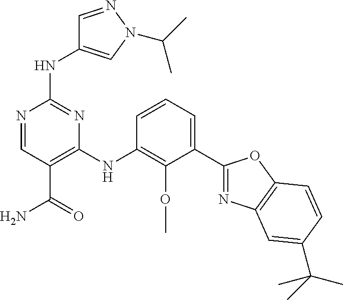 [SMILES: COc1c(Nc2nc(Nc3cnn(C(C)C)c3)ncc2C(N)=O)cccc1-c1nc2cc(C(C)(C)C)ccc2o1] | 4-[3-(5-tert-butyl-1,3- benzoxazol-2-yl)-2-methoxy- anilino]-2-[(1-isopropylpyrazol- 4-yl)amino]pyrimidine-5- carboxamide |
| 63 | 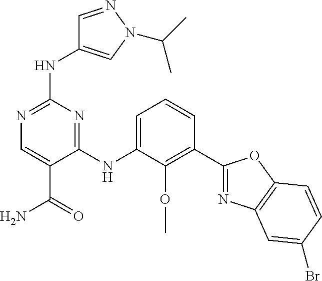 [SMILES: COc1c(Nc2nc(Nc3cnn(C(C)C)c3)ncc2C(N)=O)cccc1-c1nc2cc(Br)ccc2o1] | 4-[3-(5-bromo-1,3-benzoxazol- 2-yl)-2-methoxy-anilino]-2-[(1- isopropylpyrazol-4- yl)amino]pyrimidine-5- carboxamide |
| 64 | 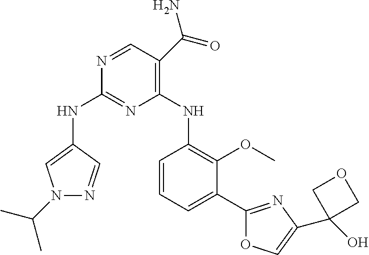 [SMILES: COc1c(Nc2nc(Nc3cnn(C(C)C)c3)ncc2C(N)=O)cccc1-c1nc(C2(O)COC2)co1] | 4-[3-[4-(3-hydroxyoxetan-3- yl)oxazol-2-yl]-2-methoxy- anilino]-2-[(1-isopropylpyrazol- 4-yl)amino]pyrimidine-5- carboxamide |
| 65 | 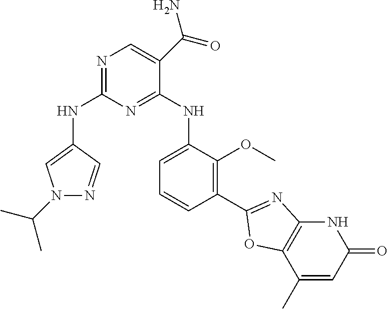 [SMILES: COc1c(Nc2nc(Nc3cnn(C(C)C)c3)ncc2C(N)=O)cccc1-c1nc2[nH]c(=O)cc(C)c2o1] | 2-[(1-isopropylpyrazol-4- yl)amino]-4-[2-methoxy-3-(7- methyl-5-oxo-4H-oxazolo[4,5- b]pyridin-2- yl)anilino]pyrimidine-5- carboxamide |
| 66 | 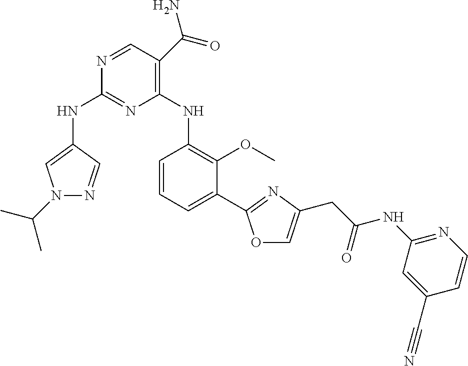 [SMILES: COc1c(Nc2nc(Nc3cnn(C(C)C)c3)ncc2C(N)=O)cccc1-c1nc(CC(=O)Nc2cc(C#N)ccn2)co1] | 4-[3-[4-[2-[(4-cyano-2- pyridyl)amino]-2-oxo- ethyl]oxazol-2-yl]-2-methoxy- anilino]-2-[(1-isopropylpyrazol- 4-yl)amino]pyrimidine-5- carboxamide |
| 67 | 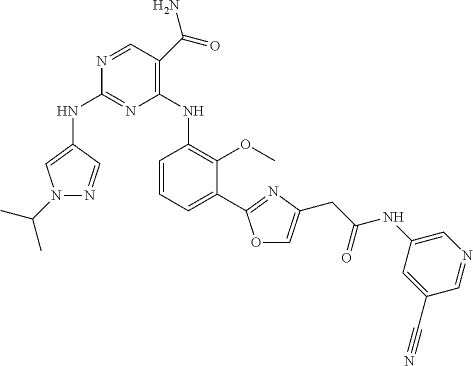 [SMILES: COc1c(Nc2nc(Nc3cnn(C(C)C)c3)ncc2C(N)=O)cccc1-c1nc(CC(=O)Nc2cncc(C#N)c2)co1] | 4-[3-[4-[2-[(5-cyano-3- pyridyl)amino]-2-oxo- ethyl]oxazol-2-yl]-2-methoxy- anilino]-2-[(1-isopropylpyrazol- 4-yl)amino]pyrimidine-5- carboxamide |
| 68 | 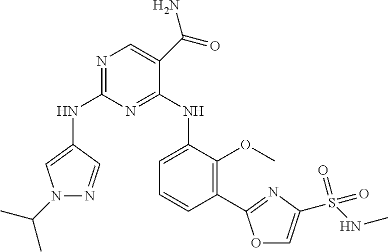 [SMILES: CNS(=O)(=O)c1coc(-c2cccc(Nc3nc(Nc4cnn(C(C)C)c4)ncc3C(N)=O)c2OC)n1] | 2-[(1-isopropylpyrazol-4- yl)amino]-4-[2-methoxy-3-[4- (methylsulfamoyl)oxazol-2- yl]anilino]pyrimidine-5- carboxamide |
| 69 | 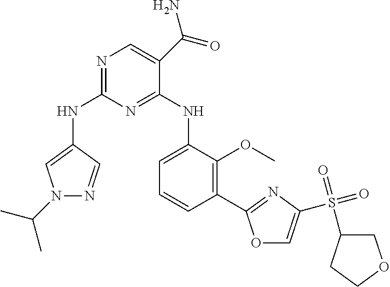 [SMILES: COc1c(Nc2nc(Nc3cnn(C(C)C)c3)ncc2C(N)=O)cccc1-c1nc(S(=O)(=O)C2CCOC2)co1] | 2-[(1-isopropylpyrazol-4- yl)amino]-4-[2-methoxy-3-(4- tetrahydrofuran-3- ylsulfonyloxazol-2- yl)anilino]pyrimidine-5- carboxamide |
| 70 | 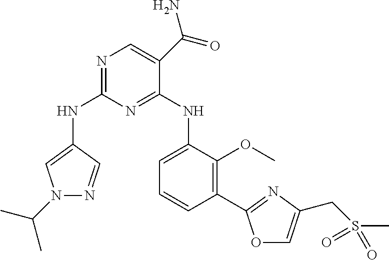 [SMILES: COc1c(Nc2nc(Nc3cnn(C(C)C)c3)ncc2C(N)=O)cccc1-c1nc(CS(C)(=O)=O)co1] | 2-[(1-isopropylpyrazol-4- yl)amino]-4-[2-methoxy-3-[4- (methylsulfonylmethyl)oxazol- 2-yl]anilino]pyrimidine-5- carboxamide |
| 71 | 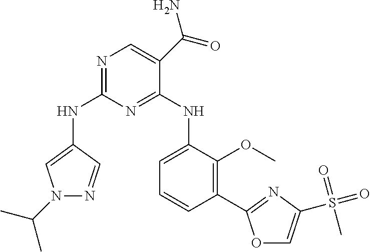 [SMILES: COc1c(Nc2nc(Nc3cnn(C(C)C)c3)ncc2C(N)=O)cccc1-c1nc(S(C)(=O)=O)co1] | 2-[(1-isopropylpyrazol-4- yl)amino]-4-[2-methoxy-3-(4- methylsulfonyloxazol-2- yl)anilino]pyrimidine-5- carboxamide |
| 72 | 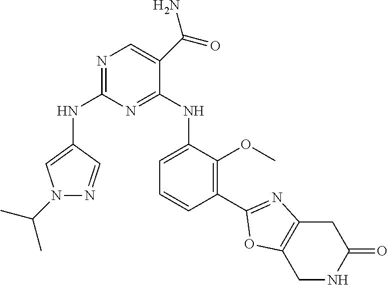 [SMILES: COc1c(Nc2nc(Nc3cnn(C(C)C)c3)ncc2C(N)=O)cccc1-c1nc2c(o1)CNC(=O)C2] | 2-[(1-isopropylpyrazol-4- yl)amino]-4-[2-methoxy-3-(6- oxo-5,7-dihydro-4H- oxazolo[5,4-c]pyridin-2- yl)anilino]pyrimidine-5- carboxamide |
| 73 | 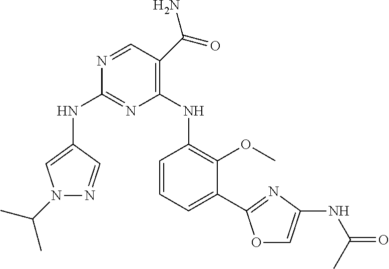 [SMILES: COc1c(Nc2nc(Nc3cnn(C(C)C)c3)ncc2C(N)=O)cccc1-c1nc(NC(C)=O)co1] | 4-[3-(4-acetamidooxazol-2-yl)- 2-methoxy-anilino]-2-[(1- isopropylpyrazol-4- yl)amino]pyrimidine-5- carboxamide |
| 74 | 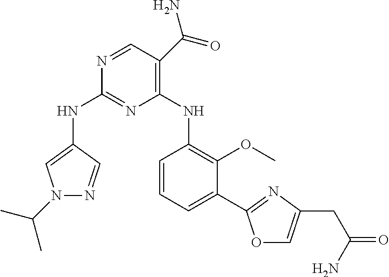 [SMILES: COc1c(Nc2nc(Nc3cnn(C(C)C)c3)ncc2C(N)=O)cccc1-c1nc(CC(N)=O)co1] | 4-[3-[4-(2-amino-2-oxo- ethyl)oxazol-2-yl]-2-methoxy- anilino]-2-[(1-isopropylpyrazol- 4-yl)amino]pyrimidine-5- carboxamide |
| 75 | 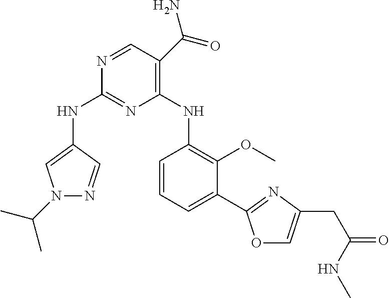 [SMILES: CNC(=O)Cc1coc(-c2cccc(Nc3nc(Nc4cnn(C(C)C)c4)ncc3C(N)=O)c2OC)n1] | 2-[(1-isopropylpyrazol-4- yl)amino]-4-[2-methoxy-3-[4- [2-(methylamino)-2-oxo- ethyl]oxazol-2- yl]anilino]pyrimidine-5- carboxamide |
| 76 | 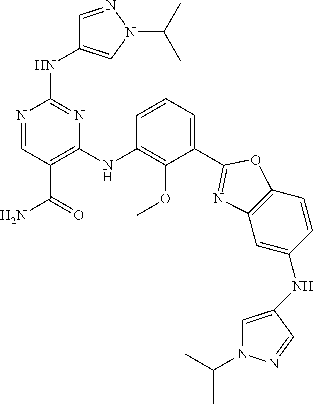 [SMILES: COc1c(Nc2nc(Nc3cnn(C(C)C)c3)ncc2C(N)=O)cccc1-c1nc2cc(Nc3cnn(C(C)C)c3)ccc2o1] | 2-[(1-isopropylpyrazol-4- yl)amino]-4-[3-[5-[(1- isopropylpyrazol-4-yl)amino]- 1,3-benzoxazol-2-yl]-2- methoxy-anilino]pyrimidine-5- carboxamide |

In some embodiments, the compound is a compound 16:

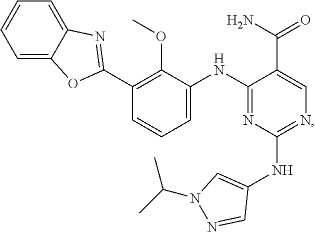 [SMILES: COc1c(Nc2nc(Nc3cnn(C(C)C)c3)ncc2C(N)=O)cccc1-c1nc2ccccc2o1]

or pharmaceutically acceptable salts, stereoisomers, solvates, prodrugs, isotopic derivatives, or tautomers thereof.

In some embodiments, the compound is 4-[3-(1,3-benzoxazol-2-yl)-2-methoxy-anilino]-2-[(1-isopropylpyrazol-4-yl)amino]pyrimidine-5-carboxamide, or pharmaceutically acceptable salts, stereoisomers, solvates, prodrugs, isotopic derivatives, or tautomers thereof.

In some embodiments, the compound is a neutral form (i.e., not a salt) of any one of the compounds described in Table 2.

In some embodiments, the compound is a pharmaceutically acceptable salt of any one of the compounds described in Table 2.

In some embodiments, the compound is a lithium salt, sodium salt, potassium salt, calcium salt, or magnesium salt of any one of the compounds described in Table 2.

In some embodiments, the compound is a sodium salt or potassium salt of any one of the compounds described in Table 2.

In some embodiments, the compound is a sodium salt of any one of the compounds described in Table 2.

In some embodiments, the compound is a potassium salt of any one of the compounds described in Table 2.

In some embodiments, the compound is a salt of acetic acid and any one of the compounds described in Table 2.

In some embodiments, the compound is a salt of adipic acid and any one of the compounds described in Table 2.

In some embodiments, the compound is a salt of ascorbic acid (L) and any one of the compounds described in Table 2.

In some embodiments, the compound is a salt of hydrobromic acid and any one of the compounds described in Table 2.

In some embodiments, the compound is a salt of hydrochloric acid and any one of the compounds described in Table 2.

In some embodiments, the compound is a salt of citric acid and any one of the compounds described in Table 2.

In some embodiments, the compound is a salt of glutamic acid and any one of the compounds described in Table 2.

In some embodiments, the compound is a salt of oxalic acid and any one of the compounds described in Table 2.

In some embodiments, the compound is a salt of formic acid and any one of the compounds described in Table 2.

In some embodiments, the compound is a salt of sulfuric acid and any one of the compounds described in Table 2.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 2 and prodrugs and pharmaceutically acceptable salts thereof.

In some embodiments, the compound is an isotopic derivative of any one of the compounds described in Table 2 and pharmaceutically acceptable salts thereof.

In some embodiments, the compound is an isotopic derivative of any one of prodrugs of the compounds described in Table 2 and pharmaceutically acceptable salts thereof.

In some embodiments, the compound is an isotopic derivative of any one of the compounds described in Table 2.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (I) is isotopically enriched with regard to, or labelled with, one or more isotopes as compared to the corresponding compound of Formula (I). In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled compound (i.e., being enriched with 2H with regard to one or more atoms thereof).

In some embodiments, the compound is a deuterium labeled compound of any one of the compounds described in Table 2 and prodrugs and pharmaceutically acceptable salts thereof.

In some embodiments, the compound is a deuterium labeled compound of any one of the compounds described in Table 2 and pharmaceutically acceptable salts thereof.

In some embodiments, the compound is a deuterium labeled compound of any one of the prodrugs of the compounds described in Table 2 and pharmaceutically acceptable salts thereof.

In some embodiments, the compound is a deuterium labeled compound of any one of the compounds described in Table 2.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the disclosure or a pharmaceutically acceptable salt or solvate thereof that contains the aforementioned deuterium atom(s) is within the scope of the disclosure. Further, substitution with deuterium (i.e., ²H) may afford certain therapeutic advantages resulting from greater metabolic stability, e.g., increased in vivo half-life or reduced dosage requirements.

In some embodiments, the compound is a ¹⁸F labeled compound.

In some embodiments, the compound is a ¹²³I labeled compound, a ¹²⁴I labeled compound, a ¹²⁵I labeled compound, a ¹²⁹I labeled compound, a ¹³¹I labeled compound, a ¹³⁵I labeled compound, or any combination thereof.

In some embodiments, the compound is a ³³S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵I, ¹²⁹I, ¹³¹I, ¹³⁵I, ³S, ³⁴S, ³⁵S, and/or ³⁶S labeled compound, can be prepared using any of a variety of art-recognized techniques. For example, the deuterium labeled compound can generally be prepared by carrying out the procedures disclosed in the Schemes and/or in the Examples described herein, by substituting a ¹⁸F, ¹²³I, ¹²⁴I, ¹²⁵I, ¹²⁹I, ¹³¹I, ¹³⁵I, ³S, ³⁴S, ³⁵S, and/or ³⁶S labeled reagent for a non-isotope labeled reagent.

A compound of the disclosure or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³S, ³⁴S, ³⁵S, and ³⁶S atom(s) is within the scope of the disclosure. Further, substitution with isotope (e.g., ¹⁸F, ¹²³I, ¹²⁴I, ¹²⁵I, ¹²⁹I, ¹³¹I, ¹³⁵I, ³S, ³⁴S, ³⁵S, and/or ³⁶S) may afford certain therapeutic advantages resulting from greater metabolic stability, e.g., increased in vivo half-life or reduced dosage requirements.

For the avoidance of doubt, it is to be understood that, where in this specification a group is qualified by “described herein”, the said group encompasses the first occurring and broadest definition as well as each and all of the particular definitions for that group.

The various functional groups and substituents making up the compounds of the Formula (I) are typically chosen such that the molecular weight of the compound does not exceed 1000 Daltons. More usually, the molecular weight of the compound will be less than 900, for example less than 800, or less than 750, or less than 700, or less than 650 Daltons. More conveniently, the molecular weight is less than 600 and, for example, is 550 Daltons or less.

A suitable pharmaceutically acceptable salt of a compound of the disclosure is, for example, an acid-addition salt of a compound of the disclosure, which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl) amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent, and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds of any one of the Formulae disclosed herein may exist in a number of different tautomeric forms and references to compounds of Formula (I) include all such forms. For the avoidance of doubt, where a compound can exist in one of several tautomeric forms, and only one is specifically described or shown, all others are nevertheless embraced by Formula (I) or (II). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

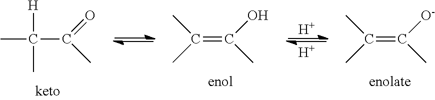

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a monohydrate, a di-hydrate, or a tri-hydrate. It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein containing an amine function may also form N-oxides. A reference herein to a compound of Formula (I) or (II) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl) piperazin-1-ylmethyl. Suitable pharmaceutically acceptable ether forming groups for a hydroxy group include a-acyloxyalkyl groups s acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl,morpholinomethyl,piperazin-1-ylmethyl and 4-(C₁-C₄ alkyl) piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

### Method of Synthesizing the Compounds

The compounds of the present invention may be made by a variety of methods, including standard chemistry. Suitable synthetic routes are depicted in the Schemes given below.

The compounds of Formula (I) may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of those skilled in the art will recognize if a stereocenter exists in the compounds of Formula (I). Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

### Preparation of Compounds

The compounds of the present invention can be prepared in a number of ways well known to those skilled in the art of organic synthesis. By way of example, compounds of the present invention can be synthesized using the methods described below, together with synthetic methods known in the art of synthetic organic chemistry, or variations thereon as appreciated by those skilled in the art. Suitable methods include but are not limited to those methods described below. Compounds of the present invention can be synthesized by following the steps outlined in General Procedure which comprise different sequences of assembling intermediates or compounds. Starting materials are either commercially available or made by known procedures in the reported literature or as illustrated below.

### GENERAL PROCEDURE

In general, the compound of the Formula (I) can be prepared using the sequence of the reaction presented below:

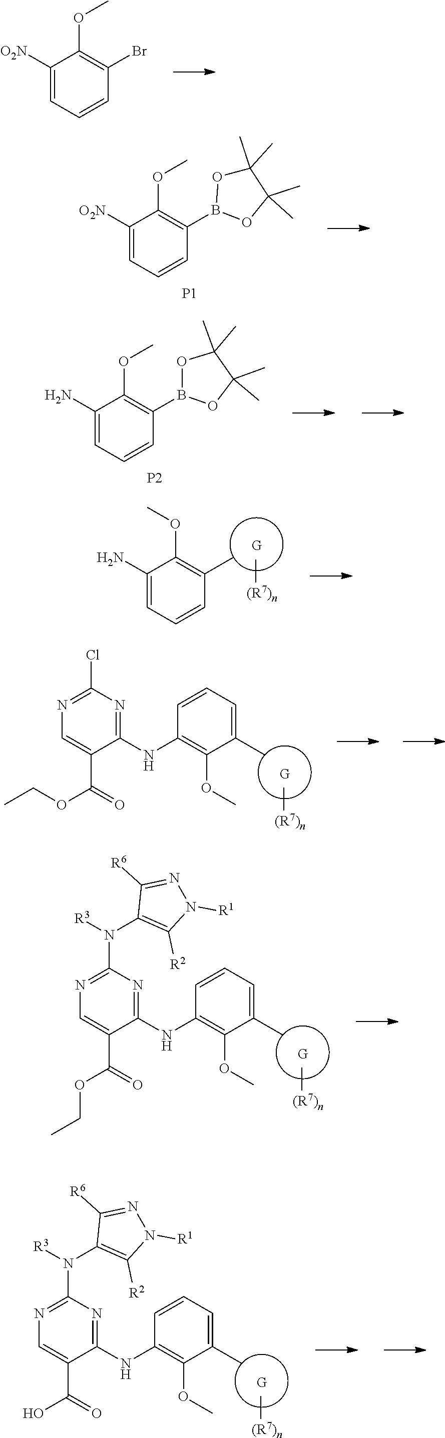

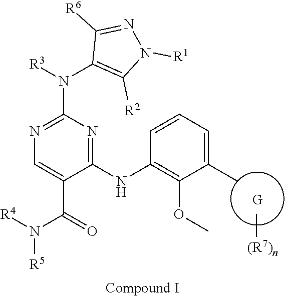

All reagents may be commercially available compounds itself or products of synthesis from commercially available reagents. For preparation of final compounds of the Formula (I) and intermediates may be used one step or multistep synthetic procedures, including but not limited procedures described herein in preparative part.

It should be obvious for specialist in this field that any of compound of Formula (I) obtained according to the procedures described above may be a subject for further transformation and modification that will led to obtain other compound of Formula (I).

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 2.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio) propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

In some embodiments, examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof. In some embodiments, the tonicity agent is selected from the group consisting of a glycol (such as propylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols-such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base-depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ¿-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

In some embodiments, a pharmaceutical composition described herein may further comprise one or more additional pharmaceutically active agents.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

A therapeutically effective amount of a compound of the present disclosure for use in therapy is an amount sufficient to treat or prevent a JAK2 related condition referred to herein, slow its progression and/or reduce the symptoms associated with the condition.

A therapeutically effective amount of a compound of the present disclosure for use in therapy is an amount sufficient to treat an JAK2 related condition referred to herein, slow its progression and/or reduce the symptoms associated with the condition.

The size of the dose for therapeutic or prophylactic purposes of a compound of Formula (I) will naturally vary according to the nature and severity of the conditions, the age and sex of the animal or subject and the route of administration, according to well-known principles of medicine.

### Methods of Use

In some aspects, the present disclosure provides a method of inhibiting JAK2 (e.g., in vitro or in vivo), comprising contacting a cell with a therapeutically effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some embodiments, the disease or disorder is associated with JAK2. In some embodiments, the disease or disorder is a disease or disorder in which JAK2 is implicated.

The compounds of the invention are inhibitors of JAK2. In some embodiments, the present invention is directed to a method of inhibiting JAK2 by contacting JAK2 with a compound of the invention. The contacting can be carried out in vitro or in vivo. In some embodiments, the compounds of the invention can bind to JAK2, thereby interfering with JAK2. In some embodiments, the present invention provides a method of inhibiting an activity of JAK2 by contacting JAK2 with a compound of the invention.

The compounds of the invention are also useful in treating diseases associated with JAK2. For example, diseases and conditions treatable according to the methods of the invention include polycythemia vera, essential thrombocytosis, thrombocythemia 3, primary myelofibrosis, chronic myelomonocytic leukemia, acute myeloid leukemia, myelodysplasia, acute myeloid leukemia, Budd-Chiari syndrome.

In some embodiments, the disease or disorder is selected from the group consisting of polycythemia vera; essential thrombocytosis; thrombocythemia 3; primary myelofibrosis; chronic myelomonocytic leukemia; acute myeloid leukemia; myelodysplasia; acute myeloid leukemia; Budd-Chiari syndrome; erythrocytosis, familial, 1 (ECYT1); myeloproliferative neoplasm (MPD); polycythemia; lymphoblastic leukemia, acute, with lymphomatous features (LALL); essential thrombocythemia (ET); premature menopause; thrombocytosis; hypereosinophilic syndrome (HES); splenomegaly; acute leukemia; thrombosis; portal hypertension; papilledema; acquired polycythemia; systemic mastocytosis (SMCD); chronic myelomonocytic leukemia (CMML); primary polycythemia; neutrophilia, hereditary (NEUTROPHILIA); thrombophilia; leukemia; hematologic cancer; myelophthisic anemia; erythroleukemia; myeloid leukemia; antithrombin III Deficiency (AT3D); severe congenital neutropenia; leukemia, chronic myeloid (CML); fibrosarcoma; mastocytosis; myelodysplastic syndrome (MDS); chronic eosinophilic leukemia; bone marrow cancer; behcet syndrome (BD); pancreatic adenocarcinoma; adenocarcinoma; stress polycythemia; B-cell 1 lymphoma; myelodysplastic/myeloproliferative neoplasm; hemangioblastoma; atypical chronic myeloid leukemia, Bcr-Abl1 negative (ACML); chronic neutrophilic leukemia (CNL); gastrointestinal stromal tumor (GIST); beta-thalassemia (B-THAL); acquired von Willebrand syndrome (AVWS); splenic infarction; B-lymphoblastic leukemia/lymphoma, Bcr-Abl1-like; lymphoma, Hodgkin, classic (CHL); Down syndrome; Wernicke encephalopathy; hepatic vascular disease; primary mediastinal B-Cell lymphoma; hyperglycemia; chronic leukemia (CLL); portal vein thrombosis; Diamond-Blackfan anemia (BDA); leptin deficiency or dysfunction (LEPD); deficiency anemia; hepatocellular carcinoma (HCC); Sm-Ahnmd; leukemia, acute lymphoblastic (ALL); sagittal sinus thrombosis; blood coagulation disease; acute erythroid leukemia; immunodeficiency 35 (IMD35); thrombocytopenia; colorectal cancer (CRC); blood platelet disease; temporal arteritis (GCA); amegakaryocytic thrombocytopenia, congenital (CAMT); inflammatory bowel disease; vein disease; erythromelalgia; erythrocytosis, familial, 2 (ECYT2); prostate cancer (PC); erythrocytosis, familial, 6 (ECYT6); aggressive systemic mastocytosis (ASM); myeloid and lymphoid neoplasms associated with pdgfra rearrangement; pancreatic cancer (PNCA); ovarian cancer (OC); juvenile myelomonocytic leukemia (JMML); breast cancer (BC); gastric cancer (GASC); medulloblastoma (MDB); lymphoma, non-Hodgkin, familial (NHL); myocardial infarction (MCI1); body mass index quantitative trait locus 11 (BMIQ11); acute salpingo-oophoritis; autism spectrum disorder (ASD); esophageal cancer (ESCR); leukemia, chronic lymphocytic (CLL); myeloma, multiple (MM); hypertension, essential (EHT); type 2 diabetes mellitus (T2D); skin disease; Rasopathy; connective tissue disease; peripheral nervous system disease; nervous system disease.

In some embodiments, the disease or disorder is a polycythemia vera.

In some embodiments, the disease or disorder is an essential thrombocytosis.

In some embodiments, the disease or disorder is a thrombocythemia 3.

In some embodiments, the disease or disorder is a primary myelofibrosis.

In some embodiments, the disease or disorder is a chronic myelomonocytic leukemia.

In some embodiments, the disease or disorder is an acute myeloid leukemia.

In some embodiments, the disease or disorder is a Budd-Chiari syndrome.

In some embodiments, the disease or disorder is erythrocytosis, familial, 1 (ECYT1).

In some embodiments, the disease or disorder is myeloproliferative neoplasm (MPD).

In some embodiments, the disease or disorder is polycythemia.

In some embodiments, the disease or disorder is lymphoblastic leukemia, acute, with lymphomatous features (LALL).

In some embodiments, the disease or disorder is essential thrombocythemia (ET).

In some embodiments, the disease or disorder is premature menopause.

In some embodiments, the disease or disorder is thrombocytosis.

In some embodiments, the disease or disorder is hypereosinophilic syndrome (HES).

In some embodiments, the disease or disorder is splenomegaly.

In some embodiments, the disease or disorder is acute leukemia.

In some embodiments, the disease or disorder is thrombosis.

In some embodiments, the disease or disorder is portal hypertension.

In some embodiments, the disease or disorder is papilledema.

In some embodiments, the disease or disorder is acquired polycythemia.

In some embodiments, the disease or disorder is systemic mastocytosis (SMCD).

In some embodiments, the disease or disorder is chronic myelomonocytic leukemia (CMML).

In some embodiments, the disease or disorder is primary polycythemia.

In some embodiments, the disease or disorder is neutrophilia, hereditary (NEUTROPHILIA).

In some embodiments, the disease or disorder is thrombophilia.

In some embodiments, the disease or disorder is leukemia.

In some embodiments, the disease or disorder is hematologic cancer.

In some embodiments, the disease or disorder is myelophthisic anemia.

In some embodiments, the disease or disorder is erythroleukemia.

In some embodiments, the disease or disorder is myeloid leukemia.

In some embodiments, the disease or disorder is antithrombin III Deficiency (AT3D).

In some embodiments, the disease or disorder is severe congenital neutropenia.

In some embodiments, the disease or disorder is leukemia, chronic myeloid (CML).

In some embodiments, the disease or disorder is fibrosarcoma.

In some embodiments, the disease or disorder is mastocytosis.

In some embodiments, the disease or disorder is myelodysplastic syndrome (MDS).

In some embodiments, the disease or disorder is chronic eosinophilic leukemia.

In some embodiments, the disease or disorder is bone marrow cancer.

In some embodiments, the disease or disorder is behcet syndrome (BD).

In some embodiments, the disease or disorder is pancreatic adenocarcinoma.

In some embodiments, the disease or disorder is adenocarcinoma.

In some embodiments, the disease or disorder is stress polycythemia.

In some embodiments, the disease or disorder is B-cell lymphoma.

In some embodiments, the disease or disorder is myelodysplastic/myeloproliferative neoplasm.

In some embodiments, the disease or disorder is hemangioblastoma.

In some embodiments, the disease or disorder is atypical chronic myeloid leukemia, Bcr-Abl1 negative (ACML).

In some embodiments, the disease or disorder is chronic neutrophilic leukemia (CNL).

In some embodiments, the disease or disorder is gastrointestinal stromal tumor (GIST).

In some embodiments, the disease or disorder is beta-thalassemia (B-THAL).

In some embodiments, the disease or disorder is acquired von Willebrand syndrome (AVWS).

In some embodiments, the disease or disorder is splenic infarction.

In some embodiments, the disease or disorder is B-lymphoblastic leukemia/lymphoma, Bcr-Abl1-like.

In some embodiments, the disease or disorder is lymphoma, Hodgkin, classic (CHL).

In some embodiments, the disease or disorder is Down syndrome.

In some embodiments, the disease or disorder is Wernicke encephalopathy.

In some embodiments, the disease or disorder is hepatic vascular disease.

In some embodiments, the disease or disorder is primary mediastinal B-Cell lymphoma.

In some embodiments, the disease or disorder is hyperglycemia.

In some embodiments, the disease or disorder is chronic leukemia (CLL).

In some embodiments, the disease or disorder is portal vein thrombosis.

In some embodiments, the disease or disorder is Diamond-Blackfan anemia (BDA).

In some embodiments, the disease or disorder is leptin deficiency or dysfunction (LEPD).

In some embodiments, the disease or disorder is deficiency anemia.

In some embodiments, the disease or disorder is hepatocellular carcinoma (HCC).

In some embodiments, the disease or disorder is Sm-Ahnmd; leukemia, acute lymphoblastic (ALL).

In some embodiments, the disease or disorder is sagittal sinus thrombosis.

In some embodiments, the disease or disorder is blood coagulation disease.

In some embodiments, the disease or disorder is acute erythroid leukemia.

In some embodiments, the disease or disorder is immunodeficiency 35 (IMD35).

In some embodiments, the disease or disorder is thrombocytopenia.

In some embodiments, the disease or disorder is colorectal cancer (CRC).

In some embodiments, the disease or disorder is blood platelet disease.

In some embodiments, the disease or disorder is temporal arteritis (GCA).

In some embodiments, the disease or disorder is amegakaryocytic thrombocytopenia, congenital (CAMT).

In some embodiments, the disease or disorder is inflammatory bowel disease.

In some embodiments, the disease or disorder is vein disease.

In some embodiments, the disease or disorder is erythromelalgia.

In some embodiments, the disease or disorder is erythrocytosis, familial, 2 (ECYT2).

In some embodiments, the disease or disorder is prostate cancer (PC).

In some embodiments, the disease or disorder is erythrocytosis, familial, 6 (ECYT6).

In some embodiments, the disease or disorder is aggressive systemic mastocytosis (ASM).

In some embodiments, the disease or disorder is myeloid and lymphoid neoplasms associated with pdgfra rearrangement.

In some embodiments, the disease or disorder is pancreatic cancer (PNCA).

In some embodiments, the disease or disorder is ovarian cancer (OC).

In some embodiments, the disease or disorder is juvenile myelomonocytic leukemia (JMML).

In some embodiments, the disease or disorder is breast cancer (BC).

In some embodiments, the disease or disorder is gastric cancer (GASC).

In some embodiments, the disease or disorder is medulloblastoma (MDB).

In some embodiments, the disease or disorder is lymphoma, non-Hodgkin, familial (NHL).

In some embodiments, the disease or disorder is myocardial infarction (MCI1).

In some embodiments, the disease or disorder is body mass index quantitative trait locus 11 (BMIQ11).

In some embodiments, the disease or disorder is acute salpingo-oophoritis; autism spectrum disorder (ASD).

In some embodiments, the disease or disorder is esophageal cancer (ESCR).

In some embodiments, the disease or disorder is leukemia, chronic lymphocytic (CLL).

In some embodiments, the disease or disorder is myeloma, multiple (MM).

In some embodiments, the disease or disorder is hypertension, essential (EHT).

In some embodiments, the disease or disorder is type 2 diabetes mellitus (T2D).

In some embodiments, the disease or disorder is skin disease.

In some embodiments, the disease or disorder is Rasopathy.

In some embodiments, the disease or disorder is connective tissue disease.

In some embodiments, the disease or disorder is peripheral nervous system disease.

In some embodiments, the disease or disorder is nervous system disease.

In some aspects, the present disclosure provides a method of treating or preventing a cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a leukemia in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a leukemia in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in inhibiting JAK2 (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing a cancer in a subject in need thereof.

In some aspects, the present disclosure provides a compound of the present disclosure or a pharmaceutically acceptable salt thereof for use in treating or preventing a leukemia in a subject in need thereof.

In some aspects, the present disclosure provides a compound of the present disclosure or a pharmaceutically acceptable salt thereof for use in treating a cancer in a subject in need thereof.

In some aspects, the present disclosure provides a compound of the present disclosure or a pharmaceutically acceptable salt thereof for use in treating a leukemia in a subject in need thereof.

In some aspects, the present disclosure provides use of a compound of the present disclosure or a pharmaceutically acceptable salt thereof in the manufacture of a medicament for inhibiting of JAK2 (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing a cancer in a subject in need thereof.

In some aspects, the present disclosure provides use of a compound of the present disclosure or a pharmaceutically acceptable salt thereof in the manufacture of a medicament for treating or preventing a leukemia in a subject in need thereof.

In some aspects, the present disclosure provides use of a compound of the present disclosure or a pharmaceutically acceptable salt thereof in the manufacture of a medicament for treating a cancer in a subject in need thereof.

In some aspects, the present disclosure provides use of a compound of the present disclosure or a pharmaceutically acceptable salt thereof in the manufacture of a medicament for treating a leukemia in a subject in need thereof.

The present disclosure provides compounds that function as inhibitors of JAK2 (e.g., in vitro or in vivo). The present disclosure therefore provides a method of JAK2 in vitro or in vivo, said method comprising contacting a cell with a therapeutically effective amount of a compound, or a pharmaceutically acceptable salt thereof, as defined herein.

In some embodiments, the JAK2 inhibitor is a compound of the present disclosure.

Effectiveness of compounds of the disclosure can be determined by industry-accepted assays/disease models according to standard practices of elucidating the same as described in the art and are found in the current general knowledge.

The present disclosure also provides a method of treating a disease or disorder in which JAK2 is implicated in a subject in need of such treatment, said method comprising administering to said subject a therapeutically effective amount of a compound, or a pharmaceutically acceptable salt thereof, or a pharmaceutical composition as defined herein.

In some embodiments, the subject is a mammal. In some embodiments, the subject is a human.

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

Abbreviations used in the following examples and elsewhere herein are:
ACN acetonitrile
AcOH acetic acid
br. broad
BSA bovine serum albumin
d duplet
DCM dichloromethane
DIPEA N,N-diisopropylethylamine
DMF N,N-dimethyl formamide
DMSO dimethyl sulfoxide
DTT dithiothreitol
FBS fetal bovine serum
h hour(s)
HPLC high pressure (or performance) liquid chromatography
LCMS liquid chromatography mass spectrometry
m multiplet
M molar
MHz megahertz
min minutes
NBS N-bromosuccinimide
NMR nuclear magnetic resonance
q quadruplet
rt room temperature
S singlet
t temperature, triplet
TFA trifluoroacetic acid
THF tetrahydrofuran
TRIS 2-amino-2-(hydroximethyl) propane-1,3-diol
Xantphos 4,5-Bis(diphenylphosphino)-9,9-dimethylxanthene

### EXAMPLES

### General synthetical procedures and examples of the compound's preparation

### Synthesis of Building Blocks

### Synthesis of 4-[(2-methoxy-3-pyridin-2-ylphenyl)amino]-2-[(1-methyl-1H-pyrazol-4-yl)amino]pyrimidine-5-carboxylic Acid (P6)

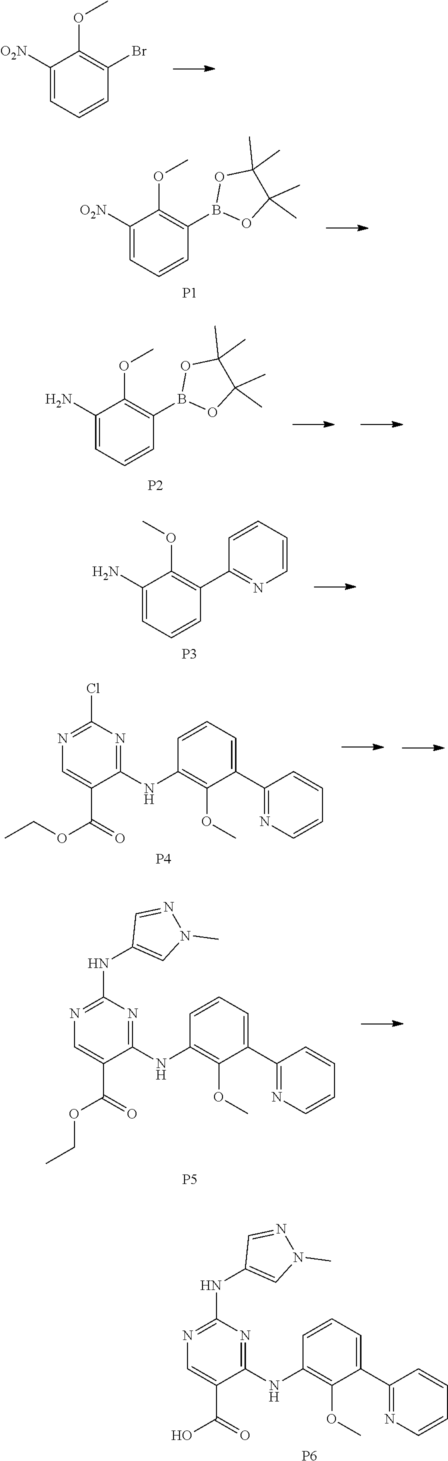

### Preparation 1. 2-(2-Methoxy-3-nitrophenyl)-4,4,5,5-tetramethyl-1,3,2-dioxaborolane (P1)

Under argon a mixture of 1-bromo-2-methoxy-3-nitro-benzene (11.1 g, 47.8 mmol), tetrakis(triphenylphosphine) palladium (0) (2.76 g, 2.4 mmol), potassium acetate (11.7 g, 119 mmol), 2,2′-oxybis(4,4,5,5-tetramethyl-1,3,2-dioxaborolane) in 1,4-dioxane (200 ml) was stirred at 100° C. overnight. Then the precipitate was filtered, the solvent was removed under reduced pressure, the residue was purified by column chromatography on silica gel (hexane/Et₂O 4:1) to provide the product P1 (6.3 g, 47% yield). ¹H NMR (400 MHz, DMSO-d₆), δ: 7.97 (dd, J=8.0, 1.2 Hz, 1H), 7.86 (dd, J=7.4, 1.2 Hz, 1H), 7.32 (t, J=7.7 Hz, 1H), 3.87 (s, 3H), 1.32 (s, 12H).

### Preparation 2. 2-Methoxy-3-(4,4,5,5-tetramethyl-1,3,2-dioxaborolan-2-yl)aniline (P2)

A mixture of P1 (6.3 g, 22.6 mmol) and 10% Pd/C (0.63 g, 0.1 mass.) in EtOH (100 ml) was stirred under flow of H₂ at rt for 12 h. Then Pd/C was filtered off, the solvent was removed under reduced pressure to provide the product P2 (5.4 g, 96% yield). ¹H NMR (400 MHz, DMSO-d₆), δ: 7.28-6.43 (m, 3H), 4.79 (s, 2H), 3.64 (s, 3H), 1.28 (s, 12H).

### Preparation 3. (2-Methoxy-3-pyridin-2-ylphenyl)amine (P3)

Under argon a mixture of P2 (3.5 g, 14 mmol), 2-chloropyridine (1.6 g, 14 mmol), tetrakis(triphenylphosphine) palladium (0) (0.81 g, 0.7 mmol) and cesium carbonate (11.4 g, 5 mmol) in 1,4-dioxane (100 ml) and water (30 ml) were stirred at 100° C. overnight. Then 300 ml of water was added, the product was extracted with DCM (2×100 ml), the solvents were removed under reduced pressure, the residue was purified by column chromatography on silica gel (hexane/Et₂O 2:1, 1:1) to provide the product P3 (2 g, 71% yield). ¹H NMR (400 MHz, DMSO-d₆), δ: 8.64 (d, J=4.1 Hz, 1H), 7.81 (ddd, J=17.6, 11.9, 4.8 Hz, 2H), 7.31 (ddd, J=7.2, 4.8, 1.4 Hz, 1H), 6.90 (t, J=7.7 Hz, 1H), 6.84 (dd, J=7.7, 1.8 Hz, 1H), 6.76 (dd, J=7.6, 1.8 Hz, 1H), 5.00 (s, 2H), 3.41 (s, 3H).

### Preparation 4. Ethyl 2-chloro-4-[(2-methoxy-3-pyridin-2-ylphenyl)amino]pyrimidine-5-carboxylate (P4)

A mixture of P3 (2 g, 10 mmol), ethyl 2,4-dichloropyrimidine-5-carboxylate (2.21 g, 10 mmol) and DIPEA (3.5 ml, 20 mmol) in acetonitrile (50 mL) was stirred at rt overnight. The solvent was removed under reduced pressure, the residue was dissolved in DCM (50 ml), the obtained solution was washed with water, dried over Na₂SO₄, the solvent was removed under reduced pressure. The residue was washed with Et₂O and dried to provide the product P4 (2.4 g, 62% yield). ¹H NMR (400 MHz, DMSO-d₆), δ: 11.02 (s, 1H), 8.86 (s, 1H), 8.72 (s, 1H), 8.46 (d, J=8.4 Hz, 1H), 8.00-7.80 (m, 2H), 7.48 (d, J=7.9 Hz, 1H), 7.44-7.37 (m, 1H), 7.33 (t, J=8.1 Hz, 1H), 4.40 (q, J=7.1 Hz, 2H), 3.52 (s, 3H), 1.36 (t, J=7.0 Hz, 3H).

### Preparation 5. Ethyl 4-[(2-methoxy-3-pyridin-2-ylphenyl)amino]-2-[(1-methyl-1H-pyrazol-4-yl)amino]pyrimidine-5-carboxylate (P5)

Under argon atmosphere a mixture of P4 (1.2 g, 3.12 mmol), 1-methyl-1H-pyrazol-4-amine (1.25 g, 9.36 mmol), palladium acetate (0.15 g, 0.668 mmol), Xantphos (0.45 g, 0.778 mmol) and cesium carbonate (1.52 g, 4.66 mmol) in dioxane (50 ml) was stirred at 100° C. overnight. The mixture was cooled and filtered through a pad of celite, the solvent from mother liquor was removed under reduced pressure. The residue was purified by column chromatography on silica gel (2%, 5%, 10% of MeOH in DCM) to provide the product P5 (0.39 g, 28% yield). LCMS (ESI) (C18 column 100×4.6 mm, 5.0 μm, pore size 100 Å, water-acetonitrile+0.1% TFA, gradient 5 to 87% for 3 min), m/z: 446.4 [M+H]⁺; Rt=1.16 min.

### Preparation 6. 4-[(2-Methoxy-3-pyridin-2-ylphenyl)amino]-2-[(1-methyl-1H-pyrazol-4-yl)amino]pyrimidine-5-carboxylic Acid (P6)

A solution of lithium hydroxide monohydrate (0.184 g, 4.38 mmol) in water (10 ml) was added to a suspension of P5 (0.39 g, 0.875 mmol) in THF (20 mL) at rt, the reaction mixture was stirred overnight, the solvents were removed under reduced pressure, the residue was dissolved in water (10 ml), the obtained solution was acidified to pH 5-6 with 10% HCl, the formed precipitate was filtered, washed with water and dried under reduced pressure to provide the product P6 (0.36 g, 99%). LCMS (ESI) (C18 column 100×4.6 mm, 5.0 μm, pore size 100 Å, water-acetonitrile+0.1% TFA, gradient 5 to 87% for 3 min), m/z: 418.8 [M+H]⁺; Rt=1.10 min.

### Synthesis of 4-{[2-methoxy-3-(5-methyl-1,3,4-oxadiazol-2-yl)phenyl]amino}-2-[(1-methyl-1H-pyrazol-4-yl)amino]pyrimidine-5-carboxylic Acid (P13)

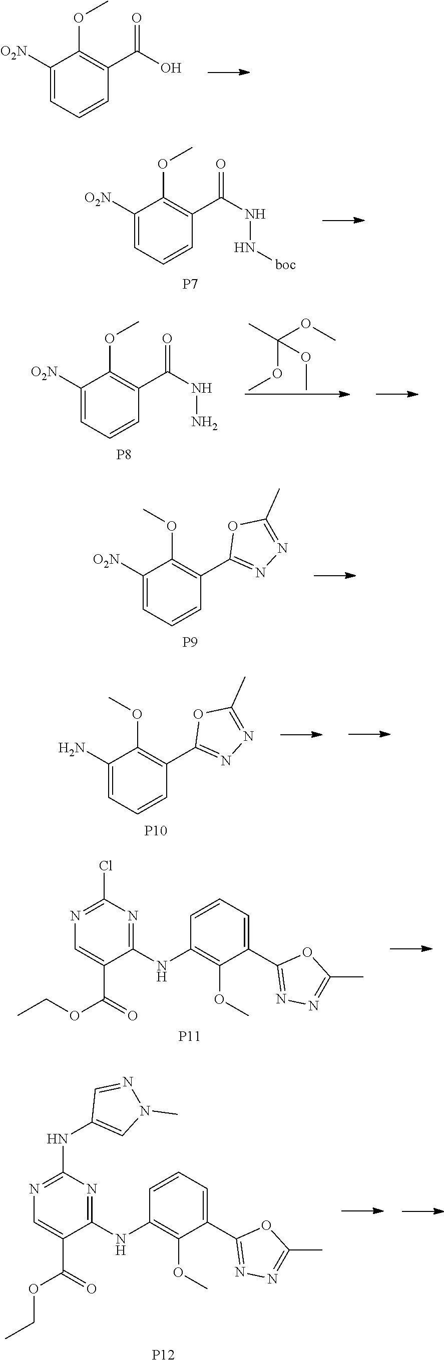

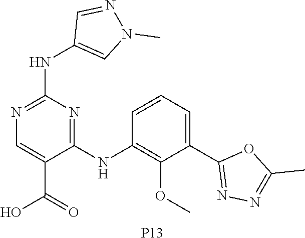

### Preparation 7. tert-Butyl 2-(2-methoxy-3-nitrobenzoyl)hydrazinecarboxylate (P7)

A mixture of 2-methoxy-3-nitro-benzoic acid (4.2 g, 21.3 mmol), tert-butyl hydrazinecarboxylate (2.814 g, 21.3 mmol), TBTU (10.2 g, 32 mmol) and DIPEA (5.7 ml, 32 mmol) in DCM (200 ml) was stirred at rt overnight. The solvent was removed under reduced pressure, the residue was purified by column chromatography on silica gel (hexane/Et₂O 1:1) to provide the product P7 (5.8 g, 78% yield). ¹H NMR (400 MHz, DMSO-d₆), δ: 10.16 (s, 1H), 9.05 (s, 1H), 8.00 (d, J=8.1 Hz, 1H), 7.67 (d, J=6.8 Hz, 1H), 7.39 (t, J=7.7 Hz, 1H), 3.89 (s, 3H), 1.43 (s, 9H).

### Preparation 8. 2-Methoxy-3-nitrobenzohydrazide (P8)

A solution of HCl in 1,4-dioxane (3M, 20 ml) was added to a solution of P7 (4.4 g, 14.1 mmol) in DCM/MeOH 1:1 (100 ml) at rt, the reaction mixture was stirred for 48 h. The formed precipitate was filtered, washed with Et₂O and dried on air to provide the product P8 (2.5 g, 84% yield). ¹H NMR (400 MHz, DMSO-d₆), δ: 11.60 (s, 1H), 8.09 (dd, J=8.1, 1.7 Hz, 1H), 7.80 (dd, J=7.7, 1.7 Hz, 1H), 7.44 (t, J=7.9 Hz, 1H), 3.88 (s, 3H).

### Preparation 9. 2-(2-methoxy-3-nitrophenyl)-5-methyl-1,3,4-oxadiazole (P9)

A mixture of P8 (2 g, 9.48 mmol), 1,1,1-trimethoxyethane (50 ml) and ammonium chloride (0.152 g, 2.84 mmol) was stirred at 150° C. overnight. Then the reaction mixture was concentrated under reduced pressure, the residue was purified by column chromatography on silica gel (hexane/EtOAc 2:1, 1:1) to provide the product P9 (0.943 g, 42% yield). ¹H NMR (400 MHz, DMSO-d₆), δ: 8.18 (dd, J=17.2, 8.0 Hz, 2H), 7.54 (t, J=8.0 Hz, 1H), 3.89 (s, 3H), 2.62 (s, 3H).

### Preparation 10. 2-Methoxy-3-(5-methyl-1,3,4-oxadiazol-2-yl)aniline (P10)

A mixture of P9 (1.075 g, 4.57 mmol) and 10% Pd/C (0.1 g, 0.1 mass.) in EtOH (50 ml) was stirred under flow of H₂ at rt for 12 h. Then Pd/C was filtered, the solvent was removed under reduced pressure to provide the product P10 (0.926 g, 99% yield). ¹H NMR (400 MHz, DMSO-d₆), δ: 6.99-6.95 (m, 2H), 6.93-6.88 (m, 1H), 5.26 (s, 2H), 5.26 (s, 2H), 3.69 (s, 3H), 2.57 (s, 3H).

### Preparation 11. Ethyl 2-chloro-4-{[2-methoxy-3-(5-methyl-1,3,4-oxadiazol-2-yl)phenyl]amino}pyrimidine-5-carboxylate (P11)

A mixture of P10 (0.926 g, 4.52 mmol), ethyl 2,4-dichloropyrimidine-5-carboxylate (0.998 g, 4.52 mmol) and DIPEA (1.6 ml, 9.04 mmol) in acetonitrile (50 mL) was stirred at rt overnight. The solvent was removed under reduced pressure, the residue was dissolved in DCM (50 ml), the obtained solution was washed with water, dried over Na₂SO₄, the solvent was removed under reduced pressure, the residue was washed with Et₂O and dried to provide the product P11 (1.365 g, 77% yield). ¹H NMR (400 MHz, DMSO-d₆), δ: 10.99 (s, 1H), 8.89 (s, 1H), 8.60 (d, J=8.2 Hz, 1H), 7.67 (d, J=7.9 Hz, 1H), 7.42 (t, J=8.1 Hz, 1H), 4.42 (q, J=7.0 Hz, 2H), 3.85 (s, 3H), 2.60 (s, 3H), 1.37 (t, J=7.1 Hz, 3H).

### Preparation 12. Ethyl 4-{[2-methoxy-3-(5-methyl-1,3,4-oxadiazol-2-yl)phenyl]amino}-2-[(1-methyl-1H-pyrazol-4-yl)amino]pyrimidine-5-carboxylate (P12)

Under argon a mixture of P11 (1 g, 2.57 mmol), 1-methyl-1H-pyrazol-4-amine (1.03 g, 7.74 mmol), palladium acetate (0.15 g, 0.66 mmol), Xantphos (0.45 g, 0.78 mmol) and cesium carbonate (0.514 g, 3.85 mmol) in 1,4-dioxane (50 ml) was stirred at 100° C. overnight. The mixture was cooled and filtered through a pad of celite, the solvent was removed under reduced pressure. The residue was purified by column chromatography on silica gel (2%, 5%, 10% of MeOH in DCM) to provide the product P12 (0.5 g, 43% yield). LCMS (ESI) (C18 column 100×4.6 mm, 5.0 μm, pore size 100 Å, water-acetonitrile+0.1% TFA, gradient 5 to 87% for 10 min), m/z: 451.0 [M+H]⁺; Rt=5.16 min.

### Preparation 13. 4-{[2-Methoxy-3-(5-methyl-1,3,4-oxadiazol-2-yl)phenyl]amino}-2-[(1-methyl-1H-pyrazol-4-yl)amino]pyrimidine-5-carboxylic Acid (P13)

A solution of lithium hydroxide monohydrate (0.14 g, 3.3 mmol) in water (10 ml) was added to a suspension of P12 (0.5 g, 1.1 mmol) in THF (25 mL) at rt, the reaction mass was stirred overnight, the solvents were removed under reduced pressure, the residue was dissolved in water (10 ml), the obtained solution was acidified to pH 5-6 with 10% HCl, the formed precipitate was filtered, washed with water and dried under reduced pressure to provide the product P13 (0.4 g, 87%). LCMS (ESI) (C18 column 100×4.6 mm, 5.0 μm, pore size 100 Å, water-acetonitrile+0.1% TFA, gradient 5 to 87% for 3 min), m/z: 423.4 [M+H]⁺; Rt=0.94 min.

### Synthesis of tert-butyl 4-{4-[(5-(aminocarbonyl)-4-{[3-(1,3-benzoxazol-2-yl)-2-methoxyphenyl]amino}pyrimidin-2-yl)amino]-1H-pyrazol-1-yl}piperidine-1-carboxylate (P18)

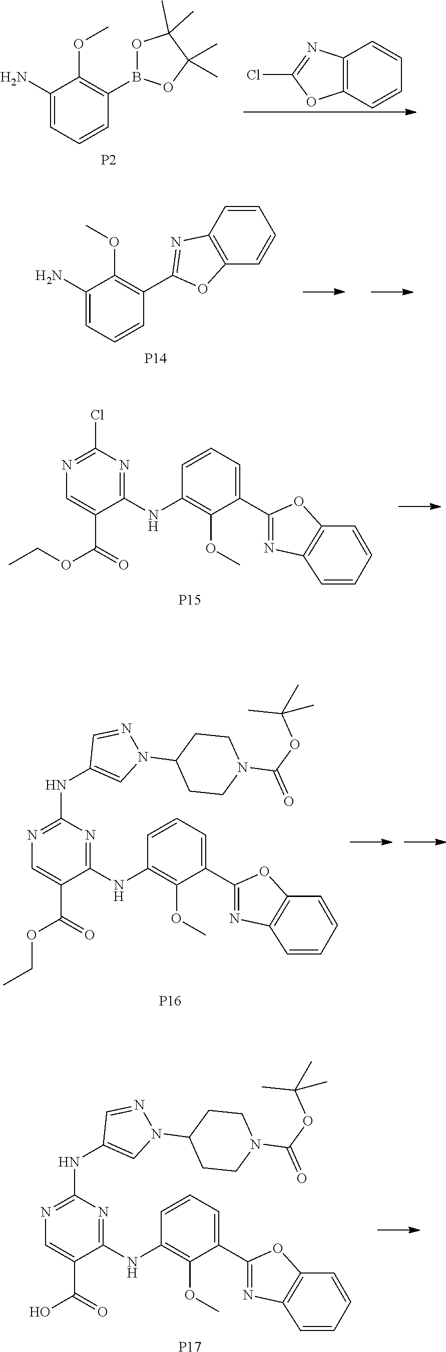

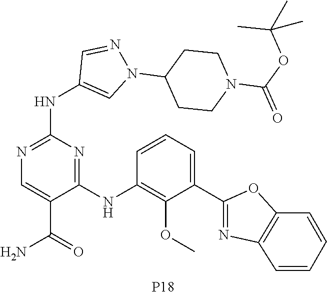

### Preparation 14. 3-(1,3-Benzoxazol-2-yl)-2-methoxyaniline (P14)

Under argon atmosphere a mixture of P2 (1 g, 4 mmol), 2-chloro-1,3-benzoxazole (0.614 g, 4 mmol), tetrakis(triphenylphosphine) palladium (0) (0.23 g, 0.2 mmol) and cesium carbonate (3.25 g, 119 mmol) in 1,4-dioxane (50 ml) and water (20 ml) was stirred at 100° C. overnight. Then 200 ml of water was added, the product was extracted with DCM (2×100 ml), the solvents from mother liquor were removed under reduced pressure, the residue was purified by column chromatography on silica gel (hexane/Et₂O 2:1, 1:1) to provide the product P14 (0.594 g, 62% yield). ¹H NMR (400 MHz, DMSO-d₆), δ: 7.84-7.74 (m, 2H), 7.46-7.37 (m, 2H), 7.23 (dd, J=7.7, 1.4 Hz, 1H), 7.01 (t, J=7.8 Hz, 1H), 6.94 (dd, J=7.9, 1.5 Hz, 1H), 5.26 (s, 2H), 3.78 (s, 3H).

### Preparation 15. Ethyl 4-{[3-(1,3-benzoxazol-2-yl)-2-methoxyphenyl]amino}-2-chloropyrimidine-5-carboxylate (P15)

A mixture of P14 (1.248 g, 5.2 mmol), ethyl 2,4-dichloropyrimidine-5-carboxylate (1.149 g, 5.2 mmol) and DIPEA (1.84 ml, 10.4 mmol) in acetonitrile (5 mL) was stirred at rt overnight. The solvent was removed under reduced pressure, the residue was solved in DCM (50 ml), the obtained solution was washed with water, dried over Na₂SO₄, the solvent was removed under reduced pressure, the residue was washed with Et₂O and dried to provide the product P15 (1.529 g, 69% yield). ¹H NMR (400 MHz, DMSO-d₆), δ: 11.04 (s, 1H), 8.89 (s, 1H), 8.62 (d, J=8.2 Hz, 1H), 7.93-7.78 (m, 3H), 7.46 (dt, J=13.0, 7.7 Hz, 3H), 4.43 (q, J=7.1 Hz, 2H), 3.92 (s, 3H), 1.38 (t, J=7.1 Hz, 3H).

### Preparation 16. Ethyl 4-{[3-(1,3-benzoxazol-2-yl)-2-methoxyphenyl]amino}-2-({1-[1-(tert-butoxycarbonyl) piperidin-4-yl]-1H-pyrazol-4-yl}amino)pyrimidine-5-carboxylate (P16)

Under argon a mixture of P15 (0.5 g, 1.18 mmol), tert-butyl 4-(4-amino-1H-pyrazol-1-yl)piperidine-1-carboxylate (0.941 g, 3.54 mmol), palladium acetate (0.08 g, 0.356 mmol), Xantphos (0.25 g, 0.432 mmol) and cesium carbonate (0.57 g, 1.77 mmol) in dioxane (25 ml) was stirred at 100° C. overnight. The mixture was cooled and filtered through a pad of celite, the solvent from mother liquor was removed under reduced pressure. The residue was purified by column chromatography on silica gel (2%, 5%, 10% of MeOH in DCM) to provide the product P16 (0.235 g, 30% yield). LCMS (ESI) (C18 column 100×4.6 mm, 5.0 μm, pore size 100 Å, water-acetonitrile+0.1% TFA, gradient 5 to 87% for 3 min), m/z: 655.7 [M+H]⁺; Rt=1.97 min.

### Preparation 17. 4-{[3-(1,3-Benzoxazol-2-yl)-2-methoxyphenyl]amino}-2-({1-[1-(tert-butoxycarbonyl) piperidin-4-yl]-1H-pyrazol-4-yl}amino)pyrimidine-5-carboxylic Acid (P17)

A solution of lithium hydroxide monohydrate (0.151 g, 3.6 mmol) in water (10 ml) was added to a suspension of P16 (0.235 g, 0.36 mmol) in THF (10 mL) at rt, the reaction mixture was stirred overnight, the solvents were removed under reduced pressure, the residue was dissolved in water (10 ml), the obtained solution was acidified to pH 6-7 with 3% HCl, the formed precipitate was filtered, washed with water and dried under reduced pressure to provide the product P17 (0.22 g, 99%). LCMS (ESI) (C18 column 100×4.6 mm, 5.0 μm, pore size 100 Å, water-acetonitrile+0.1% TFA, gradient 5 to 87% for 3 min), m/z: 627.7 [M+H]⁺; Rt=1.53 min.

### Preparation 18. tert-Butyl 4-{4-[(5-(aminocarbonyl)-4-{[3-(1,3-benzoxazol-2-yl)-2-methoxyphenyl]amino}pyrimidin-2-yl)amino]-1H-pyrazol-1-yl}piperidine-1-carboxylate (P18)

An ammonia solution (7N in MeOH, 3 ml), TBTU (0.173 g, 0.54 mmol) and DIPEA (0.2 ml, 1.08 mmol) were added to a solution of P17 (0.22 g, 0.36 mmol) in DMF (10 mL) at rt. The reaction mixture was stirred at rt overnight, then 50 ml of water was added, the product was extracted with DCM (3×30 ml), the combined organic layers were dried over Na₂SO₄, the solvents were removed under reduced pressure, the residue was washed with Et₂O and dried to provide the product P18 (0.17 g, 75% yield). LCMS (ESI) (C18 column 100×4.6 mm, 5.0 μm, pore size 100 Å, water-acetonitrile+0.1% TFA, gradient 5 to 87% for 3 min), m/z: 626.3 [M+H]⁺; Rt=1.48 min.

### Synthesis of 4-{[3-(1,3-benzoxazol-2-yl)-2-methoxyphenyl]amino}-2-({1-[2-(dimethylamino)ethyl]-1H-pyrazol-4-yl}amino)pyrimidine-5-carboxylic Acid (P20)

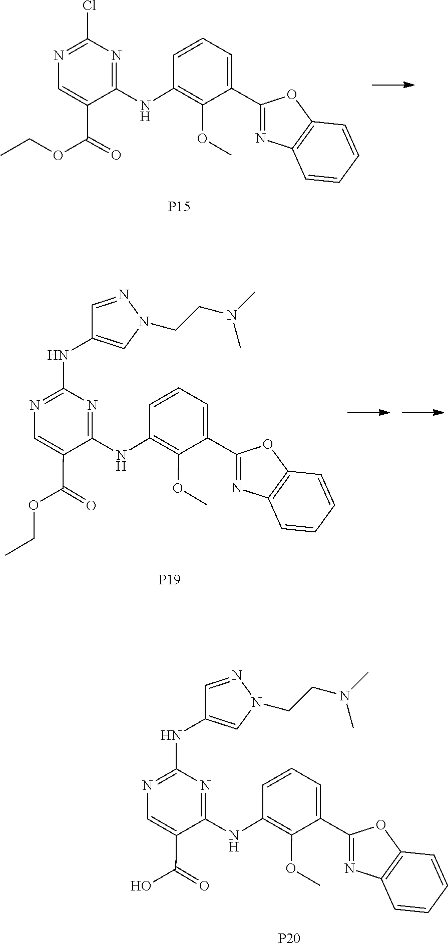

### Preparation 19. Ethyl 4-{[3-(1,3-benzoxazol-2-yl)-2-methoxyphenyl]amino}-2-({1-[2-(dimethylamino)ethyl]-1H-pyrazol-4-yl}amino)pyrimidine-5-carboxylate (P19)

Under argon a mixture of P15 (0.5 g, 1.18 mmol), 1-[2-(dimethylamino)ethyl]-1H-pyrazol-4-amine (0.545 g, 3.54 mmol), palladium acetate (0.08 g, 0.356 mmol), Xantphos (0.25 g, 0.432 mmol) and cesium carbonate (0.57 g, 1.77 mmol) in dioxane (25 ml) was stirred at 100° C. overnight. The mixture was cooled and filtered through a pad of celite, the solvent from mother liquor was removed under reduced pressure. The residue was purified by column chromatography on silica gel (2%, 5%, 10% of MeOH in DCM) to provide the product P19 (0.2 g, 31% yield). LCMS (ESI) (C18 column 100×4.6 mm, 5.0 μm, pore size 100 Å, water-acetonitrile+0.1% TFA, gradient 5 to 87% for 3 min), m/z: 543.8 [M+H]⁺; Rt=1.4 min.

### Preparation 20. 4-{[3-(1,3-Benzoxazol-2-yl)-2-methoxyphenyl]amino}-2-({1-[2-(dimethylamino)ethyl]-1H-pyrazol-4-yl}amino)pyrimidine-5-carboxylic Acid (P20)

A solution of lithium hydroxide monohydrate (0.151 g, 3.6 mmol) in water (10 ml) was added to a suspension of P19 (0.2 g, 0.369 mmol) in THF (10 mL) at rt, the reaction mass was stirred overnight, the solvents were removed under reduced pressure, the residue was dissolved in water (10 ml), the obtained solution was acidified to pH 5-6 with 10% HCl, the formed precipitate was filtered, washed with water and dried under reduced pressure to provide the product P20 (0.19 g, 99%). LCMS (ESI) (C18 column 100×4.6 mm, 5.0 μm, pore size 100 Å, water-acetonitrile+0.1% TFA, gradient 5 to 87% for 3 min), m/z: 515.8 [M+H]⁺; Rt=1.11 min.

### Synthesis of 4-(2-Methoxy-3-(1-methyl-1H-1,2,4-triazol-3-yl)phenylamino)-2-(1-(1-methylpiperidin-4-yl)-1H-pyrazol-4-ylamino)pyrimidine-5-carboxylic Acid (P23)

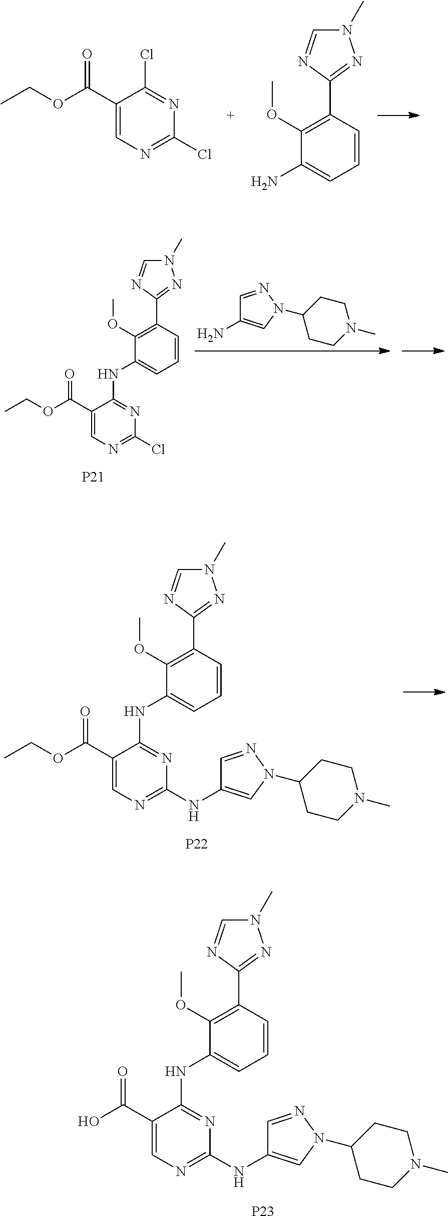

### Preparation 21. Ethyl 2-chloro-4-((2-methoxy-3-(1-methyl-1H-1,2,4-triazol-3-yl)phenyl)amino)pyrimidine-5-carboxylate (P21)

Ethyl 2,4-dichloropyrimidine-5-carboxylate (490 mg, 2.22 mmol) was dissolved in ACN (5 mL) and 2-methoxy-3-(1-methyl-1H-1,2,4-triazol-3-yl)aniline (450 mg, 2.20 mmol) and DIPEA (1.0 mL) were added at rt. After the reaction mixture was stirred for 3 h, the mixture was concentrated to give a residue. The residue was purified by silica gel column chromatography (EtOAc:DCM=3:2) to give P21 (785 mg, 92% yield) as a white solid. ¹H NMR (400 MHz, CDCl₃), δ: 11.22 (s, 1H), 8.85 (s, 1H), 8.67 (dd, J=8.0, 1.6 Hz, 1H), 8.12 (s, 1H), 7.76 (dd, J=8.0, 1.6 Hz, 1H), 7.27 (t, J=8.0 Hz, 1H), 4.44 (q, J=7.2 Hz, 2H), 4.02 (s, 3H), 3.87 (s, 3H), 1.42 (t, J=7.2 Hz, 3H).

### Preparation 22. Ethyl 4-(2-methoxy-3-(1-methyl-1H-1,2,4-triazol-3-yl)phenylamino)-2-(1-(1-methylpiperidin-4-yl)-1H-pyrazol-4-ylamino)pyrimidine-5-carboxylate (P22)

1-(1-methylpiperidin-4-yl)-1H-pyrazol-4-amine (600 mg, 3.3 mmol), cesium carbonate (390 mg, 1.2 mmol), Xantphos (23 mg, 0.04 mmol), and palladium acetate (9 mg, 0.04 mmol) were added to solution of P21 (150 mg, 0.38 mmol) in dioxane (40 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (DCM/MeOH 10:1) to give P22 (110 mg, 54% yield) as a white solid. ¹H NMR (400 MHz, DMSO-d₆), δ: 10.53 (br. s, 1H), 9.88 (s, 1H), 8.80 (d, J=6.8 Hz, 1H), 8.56 (s, 1H), 8.15 (d, 1H), 7.83 (s, 1H), 7.66-7.59 (m, 1H), 7.38 (s, 1H), 7.22 (t, J=8.0 Hz, 1H), 4.29 (q, J=7.2 Hz, 2H), 4.08-4.03 (m, 1H), 3.95 (s, 3H), 3.75 (s, 3H), 2.05-1.85 (m, 11H), 1.34 (t, J=7.2 Hz, 3H).

### Preparation 23. 4-(2-Methoxy-3-(1-methyl-1H-1,2,4-triazol-3-yl)phenylamino)-2-(1-(1-methylpiperidin-4-yl)-1H-pyrazol-4-ylamino)pyrimidine-5-carboxylic Acid (P23)

A solution of P22 (110 mg, 0.207 mmol) in THF (15 mL) was mixed with water (15 mL) and lithium hydroxide (86 mg, 2.07 mmol) at rt. The reaction mixture was stirred overnight, concentrated, and acidified with 1M HCl to pH=6. The resulting precipitate was centrifugated and dried to give P23 (107 mg, 99% yield) as a yellow solid. LCMS (ESI) (C18 column 20×2 mm, particle size 2.5 μm, pore size 100A, water-acetonitrile+0.1% TFA, gradient 5 to 87% for 3 min), m/z: 505.5 [M+H]⁺; Rt=0.75 min.

### Synthesis of 2-(1-Cyclohexyl-1H-pyrazol-4-ylamino)-4-(2-methoxy-3-(1-methyl-1H-1,2,4-triazol-3-yl)phenylamino)pyrimidine-5-carboxylic Acid (P25)

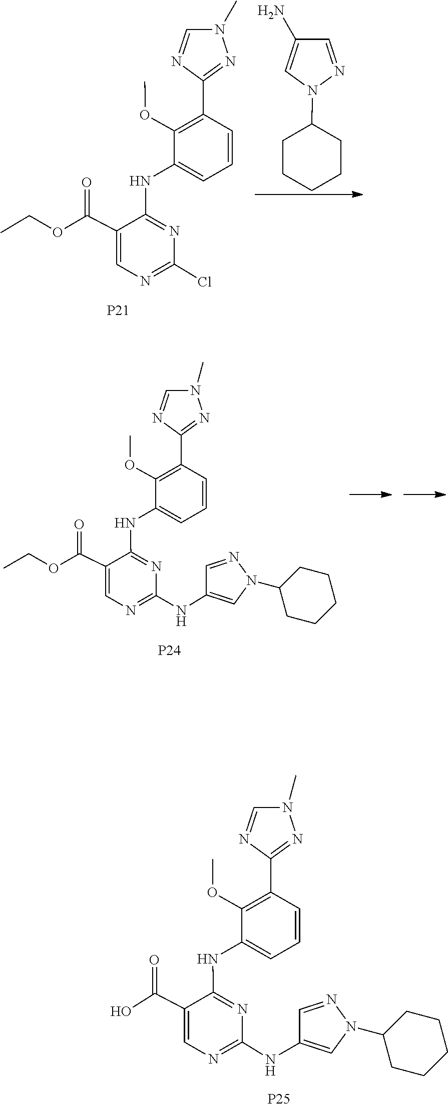

### Preparation 24. Ethyl 2-(1-cyclohexyl-1H-pyrazol-4-ylamino)-4-(2-methoxy-3-(1-methyl-1H-1,2,4-triazol-3-yl)phenylamino)pyrimidine-5-carboxylate (P24)

1-cyclohexyl-1H-pyrazol-4-amine (318 mg, 1.93 mmol), cesium carbonate (502 mg, 1.54 mmol), Xantphos (23 mg, 0.04 mmol), and palladium acetate (9 mg, 0.04 mmol) were added to solution of P21 (150 mg, 0.38 mmol) in dioxane (40 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (ethyl acetate/THF 50:50) to give P24 (56 mg, 28% yield) as a white solid. ¹H NMR (400 MHz, DMSO-d₆), δ: 10.50 (br. s, 1H), 9.84 (s, 1H), 8.70 (s, 1H), 8.56 (s, 1H), 8.13 (d, 1H), 7.69-7.52 (m, 1H), 7.39 (s, 1H), 7.23 (t, J=8.0 Hz, 1H), 4.30 (q, J=7.2 Hz, 2H), 4.16-4.09 (m, 1H), 3.95 (s, 3H), 3.78 (s, 3H), 2.20-2.10 (m, 2H), 1.93-1.88 (m, 2H), 1.77-1.66 (m, 6H), 1.32 (t, J=7.2 Hz, 3H).

### Preparation 25. 2-(1-Cyclohexyl-1H-pyrazol-4-ylamino)-4-(2-methoxy-3-(1-methyl-1H-1,2,4-triazol-3-yl)phenylamino)pyrimidine-5-carboxylic Acid (P25)

A solution of P24 (56 mg, 0.107 mmol) in THF (15 mL) was mixed with water (15 mL) and lithium hydroxide (27 mg, 0.65 mmol) at rt. The reaction mixture was stirred overnight, concentrated, and acidified with 1M HCl to pH=6. The resulting precipitate was centrifugated and dried to give P25 (44 mg, 83% yield) as a yellow solid. LCMS (ESI) (C18 column 100×4.6 mm, 5.0 μm, pore size 100 Å, water-acetonitrile+0.1% TFA, gradient 5 to 87% for 10 min), m/z: 490.8 [M+H]⁺; Rt=4.99 min.

### Synthesis of 4-((3-(1,3-benzoxazol-2-yl)-2-methoxyphenyl)amino)-2-((1-methyl-1H-pyrazol-4-yl)amino)pyrimidine-5-carboxylic Acid (P27)

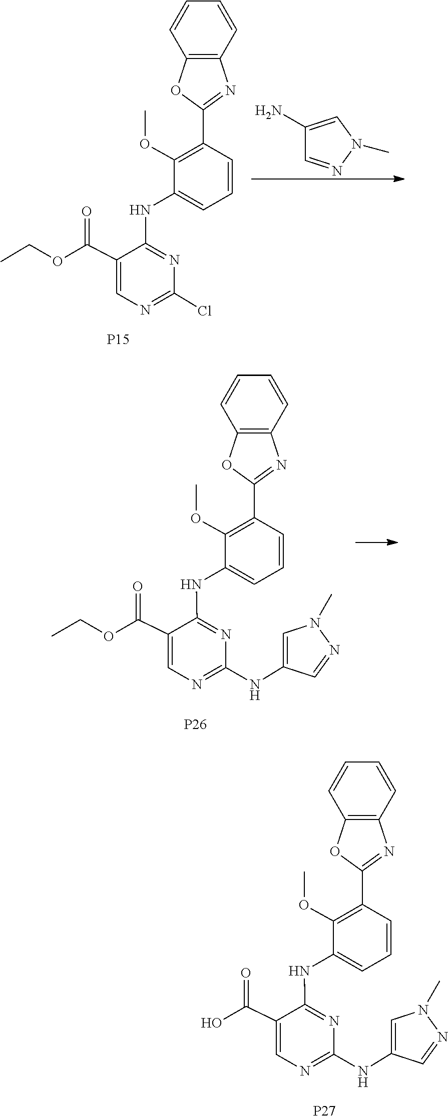

### Preparation 26. Ethyl 4-((3-(1,3-benzoxazol-2-yl)-2-methoxyphenyl)amino)-2-((1-methyl-1H-pyrazol-4-yl)amino)pyrimidine-5-carboxylate (P26)

1-methyl-1H-pyrazol-4-amine (100 mg, 1.03 mmol), cesium carbonate (300 mg, 0.92 mmol), Xantphos (27 mg, 0.04 mmol), and palladium acetate (10 mg, 0.04 mmol) were added to solution of P15 (200 mg, 0.47 mmol) in dioxane (10 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (ethyl acetate/THF 50:50) to give P26 (140 mg, 61% yield) as a white solid. LCMS (ESI) (C18 column 100×4.6 mm, 5.0 μm, pore size 100 Å, water-acetonitrile+0.1% TFA, gradient 5 to 87% for 3 min), m/z: 486.5 [M+H]⁺; Rt=1.70 min.

### Preparation 27. 4-((3-(1,3-Benzoxazol-2-yl)-2-methoxyphenyl)amino)-2-((1-methyl-1H-pyrazol-4-yl)amino)pyrimidine-5-carboxylic Acid (P27)

A solution of P26 (142 mg, 0.29 mmol) in THF (15 mL) was mixed with water (15 mL) and lithium hydroxide (70 mg, 1.7 mmol) at rt. The reaction mixture was stirred overnight, concentrated, and acidified with 1M HCl to pH=6. The resulting precipitate was centrifugated and dried to give P27 (130 mg, 97% yield) as a yellow solid. LCMS (ESI) (C18 column 100×4.6 mm, 5.0 μm, pore size 100 Å, water-acetonitrile+0.1% TFA, gradient 5 to 87% for 3 min), m/z: 458.9 [M+H]⁺; Rt=1.31 min.

### Synthesis of 2-(1,3-Dimethyl-1H-pyrazol-4-ylamino)-4-(2-methoxy-3-(1-methyl-1H-1,2,4-triazol-3-yl)phenylamino)pyrimidine-5-carboxylic Acid (P29)

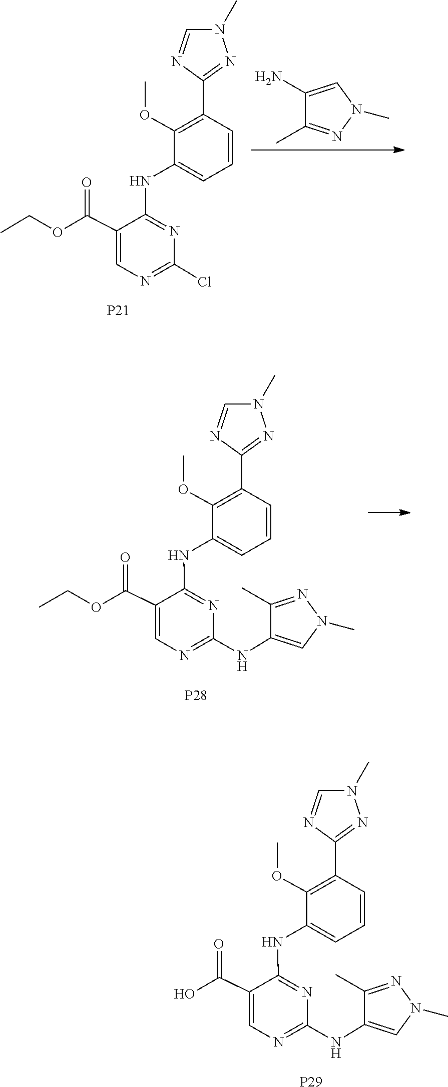

### Preparation 28. Ethyl 2-(1,3-dimethyl-1H-pyrazol-4-ylamino)-4-(2-methoxy-3-(1-methyl-1H-1,2,4-triazol-3-yl)phenylamino)pyrimidine-5-carboxylate (P28)

1,3-dimethyl-1H-pyrazol-4-amine (900 mg, 8.1 mmol), cesium carbonate (600 mg, 1.85 mmol), Xantphos (44 mg, 0.07 mmol), and palladium acetate (16 mg, 0.07 mmol) were added to solution of P21 (300 mg, 0.77 mmol) in dioxane (50 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (ethyl acetate/THF 50:50) to give P28 (70 mg, 19% yield) as a white solid. ¹H NMR (400 MHz, CDCl₃), δ: 10.78 (br. s, 1H), 8.78 (d, J=6.8 Hz, 1H), 8.25 (br. s, 1H), 8.12 (s, 1H), 7.75 (d, J=7.1 Hz 1H), 7.65 (s, 1H), 7.14 (t, J=7.0 Hz, 1H), 6.88 (s, 1H), 4.38 (q, J=7.2 Hz, 2H), 4.03 (s, 3H), 3.86 (s, 3H), 3.79 (s, 3H), 1.63 (s, 3H), 1.41 (t, J=7.2 Hz, 3H).

### Preparation 29. 2-(1,3-Dimethyl-1H-pyrazol-4-ylamino)-4-(2-methoxy-3-(1-methyl-1H-1,2,4-triazol-3-yl)phenylamino)pyrimidine-5-carboxylic acid (P29)

A solution of P28 (70 mg, 0.15 mmol) in THF (15 mL) was mixed with water (15 mL) and lithium hydroxide (63 mg, 1.51 mmol) at rt. The reaction mixture was stirred overnight, concentrated, and acidified with 1M HCl to pH=6. The resulting precipitate was centrifugated and dried to give P29 (66 mg, 97% yield) as a yellow solid. LCMS (ESI) (C18 column 20×2 mm, particle size 2.5 μm, pore size 100A, water-acetonitrile+0.1% TFA, gradient 5 to 87% for 3 min), m/z: 436.4 [M+H]⁺; Rt=1.11 min.

### Synthesis of 4-(2,7-diazaspiro[3.5]non-2-yl)-6-(2,2,2-trifluoroethyl)pyrido[2,3-d]pyrimidine (P31)

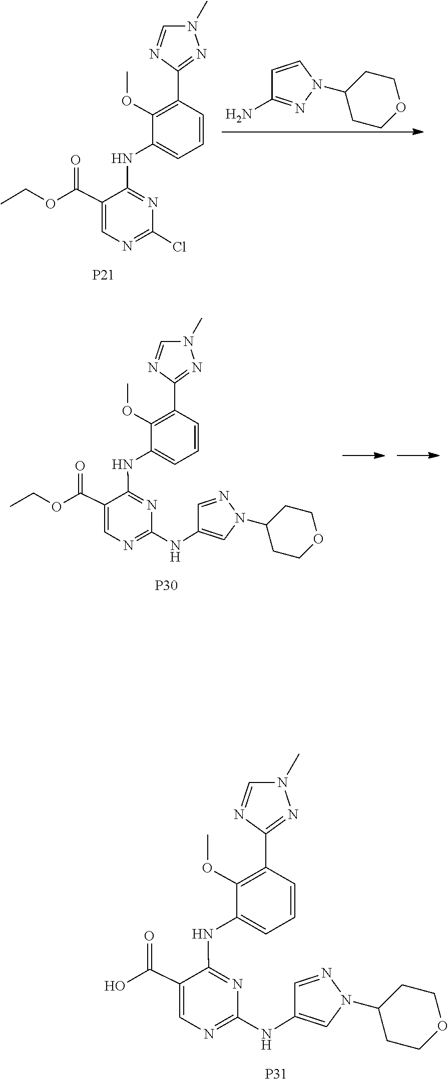

### Preparation 30. Ethyl 4-(2-methoxy-3-(1-methyl-1H-1,2,4-triazol-3-yl)phenylamino)-2-(1-(tetrahydro-2H-pyran-4-yl)-1H-pyrazol-4-ylamino)pyrimidine-5-carboxylate (P30)

1-(Tetrahydro-2H-pyran-4-yl)-1H-pyrazol-4-amine (1291 mg, 7.7 mmol), cesium carbonate (1000 mg, 3.07 mmol), Xantphos (70 mg, 0.07 mmol), and palladium acetate (17 mg, 0.07 mmol) were added to solution of P21 (300 mg, 0.77 mmol) in dioxane (40 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (ethyl acetate/THF 50:50) to give P30 (144 mg, 36% yield) as a white solid. ¹H NMR (400 MHz, DMSO-d₆), δ: 10.60 (br. s, 1H), 9.89 (s, 1H), 8.70 (s, 1H), 8.55 (s, 1H), 8.20 (d, J=Hz, 1H), 7.68-7.66 (m, 1H), 7.55 (s, 1H), 7.43 (s, 1H), 7.22 (t, J=8.0 Hz, 1H), 4.35 (q, J=7.2 Hz, 2H), 3.92-3.88 (m, 5H), 3.78 (s, 3H), 3.48-3.39 (m, 3H), 1.99-1.93 (m, 2H), 1.85-1.75 (m, 2H), 1.32 (t, J=7.2 Hz, 3H).

### Preparation 31. 4-(2-Methoxy-3-(1-methyl-1H-1,2,4-triazol-3-yl)phenylamino)-2-(1-(tetrahydro-2H-pyran-4-yl)-1H-pyrazol-4-ylamino)pyrimidine-5-carboxylic Acid (P31)

A solution of P30 (144 mg, 0.257 mmol) in THF (15 mL) was mixed with water (15 mL) and lithium hydroxide (108 mg, 2.57 mmol) at rt. The reaction mixture was stirred overnight, concentrated, and acidified with 1M HCl to pH=6. The resulting precipitate was centrifugated and dried to give P31 (62 mg, 51% yield) as a yellow solid. LCMS (ESI) (C18 column 100×4.6 mm, 5.0 μm, pore size 100 Å, water-acetonitrile+0.1% TFA, gradient 5 to 87% for 10 min), m/z: 492.2 [M+H]⁺; Rt=4.28 min.

### Synthesis of 4-{[3-(1,3-benzoxazol-2-yl)-2-methoxyphenyl]amino}-2-[(1-isopropyl-1H-pyrazol-4-yl)amino]pyrimidine-5-carboxylic Acid (P33)

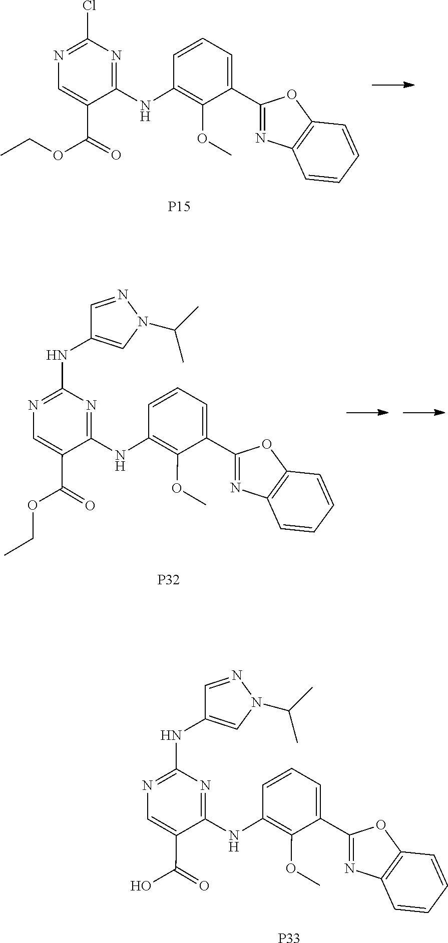

### Preparation 32. Ethyl 4-{[3-(1,3-benzoxazol-2-yl)-2-methoxyphenyl]amino}-2-[(1-isopropyl-1H-pyrazol-4-yl)amino]pyrimidine-5-carboxylate (P32)

Under argon a mixture of P15 (0.5 g, 1.18 mmol), 1-isopropyl-1H-pyrazol-4-amine (0.442 g, 3.54 mmol), palladium acetate (0.08 g, 0.356 mmol), Xantphos (0.25 g, 0.432 mmol) and cesium carbonate (0.54 g, 1.77 mmol) in dioxane (25 ml) was stirred at 100° C. overnight. The mixture was cooled and filtered through a pad of celite, the solvent from mother liquor was removed under reduced pressure. The residue was purified by column chromatography on silica gel (2%, 5%, 10% of MeOH in DCM) to provide the product P32 (0.23 g, 38% yield). LCMS (ESI) (C18 column 100×4.6 mm, 5.0 μm, pore size 100 Å, water-acetonitrile+0.1% TFA, gradient 5 to 87% for 3 min), m/z: 514.5 [M+H]⁺; Rt=1.78 min.

### Preparation 33. 4-{[3-(1,3-benzoxazol-2-yl)-2-methoxyphenyl]amino}-2-[(1-isopropyl-1H-pyrazol-4-yl)amino]pyrimidine-5-carboxylic Acid (P33)

A solution of lithium hydroxide monohydrate (0.223 g, 4.48 mmol) in water (10 ml) was added to a suspension of P32 (0.23 g, 0.448 mmol) in THF (10 mL) at rt, the reaction mass was stirred overnight, the solvents were removed under reduced pressure, the residue was dissolved in water (10 ml), the obtained solution was acidified to pH 5-6 with 10% HCl, the formed precipitate was filtered, washed with water and dried under reduced pressure to provide the product P33 (0.2 g, 92%). LCMS (ESI) (C18 column 100×4.6 mm, 5.0 μm, pore size 100 Å, water-acetonitrile+0.1% TFA, gradient 5 to 87% for 3 min), m/z: 486.5 [M+H]⁺; Rt=1.39 min.

### Synthesis of 4-(2-methoxy-3-(1-methyl-1H-1,2,4-triazol-3-yl)phenylamino)-2-(1,3,5-trimethyl-1H-pyrazol-4-ylamino)pyrimidine-5-carboxylic Acid (P35)

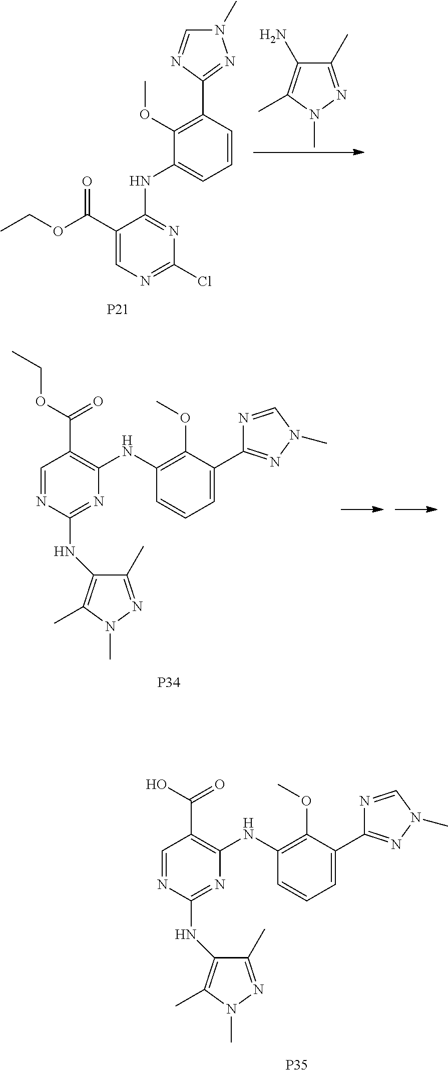

### Preparation 34. Ethyl 4-(2-methoxy-3-(1-methyl-1H-1,2,4-triazol-3-yl)phenylamino)-2-(1,3,5-trimethyl-1H-pyrazol-4-ylamino)pyrimidine-5-carboxylate (P34)

1,3,5-trimethyl-1H-pyrazol-4-amine (483 mg, 3.86 mmol), cesium carbonate (390 mg, 2.5 mmol), Xantphos (23 mg, 0.04 mmol), and palladium acetate (9 mg, 0.04 mmol) were added to solution of P21 (150 mg, 0.386 mmol) in dioxane (40 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (ethyl acetate/THF 2:1) to give P34 (150 mg, 81% yield) as a white solid. ¹H NMR (400 MHz, DMSO-d₆), δ: 10.91 (br. s, 1H), 9.06 (s, 1H), 8.70 (s, 1H), 8.54 (s, 1H), 8.21 (d, J=8.0 Hz, 1H), 7.45 (d, J=6.5 Hz, 1H), 6.86 (t, J=8.0 Hz, 1H), 4.30 (q, J=7.2 Hz, 2H), 3.93 (s, 3H), 3.76 (s, 3H), 3.50 (s, 3H), 2.04 ((s, 3H), 1.95 (s, 3H), 1.32 (t, J=7.2 Hz, 3H).

### Preparation 35. 4-(2-Methoxy-3-(1-methyl-1H-1,2,4-triazol-3-yl)phenylamino)-2-(1,3,5-trimethyl-1H-pyrazol-4-ylamino)pyrimidine-5-carboxylic Acid (P35)

A solution of P34 (150 mg, 0.314 mmol) in THF (15 mL) was mixed with water (15 mL) and lithium hydroxide (132 mg, 3.14 mmol) at rt. The reaction mixture was stirred overnight, concentrated, and acidified with 1M HCl to pH=6. The resulting precipitate was centrifugated and dried to give P35 (140 mg, 98% yield) as a yellow solid. LCMS (ESI) (C18 column 100×4.6 mm, 5.0 μm, pore size 100 Å, water-acetonitrile+0.1% TFA, gradient 5 to 87% for 10 min), m/z: 450.2 [M+H]⁺; Rt=4.14 min.

### Synthesis of 2-(1-ethyl-3,5-dimethyl-1H-pyrazol-4-ylamino)-4-(2-methoxy-3-(1-methyl-1H-1,2,4-triazol-3-yl)phenylamino)pyrimidine-5-carboxylic Acid (P37)

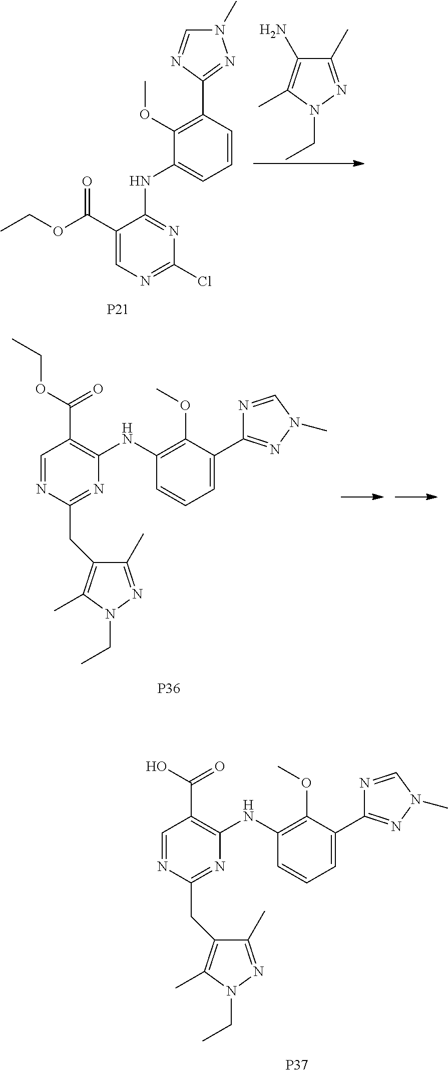

### Preparation 36. Ethyl 2-(1-ethyl-3,5-dimethyl-1H-pyrazol-4-ylamino)-4-(2-methoxy-3-(1-methyl-1H-1,2,4-triazol-3-yl)phenylamino)pyrimidine-5-carboxylate (P36)

1-Ethyl-3,5-dimethyl-1H-pyrazol-4-amine (514 mg, 3.70 mmol), cesium carbonate (812 mg, 2.5 mmol), Xantphos (35 mg, 0.06 mmol), and palladium acetate (13 mg, 0.06 mmol) were added to solution of P21 (150 mg, 0.386 mmol) in dioxane (40 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (ethyl acetate/THF 1:1) to give P36 (206 mg, 65% yield) as a white solid. ¹H NMR (400 MHz, DMSO-d₆), δ: 10.93 (br. s, 1H), 9.04 (s, 1H), 8.71 (s, 1H), 8.54 (s, 1H), 8.23 (d, J=8.0 Hz, 1H), 7.43 (d, J=6.5 Hz, 1H), 6.83 (t, J=8.0 Hz, 1H), 4.31 (q, J=7.2 Hz, 2H), 3.94 (s, 3H), 3.84 (q, J=7.2 Hz, 2H), 3.77 (s, 3H), 2.05 (s, 3H), 1.96 (s, 3H), 1.32 (t, J=7.2 Hz, 3H), 1.24 (t, J=7.2 Hz, 3H).

### Preparation 37. 2-(1-Ethyl-3,5-dimethyl-1H-pyrazol-4-ylamino)-4-(2-methoxy-3-(1-methyl-1H-1,2,4-triazol-3-yl)phenylamino)pyrimidine-5-carboxylic Acid (P37)

A solution of P36 (200 mg, 0.40 mmol) in THF (15 mL) was mixed with water (15 mL) and lithium hydroxide (132 mg, 3.14 mmol) at rt. The reaction mixture was stirred overnight, concentrated, and acidified with 1M HCl to pH=6. The resulting precipitate was centrifugated and dried to give P37 (100 mg, 54% yield) as a yellow solid. LCMS (ESI) (C18 column 100×4.6 mm, 5.0 μm, pore size 100 Å, water-acetonitrile+0.1% TFA, gradient 5 to 87% for 10 min), m/z: 464.5 [M+H]⁺; Rt=4.30 min.

### Synthesis of 2-(3,5-dimethyl-1-propyl-1H-pyrazol-4-ylamino)-4-(2-methoxy-3-(1-methyl-1H-1,2,4-triazol-3-yl)phenylamino)pyrimidine-5-carboxylic Acid (P39)

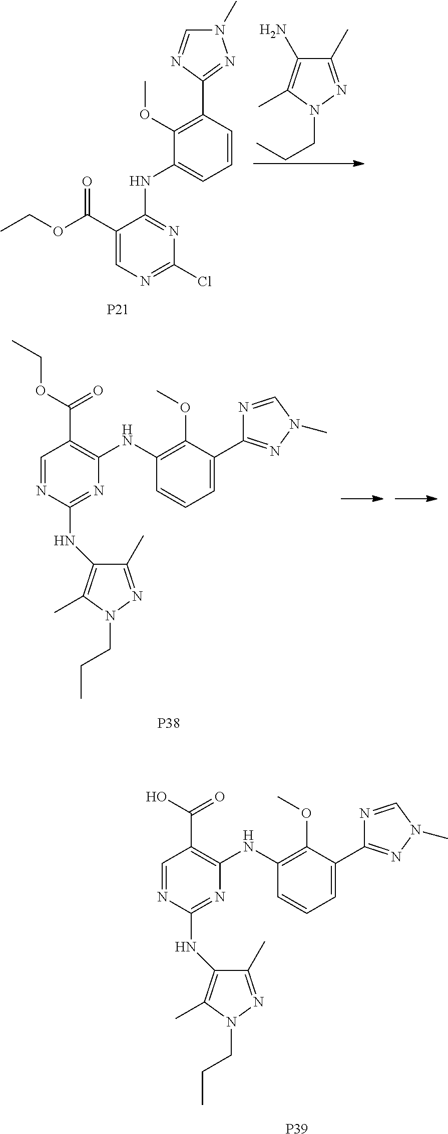

### Preparation 38. Ethyl 2-(1-propyl-3,5-dimethyl-1H-pyrazol-4-ylamino)-4-(2-methoxy-3-(1-methyl-1H-1,2,4-triazol-3-yl)phenylamino)pyrimidine-5-carboxylate (P38)

3,5-Dimethyl-1-propyl-1H-pyrazol-4-amine (979 mg, 6.40 mmol), cesium carbonate (812 mg, 2.5 mmol), Xantphos (35 mg, 0.06 mmol), and palladium acetate (13 mg, 0.06 mmol) were added to solution of P21 (250 mg, 0.64 mmol) in dioxane (40 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (ethyl acetate/THF 1:1) to give P38 (206 mg, 63% yield) as a white solid. ¹H NMR (400 MHz, DMSO-d₆), δ: 10.95 (br. s, 1H), 9.05 (s, 1H), 8.71 (s, 1H), 8.52 (s, 1H), 8.26 (d, J=8.0 Hz, 1H), 7.43 (d, J=6.5 Hz, 1H), 6.82 (t, J=8.0 Hz, 1H), 4.31 (q, J=7.2 Hz, 2H), 3.94 (s, 3H), 3.75 (t, J=7.0 Hz, 2H), 3.77 (s, 3H), 2.05 (s, 3H), 1.97 (s, 3H), 1.64-1.55 (m, 2H), 1.32 (t, J=7.2 Hz, 3H), 0.80 (t, J=7.2 Hz, 3H).

### Preparation 39. 2-(3,5-Dimethyl-1-propyl-1H-pyrazol-4-ylamino)-4-(2-methoxy-3-(1-methyl-1H-1,2,4-triazol-3-yl)phenylamino)pyrimidine-5-carboxylic Acid (P39)

A solution of P38 (206 mg, 0.40 mmol) in THF (15 mL) was mixed with water (15 mL) and lithium hydroxide (132 mg, 3.14 mmol) at rt. The reaction mixture was stirred overnight, concentrated, and acidified with 1M HCl to pH=6. The resulting precipitate was centrifugated and dried to give P38 (190 mg, 95% yield) as a yellow solid. LCMS (ESI) (C18 column 20×2 mm, particle size 2.5 μm, pore size 100A, water-acetonitrile+0.1% TFA, gradient 5 to 87% for 3 min), m/z: 478.3 [M+H]⁺; Rt=1.02 min.

### Synthesis of 2-(1-isopropyl-3,5-dimethyl-1H-pyrazol-4-ylamino)-4-(2-methoxy-3-(1-methyl-1H-1,2,4-triazol-3-yl)phenylamino)pyrimidine-5-carboxylic Acid (P41)

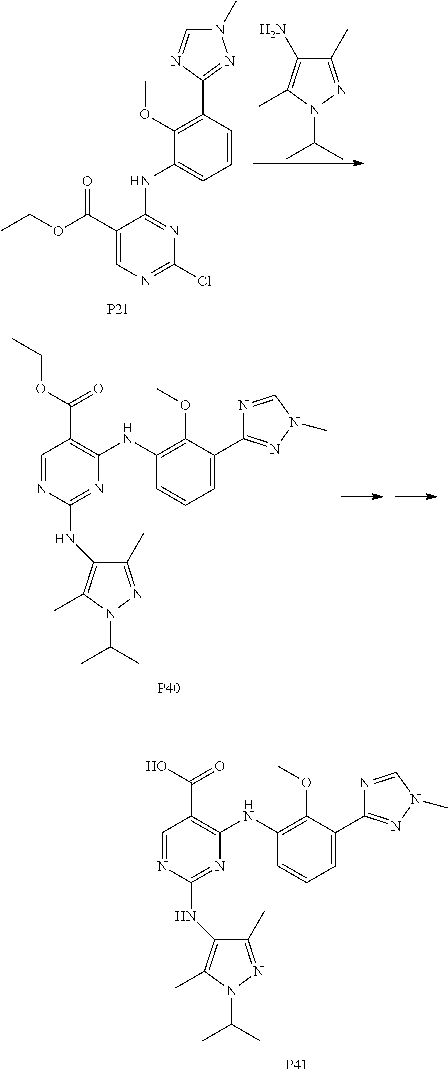

### Preparation 40. Ethyl 2-(1-isopropyl-3,5-dimethyl-1H-pyrazol-4-ylamino)-4-(2-methoxy-3-(1-methyl-1H-1,2,4-triazol-3-yl)phenylamino)pyrimidine-5-carboxylate (P40)

1-Isopropyl-3,5-dimethyl-1H-pyrazol-4-amine (787 mg, 5.15 mmol), cesium carbonate (747 mg, 2.3 mmol), Xantphos (29 mg, 0.05 mmol), and palladium acetate (11 mg, 0.05 mmol) were added to solution of P21 (200 mg, 0.515 mmol) in dioxane (40 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (ethyl acetate/THF 1:1) to give P40 (150 mg, 57% yield) as a white solid. ¹H NMR (400 MHz, DMSO-d₆), δ:10.95 (br. s, 1H), 9.05 (s, 1H), 8.71 (s, 1H), 8.52 (s, 1H), 8.26 (d, J=8.0 Hz, 1H), 7.43 (d, J=6.5 Hz, 1H), 6.82 (t, J=8.0 Hz, 1H), 4.31 (q, J=7.2 Hz, 2H), 3.94 (s, 3H), 3.81 (q, J=7.0 Hz, 1H), 3.77 (s, 3H), 2.05 (s, 3H), 1.97 (s, 3H), 1.32 (t, J=7.2 Hz, 3H), 1.21 (d, J=7.2 Hz, 6H).

### Preparation 41. 2-(1-Isopropyl-3,5-dimethyl-1H-pyrazol-4-ylamino)-4-(2-methoxy-3-(1-methyl-1H-1,2,4-triazol-3-yl)phenylamino)pyrimidine-5-carboxylic Acid (P41)

A solution of P40 (150 mg, 0.30 mmol) in THF (15 mL) was mixed with water (15 mL) and lithium hydroxide (124 mg, 3.0 mmol) at rt. The reaction mixture was stirred overnight, concentrated, and acidified with 1M HCl to pH=6. The resulting precipitate was centrifugated and dried to give P41 (130 mg, 84% yield) as a yellow solid. LCMS (ESI) (C18 column 20×2 mm, particle size 2.5 μm, pore size 100A, water-acetonitrile+0.1% TFA, gradient 5 to 87% for 3 min), m/z: 478.4 [M+H]⁺; Rt=1.04 min.

### Synthesis of 2-[(1-cyclopropyl-1H-pyrazol-4-yl)amino]-4-{[2-methoxy-3-(1-methyl-1H-1,2,4-triazol-3-yl)phenyl]amino}pyrimidine-5-carboxylic Acid (P43)

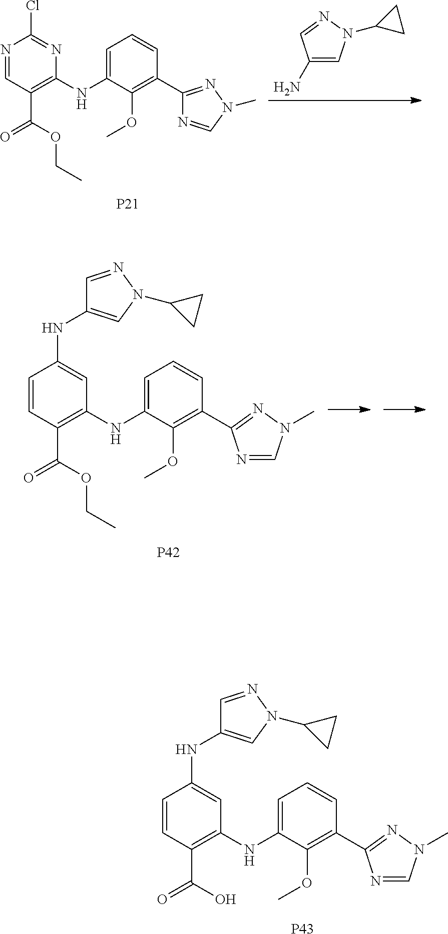

### Preparation 42. Ethyl 2-[(1-cyclopropyl-1H-pyrazol-4-yl)amino]-4-{[2-methoxy-3-(1-methyl-1H-1,2,4-triazol-3-yl)phenyl]amino}pyrimidine-5-carboxylate (P42)

Under argon atmosphere a mixture of P21 (0.25 g, 0.643 mmol), 1-cyclopropyl-1H-pyrazol-4-amine (0.236 g, 6.43 mmol), palladium acetate (0.05 g, 0.222 mmol), Xantphos (0.15 g, 0.259 mmol) and cesium carbonate (0.314 g, 0.964 mmol) in dioxane (25 ml) was stirred at 100° C. overnight. The mixture was cooled and filtered through a pad of celite, the solvent from mother liquor was removed under reduced pressure. The residue was purified by column chromatography on silica gel (2%, 5%, 10% of MeOH in DCM) to provide the product P42 (0.242 g, 79% yield). LCMS (ESI) (C18 column 100×4.6 mm, 5.0 μm, pore size 100 Å, water-acetonitrile+0.1% TFA, gradient 5 to 87% for 3 min), m/z: 476.3 [M+H]⁺; Rt=1.23 min.

### Preparation 43. 2-[(1-Cyclopropyl-1H-pyrazol-4-yl)amino]-4-{[2-methoxy-3-(1-methyl-1H-1,2,4-triazol-3-yl)phenyl]amino}pyrimidine-5-carboxylic Acid (P43)

A solution of lithium hydroxide monohydrate (0.213 g, 5.09 mmol) in water (10 ml) was added to a suspension of P42 (0.242 g, 0.509 mmol) in THF (10 mL) at rt, the reaction mixture was stirred overnight, the solvents were removed under reduced pressure, the residue was dissolved in water (10 ml), the obtained solution was acidified to pH 5-6 with 10% HCl, the formed precipitate was filtered, washed with water and dried under reduced pressure to provide the product P43 (0.227 g, 100%). LCMS (ESI) (C18 column 100×4.6 mm, 5.0 μm, pore size 100 Å, water-acetonitrile+0.1% TFA, gradient 5 to 87% for 3 min), m/z: 447.4 [M+H]⁺; Rt=1.07 min.

### Synthesis of 2-(1-(cyclohexylmethyl)-1H-pyrazol-4-ylamino)-4-(2-methoxy-3-(1-methyl-1H-1,2,4-triazol-3-yl)phenylamino)pyrimidine-5-carboxylic Acid (P45)

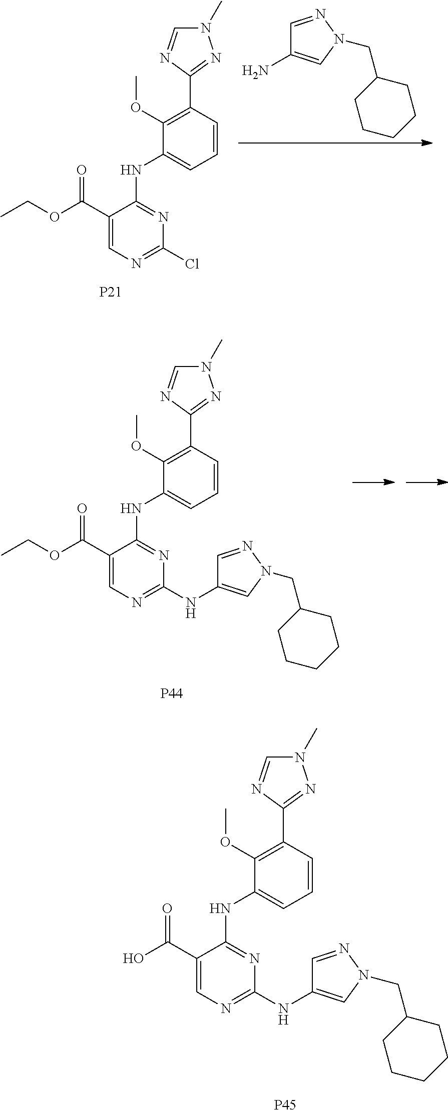

### Preparation 44. Ethyl 2-(1-(cyclohexylmethyl)-1H-pyrazol-4-ylamino)-4-(2-methoxy-3-(1-methyl-1H-1,2,4-triazol-3-yl)phenylamino)pyrimidine-5-carboxylate (P44)

1-(Cyclohexylmethyl)-1H-pyrazol-4-amine (1145 mg, 6.4 mmol), cesium carbonate (975 mg, 3.00 mmol), Xantphos (35 mg, 0.06 mmol), and palladium acetate (13 mg, 0.06 mmol) were added to solution of P21 (250 mg, 0.64 mmol) in dioxane (40 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (ethyl acetate/THF 50:50) to give P44 (312 mg, 91% yield) as a white solid. ¹H NMR (400 MHz, DMSO-d₆), δ: 10.40 (br. s, 1H), 9.83 (s, 1H), 8.69 (s, 1H), 8.56 (s, 1H), 8.06 (d, 1H), 7.83 (s, 1H), 7.70 (d, J=6.7 Hz, 1H), 7.38 (s, 1H), 7.21 (t, J=8.0 Hz, 1H), 4.29 (q, J=7.2 Hz, 2H), 3.95 (s, 3H), 3.80-3.74 (m, 5H), 1.66-1.41 (m, 5H), 1.34 (t, J=7.2 Hz, 3H), 1.20-1.09 (m, 4H), 0.97-0.79 (m, 2H).

### Preparation 45. 2-(1-(Cyclohexylmethyl)-1H-pyrazol-4-ylamino)-4-(2-methoxy-3-(1-methyl-1H-1,2,4-triazol-3-yl)phenylamino)pyrimidine-5-carboxylic Acid (P45)

A solution of P44 (300 mg, 0.531 mmol) in THF (15 mL) was mixed with water (15 mL) and lithium hydroxide (238 mg, 5.65 mmol) at rt. The reaction mixture was stirred overnight, concentrated, and acidified with 1M HCl to pH=6. The resulting precipitate was centrifugated and dried to give P45 (230 mg, 81% yield) as a yellow solid. LCMS (ESI) (C18 column 100×4.6 mm, 5.0 μm, pore size 100 Å, water-acetonitrile+0.1% TFA, gradient 5 to 87% for 10 min), m/z: 504.5 [M+H]⁺; Rt=5.14 min.

### Synthesis of 2-(1-(adamantylmethyl)-1H-pyrazol-4-ylamino)-4-(2-methoxy-3-(1-methyl-1H-1,2,4-triazol-3-yl)phenylamino)pyrimidine-5-carboxylic Acid (P47)

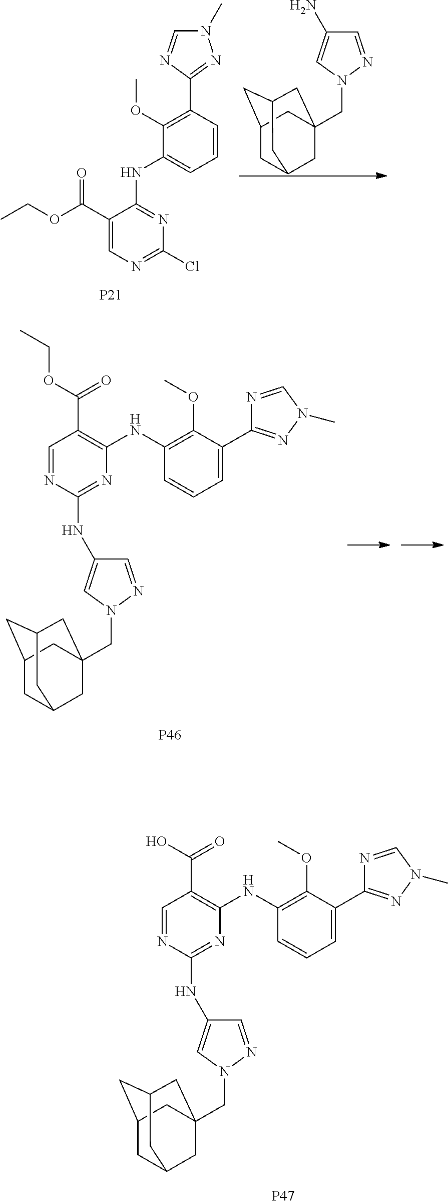

### Preparation 46. Ethyl 2-(1-(adamantylmethyl)-1H-pyrazol-4-ylamino)-4-(2-methoxy-3-(1-methyl-1H-1,2,4-triazol-3-yl)phenylamino)pyrimidine-5-carboxylate (P46)

1-(adamantylmethyl)-1H-pyrazol-3-amine (952 mg, 4.12 mmol), cesium carbonate (660 mg, 2.03 mmol), Xantphos (30 mg, 0.05 mmol), and palladium acetate (11 mg, 0.05 mmol) were added to solution of P21 (200 mg, 0.515 mmol) in dioxane (40 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (ethyl acetate/THF 2:1) to give P46 (243 mg, 81% yield) as a white solid. ¹H NMR (400 MHz, DMSO-d₆), δ: 10.35 (br. s, 1H), 9.87 (s, 1H), 8.69 (s, 1H), 8.55 (s, 1H), 8.02 (d, J=Hz, 1H), 7.74-7.69 (m, 1H), 7.50 (s, 1H), 7.39 (s, 1H), 7.24 (t, J=8.0 Hz, 1H), 4.33 (q, J=7.2 Hz, 2H), 3.93 (s, 3H), 3.73 (s, 3H), 3.57 (s, 2H), 1.91 (br. s, 4H), 1.65-1.62 (m, 3H), 1.57-1.51 (m, 3H), 1.44-1.42 (m, 5H), 1.34 (t, J=7.2 Hz, 3H).

### Preparation 47. 2-(1-(Adamantylmethyl)-1H-pyrazol-4-ylamino)-4-(2-methoxy-3-(1-methyl-1H-1,2,4-triazol-3-yl)phenylamino)pyrimidine-5-carboxylic Acid (P47)

A solution of P46 (300 mg, 0.51 mmol) in THF (15 mL) was mixed with water (15 mL) and lithium hydroxide (216 mg, 5.10 mmol) at rt. The reaction mixture was stirred overnight, concentrated, and acidified with 1M HCl to pH=6. The resulting precipitate was centrifugated and dried to give P47 (210 mg, 74% yield) as a yellow solid. LCMS (ESI) (C18 column 20×2 mm, particle size 2.5 μm, pore size 100A, water-acetonitrile+0.1% TFA, gradient 5 to 87% for 3 min), m/z: 556.5 [M+H]⁺; Rt=1.43 min.

### Synthesis of 2-(1-isopentyl-1H-pyrazol-4-ylamino)-4-(2-methoxy-3-(1-methyl-1H-1,2,4-triazol-3-yl)phenylamino)pyrimidine-5-carboxylic Acid (P49)

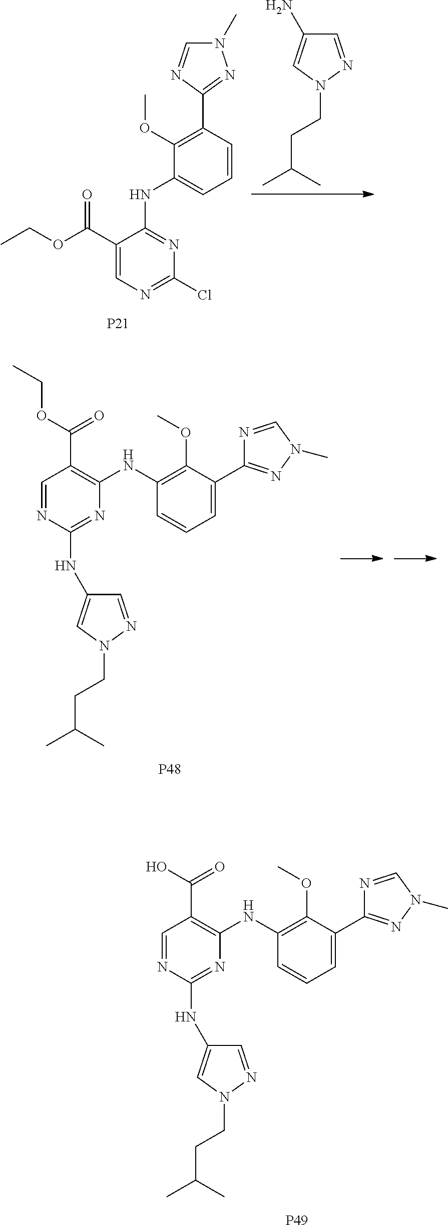

### Preparation 48. Ethyl 2-(1-isopentyl-1H-pyrazol-4-ylamino)-4-(2-methoxy-3-(1-methyl-1H-1,2,4-triazol-3-yl)phenylamino)pyrimidine-5-carboxylate (P48)

1-Isopentyl-1H-pyrazol-4-amine (985 mg, 6.44 mmol), cesium carbonate (812 mg, 2.5 mmol), Xantphos (40 mg, 0.07 mmol), and palladium acetate (16 mg, 0.07 mmol) were added to solution of P21 (250 mg, 0.64 mmol) in dioxane (40 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (ethyl acetate/THF 2:1) to give P48 (250 mg, 77% yield) as a white solid. ¹H NMR (400 MHz, DMSO-d₆), δ: 10.54 (br. s, 1H), 9.87 (s, 1H), 8.70 (s, 1H), 8.56 (s, 1H), 8.19 (d, J=Hz, 1H), 7.74-7.69 (m, 1H), 7.57 (s, 1H), 7.41 (s, 1H), 7.22 (t, J=8.0 Hz, 1H), 4.32 (q, J=7.2 Hz, 2H), 3.93-3.95 (m, 5H), 3.76 (s, 3H), 1.69-1.64 (m, 2H), 1.57-1.49 (m, 1H), 1.34 (t, J=7.2 Hz, 3H), 0.90 (d, J=6.6 Hz, 6H).

### Preparation 49. 2-(1-Isopentyl-1H-pyrazol-4-ylamino)-4-(2-methoxy-3-(1-methyl-1H-1,2,4-triazol-3-yl)phenylamino)pyrimidine-5-carboxylic Acid (P49)

A solution of P48 (200 mg, 0.49 mmol) in THF (15 mL) was mixed with water (15 mL) and lithium hydroxide (206 mg, 4.9 mmol) at rt. The reaction mixture was stirred overnight, concentrated, and acidified with 1M HCl to pH=6. The resulting precipitate was centrifugated and dried to give P49 (110 mg, 58% yield) as a yellow solid. LCMS (ESI) (C18 column 20×2 mm, particle size 2.5 μm, pore size 100A, water-acetonitrile+0.1% TFA, gradient 5 to 87% for 3 min), m/z: 478.4 [M+H]⁺; Rt=1.25 min.

### Synthesis of 4-((2-methoxy-3-(1-methyl-1H-1,2,4-triazol-3-yl)phenyl)amino)-2-((1-octyl-1H-pyrazol-4-yl)amino)pyrimidine-5-carboxylic Acid (P51)

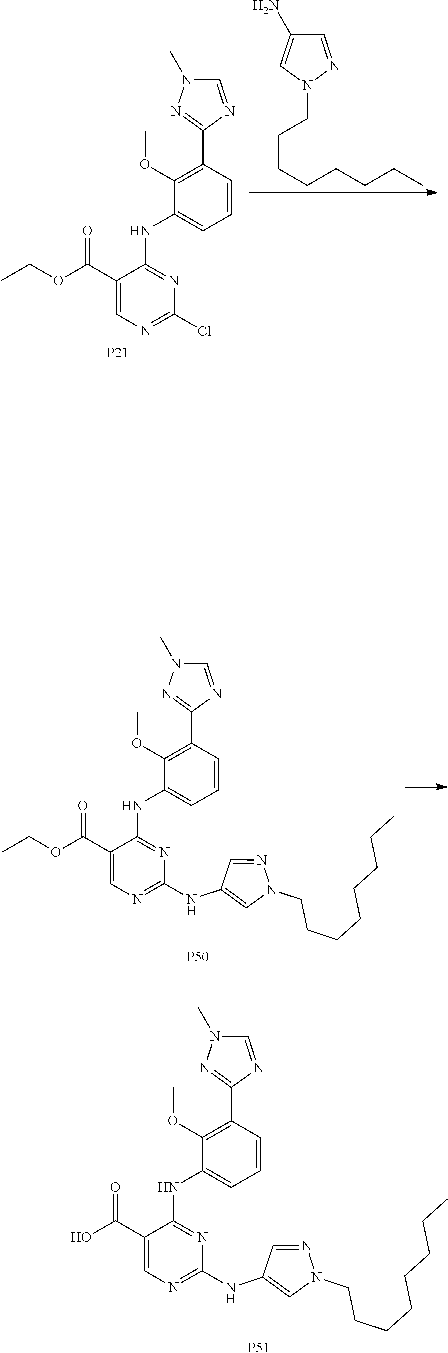

### Preparation 50. Ethyl 4-{[2-methoxy-3-(1-methyl-1H-1,2,4-triazol-3-yl)phenyl]amino}-2-{methyl[(1-phenyl-1H-pyrazol-4-yl)methyl]amino}pyrimidine-5-carboxylate (P50)

To a solution of P21 (150 mg, 0.38 mmol) in dioxane (10 mL, degassed) was added 1-octyl-1H-pyrazol-4-amine (150 mg, 0.76 mmol), cesium carbonate (0.5 g, 1.53 mmol), Xantphos (25 mg, 0.04 mmol), and palladium acetate (10 mg, 0.04 mmol) at rt under argon atmosphere. After the solution was stirred at 100° C. for overnight, the mixture was cooled and filtered through a pad of celite. The filtrate was then concentrated under reduced pressure. The residue was purified by silica gel column chromatography (CHCl₃/MeOH=95:5) to give P50 (180 mg, 86% yield) as a white solid. LCMS (ESI), m/z: 548.8 [M+H]⁺; Rt=1.74 min.

### Preparation 51. 4-((2-Methoxy-3-(1-methyl-1H-1,2,4-triazol-3-yl)phenyl)amino)-2-((1-octyl-1H-pyrazol-4-yl)amino)pyrimidine-5-carboxylic Acid (P51)

To a solution of P50 (140 mg, 0.25 mmol) in THF (8.5 mL) was added water (8.5 mL) and lithium hydroxide (100 mg, 2.38 mmol) at rt. After it was stirred for overnight, the mixture was concentrated and then acidified with diluted HCl. Precipitated solid was filtered and dried on air to give a yellow solid. Yield 0.075 g (56%). LCMS (ESI), m/z: 520.8 [M+H]⁺; Rt=1.41 min.

### Synthesis of 2-((1-hexyl-1H-pyrazol-4-yl)amino)-4-((2-methoxy-3-(1-methyl-1H-1,2,4-triazol-3-yl)phenyl)amino)pyrimidine-5-carboxylic Acid (P53)

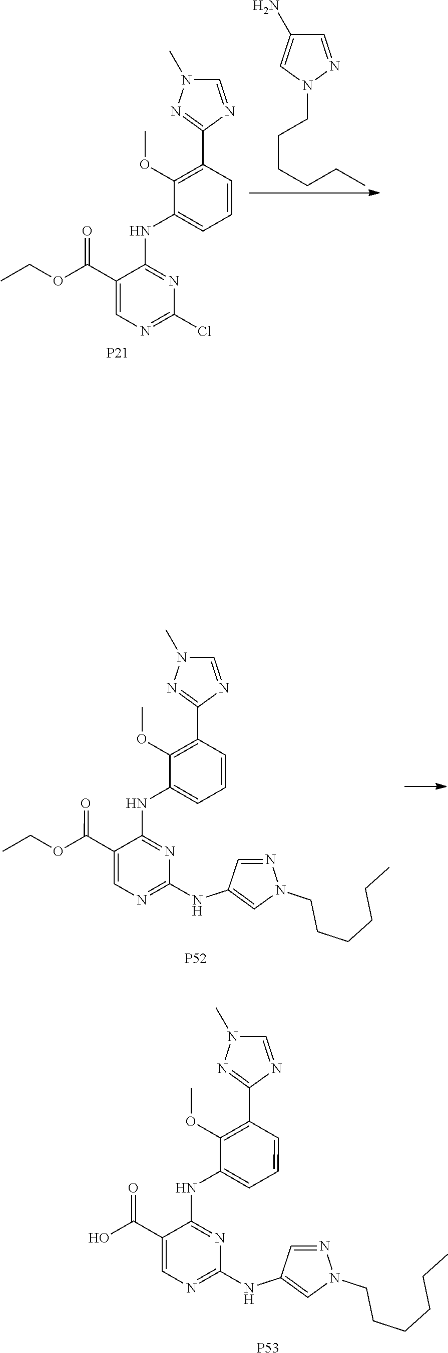

### Preparation 52. Ethyl 2-((1-hexyl-1H-pyrazol-4-yl)amino)-4-((2-methoxy-3-(1-methyl-1H-1,2,4-triazol-3-yl)phenyl)amino)pyrimidine-5-carboxylate (P52)

To a solution of P21 (150 mg, 0.38 mmol) in dioxane (10 mL, degassed) was added 1-hexyl-1H-pyrazol-4-amine (120 mg, 0.72 mmol), cesium carbonate (0.5 g, 1.53 mmol), Xantphos (25 mg, 0.04 mmol), and palladium acetate (10 mg, 0.04 mmol) at rt under argon atmosphere. After the solution was stirred at 100° C. for overnight, the mixture was cooled and filtered through a pad of celite. The filtrate was then concentrated under reduced pressure. The residue was purified by silica gel column chromatography (CHCl₃/MeOH=95:5) to give P52 (96 mg, 47% yield) as a white solid. LCMS (ESI), m/z: 520.5 [M+H]⁺; Rt=1.52 min.

### Preparation 53. 2-((1-hexyl-1H-pyrazol-4-yl)amino)-4-((2-methoxy-3-(1-methyl-1H-1,2,4-triazol-3-yl)phenyl)amino)pyrimidine-5-carboxylic Acid (P53)

To a solution of P52 (96 mg, 0.18 mmol) in THF (8.5 mL) was added water (8.5 mL) and lithium hydroxide (91 mg, 2.16 mmol) at rt. After it was stirred for overnight, the mixture was concentrated and then acidified with diluted HCl. Precipitated solid was filtered and dried on air to give a yellow solid. Yield 0.083 g (91%). LCMS (ESI), m/z: 492.4 [M+H]⁺; Rt=1.26 min.

### Synthesis of 4-(2-methoxy-3-(1-methyl-1H-1,2,4-triazol-3-yl)phenylamino)-2-(1-(2-(1-methylpiperidin-2-yl)ethyl)-1H-pyrazol-4-ylamino)pyrimidine-5-carboxylic Acid (P55)

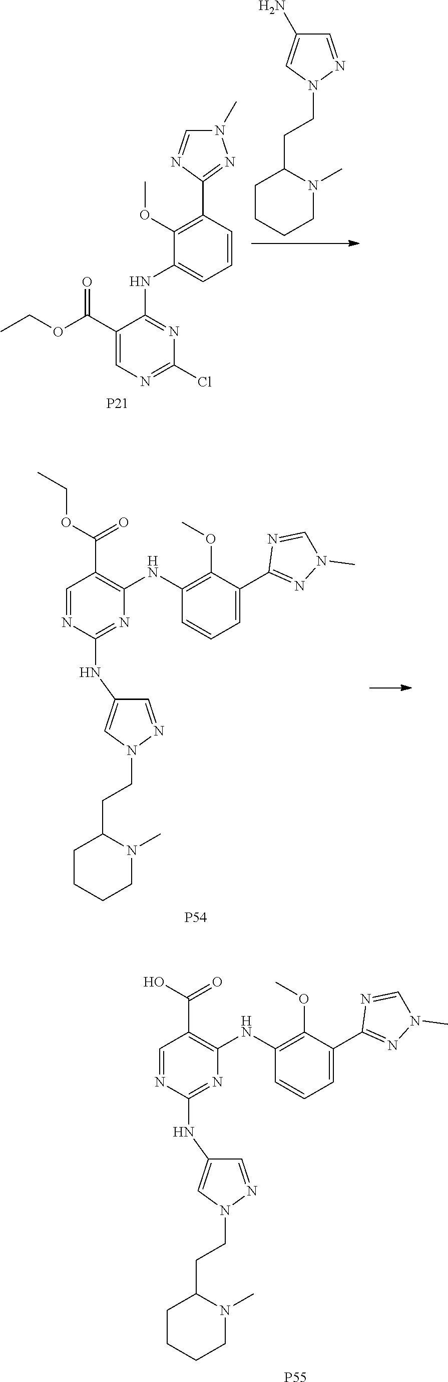

### Preparation 54. Ethyl 4-(2-methoxy-3-(1-methyl-1H-1,2,4-triazol-3-yl)phenylamino)-2-(1-(2-(1-methylpiperidin-2-yl)ethyl)-1H-pyrazol-4-ylamino)pyrimidine-5-carboxylate (P54)

1-(2-(1-methylpiperidin-2-yl)ethyl)-1H-pyrazol-4-amine (1071 mg, 5.14 mmol), cesium carbonate (812 mg, 2.5 mmol), Xantphos (30 mg, 0.05 mmol), and palladium acetate (11 mg, 0.05 mmol) were added to solution of P21 (250 mg, 0.64 mmol) in dioxane (40 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (DCM/MeOH/Et₃N 100:5:0.5) to give P54 (170 mg, 47% yield) as a white solid. ¹H NMR (400 MHz, DMSO-d₆), δ: 10.54 (br. s, 1H), 9.87 (s, 1H), 8.70 (s, 1H), 8.56 (s, 1H), 8.19 (d, J=Hz, 1H), 7.74-7.69 (m, 1H), 7.57 (s, 1H), 7.41 (s, 1H), 7.22 (t, J=8.0 Hz, 1H), 4.32 (q, J=7.2 Hz, 2H), 3.93-3.95 (m, 5H), 3.76 (s, 3H), 2.78-2.71 (m, 1H), 2.56-2.53 (m, 2H), 2.19-2.10 (m, 4H), 2.01-1.86 (m, 2H), 1.63-1.46 (m, 2H), 1.34 (t, J=7.2 Hz, 3H), 1.26-1.15 (m, 3H).

### Preparation 55. 4-(2-Methoxy-3-(1-methyl-1H-1,2,4-triazol-3-yl)phenylamino)-2-(1-(2-(1-methylpiperidin-2-yl)ethyl)-1H-pyrazol-4-ylamino)pyrimidine-5-carboxylic Acid (P55)

A solution of P54 (170 mg, 0.30 mmol) in THF (15 mL) was mixed with water (15 mL) and lithium hydroxide (127 mg, 3.0 mmol) at rt. The reaction mixture was stirred overnight, concentrated, and acidified with 1M HCl to pH=6. The resulting precipitate was centrifugated and dried to give P55 (160 mg, 99% yield) as a yellow solid. LCMS (ESI) (C18 column 20×2 mm, particle size 2.5 μm, pore size 100A, water-acetonitrile+0.1% TFA, gradient 5 to 87% for 3 min), m/z: 533.3 [M+H]⁺; Rt=0.83 min.

### Synthesis of 2-(1-(3-(dimethylamino) propyl)-1H-pyrazol-4-ylamino)-4-(2-methoxy-3-(1-methyl-1H-1,2,4-triazol-3-yl)phenylamino)pyrimidine-5-carboxylic Acid (P57)

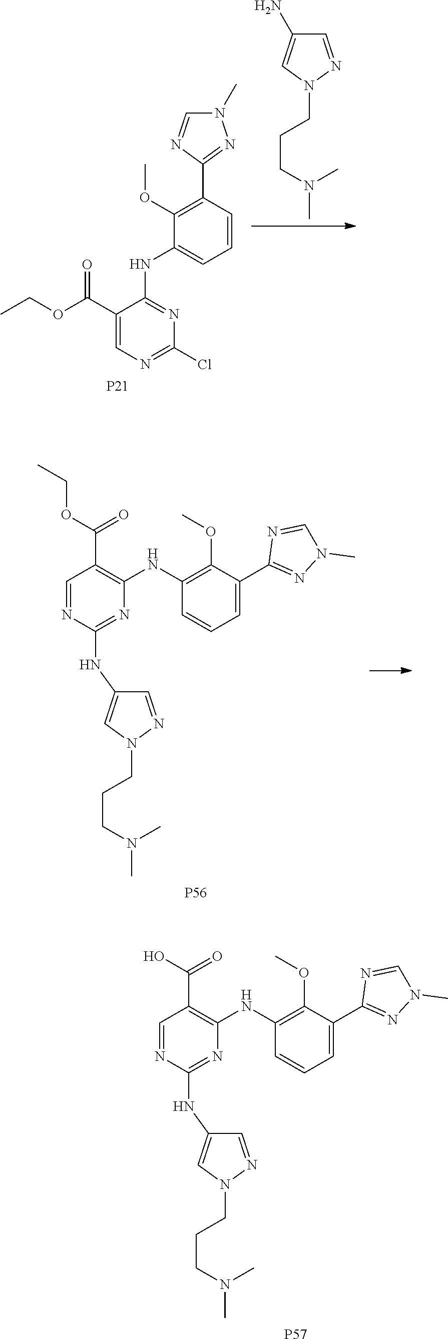

### Preparation 56. Ethyl 2-(1-(3-(dimethylamino) propyl)-1H-pyrazol-4-ylamino)-4-(2-methoxy-3-(1-methyl-1H-1,2,4-triazol-3-yl)phenylamino)pyrimidine-5-carboxylate (P56)

1-(3-(dimethylamino) propyl)-1H-pyrazol-4-amine (1075 mg, 6.4 mmol), cesium carbonate (975 mg, 3.0 mmol), Xantphos (35 mg, 0.06 mmol), and palladium acetate (13 mg, 0.06 mmol) were added to solution of P21 (250 mg, 0.64 mmol) in dioxane (40 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (DCM/MeOH/Et₃N 100:5:0.5) to give P56 (120 mg, 63% yield) as a white solid. ¹H NMR (400 MHz, DMSO-d₆), δ: 10.56 (br. s, 1H), 9.86 (s, 1H), 8.70 (s, 1H), 8.56 (s, 1H), 8.19 (d, J=Hz, 1H), 7.66-7.64 (m, 1H), 7.57 (s, 1H), 7.41 (s, 1H), 7.22 (t, J=8.0 Hz, 1H), 4.32 (q, J=7.2 Hz, 2H), 4.12-4.04 (m, 2H), 3.98-3.91 (m, 5H), 3.76 (s, 3H), 1.85-1.77 (m, 2H), 1.34 (t, J=7.2 Hz, 3H), 1.06 (d, J=6.2 Hz, 6H).

### Preparation 57. 2-(1-(3-(Dimethylamino) propyl)-1H-pyrazol-4-ylamino)-4-(2-methoxy-3-(1-methyl-1H-1,2,4-triazol-3-yl)phenylamino)pyrimidine-5-carboxylic Acid (P57)

A solution of P56 (120 mg, 0.23 mmol) in THF (15 mL) was mixed with water (15 mL) and lithium hydroxide (97 mg, 2.3 mmol) at rt. The reaction mixture was stirred overnight, concentrated, and acidified with 1M HCl to pH=6. The resulting precipitate was centrifugated and dried to give P57 (110 mg, 99% yield) as a yellow solid. LCMS (ESI) (C18 column 100×4.6 mm, 5.0 μm, pore size 100 Å, water-acetonitrile+0.1% TFA, gradient 5 to 87% for 10 min), m/z: 493.8 [M+H]⁺; Rt=3.66 min.

### Synthesis of 2-(1-(2-(Diisobutylamino)ethyl)-1H-pyrazol-4-ylamino)-4-(2-methoxy-3-(1-methyl-1H-1,2,4-triazol-3-yl)phenylamino)pyrimidine-5-carboxylic Acid (P59)

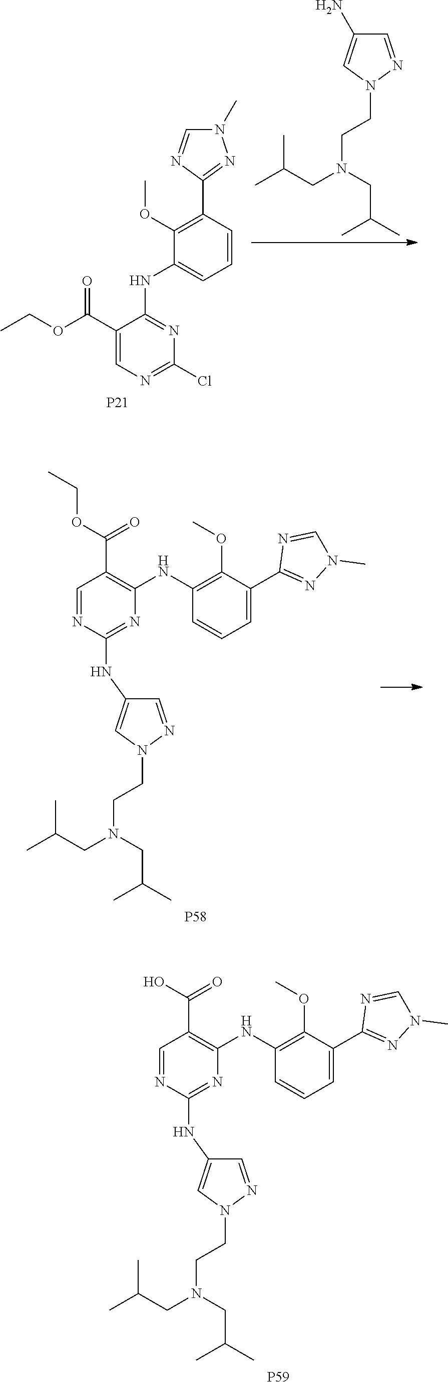

### Preparation 58. Ethyl 2-(1-(2-(diisobutylamino)ethyl)-1H-pyrazol-4-ylamino)-4-(2-methoxy-3-(1-methyl-1H-1,2,4-triazol-3-yl)phenylamino)pyrimidine-5-carboxylate (P58)

1-(2-(Diisobutylamino)ethyl)-1H-pyrazol-4-amine (1533 mg, 6.4 mmol), cesium carbonate (975 mg, 3.0 mmol), Xantphos (35 mg, 0.06 mmol), and palladium acetate (13 mg, 0.06 mmol) were added to solution of P21 (250 mg, 0.64 mmol) in dioxane (40 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (EtOAc/THF 50:50) to give P58 (215 mg, 57% yield) as a white solid. ¹H NMR (400 MHz, DMSO-d₆), δ: 10.60 (br. s, 1H), 9.89 (s, 1H), 8.70 (s, 1H), 8.55 (s, 1H), 8.20 (d, J=Hz, 1H), 7.68-7.66 (m, 1H), 7.55 (s, 1H), 7.43 (s, 1H), 7.22 (t, J=8.0 Hz, 1H), 4.32 (q, J=7.2 Hz, 2H), 4.16-4.11 (m, 2H), 3.95 (s, 3H), 3.80 (s, 3H), 2.83 (t, 2H), 2.17-2.12 (m, 4H), 1.67-1.63 (m, 2H), 1.34 (t, J=7.2 Hz, 3H), 0.84 (d, J=6.2 Hz, 12H).

### Preparation 59. 2-(1-(2-(Diisobutylamino)ethyl)-1H-pyrazol-4-ylamino)-4-(2-methoxy-3-(1-methyl-1H-1,2,4-triazol-3-yl)phenylamino)pyrimidine-5-carboxylic Acid (P59)

A solution of P58 (300 mg, 0.51 mmol) in THF (15 mL) was mixed with water (15 mL) and lithium hydroxide (119 mg, 2.8 mmol) at rt. The reaction mixture was stirred overnight, concentrated, and acidified with 1M HCl to pH=6. The resulting precipitate was centrifugated and dried to give P59 (200 mg, 70% yield) as a yellow solid. LCMS (ESI) (C18 column 20×2 mm, particle size 2.5 μm, pore size 100A, water-acetonitrile+0.1% TFA, gradient 5 to 87% for 3 min), m/z: 563.8 [M+H]⁺; Rt=1.01 min.

### Synthesis of 2-[(1-isopropyl-1H-pyrazol-4-yl)amino]-4-{[2-methoxy-3-(1-methyl-1H-1,2,4-triazol-3-yl)phenyl]amino}pyrimidine-5-carboxylic Acid (P61)

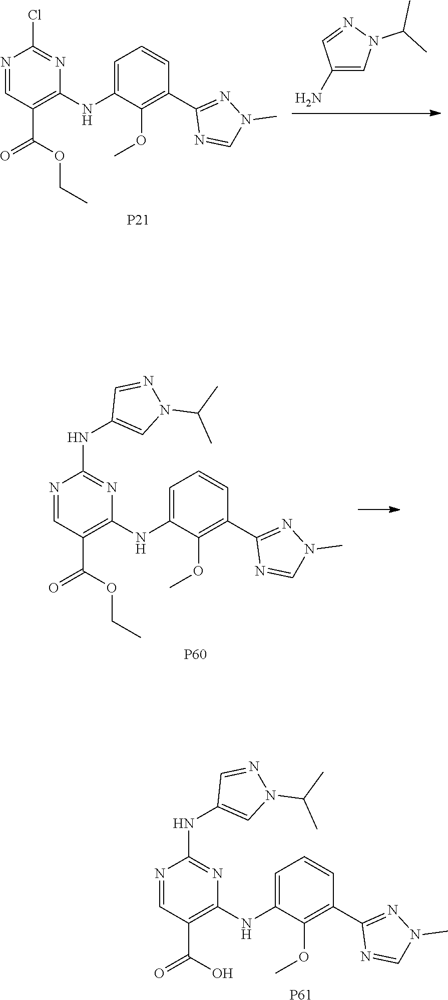

### Preparation 60. Ethyl 2-[(1-isopropyl-1H-pyrazol-4-yl)amino]-4-{[2-methoxy-3-(1-methyl-1H-1,2,4-triazol-3-yl)phenyl]amino}pyrimidine-5-carboxylate (P60)

Under argon a mixture of P21 (0.778 g, 2 mmol), 1-isopropyl-1H-pyrazol-4-amine (2.5 g, 20 mmol), palladium acetate (0.15 g, 0.668 mmol), Xantphos (0.45 g, 0.778 mmol) and cesium carbonate (0.977 g, 3 mmol) in dioxane (75 ml) was stirred at 100° C. overnight. The mixture was cooled and filtered through a pad of celite, the solvent from mother liquor was removed under reduced pressure. The residue was purified by column chromatography on silica gel (2%, 5%, 10% of MeOH in DCM) to provide the product P60 (0.8 g, 83% yield). LCMS (ESI) (C18 column 100×4.6 mm, 5.0 μm, pore size 100 Å, water-acetonitrile+0.1% TFA, gradient 5 to 87% for 3 min), m/z: 478.3 [M+H]⁺; Rt=1.32 min.

### Preparation 61. 2-[(1-Isopropyl-1H-pyrazol-4-yl)amino]-4-{[2-methoxy-3-(1-methyl-1H-1,2,4-triazol-3-yl)phenyl]amino}pyrimidine-5-carboxylic Acid (P61)

A solution of lithium hydroxide monohydrate (1 g, 20 mmol) in water (20 ml) was added to a suspension of P60 (0.8 g, 1.675 mmol) in THF (50 mL) at rt, the reaction mixture was stirred overnight, the solvents were removed under reduced pressure, the residue was dissolved in water (50 ml), the obtained solution was acidified to pH 5-6 with 10% HCl, the formed precipitate was filtered, washed with water and dried under reduced pressure to provide the product P61 (0.75 g, 100%). LCMS (ESI) (C18 column 100×4.6 mm, 5.0 μm, pore size 100 Å, water-acetonitrile+0.1% TFA, gradient 5 to 87% for 3 min), m/z: 450.5 [M+H]⁺; Rt=0.97 min.

### Synthesis of 4-((2-methoxy-3-(1-methyl-1H-1,2,4-triazol-3-yl)phenyl)amino)-2-((1-methyl-1H-pyrazol-4-yl)amino)pyrimidine-5-carboxylic Acid (P63)

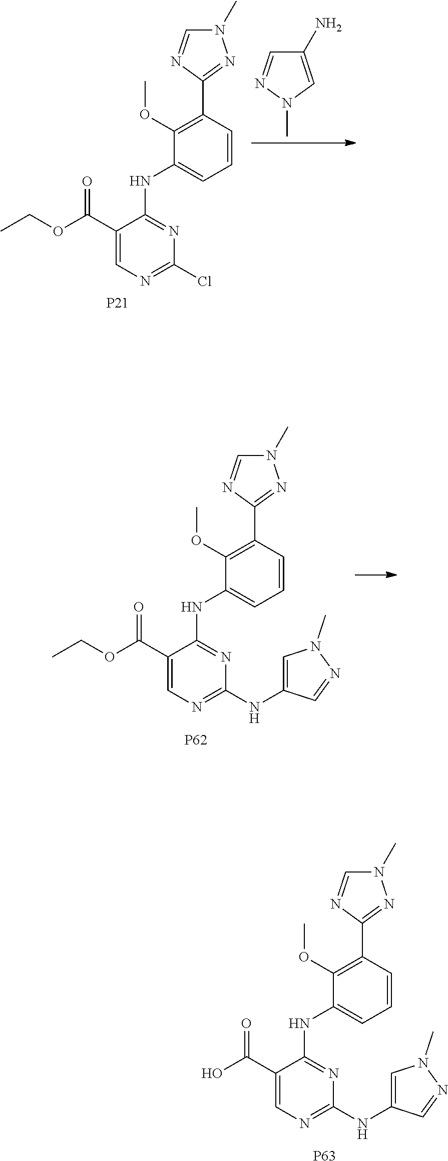

### Preparation 62 Ethyl 4-((2-methoxy-3-(1-methyl-1H-1,2,4-triazol-3-yl)phenyl)amino)-2-((1-methyl-1H-pyrazol-4-yl)amino)pyrimidine-5-carboxylate (P62)

1-methyl-1H-pyrazol-4-amine (240 mg, 2.47 mmol), cesium carbonate (600 mg, 1.85 mmol), Xantphos (44 mg, 0.07 mmol), and palladium acetate (16 mg, 0.07 mmol) were added to solution of P21 (300 mg, 0.77 mmol) in dioxane (50 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (ethyl acetate/THF 50:50) to give P62 (100 mg, 37% yield) as a white solid. LCMS (ESI) (C18 column 100×4.6 mm, 5.0 μm, pore size 100 Å, water-acetonitrile+0.1% TFA, gradient 5 to 87% for 10 min), m/z: 450.6 [M+H]⁺; Rt=4.91 min.

### Preparation 63. 4-((2-Methoxy-3-(1-methyl-1H-1,2,4-triazol-3-yl)phenyl)amino)-2-((1-methyl-1H-pyrazol-4-yl)amino)pyrimidine-5-carboxylic Acid (P63)

A solution of P62 (100 mg, 0.22 mmol) in THF (15 mL) was mixed with water (15 mL) and lithium hydroxide (50 mg, 1.2 mmol) at rt. The reaction mixture was stirred overnight, concentrated, and acidified with 1M HCl to pH=6. The resulting precipitate was centrifugated and dried to give P63 (60 mg, 64% yield) as a yellow solid. LCMS (ESI) (C18 column 100×4.6 mm, 5.0 μm, pore size 100 Å, water-acetonitrile+0.1% TFA, gradient 5 to 87% for 10 min), m/z: 422.3 [M+H]⁺; Rt=3.96 min.

### Synthesis of 2-((1-isopropyl-1H-pyrazol-4-yl)amino)-4-((2-methoxy-3-(1-methyl-1H-1,2,4-triazol-3-yl)phenyl)amino)pyrimidine-5-carboxylic Acid (P65)

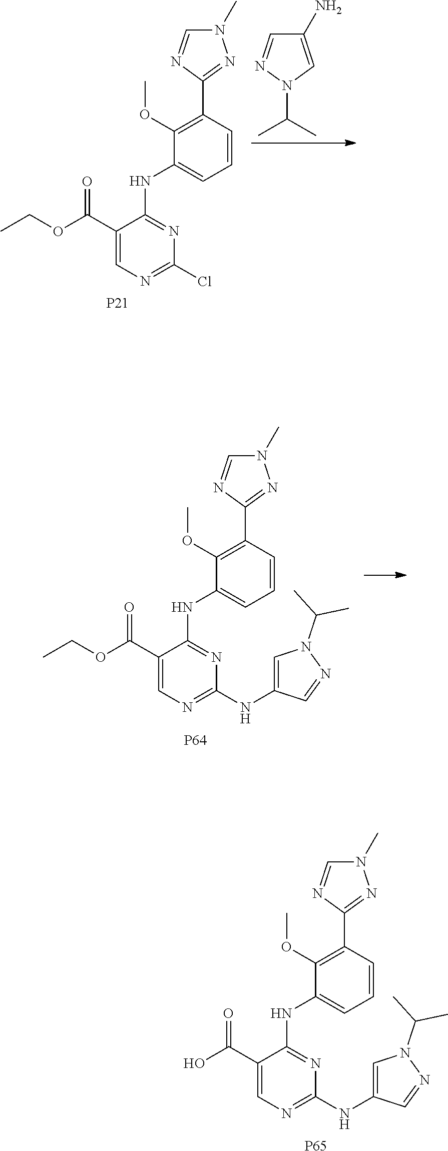

### Preparation 64. Ethyl 2-((1-isopropyl-1H-pyrazol-4-yl)amino)-4-((2-methoxy-3-(1-methyl-1H-1,2,4-triazol-3-yl)phenyl)amino)pyrimidine-5-carboxylate (P64)

1-Isopropyl-1H-pyrazol-4-amine (460 mg, 3.67 mmol), cesium carbonate (2178 mg, 6.68 mmol), Xantphos (580 mg, 1.00 mmol), and palladium acetate (225 mg, 1.00 mmol) were added to solution of P21 (1300 mg, 3.34 mmol) in dioxane (150 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (ethyl acetate/THF 50:50) to give P64 (93 mg, 6% yield) as a white solid. LCMS (ESI) (C18 column 100×4.6 mm, 5.0 μm, pore size 100 Å, water-acetonitrile+0.1% TFA, gradient 5 to 87% for 10 min), m/z: 478.2 [M+H]⁺; Rt=5.04 min.

### Preparation 65. 2-((1-Isopropyl-1H-pyrazol-4-yl)amino)-4-((2-methoxy-3-(1-methyl-1H-1,2,4-triazol-3-yl)phenyl)amino)pyrimidine-5-Carboxylic Acid (P65)

A solution of P64 (93 mg, 0.195 mmol) in THF (15 mL) was mixed with water (5 mL) and lithium hydroxide (82 mg, 1.950 mmol) at rt. The reaction mixture was stirred overnight, concentrated, and acidified with 1M HCl to pH=6. The resulting precipitate was centrifugated and dried to give P65 (87 mg, 99% yield) as a yellow solid. LCMS (ESI) (C18 column 100×4.6 mm, 5.0 μm, pore size 100 Å, water-acetonitrile+0.1% TFA, gradient 5 to 87% for 10 min), m/z: 450.3 [M+H]⁺; Rt=4.39 min.

### Synthesis of 2-((1-(2-(dimethylamino)ethyl)-1H-pyrazol-4-yl)amino)-4-((2-methoxy-3-(1-methyl-1H-1,2,4-triazol-3-yl)phenyl)amino)pyrimidine-5-carboxylic Acid (P67)

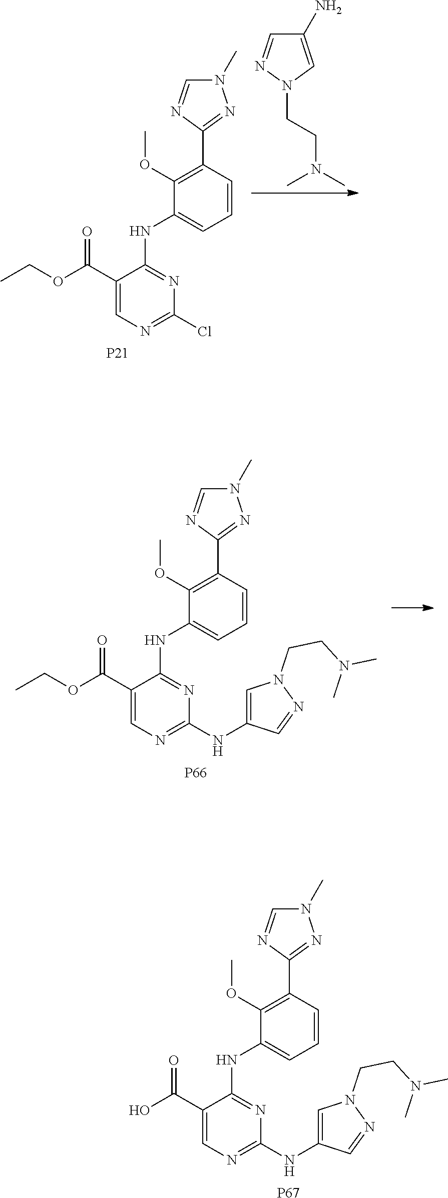

### Preparation 66. Ethyl 2-((1-(2-(dimethylamino)ethyl)-1H-pyrazol-4-yl)amino)-4-((2-methoxy-3-(1-methyl-1H-1,2,4-triazol-3-yl)phenyl)amino)pyrimidine-5-carboxylate (P66)

1-(2-(dimethylamino)ethyl)-1H-pyrazol-4-amine (436 mg, 2.83 mmol), cesium carbonate (1676 mg, 5.14 mmol), Xantphos (447 mg, 0.77 mmol), and palladium acetate (173 mg, 0.77 mmol) were added to solution of P21 (1000 mg, 2.57 mmol) in dioxane (150 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (ethyl acetate/THF 50:50) to give P66 (37 mg, 3% yield) as a white solid. LCMS (ESI) (C18 column 100×4.6 mm, 5.0 μm, pore size 100 Å, water-acetonitrile+0.1% TFA, gradient 5 to 87% for 10 min), m/z: 507.9 [M+H]⁺; Rt=3.95 min.

### Preparation 67. 2-((1-(2-(Dimethylamino)ethyl)-1H-pyrazol-4-yl)amino)-4-((2-methoxy-3-(1-methyl-1H-1,2,4-triazol-3-yl)phenyl)amino)pyrimidine-5-carboxylic Acid (P67)

A solution of P66 (37 mg, 0.073 mmol) in THF (15 mL) was mixed with water (5 mL) and lithium hydroxide (31 mg, 0.730 mmol) at rt. The reaction mixture was stirred overnight, concentrated, and acidified with 1M HC to pH=6. The resulting precipitate was centrifugated and dried to give P67 (35 mg, 100% yield) as a yellow solid. LCMS (ESI) (C18 column 100×4.6 mm, 5.0 μm, pore size 100 Å, water-acetonitrile+0.1% TFA, gradient 5 to 87% for 10 min), m/z: 479.4 [M+H]⁺; Rt=3.41 min.

### Synthesis of 4-((2-methoxy-3-(1-methyl-1H-1,2,4-triazol-3-yl)phenyl)amino)-2-((1-phenyl-1H-pyrazol-4-yl)amino)pyrimidine-5-carboxylic Acid (P69)

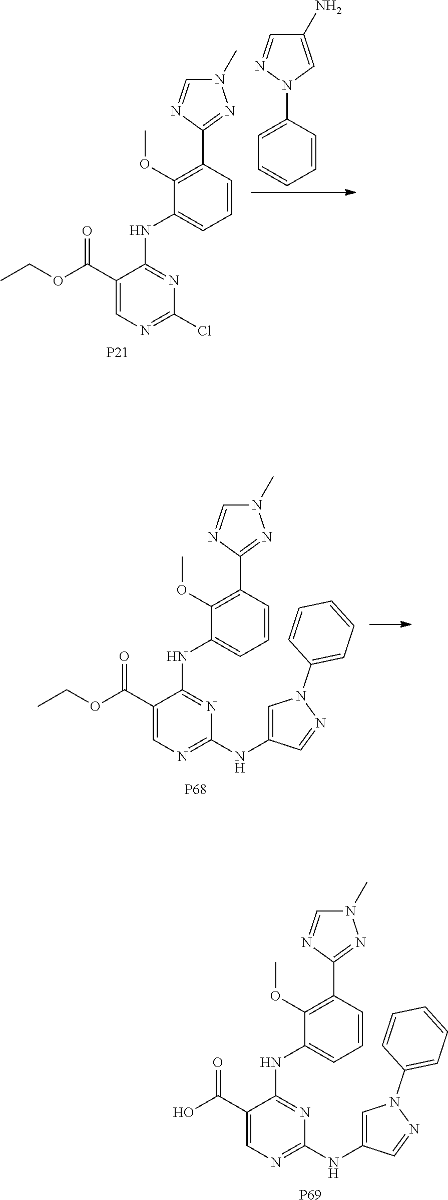

### Preparation 68 Ethyl 4-((2-methoxy-3-(1-methyl-1H-1,2,4-triazol-3-yl)phenyl)amino)-2-((1-phenyl-1H-pyrazol-4-yl)amino)pyrimidine-5-carboxylate (P68)

1-Phenyl-1H-pyrazol-4-amine (409 mg, 2.57 mmol), cesium carbonate (1676 mg, 5.14 mmol), Xantphos (447 mg, 0.77 mmol), and palladium acetate (173 mg, 0.77 mmol) were added to solution of P21 (1000 mg, 2.57 mmol) in dioxane (150 mL, degassed) at rt under argon atmosphere. After the solution was stirred at 85° C. overnight, the mixture was cooled and filtered through a pad of celite. The filtrate was then concentrated under reduced pressure. The residue was purified by silica gel column chromatography (ethyl acetate/THF 50:50) to give P68 (194 mg, 15% yield) as a white solid. LCMS (ESI) (C18 column 100×4.6 mm, 5.0 μm, pore size 100 Å, water-acetonitrile+0.1% TFA, gradient 5 to 87% for 10 min), m/z: 512.3 [M+H]⁺; Rt=6.04 min.

### Preparation 69. 4-((2-Methoxy-3-(1-methyl-1H-1,2,4-triazol-3-yl)phenyl)amino)-2-((1-phenyl-1H-pyrazol-4-yl)amino)pyrimidine-5-carboxylic Acid (P69)

A solution of P68 (194 mg, 0.379 mmol) in THF (20 mL) was mixed with water (5 mL) and lithium hydroxide (159 mg, 3.790 mmol) at rt. The reaction mixture was stirred overnight, concentrated, and acidified with 1M HCl to pH=6. The resulting precipitate was centrifugated and dried to give P69 (100 mg, 55% yield) as a yellow solid. LCMS (ESI) (C18 column 100×4.6 mm, 5.0 μm, pore size 100 Å, water-acetonitrile+0.1% TFA, gradient 5 to 87% for 10 min), m/z: 484.4 [M+H]⁺; Rt=5.05 min.

### Synthesis of tert-butyl 4-(4-((5-carbamoyl-4-((2-methoxy-3-(1-methyl-1H-1,2,4-triazol-3-yl)phenyl)amino)pyrimidin-2-yl)amino)-1H-pyrazol-1-yl)piperidine-1-carboxylate (P71)

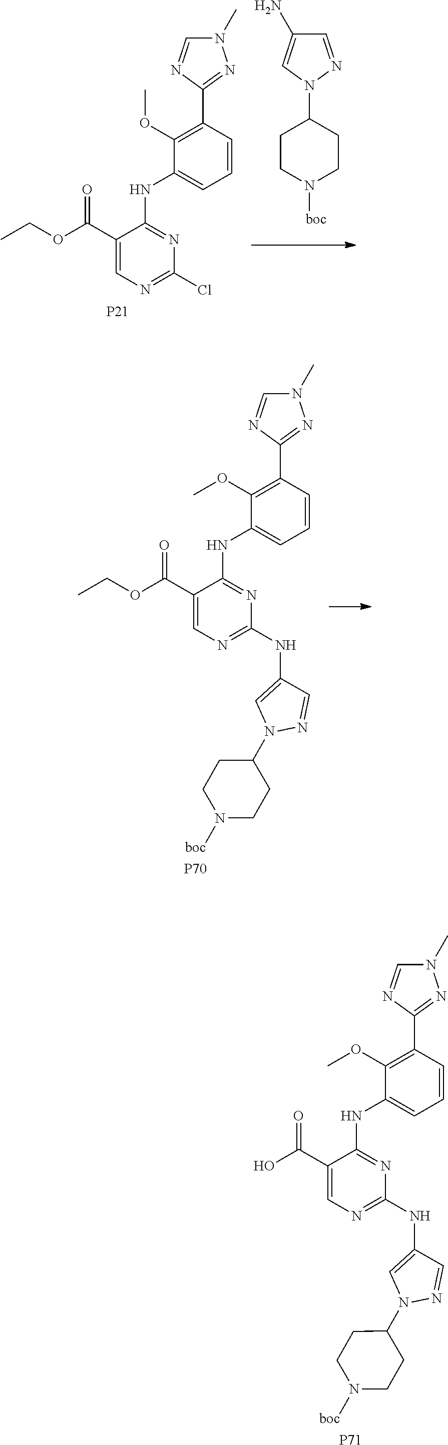

### Preparation 70. Ethyl 2-((1-(1-(tert-butoxycarbonyl) piperidin-4-yl)-1H-pyrazol-4-yl)amino)-4-((2-methoxy-3-(1-methyl-1H-1,2,4-triazol-3-yl)phenyl)amino)pyrimidine-5-carboxylate (P70)

To a solution of P21 (600 mg, 1.54 mmol) in dioxane (150 mL, degassed) tert-butyl 4-(4-amino-1H-pyrazol-1-yl)piperidine-1-carboxylate (411 mg, 1.54 mmol), cesium carbonate (1.01 g, 3.09 mmol), Xantphos (268 mg, 0.46 mmol), and tris(dibenzylideneacetone) dipalladium (0) (104 mg, 0.46 mmol) were added at rt under argon atmosphere. The solution was stirred at 100° C. overnight, the mixture was cooled and filtered through a pad of Celite®. The filtrate was then concentrated under reduced pressure. The residue was purified by silica gel column chromatography (DCM/MeOH=95:5) to give P70 (131 mg, 14% yield) as a white solid. LCMS (ESI) (C18 column 100×4.6 mm, 5.0 μm, pore size 100 Å, water-acetonitrile+0.1% TFA, gradient 5 to 87% for 10 min), m/z: 619.6 [M+H]⁺; Rt=5.82 min.

### Preparation 71. 2-((1-(1-(tert-Butoxycarbonyl) piperidin-4-yl)-1H-pyrazol-4-yl)amino)-4-((2-methoxy-3-(1-methyl-1H-1,2,4-triazol-3-yl)phenyl)amino)pyrimidine-5-carboxylic Acid (P71)

To a solution of P70 (131 mg, 0.212 mmol) in THF (20 mL) water (5 mL) and lithium hydroxide (89 mg, 2.12 mmol) were added at rt. The reaction mixture was stirred overnight, concentrated, and acidified with 1M HCl to pH=7. The resulting precipitate was centrifugated and dried to give P71 (125 mg, 99% yield) as a yellow solid. LCMS (ESI) (C18 column 100×4.6 mm, 5.0 μm, pore size 100 Å, water-acetonitrile+0.1% TFA, gradient 5 to 87% for 10 min), m/z: 591.6 [M+H]⁺; Rt=5.10 min.

### Synthesis of 2-((1-(1-adamantyl)-1H-pyrazol-4-yl)amino)-4-((2-methoxy-3-(1-methyl-1H-1,2,4-triazol-3-yl)phenyl)amino)pyrimidine-5-carboxylic Acid (P73)

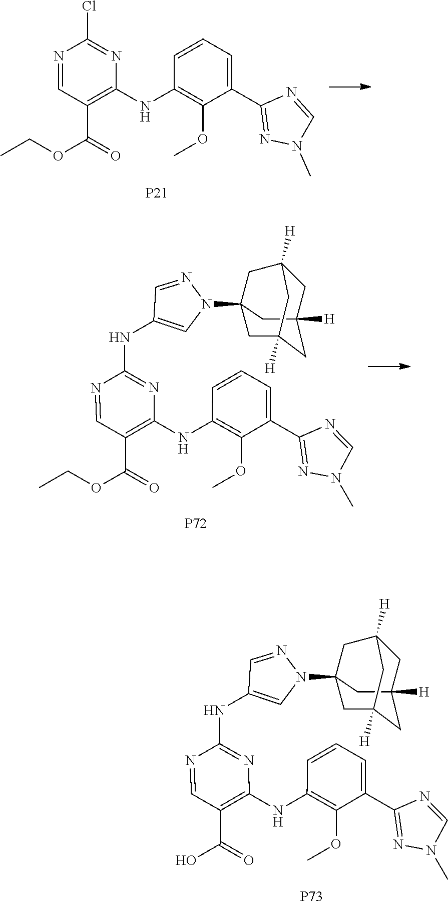

### Preparation 72. Ethyl 2-((1-(1-adamantyl)-1H-pyrazol-4-yl)amino)-4-((2-methoxy-3-(1-methyl-1H-1,2,4-triazol-3-yl)phenyl)amino)pyrimidine-5-carboxylate (P72)

To a solution of P21 (150 mg, 0.38 mmol) in dioxane (10 mL, degassed) was added 1-(1-adamantyl)-1H-pyrazol-4-amine (P76, 160 mg, 0.73 mmol), cesium carbonate (0.5 g, 1.53 mmol), Xantphos (25 mg, 0.04 mmol), and palladium acetate (10 mg, 0.04 mmol) at rt under argon atmosphere. After the solution was stirred at 100° C. for overnight, the mixture was cooled and filtered through a pad of celite. The filtrate was then concentrated under reduced pressure. The residue was purified by silica gel column chromatography (CHCl₃/MeOH=95:5) to give P72 (159 mg, 72% yield) as a white solid. LCMS (ESI), m/z: 570.5 [M+H]⁺; Rt=1.65 min.

### Preparation 73. 2-((1-(1-Adamantyl)-1H-pyrazol-4-yl)amino)-4-((2-methoxy-3-(1-methyl-1H-1,2,4-triazol-3-yl)phenyl)amino)pyrimidine-5-carboxylic Acid (P73)

To a solution of P72 (159 mg, 0.25 mmol) in THF (8.5 mL) was added water (8.5 mL) and lithium hydroxide (120 mg, 2.85 mmol) at rt. After it was stirred for overnight, the mixture was concentrated and then acidified with diluted HCl. Precipitated solid was filtered and dried on air to give a yellow solid. Yield 0.143 g (94%). LCMS (ESI), m/z: 542.8 [M+H]⁺; Rt=1.30 min.

### Synthesis of 1-(1-adamantyl)-1H-pyrazol-4-amine (P76)

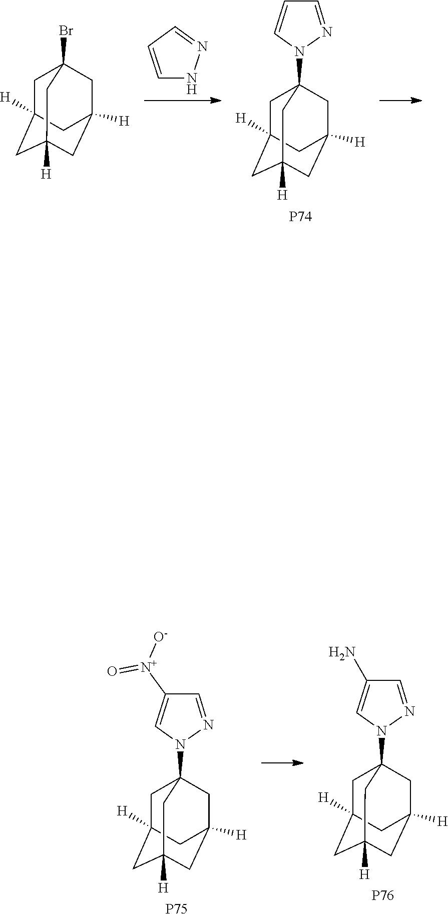

### Preparation 74. 1-(1-Adamantyl)-1H-pyrazole (P74)

1-Bromoadamantane (0.6 g, 2.79 mmol) and 1H-pyrazole (0.6 g, 8.81 mmol) were melted at 190° C. for 2 h and the reaction mixture after cooling to rt was partitioned between aqueous NaHCO₃ and DCM. Organic phase was separated; aqueous one was extracted with DCM (2 by 5 ml). Combined organic phases were dried over MgSO₄ and concentrated. The product was purified by chromatography on silica using CHCl₃ as an eluent. Yield 0.45 g (79%). ¹H NMR (400 MHz, CDCl₃) § 7.55 (s, 1H), 7.53 (d, J=2.1 Hz, 1H), 6.25 (t, J=2.0 Hz, 1H), 2.24 (s, 3H), 2.19 (d, J=2.6 Hz, 6H), 1.84-1.73 (m, 6H).

### Preparation 75. 1-(1-Adamantyl)-4-nitro-1H-pyrazole (P75)

Compound P74 (0.61 g, 3.0 mmol) was added in portions to ice cold conc H₂SO₄ (2 ml), and then 70% HNO₃ (0.25 ml) was added dropwise. The mixture was heated at 50° C. for 2 h and then poured onto ice. The product was extracted with DCM (3 by 10 ml). Organic phase was dried over MgSO₄ and concentrated. Yield 0.67 g (89%). ¹H NMR (400 MHz, CDCl₃), δ: 8.23 (s, 1H), 8.12 (s, 1H), 2.29 (s, 2H), 2.29 (s, 2H), 2.19-2.07 (m, 7H), 1.87-1.72 (m, 6H).

### Preparation 76. 1-(1-Adamantyl)-1H-pyrazol-4-amine (P76)

To a solution of compound P75 (0.6 g, 2.42 mmol) in MeOH (10 ml) 10% Pd on C (0.06 g) was added. The reaction mixture was flushed with argon and hydrogenated at rt overnight. The catalyst was filtered, washed with MeOH and combined organic phases were evaporated dryness. The product was used in the next step without additional purification. Yield 0.49 g (93%). ¹H NMR (400 MHz, CDCl₃), δ: 7.20 (s, 1H), 7.17 (s, 1H), 3.75 (s, 2H), 2.21 (s, 2H), 2.11 (s, 5H), 1.86 (s, 2H), 1.75 (s, 6H).

### Synthesis of tert-butyl 4-bromopiperidine-1-carboxylate (P77)

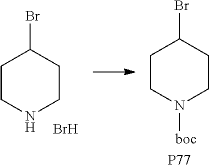

### Preparation 77. tert-Butyl 4-bromopiperidine-1-carboxylate (P77)

4-Bromopiperidine hydrobromide (10 g, 40.8 mmol) was dissolved in DMF (50 ml), DIPEA was added (14.2 ml, 10.55 g, 81.7 mmol) and the reaction mixture was stirred at ambient temperature for 30 min. The reaction mixture was cooled to 0° C. and di-tert-butyl bicarbonate (13.36 g, 61.2 mmol) was added portionwise. Then the reaction mixture was stirred at rt for 18 h. The reaction mixture was poured into water (250 ml), acidified to slightly acidic pH, extracted with DCM (3×50 ml). The combined organic phases were washed with water (100 ml) and dried over Na₂SO₄. The solvent was evaporated in vacuo, and the product P77 was used further without additional purification. Yield 10.2 g (95%). ¹H NMR (400 MHz, DMSO-d₆), δ: 4.55-4.50 (tt, J=7.7, 3.8 Hz, 1H), 3.60-3.53 (m, 2H), 3.23-3.17 (m, 2H), 2.09-2.04 (m, 2H), 1.81-1.73 (m, 2H), 1.47 (s, 9H).

### Synthesis of Amines P80, P82, P84

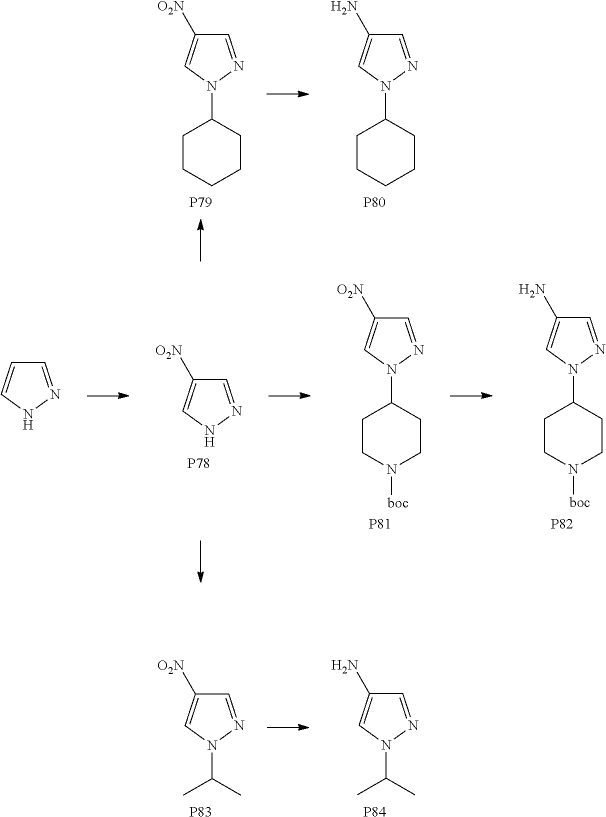

### Preparation 78. 4-Nitro-1H-pyrazole (P78)

Pyrazole (10 g, 147.05 mmol) was dissolved in concentrated sulfuric acid (60 ml) and the reaction mixture was cooled to −5° C. Nitric acid (6.74 ml, 161.6 mmol) was added dropwise. The reaction mixture was stirred for 18 h at ambient temperature, then poured on 250 g of ice, and extracted with ethyl acetate (3×100 ml). The combined organic layer was washed with 10% sodium bicarbonate solution until the evolution of CO₂ ceased, and then dried over Na₂SO₄ and concentrated. Yield 13.35 g (81%). ¹H-NMR (400 MHz, DMSO-d₆), δ: 13.98 (br. s, 1H), 8.59 (br. s, 2H).

### Preparation 79. 1-Cyclohexyl-4-nitro-1H-pyrazole (P79)

4-Nitro-1H-pyrazole P78 (10 g, 88.5 mmol) and cesium carbonate (57.62 g, 176.86 mmol) were suspended in DMF and cyclohexyl bromide (14.42 g, 88.5 mmol) was added. The reaction mixture was stirred at 80° C. for 15 h. The reaction mixture was poured into water, extracted with ethyl acetate, and the combined organic layers were washed with water dried over Na₂SO₄ and concentrated. The residue was purified by column chromatography using ethyl acetate/hexane (1:4) to give P79. Yield 9.1 g (53%). ¹H-NMR (400 MHz, DMSO-d₆), δ: 8.89 (s, 1H), 8.24 (s, 1H), 4.17-4.27 (m, 1H), 1.99-2.04 (d, J=12.6 Hz, 2H), 1.63-1.83 (m, 5H), 1.33-1.43 (dd, J=13.0 Hz, 2H), 1.14-1.25 (m, 1H).

### Preparation 80. 1-Cyclohexyl-1H-pyrazol-4amine (P80)

1-Cyclohexyl-4-nitro-1H-pyrazole P79 (9.1 g, 46.67 mmol) was dissolved in dry methanol and 0.496 g 10% Pd/C was added. The reaction mixture was stirred in hydrogen atmosphere (20 atm) for 15 h at 25° C. The reaction mixture was filtered through a celite pad and evaporated to provide the product P80. Yield 7.67 g (99%). ¹H-NMR (400 MHz, DMSO-d₆), δ: 7.03 (s, 1H), 6.88 (s, 1H), 3.85-3.93 (m, 1H), 3.70 (br. s, 2H), 1.90-1.93 (d, J=12.3 Hz, 2H), 1.74-1.84 (d, J=13.7 Hz, 2H), 1.53-1.64 (m, 3H), 1.28-1.40 (dd, J=12.7 Hz, 2H), 1.10-1.23 (m, 1H).

### Preparation 81. tert-Butyl 4-(4-nitro-1H-pyrazol-1-yl)piperidine-1carboxylate (P81)

4-Nitro-1H-pyrazole P78 (2 g, 17.7 mmol) and cesium carbonate (11.53 g, 35.37 mmol) were suspended in DMF and tert-butyl 4-bromopiperidine-1-carboxylate P77 (4.67 g, 17.7 mmol) was added and the reaction mixture was stirred at 80° C. for 15 h. The reaction mixture was poured into water, extracted with ethyl acetate, and the combined organic layers were washed with water, dried over Na₂SO₄ and concentrated. The residue was purified by column chromatography using ethyl acetate/hexane (1:9). Yield of P81-2.21 g (42%). ¹H-NMR (400 MHz, DMSO-d₆), δ: 8.94 (s, 1H), 8.26 (s, 1H), 4.42-4.51 (m, 1H), 3.99-4.01 (m, 2H), 2.82-2.98 (m, 2H), 2.01-2.06 (d, J=12.3 Hz, 2H), 1.75-1.87 (m, 2H), 1.41 (s, 9H).

### Preparation 82. tert-Butyl 4-(4-amino-1H-pyrazol-1-yl)piperidine-1-carboxylate (P82)

tert-Butyl 4-(4-nitro-1H-pyrazol-1-yl)piperidine-1carboxylate P81 (1.15 g, 3.88 mmol) was dissolved in dry methanol, 0.041 g 10% Pd/C was added. The reaction mixture was stirred at 20 atm in a hydrogen atmosphere for 15 h at a temperature of 25° C. The reaction mass was filtered through a celite pad and evaporated to provide the product P82. Yield 1.03 g (99%). ¹H NMR (400 MHz, CDCl₃), δ: 7.16 (s, 1H), 7.03 (s, 1H), 4.22 (br. s, 2H), 4.09-4.17 (m, 1H), 2.83-2.90 (m, 2H), 2.78 (br. s, 2H), 2.05-2.09 (d, J=12.2 Hz, 2H), 1.78-1.89 (m, 2H), 1.47 (s, 9H).

### Preparation 83. 1-Isopropyl-4-nitro-1H-pyrazole (P83)

4-Nitro-1H-pyrazole P78 (3 g, 26.53 mmol) and cesium carbonate (17.29 g, 49 mmol) were suspended in DMF and isopropyl chloride (2.42 ml, 2.08 g, 26.53 mmol) was added dropwise. The reaction mixture was stirred at 80° C. for 15 h. The reaction mixture was poured into water, extracted with ethyl acetate, and the combined organic layers were washed with water, dried over Na₂SO₄ and concentrated. The residue was purified by column chromatography using ethyl acetate/hexane (3:6) to give P83. Yield 2.67 g (65%). ¹H-NMR (400 MHz, DMSO-d₆), δ: 8.91 (s, 1H), 8.25 (s, 1H), 4.53-4.64 (m, 1H), 1.43-1.45 (d, J=6.7 Hz, 6H).

### Preparation 84. 1-Isopropyl-1H-pyrazol-4-amine (P84)

1-Isopropyl-4-nitro-1H-pyrazole P83 (14.35 g, 92.48 mmol) was dissolved in dry methanol, 0.984 g 10% Pd/C was added. The reaction mixture was stirred in hydrogen atmosphere (20 atm) for 15 h at 25° C. The reaction mass was filtered through celite pad and evaporated to provide the product P84. Yield 11.37 g (99%). ¹H NMR (400 MHz, DMSO-d₆), δ: 7.03 (s, 1H), 6.90 (s, 1H), 4.22-4.32 (m, 1H), 3.73 (br s, 2H), 1.31-1.33 (d, J=6.6 Hz, 6H).

In the Table 3 presented certain examples of key intermediates.

TABLE 3
Certain examples of key intermediates.

| | | [MH]⁺ | [MH]⁺ |
|---|---|---|---|
| # | Structure | Calc. | Found |
| P6 | 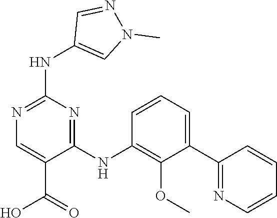 [SMILES: COc1c(Nc2nc(Nc3cnn(C)c3)ncc2C(=O)O)cccc1-c1ccccn1] | 418.163 | 418.8 |
| P13 | 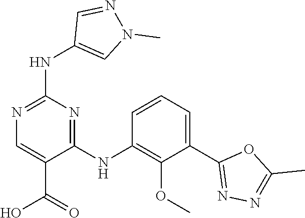 [SMILES: COc1c(Nc2nc(Nc3cnn(C)c3)ncc2C(=O)O)cccc1-c1nnc(C)o1] | 423.153 | 423.4 |
| P17 | 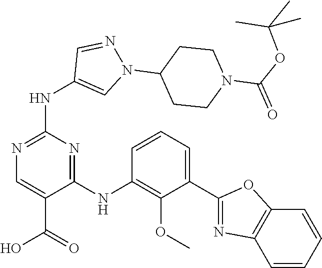 [SMILES: COc1c(Nc2nc(Nc3cnn(C4CCN(C(=O)OC(C)(C)C)CC4)c3)ncc2C(=O)O)cccc1-c1nc2ccccc2o1] | 627.268 | 627.7 |
| P20 | 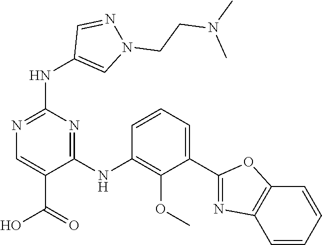 | 515.216 | 515.8 |
| P23 | 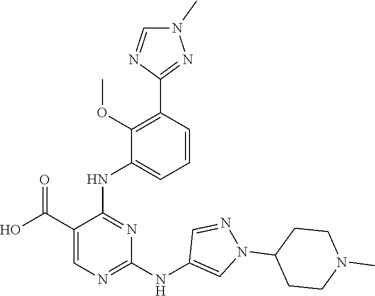 | 505.242 | 505.5 |
| P27 | 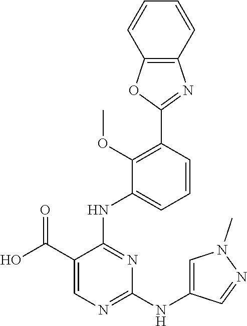 [SMILES: COc1c(Nc2nc(Nc3cnn(C)c3)ncc2C(=O)O)cccc1-c1nc2ccccc2o1] | 548.158 | 458.9 |
| P29 | 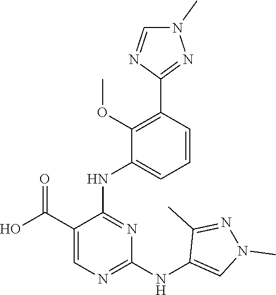 [SMILES: COc1c(Nc2nc(Nc3cn(C)nc3C)ncc2C(=O)O)cccc1-c1ncn(C)n1] | 436.185 | 436.4 |
| P31 | 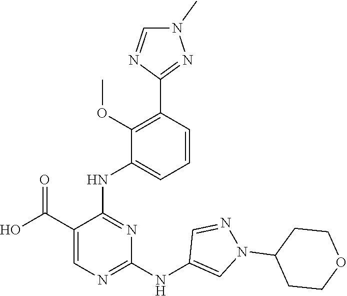 | 492.211 | 492.2 |
| P33 | 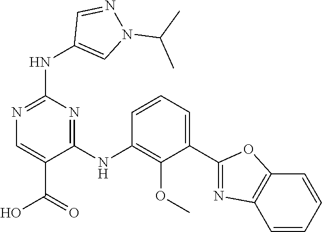 | 486.189 | 486.5 |
| P35 | 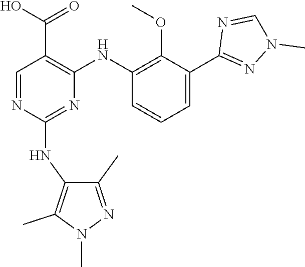 [SMILES: COc1c(Nc2nc(Nc3c(C)nn(C)c3C)ncc2C(=O)O)cccc1-c1ncn(C)n1] | 450.2 | 450.2 |
| P37 | 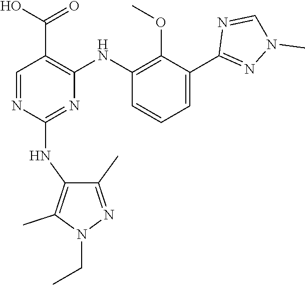 [SMILES: CCn1nc(C)c(Nc2ncc(C(=O)O)c(Nc3cccc(-c4ncn(C)n4)c3OC)n2)c1C] | 464.216 | 464.5 |
| P39 | 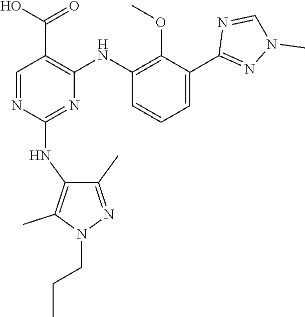 [SMILES: CCCn1nc(C)c(Nc2ncc(C(=O)O)c(Nc3cccc(-c4ncn(C)n4)c3OC)n2)c1C] | 478.232 | 478.3 |
| P41 | 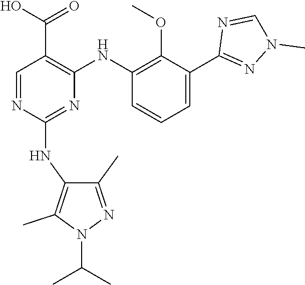 [SMILES: COc1c(Nc2nc(Nc3c(C)nn(C(C)C)c3C)ncc2C(=O)O)cccc1-c1ncn(C)n1] | 478.232 | 478.4 |
| P43 | 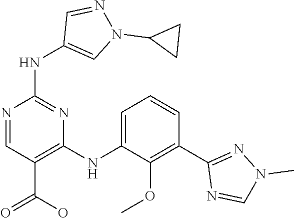 | 447.185 | 447.4 |
| P45 | 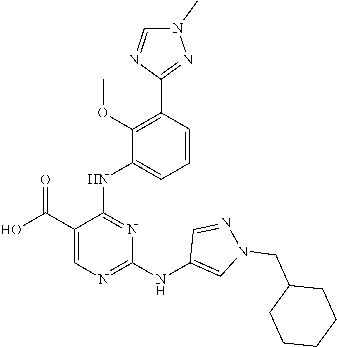 | 504.247 | 504.5 |
| P47 | 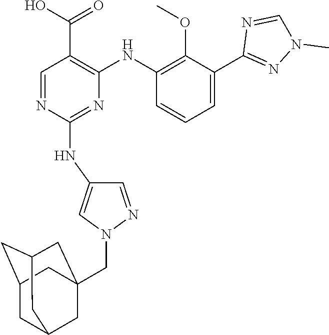 [SMILES: COc1c(Nc2nc(Nc3cnn(CC45CC6CC(CC(C6)C4)C5)c3)ncc2C(=O)O)cccc1-c1ncn(C)n1] | 556.278 | 556.5 |
| P49 | 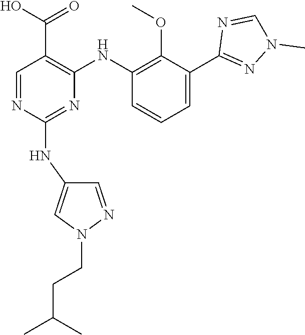 [SMILES: COc1c(Nc2nc(Nc3cnn(CCC(C)C)c3)ncc2C(=O)O)cccc1-c1ncn(C)n1] | 478.232 | 478.4 |
| P51 | 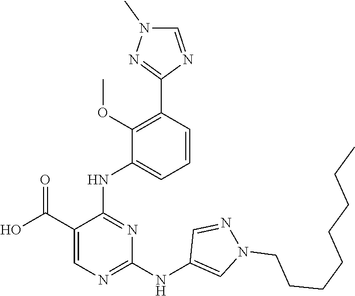 [SMILES: CCCCCCCCn1cc(Nc2ncc(C(=O)O)c(Nc3cccc(-c4ncn(C)n4)c3OC)n2)cn1] | 520.278 | 520.8 |
| P53 | 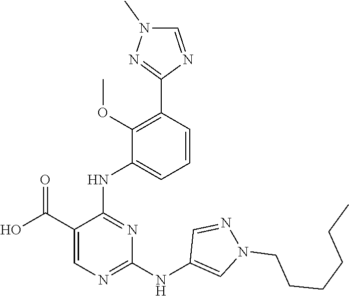 [SMILES: CCCCCCn1cc(Nc2ncc(C(=O)O)c(Nc3cccc(-c4ncn(C)n4)c3OC)n2)cn1] | 492.247 | 492.4 |
| P55 | 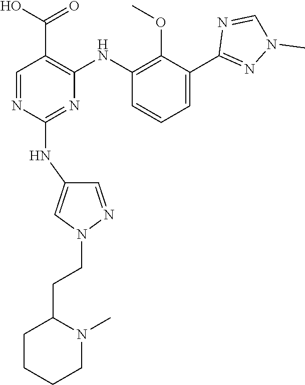 [SMILES: COc1c(Nc2nc(Nc3cnn(CCC4CCCCN4C)c3)ncc2C(=O)O)cccc1-c1ncn(C)n1] | 533.274 | 533.3 |
| P57 | 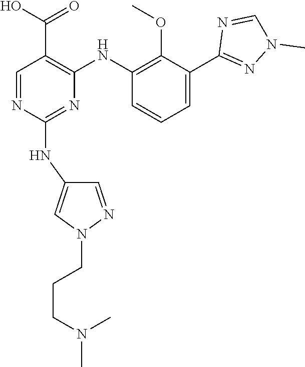 [SMILES: COc1c(Nc2nc(Nc3cnn(CCCN(C)C)c3)ncc2C(=O)O)cccc1-c1ncn(C)n1] | 493.242 | 493.8 |
| P59 | 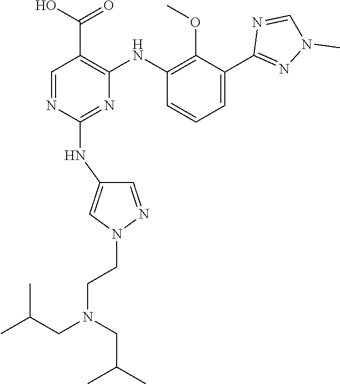 [SMILES: COc1c(Nc2nc(Nc3cnn(CCN(CC(C)C)CC(C)C)c3)ncc2C(=O)O)cccc1-c1ncn(C)n1] | 563.321 | 563.8 |
| P61 | 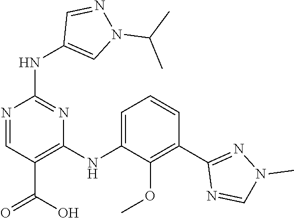 [SMILES: COc1c(Nc2nc(Nc3cnn(C(C)C)c3)ncc2C(=O)O)cccc1-c1ncn(C)n1] | 450.2 | 450.5 |
| P63 | 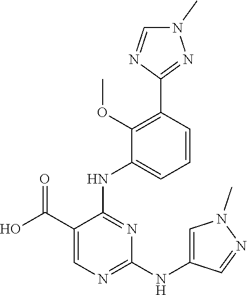 [SMILES: COc1c(Nc2nc(Nc3cnn(C)c3)ncc2C(=O)O)cccc1-c1ncn(C)n1] | 422.169 | 422.3 |
| P65 | 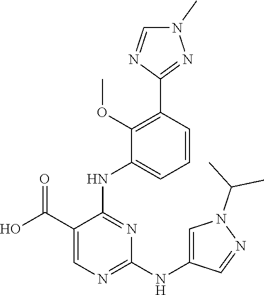 [SMILES: COc1c(Nc2nc(Nc3cnn(C(C)C)c3)ncc2C(=O)O)cccc1-c1ncn(C)n1] | 450.2 | 450.3 |
| P67 | 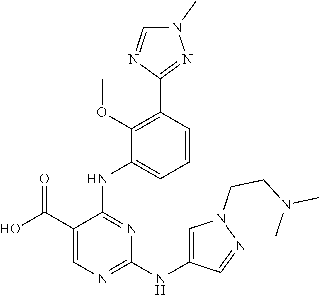 [SMILES: COc1c(Nc2nc(Nc3cnn(CCN(C)C)c3)ncc2C(=O)O)cccc1-c1ncn(C)n1] | 479.227 | 479.4 |
| P69 | 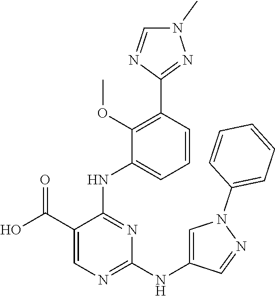 [SMILES: COc1c(Nc2nc(Nc3cnn(-c4ccccc4)c3)ncc2C(=O)O)cccc1-c1ncn(C)n1] | 484.185 | 484.4 |
| P71 | 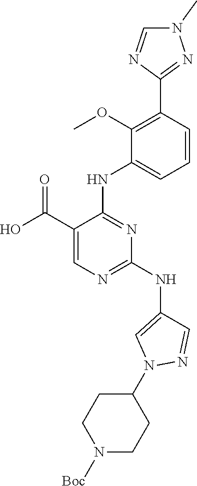 [SMILES: COc1c(Nc2nc(Nc3cnn(C4CCN(C(=O)OC(C)(C)C)CC4)c3)ncc2C(=O)O)cccc1-c1ncn(C)n1] | 591.279 | 591.6 |
| P73 | 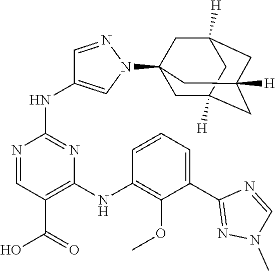 [SMILES: COc1c(Nc2nc(Nc3cnn([C@]45C[C@H]6C[C@H](C[C@H](C6)C4)C5)c3)ncc2C(=O)O)cccc1-c1ncn(C)n1] | 542.263 | 542.8 |

### EXAMPLES OF THE FINAL COMPOUND

Table 4 presents certain non-limiting examples of a compound of Formula (I).

TABLE 4
Selected examples of a compound of Formula (I)

| | | [MH]⁺ | [MH]⁺ |
|---|---|---|---|
| # | IUPAC Name | Calc. | Found |
| 1 | 4-[2-methoxy-3-(1-methyl-1,2,4-triazol-3-yl)anilino]-2- | 421.1849 | 421 |
| | [(1-methylpyrazol-4-yl)amino]pyrimidine-5- | | |
| | carboxamide | | |
| 2 | 4-[2-methoxy-3-(2-pyridyl)anilino]-2-[(1- | 417.1787 | 417 |
| | methylpyrazol-4-yl)amino]pyrimidine-5-carboxamide | | |
| 3 | 4-[2-methoxy-3-(5-methyl-1,3,4-oxadiazol-2- | 422.1689 | 422 |
| | yl)anilino]-2-[(1-methylpyrazol-4-yl)amino]pyrimidine- | | |
| | 5-carboxamide | | |
| 4 | 2-[(1-isopropylpyrazol-4-yl)amino]-4-[2-methoxy-3-(1- | 449.2162 | 449 |
| | methyl-1,2,4-triazol-3-yl)anilino]pyrimidine-5- | | |
| | carboxamide | | |
| 5 | 2-[[1-[2-(dimethylamino)ethyl]pyrazol-4-yl]amino]-4- | 478.2427 | 478 |
| | [2-methoxy-3-(1-methyl-1,2,4-triazol-3- | | |
| | yl)anilino]pyrimidine-5-carboxamide | | |
| 6 | 4-[2-methoxy-3-(1-methyl-1,2,4-triazol-3-yl)anilino]-2- | 483.2005 | 483 |
| | [(1-phenylpyrazol-4-yl)amino]pyrimidine-5- | | |
| | carboxamide | | |
| 7 | 4-[2-methoxy-3-(1-methyl-1,2,4-triazol-3-yl)anilino]-2- | 490.2427 | 490 |
| | [[1-(4-piperidyl)pyrazol-4-yl]amino]pyrimidine-5- | | |
| | carboxamide | | |
| 8 | 4-[3-(1,3-benzoxazol-2-yl)-2-methoxy-anilino]-2-[[1- | 526.2315 | 526 |
| | (4-piperidyl)pyrazol-4-yl]amino]pyrimidine-5- | | |
| | carboxamide | | |
| 9 | 4-[3-(1,3-benzoxazol-2-yl)-2-methoxy-anilino]-2-[[1- | 514.2315 | 514 |
| | [2-(dimethylamino)ethyl]pyrazol-4- | | |
| | yl]amino]pyrimidine-5-carboxamide | | |
| 10 | 4-[2-methoxy-3-(1-methyl-1,2,4-triazol-3-yl)anilino]-2- | 504.2584 | 504 |
| | [[1-(1-methyl-4-piperidyl)pyrazol-4- | | |
| | yl]amino]pyrimidine-5-carboxamide | | |
| 11 | 2-[(1-cyclohexylpyrazol-4-yl)amino]-4-[2-methoxy-3- | 489.2475 | 489 |
| | (1-methyl-1,2,4-triazol-3-yl)anilino]pyrimidine-5- | | |
| | carboxamide | | |
| 12 | 4-[3-(1,3-benzoxazol-2-yl)-2-methoxy-anilino]-2-[(1- | 457.1736 | 457 |
| | methylpyrazol-4-yl)amino]pyrimidine-5-carboxamide | | |
| 13 | 2-[(1,3-dimethylpyrazol-4-yl)amino]-4-[2-methoxy-3- | 435.2005 | 435 |
| | (1-methyl-1,2,4-triazol-3-yl)anilino]pyrimidine-5- | | |
| | carboxamide | | |
| 14 | 4-[2-methoxy-3-(1-methyl-1,2,4-triazol-3-yl)anilino]-2- | 491.2267 | 491 |
| | [(1-tetrahydropyran-4-ylpyrazol-4- | | |
| | yl)amino]pyrimidine-5-carboxamide | | |
| 15 | 4-(2-methoxy-3-pyrimidin-2-yl-anilino)-2-[(1- | 418.174 | 418 |
| | methylpyrazol-4-yl)amino]pyrimidine-5-carboxamide | | |
| 16 | 4-[3-(1,3-benzoxazol-2-yl)-2-methoxy-anilino]-2-[(1- | 485.2049 | 485 |
| | isopropylpyrazol-4-yl)amino]pyrimidine-5-carboxamide | | |
| 17 | 4-[2-methoxy-3-(1-methyl-1,2,4-triazol-3-yl)anilino]-2- | 449.2162 | 449 |
| | [(1,3,5-trimethylpyrazol-4-yl)amino]pyrimidine-5- | | |
| | carboxamide | | |
| 18 | 2-[(1-ethyl-3,5-dimethyl-pyrazol-4-yl)amino]-4-[2- | 463.2318 | 463 |
| | methoxy-3-(1-methyl-1,2,4-triazol-3- | | |
| | yl)anilino]pyrimidine-5-carboxamide | | |
| 19 | 2-[(3,5-dimethyl-1-propyl-pyrazol-4-yl)amino]-4-[2- | 477.2475 | 477 |
| | methoxy-3-(1-methyl-1,2,4-triazol-3- | | |
| | yl)anilino]pyrimidine-5-carboxamide | | |
| 20 | 2-[(1-isopropyl-3,5-dimethyl-pyrazol-4-yl)amino]-4-[2- | 477.2475 | 477 |
| | methoxy-3-(1-methyl-1,2,4-triazol-3- | | |
| | yl)anilino]pyrimidine-5-carboxamide | | |
| 21 | 2-[(1-cyclopropylpyrazol-4-yl)amino]-4-[2-methoxy-3- | 447.2005 | 447 |
| | (1-methyl-1,2,4-triazol-3-yl)anilino]pyrimidine-5- | | |
| | carboxamide | | |
| 22 | 2-[[1-(cyclohexylmethyl)pyrazol-4-yl]amino]-4-[2- | 503.2631 | 503 |
| | methoxy-3-(1-methyl-1,2,4-triazol-3- | | |
| | yl)anilino]pyrimidine-5-carboxamide | | |
| 23 | 2-[[1-(1-adamantylmethyl)pyrazol-4-yl]amino]-4-[2- | 555.2944 | 555 |
| | methoxy-3-(1-methyl-1,2,4-triazol-3- | | |
| | yl)anilino]pyrimidine-5-carboxamide | | |
| 24 | 2-[(1-isopentylpyrazol-4-yl)amino]-4-[2-methoxy-3-(1- | 477.2475 | 477 |
| | methyl-1,2,4-triazol-3-yl)anilino]pyrimidine-5- | | |
| | carboxamide | | |
| 25 | 4-[2-methoxy-3-(1-methyl-1,2,4-triazol-3-yl)anilino]-2- | 519.2944 | 519 |
| | [(1-octylpyrazol-4-yl)amino]pyrimidine-5-carboxamide | | |
| 26 | 2-[(1-hexylpyrazol-4-yl)amino]-4-[2-methoxy-3-(1- | 491.2631 | 491 |
| | methyl-1,2,4-triazol-3-yl)anilino]pyrimidine-5- | | |
| | carboxamide | | |
| 27 | 4-[2-methoxy-3-(1-methyl-1,2,4-triazol-3-yl)anilino]-2- | 532.2897 | 532 |
| | [[1-[2-(1-methyl-2-piperidyl)ethyl]pyrazol-4- | | |
| | yl]amino]pyrimidine-5-carboxamide | | |
| 28 | 2-[[1-[3-(dimethylamino)propyl]pyrazol-4-yl]amino]-4- | 492.2584 | 492 |
| | [2-methoxy-3-(1-methyl-1,2,4-triazol-3- | | |
| | yl)anilino]pyrimidine-5-carboxamide | | |
| 29 | 2-[[1-[2-(diisobutylamino)ethyl]pyrazol-4-yl]amino]-4- | 562.3366 | 562 |
| | [2-methoxy-3-(1-methyl-1,2,4-triazol-3- | | |
| | yl)anilino]pyrimidine-5-carboxamide | | |
| 30 | 2-[[1-(1-adamantyl)pyrazol-4-yl]amino]-4-[2-methoxy- | 541.2788 | 541 |
| | 3-(1-methyl-1,2,4-triazol-3-yl)anilino]pyrimidine-5- | | |
| | carboxamide | | |
| 31 | 2-[(1-isopropylpyrazol-4-yl)amino]-4-[2-methoxy-3-(1- | 477.2475 | 477 |
| | methyl-1,2,4-triazol-3-yl)anilino]-N,N-dimethyl- | | |
| | pyrimidine-5-carboxamide | | |
| 32 | 4-[3-(1,3-benzoxazol-2-yl)-2-methoxy-anilino]-2-[[1- | 540.2471 | 540 |
| | (1-methyl-4-piperidyl)pyrazol-4-yl]amino]pyrimidine- | | |
| | 5-carboxamide | | |
| 33 | 4-[3-(1,3-benzoxazol-2-yl)-2-methoxy-anilino]-2-[[1- | 568.2784 | 568 |
| | (1-isopropyl-4-piperidyl)pyrazol-4- | | |
| | yl]amino]pyrimidine-5-carboxamide | | |
| 34 | 4-[3-(1,3-benzoxazol-2-yl)-2-methoxy-anilino]-2-[[1- | 598.3254 | 598 |
| | [2-(diisobutylamino)ethyl]pyrazol-4- | | |
| | yl]amino]pyrimidine-5-carboxamide | | |
| 35 | 2-[[1-(1-isopropyl-4-piperidyl)pyrazol-4-yl]amino]-4- | 532.2897 | 532 |
| | [2-methoxy-3-(1-methyl-1,2,4-triazol-3- | | |
| | yl)anilino]pyrimidine-5-carboxamide | | |
| 36 | 4-[2-methoxy-3-(3-pyridyl)anilino]-2-[(1- | 417.1787 | 417 |
| | methylpyrazol-4-yl)amino]pyrimidine-5-carboxamide | | |
| 37 | 4-[2-methoxy-3-(4-pyridyl)anilino]-2-[(1- | 417.1787 | 417 |
| | methylpyrazol-4-yl)amino]pyrimidine-5-carboxamide | | |
| 38 | 2-[(1-isopropylpyrazol-4-yl)amino]-4-[2-methoxy-3-(2- | 445.21 | 445 |
| | pyridyl)anilino]pyrimidine-5-carboxamide | | |
| 39 | 2-[(1-cyclohexylpyrazol-4-yl)amino]-4-[2-methoxy-3- | 485.2413 | 485 |
| | (2-pyridyl)anilino]pyrimidine-5-carboxamide | | |
| 40 | 4-[2-methoxy-3-(2-pyridyl)anilino]-2-[(1- | 487.2206 | 487 |
| | tetrahydropyran-4-ylpyrazol-4-yl)amino]pyrimidine-5- | | |
| | carboxamide | | |
| 41 | 2-[[1-(1-adamantyl)pyrazol-4-yl]amino]-4-[2-methoxy- | 537.2726 | 537 |
| | 3-(2-pyridyl)anilino]pyrimidine-5-carboxamide | | |
| 42 | 2-[[1-[2-(diisobutylamino)ethyl]pyrazol-4-yl]amino]-4- | 558.3305 | 558 |
| | [2-methoxy-3-(2-pyridyl)anilino]pyrimidine-5- | | |
| | carboxamide | | |
| 43 | 2-[[1-[3-(dimethylamino)propyl]pyrazol-4-yl]amino]-4- | 488.2522 | 488 |
| | [2-methoxy-3-(2-pyridyl)anilino]pyrimidine-5- | | |
| | carboxamide | | |
| 44 | 4-[3-(1,3-benzoxazol-2-yl)-2-methoxy-anilino]-2-[(1- | 527.2155 | 527 |
| | tetrahydropyran-4-ylpyrazol-4-yl)amino]pyrimidine-5- | | |
| | carboxamide | | |
| 45 | 4-[3-(1,3-benzoxazol-2-yl)-2-methoxy-anilino]-2-[(1- | 525.2363 | 525 |
| | cyclohexylpyrazol-4-yl)amino]pyrimidine-5- | | |
| | carboxamide | | |
| 46 | 4-[3-(1,3-benzoxazol-2-yl)-2-methoxy-anilino]-2-[[1- | 528.2471 | 528 |
| | [3-(dimethylamino)propyl]pyrazol-4- | | |
| | yl]amino]pyrimidine-5-carboxamide | | |
| 47 | 4-[2-methoxy-3-(2-pyridyl)anilino]-2-[[1-(1-methyl-4- | 500.2522 | 500 |
| | piperidyl)pyrazol-4-yl]amino]pyrimidine-5- | | |
| | carboxamide | | |
| 48 | 2-[[1-(1-isopropyl-4-piperidyl)pyrazol-4-yl]amino]-4- | 528.2835 | 528 |
| | [2-methoxy-3-(2-pyridyl)anilino]pyrimidine-5- | | |
| | carboxamide | | |
| 49 | 2-[[1-(1-adamantyl)pyrazol-4-yl]amino]-4-[3-(1,3- | 577.2676 | 577 |
| | benzoxazol-2-yl)-2-methoxy-anilino]pyrimidine-5- | | |
| | carboxamide | | |
| 50 | 4-[3-(1,3-benzoxazol-2-yl)-2-methoxy-anilino]-2-[[1- | 501.1998 | 501 |
| | (2-methoxyethyl)pyrazol-4-yl]amino]pyrimidine-5- | | |
| | carboxamide | | |
| 51 | 2-[[1-(2-methoxyethyl)pyrazol-4-yl]amino]-4-[2- | 465.2111 | 465 |
| | methoxy-3-(1-methyl-1,2,4-triazol-3- | | |
| | yl)anilino]pyrimidine-5-carboxamide | | |
| 52 | 4-[3-(1,3-benzoxazol-2-yl)-2-methoxy-anilino]-2-[[1- | 515.2155 | 515 |
| | (3-methoxypropyl)pyrazol-4-yl]amino]pyrimidine-5- | | |
| | carboxamide | | |
| 53 | 4-[2-methoxy-3-[1-(3-methoxypropyl)-1,2,4-triazol-3- | 537.2686 | 537 |
| | yl]anilino]-2-[[1-(3-methoxypropyl)pyrazol-4- | | |
| | yl]amino]pyrimidine-5-carboxamide | | |
| 54 | 4-[3-(1,3-benzoxazol-2-yl)-2-methoxy-anilino]-2-[[1- | 515.2155 | 515 |
| | (2-ethoxyethyl)pyrazol-4-yl]amino]pyrimidine-5- | | |
| | carboxamide | | |
| 55 | 2-[[1-(2-ethoxyethyl)pyrazol-4-yl]amino]-4-[2- | 479.2267 | 479 |
| | methoxy-3-(1-methyl-1,2,4-triazol-3- | | |
| | yl)anilino]pyrimidine-5-carboxamide | | |
| 56 | 4-[3-(1,3-benzoxazol-2-yl)-2-methoxy-anilino]-2-[(1- | 499.2206 | 499 |
| | isobutylpyrazol-4-yl)amino]pyrimidine-5-carboxamide | | |
| 57 | 4-[3-(1,3-benzoxazol-2-yl)-2-methoxy-anilino]-2-[[1- | 525.161 | 525 |
| | (2,2,2-trifluoroethyl)pyrazol-4-yl]amino]pyrimidine-5- | | |
| | carboxamide | | |
| 58 | 2-[(1-isobutylpyrazol-4-yl)amino]-4-[2-methoxy-3-(1- | 463.2318 | 463 |
| | methyl-1,2,4-triazol-3-yl)anilino]pyrimidine-5- | | |
| | carboxamide | | |
| 59 | 4-[2-methoxy-3-(1-methyl-1,2,4-triazol-3-yl)anilino]-2- | 489.1723 | 489 |
| | [[1-(2,2,2-trifluoroethyl)pyrazol-4- | | |
| | yl]amino]pyrimidine-5-carboxamide | | |
| 60 | 2-[(1-isopropylpyrazol-4-yl)amino]-4-[2-methoxy-3-(1- | 498.2366 | 498 |
| | methylbenzimidazol-2-yl)anilino]pyrimidine-5- | | |
| | carboxamide | | |
| 61 | 4-[3-(1,3-benzothiazol-2-yl)-2-methoxy-anilino]-2-[(1- | 501.1821 | 501 |
| | isopropylpyrazol-4-yl)amino]pyrimidine-5-carboxamide | | |
| 62 | 4-[3-(5-tert-butyl-1,3-benzoxazol-2-yl)-2-methoxy- | 541.2676 | 541 |
| | anilino]-2-[(1-isopropylpyrazol-4-yl)amino]pyrimidine- | | |
| | 5-carboxamide | | |
| 63 | 4-[3-(5-bromo-1,3-benzoxazol-2-yl)-2-methoxy- | 563.1155 | 593 |
| | anilino]-2-[(1-isopropylpyrazol-4-yl)amino]pyrimidine- | | |
| | 5-carboxamide | | |
| 64 | 4-[3-[4-(3-hydroxyoxetan-3-yl)oxazol-2-yl]-2-methoxy- | 507.2104 | 507 |
| | anilino]-2-[(1-isopropylpyrazol-4-yl)amino]pyrimidine- | | |
| | 5-carboxamide | | |
| 65 | 2-[(1-isopropylpyrazol-4-yl)amino]-4-[2-methoxy-3-(7- | 516.2107 | 516 |
| | methyl-5-oxo-4H-oxazolo[4,5-b]pyridin-2- | | |
| | yl)anilino]pyrimidine-5-carboxamide | | |
| 66 | 4-[3-[4-[2-[(4-cyano-2-pyridyl)amino]-2-oxo- | 594.2325 | 594 |
| | ethyl]oxazol-2-yl]-2-methoxy-anilino]-2-[(1- | | |
| | isopropylpyrazol-4-yl)amino]pyrimidine-5-carboxamide | | |
| 67 | 4-[3-[4-[2-[(5-cyano-3-pyridyl)amino]-2-oxo- | 594.2325 | 594 |
| | ethyl]oxazol-2-yl]-2-methoxy-anilino]-2-[(1- | | |
| | isopropylpyrazol-4-yl)amino]pyrimidine-5-carboxamide | | |
| 68 | 2-[(1-isopropylpyrazol-4-yl)amino]-4-[2-methoxy-3-[4- | 528.1777 | 528 |
| | (methylsulfamoyl)oxazol-2-yl]anilino]pyrimidine-5- | | |
| | carboxamide | | |
| 69 | 2-[(1-isopropylpyrazol-4-yl)amino]-4-[2-methoxy-3-(4- | 569.193 | 569 |
| | tetrahydrofuran-3-ylsulfonyloxazol-2- | | |
| | yl)anilino]pyrimidine-5-carboxamide | | |
| 70 | 2-[(1-isopropylpyrazol-4-yl)amino]-4-[2-methoxy-3-[4- | 527.1825 | 527 |
| | (methylsulfonylmethyl)oxazol-2-yl]anilino]pyrimidine- | | |
| | 5-carboxamide | | |
| 71 | 2-[(1-isopropylpyrazol-4-yl)amino]-4-[2-methoxy-3-(4- | 513.1669 | 513 |
| | methylsulfonyloxazol-2-yl)anilino]pyrimidine-5- | | |
| | carboxamide | | |
| 72 | 2-[(1-isopropylpyrazol-4-yl)amino]-4-[2-methoxy-3-(6- | 504.2107 | 504 |
| | oxo-5,7-dihydro-4H-oxazolo[5,4-c]pyridin-2- | | |
| | yl)anilino]pyrimidine-5-carboxamide | | |
| 73 | 4-[3-(4-acetamidooxazol-2-yl)-2-methoxy-anilino]-2- | 492.2107 | 492 |
| | [(1-isopropylpyrazol-4-yl)amino]pyrimidine-5- | | |
| | carboxamide | | |
| 74 | 4-[3-[4-(2-amino-2-oxo-ethyl)oxazol-2-yl]-2-methoxy- | 492.2107 | 492 |
| | anilino]-2-[(1-isopropylpyrazol-4-yl)amino]pyrimidine- | | |
| | 5-carboxamide | | |
| 75 | 2-[(1-isopropylpyrazol-4-yl)amino]-4-[2-methoxy-3-[4- | 506.2264 | 506 |
| | [2-(methylamino)-2-oxo-ethyl]oxazol-2- | | |
| | yl]anilino]pyrimidine-5-carboxamide | | |
| 76 | 2-[(1-isopropylpyrazol-4-yl)amino]-4-[3-[5-[(1- | 608.2846 | 608 |
| | isopropylpyrazol-4-yl)amino]-1,3-benzoxazol-2-yl]-2- | | |
| | methoxy-anilino]pyrimidine-5-carboxamide | | |

### Synthesis of the Representative Examples of the Compound

### Example 1. 4-[(2-Methoxy-3-pyridin-2-ylphenyl)amino]-2-[(1-methyl-1H-pyrazol-4-yl)amino]pyrimidine-5-carboxamide (Compound 2)

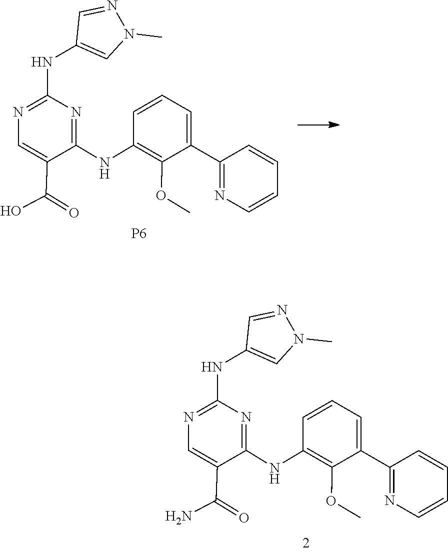

An ammonia solution (7N in MeOH, 3 ml) and TBTU (0.207 g, 0.646 mmol) were added to a solution of P6 (0.18 g, 0.43 mmol) in DMF (20 mL) at rt. The reaction mixture was stirred at rt overnight, the mixture was concentrated under reduced pressure, the residue was purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 2 (71 mg, 40% yield) as a white solid. ¹H NMR (400 MHz, DMSO-d₆), δ: 12.13 (s, 1H), 9.86 (s, 1H), 8.97 (s, 1H), 8.87-8.73 (m, 1H), 8.67 (s, 1H), 8.42-7.85 (m, 4H), 7.76-7.34 (m, 4H), 7.29 (t, J=7.8 Hz, 1H), 3.80 (s, 3H), 3.50 (s, 3H). LCMS (ESI) (C18 column 100×4.6 mm, 5.0 μm, pore size 100 Å, water-acetonitrile+0.1% TFA, gradient 5 to 87% for 10 min), m/z: 417.3 [M+H]⁺; Rt=3.58 min.

### Example 2. 4-{[2-Methoxy-3-(5-methyl-1,3,4-oxadiazol-2-yl)phenyl]amino}-2-[(1-methyl-1H-pyrazol-4-yl)amino]pyrimidine-5-carboxamide (Compound 3)

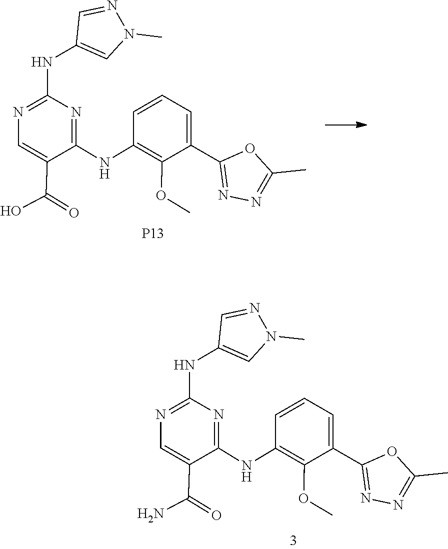

An ammonia solution (7N in MeOH, 3 ml), TBTU (0.091 g, 0.21 mmol) and DIPEA (0.05 ml, 0.285 mmol) were added to a solution of P13 (0.08 g, 0.19 mmol) in DMF (15 mL) at rt. The reaction mass was stirred at rt overnight, the mixture was concentrated under reduced pressure, the residue was purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 3 (28 mg, 35% yield) as a white solid. ¹H NMR (400 MHz, DMSO-d₆), δ: 12.09 (s, 1H), 9.68 (s, 1H), 8.87-8.62 (m, 1H), 8.47 (s, 1H), 8.22-7.83 (m, 2H), 7.72-7.37 (m, 3H), 7.32 (t, J=8.0 Hz, 1H), 3.80 (s, 6H), 2.61 (s, 3H). LCMS (ESI) (C18 column 100×4.6 mm, 5.0 μm, pore size 100 Å, water-acetonitrile+0.1% TFA, gradient 5 to 87% for 10 min), m/z: 422.2 [M+H]⁺; Rt=3.08 min.

### Example 3. 4-{[3-(1,3-Benzoxazol-2-yl)-2-methoxyphenyl]amino}-2-[(1-piperidin-4-yl-1H-pyrazol-4-yl)amino]pyrimidine-5-carboxamide (Compound 8)

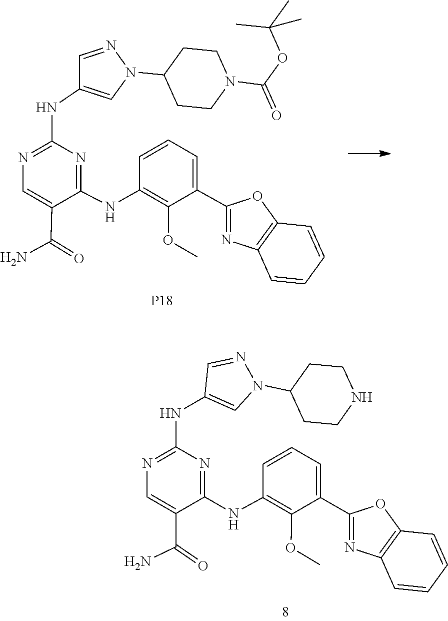

A mixture of P18 (0.05 g, 0.08 mmol) and 3M solution of HCl in 1,4-dioxane (5 ml) in MeOH (5 ml) was stirred at rt overnight. Then Et₂O (20 ml) was added, the formed precipitate was filtered, dissolved in water, saturated solution of NaHCO₃ was added to pH 8. The formed precipitate was filtered, washed with water, dried under reduced pressure to provide compound 8 (23 mg, 55% yield) as a white solid. ¹H NMR (400 MHz, DMSO-d₆), δ: 12.04 (s, 1H), 9.59 (s, 1H), 9.16 (s, 1H), 8.74 (s, 1H), 8.44 (s, 1H), 8.16-7.65 (m, 5H), 7.62-7.13 (m, 5H), 4.41-3.95 (m, 1H), 3.89 (s, 3H), 3.09-2.87 (m, 2H), 2.69-2.55 (m, 2H), 2.13-1.54 (m, 4H). LCMS (ESI) (C18 column 100×4.6 mm, 5.0 μm, pore size 100 Å, water-acetonitrile+0.1% TFA, gradient 5 to 87% for 10 min), m/z: 526.5 [M+H]⁺; Rt=4.07 min.

### Example 4-{[3-(1,3-Benzoxazol-2-yl)-2-methoxyphenyl]amino}-2-({1-[2-(dimethylamino)ethyl]-1H-pyrazol-4-yl}amino)pyrimidine-5-carboxamide (Compound 9)

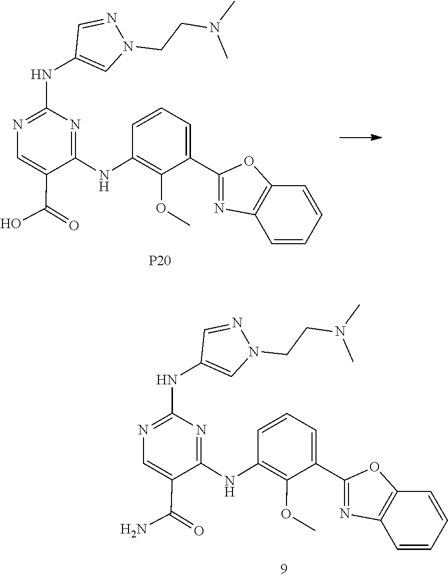

An ammonia solution (7N in MeOH, 3 ml), TBTU (0.178 g, 0.55 mmol) and DIPEA (0.2 ml, 1.08 mmol) were added to a solution of P20 (0.19 g, 0.36 mmol) in DMF (10 mL) at rt. The reaction mixture was stirred at rt overnight, the mixture was concentrated under reduced pressure, the residue was purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 9 (CF₃COOH salt) (150 mg, 20% yield) as a white solid. ¹H NMR (400 MHz, DMSO-d₆), δ: 12.19 (s, 1H), 9.78 (s, 1H), 9.42 (s, 1H), 9.13 (s, 1H), 8.74 (s, 1H), 8.22-8.03 (m, 1H), 7.91-7.72 (m, 3H), 7.72-7.61 (m, 1H), 7.55-7.41 (m, 3H), 7.36 (t, J=8.1 Hz, 1H), 4.57-4.43 (m, 2H), 3.90 (s, 3H), 3.56 (s, 2H), 2.81 (s, 6H). LCMS (ESI) (C18 column 100×4.6 mm, 5.0 μm, pore size 100 Å, water-acetonitrile+0.1% TFA, gradient 5 to 87% for 10 min), m/z: 514.5 [M+H]⁺; Rt=4.81 min.

### Example 5. 4-(2-methoxy-3-(1-methyl-1H-1,2,4-triazol-3-yl)phenylamino)-2-(1-(1-methylpiperidin-4-yl)-1H-pyrazol-4-ylamino)pyrimidine-5-carboxamide (Compound 10)

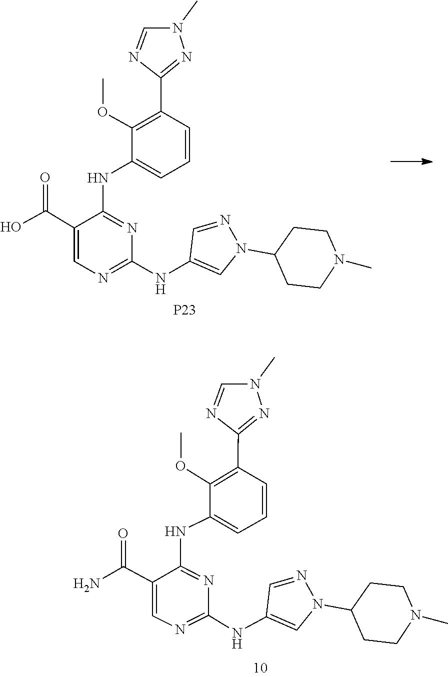

A solution of NH₃ in MeOH (1 mL, 7 mmol), TBTU (104 mg, 0.32 mmol) and DIPEA (129 mg, 1.0 mmol) were added to the solution of P23 (104 mg, 0.21 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 10 (39 mg, 37% yield) as a white solid. ¹H NMR (400 MHz, DMSO-d₆), δ: 11.90 (s, 1H), 9.88-9.33 (m, 2H), 8.69 (s, 1H), 8.56 (s, 1H), 8.35-7.84 (m, 2H), 7.84-7.33 (m, 3H), 7.19 (t, J=7.9 Hz, 1H), 4.53-4.32 (m, 1H), 3.95 (s, 3H), 3.76 (s, 3H), 3.57 (d, J=12.3 Hz, 2H), 3.27-3.04 (m, 2H), 2.84 (s, 3H), 2.37-1.97 (m, 4H). LCMS (ESI), m/z: 504.5 [M+H]⁺; Rt=3.55 min.

### Example 6. 2-(1-Cyclohexyl-1H-pyrazol-4-ylamino)-4-(2-methoxy-3-(1-methyl-1H-1,2,4-triazol-3-yl)phenylamino)pyrimidine-5-carboxamide (Compound 11)

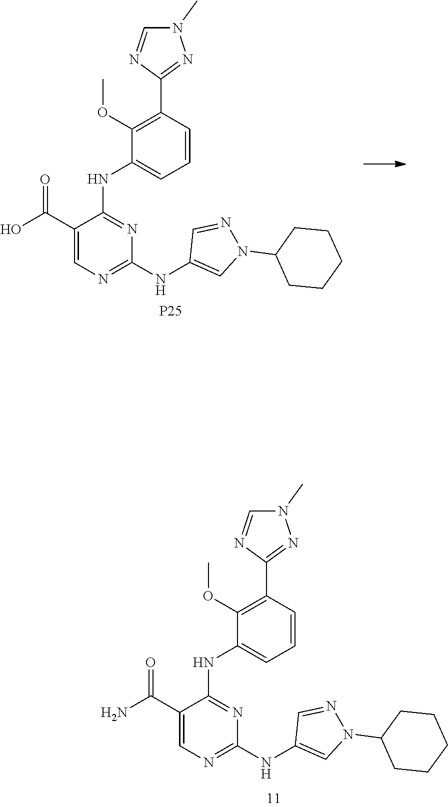

A solution of NH₃ in MeOH (1 mL, 7 mmol), TBTU (51 mg, 0.135 mmol) and DIPEA (46 mg, 0.356 mmol) were added to the solution of P25 (44 mg, 0.09 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 11 (10 mg, 23% yield) as a white solid. ¹H NMR (400 MHz, DMSO-d₆), δ: 11.96 (s, 1H), 9.84 (s, 1H), 8.91 (s, 1H), 8.66 (s, 1H), 8.57 (s, 1H), 8.19-7.89 (m, 2H), 7.76-7.34 (m, 3H), 7.30-7.08 (m, 1H), 4.33-4.12 (m, 1H), 3.95 (s, 3H), 3.75 (s, 3H), 2.12-1.04 (m, 10H). LCMS (ESI), m/z: 489.5 [M+H]⁺; Rt=4.59 min.

### Example 7. 4-((3-(1,3-Benzoxazol-2-yl)-2-methoxyphenyl)amino)-2-((1-methyl-1H-pyrazol-4-yl)amino)pyrimidine-5-carboxamide (Compound 12)

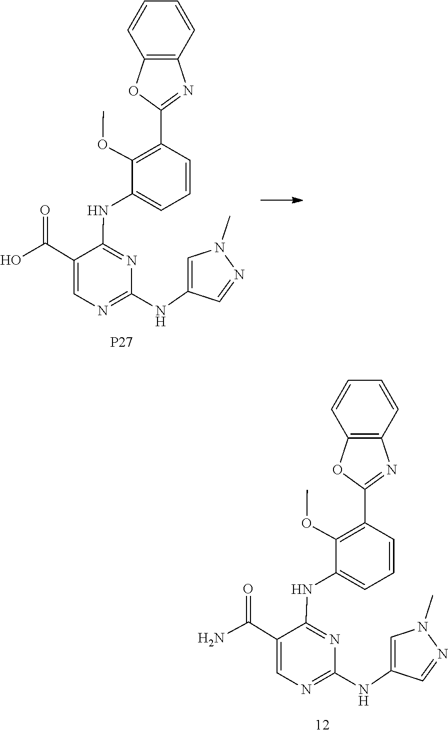

A solution of NH₃ in MeOH (1 mL, 7 mmol), TBTU (130 mg, 0.34 mmol) and DIPEA (70 mg, 0.546 mmol) were added to the solution of P27 (130 mg, 0.28 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 12 (3 mg, 2% yield) as a white solid. LCMS (ESI), m/z: 457.5 [M+H]⁺; Rt=5.49 min.

### Example 8. 2-(1,3-Dimethyl-1H-pyrazol-4-ylamino)-4-(2-methoxy-3-(1-methyl-1H-1,2,4-triazol-3-yl)phenylamino)pyrimidine-5-carboxamide (Compound 13)

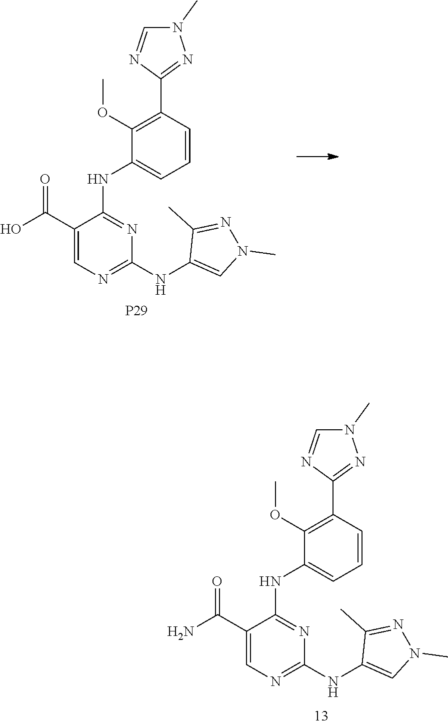

A solution of NH₃ in MeOH (1 mL, 7 mmol), TBTU (120 mg, 0.32 mmol) and DIPEA (64 mg, 0.5 mmol) were added to the solution of P29 (66 mg, 0.15 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 13 (39 mg, 59% yield) as a white solid. ¹H NMR (400 MHz, DMSO-d₆), δ: 12.24 (s, 1H), 9.48 (s, 1H), 8.88 (s, 1H), 8.76-8.36 (m, 3H), 8.36-7.95 (m, 2H), 7.95-7.42 (m, 3H), 7.42-6.90 (m, 2H), 3.95 (s, 3H), 3.77 (s, 3H), 2.08 (d, J=6.0 Hz, 3H). LCMS (ESI), m/z: 435.4 [M+H]⁺; Rt=4.08 min.

### Example 9. 4-(2-Methoxy-3-(1-methyl-1H-1,2,4-triazol-3-yl)phenylamino)-2-(1-(tetrahydro-2H-pyran-4-yl)-1H-pyrazol-4-ylamino)pyrimidine-5-carboxamide (Compound 14)

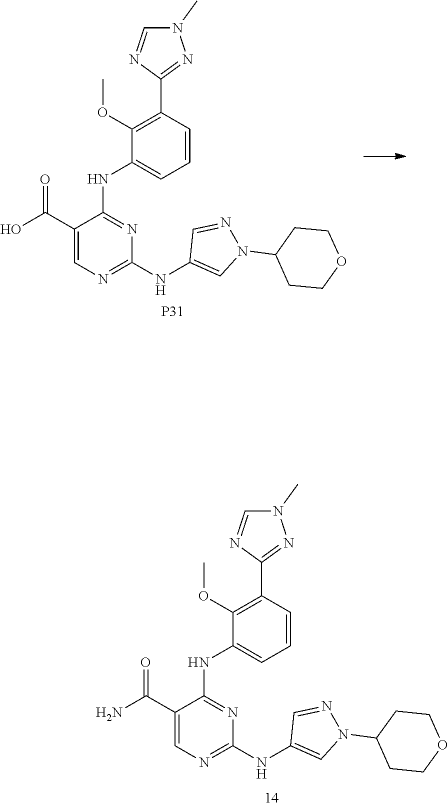

A solution of NH₃ in MeOH (1 mL, 7 mmol), TBTU (72 mg, 0.189 mmol) and DIPEA (32 mg, 0.252 mmol) were added to the solution of P31 (62 mg, 0.126 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 14 (30 mg, 48% yield) as a white solid. ¹H NMR (400 MHz, DMSO-d₆), δ: 12.27-11.81 (m, 1H), 9.82 (s, 1H), 8.66 (s, 1H), 8.56 (s, 1H), 8.24-7.84 (m, 2H), 7.84-7.32 (m, 4H), 7.32-7.07 (m, 1H), 4.49-4.16 (m, 1H), 3.95 (s, 6H), 3.89-3.71 (m, 2H), 3.51-3.33 (m, 2H), 2.07-1.72 (m, 4H). LCMS (ESI), m/z: 491.3 [M+H]⁺; Rt=4.32 min.

### Example 10. 4-{[3-(1,3-Benzoxazol-2-yl)-2-methoxyphenyl]amino}-2-[(1-isopropyl-1H-pyrazol-4-yl)amino]pyrimidine-5-carboxamide (Compound 16)

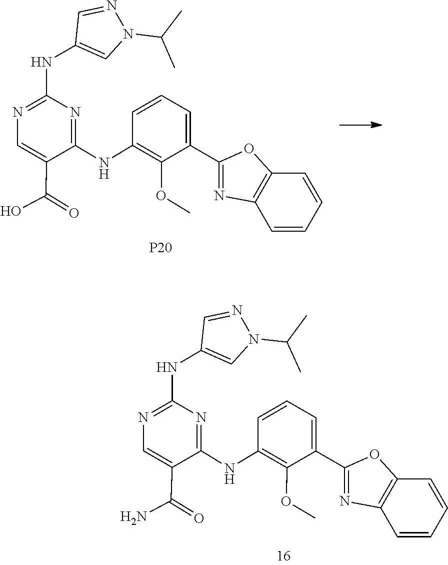

An ammonia solution (7N in MeOH, 3 ml), TBTU (0.198 g, 0.618 mmol) and DIPEA (0.1 ml, 0.618 mmol) were added to a solution of P33 (0.2 g, 0.412 mmol) in DMF (10 mL) at rt. The reaction mixture was stirred at rt overnight, the mixture was concentrated under reduced pressure, DCM was added to the residue, the formed precipitate was filtered, washed with DCM and recrystallized from EtOH to give compound 16 (112 mg, 56% yield) as a white solid. ¹H NMR (400 MHz, DMSO-d₆), δ: 12.08 (s, 1H), 9.58 (s, 1H), 8.89-8.40 (m, 2H), 8.13-7.65 (m, 5H), 7.65-7.16 (m, 5H), 4.60-4.24 (m, 1H), 3.90 (s, 3H), 1.53-1.17 (m, 6H). LCMS (ESI) (C18 column 100×4.6 mm, 5.0 μm, pore size 100 Å, water-acetonitrile+0.1% TFA, gradient 5 to 87% for 10 min), m/z: 485.5 [M+H]⁺; Rt=5.93 min.

### Example 11. 4-(2-Methoxy-3-(1-methyl-1H-1,2,4-triazol-3-yl)phenylamino)-2-(1,3,5-trimethyl-1H-pyrazol-4-ylamino)pyrimidine-5-carboxamide (Compound 17)

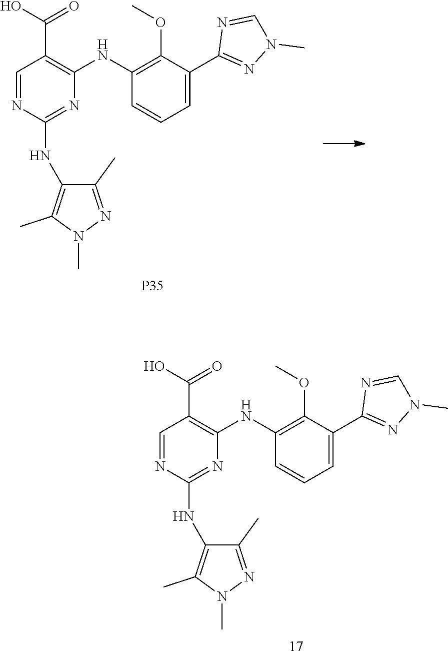

A solution of NH₃ in MeOH (1 mL, 7 mmol), TBTU (152 mg, 0.467 mmol) and DIPEA (90 mg, 0.70 mmol) were added to the solution of P35 (140 mg, 0.32 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 17 (13 mg, 10% yield) as a white solid. ¹H NMR (400 MHz, DMSO-d₆), δ: 12.50 (s, 1H), 9.41-8.48 (m, 2H), 8.38 (s, 1H), 8.21-8.04 (m, 1H), 7.96-7.39 (m, 3H), 7.39-6.77 (m, 1H), 3.95 (s, 3H), 3.84 (s, 3H), 3.73 (s, 3H), 2.16-1.95 (m, 6H). LCMS (ESI), m/z: 449.4 [M+H]⁺; Rt=3.93 min.

### Example 12. 2-(1-Ethyl-3,5-dimethyl-1H-pyrazol-4-ylamino)-4-(2-methoxy-3-(1-methyl-1H-1,2,4-triazol-3-yl)phenylamino)pyrimidine-5-carboxamide (Compound 18)

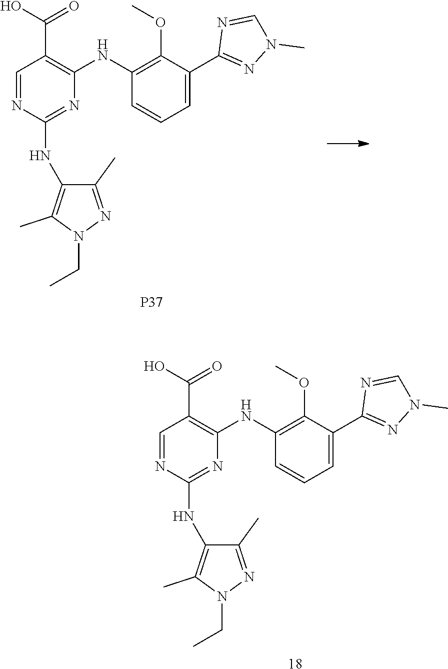 [SMILES: "P37" ; CCn1nc(C)c(Nc2ncc(C(=O)O)c(Nc3cccc(-c4ncn(C)n4)c3OC)n2)c1C ; CCn1nc(C)c(Nc2ncc(C(=O)O)c(Nc3cccc(-c4ncn(C)n4)c3OC)n2)c1C ; "18"]

A solution of NH₃ in MeOH (1 mL, 7 mmol), TBTU (350 mg, 1.08 mmol) and DIPEA (263 mg, 2.08 mmol) were added to the solution of P37 (100 mg, 0.21 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 18 (24 mg, 25% yield) as a white solid. ¹H NMR (400 MHz, DMSO-d₆), δ: 12.59 (s, 1H), 8.94-8.51 (m, 2H), 8.48-8.02 (m, 2H), 8.02-7.43 (m, 3H), 7.28 (t, J=8.0 Hz, 1H), 4.11-3.98 (m, 2H), 3.95 (s, 3H), 3.78 (s, 3H), 2.11 (s, 3H), 2.06 (s, 3H), 1.34 (t, J=7.2 Hz, 3H). LCMS (ESI), m/z: 463.9 [M+H]⁺; Rt=4.04 min.

### Example 13. 2-(3,5-Dimethyl-1-propyl-1H-pyrazol-4-ylamino)-4-(2-methoxy-3-(1-methyl-1H-1,2,4-triazol-3-yl)phenylamino)pyrimidine-5-carboxamide (Compound 19)

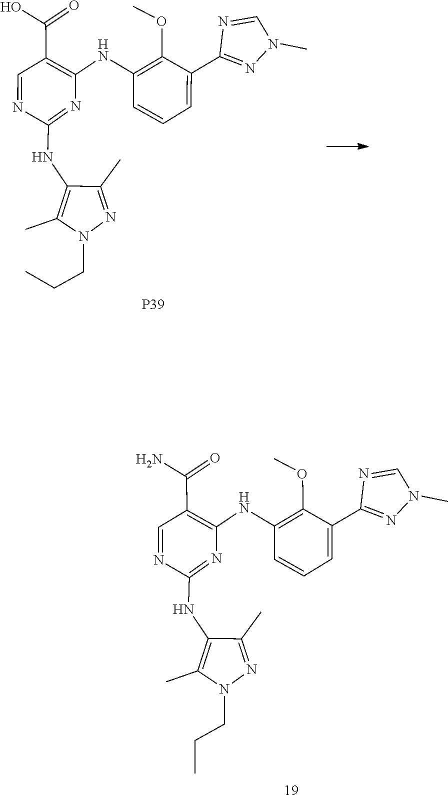

A solution of NH₃ in MeOH (1 mL, 7 mmol), TBTU (350 mg, 1.08 mmol) and DIPEA (263 mg, 2.08 mmol) were added to the solution of P39 (190 mg, 0.39 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 19 (18 mg, 9% yield) as a white solid. ¹H NMR (400 MHz, DMSO-d₆), δ: 12.63 (s, 1H), 8.90-8.61 (m, 2H), 8.58-8.12 (m, 2H), 8.02-7.53 (m, 3H), 7.22 (t, J=8.0 Hz, 1H), 4.11-3.98 (m, 2H), 3.95 (s, 3H), 3.78 (s, 3H), 2.11 (s, 3H), 2.06 (s, 3H), 1.85 (m, 2H), 1.38 (t, J=7.3 Hz, 3H). LCMS (ESI), m/z: 477.2 [M+H]⁺; Rt=4.47 min.

### Example 14. 2-(1-Isopropyl-3,5-dimethyl-1H-pyrazol-4-ylamino)-4-(2-methoxy-3-(1-methyl-1H-1,2,4-triazol-3-yl)phenylamino)pyrimidine-5-carboxamide (Compound 20)

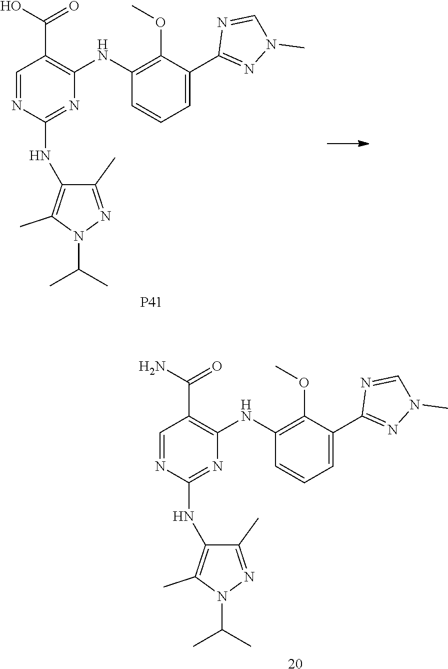

A solution of NH₃ in MeOH (1 mL, 7 mmol), TBTU (209 mg, 0.644 mmol) and DIPEA (116 mg, 0.90 mmol) were added to the solution of P41 (130 mg, 0.27 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 20 (12 mg, 9% yield) as a white solid. ¹H NMR (400 MHz, DMSO-d₆), δ: 12.58 (s, 1H), 8.88-8.51 (m, 2H), 8.52-8.16 (m, 2H), 8.09-7.47 (m, 3H), 7.26 (t, J=8.0 Hz, 1H), 4.54-4.43 (m, 1H), 3.95 (s, 3H), 3.78 (s, 3H), 2.12 (s, 3H), 2.04 (s, 3H), 1.47-1.28 (m, 6H). LCMS (ESI), m/z: 477.2 [M+H]⁺; Rt=4.46 min.

### Example 15. 2-[(1-Cyclopropyl-1H-pyrazol-4-yl)amino]-4-{[2-methoxy-3-(1-methyl-1H-1,2,4-triazol-3-yl)phenyl]amino}pyrimidine-5-carboxamide (Compound 21)

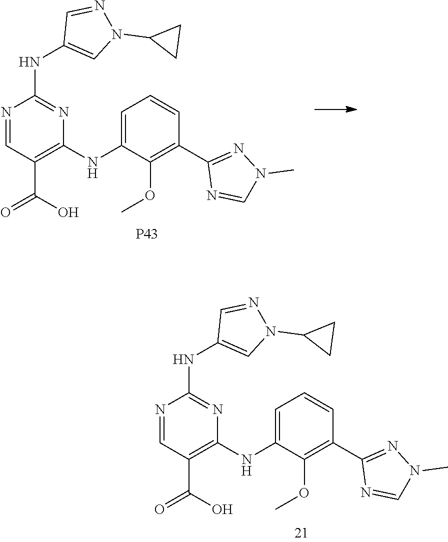

An ammonia solution (7N in MeOH, 1 ml), TBTU (0.245 g, 0.763 mmol) and DIPEA (0.27 ml, 1.52 mmol) were added to a solution of P43 (0.12 g, 0.277 mmol) in DMF (5 mL) at rt. The reaction mass was stirred at rt overnight, the mixture was concentrated under reduced pressure, the residue was purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 21 (78 mg, 34% yield) as a white solid. ¹H NMR (300 MHz, DMSO-d₆), δ: 11.99 (s, 1H), 9.75 (s, 1H), 8.66 (s, 1H), 8.55 (s, 1H), 8.23 (s, 1H), 8.11-7.70 (m, 2H), 7.70-7.34 (m, 3H), 7.20 (t, J=8.0 Hz, 1H), 3.94 (s, 3H), 3.76 (s, 3H), 3.69-3.51 (m, 1H), 1.08-0.82 (m, 4H). LCMS (ESI) (C18 column 100×4.6 mm, 5.0 μm, pore size 100 Å, water-acetonitrile+0.1% TFA, gradient 5 to 87% for 10 min), m/z: 447.2 [M+H]⁺; Rt=4.14 min.

### Example 16. 2-(1-(Cyclohexylmethyl)-1H-pyrazol-4-ylamino)-4-(2-methoxy-3-(1-methyl-1H-1,2,4-triazol-3-yl)phenylamino)pyrimidine-5-carboxamide (Compound 22)

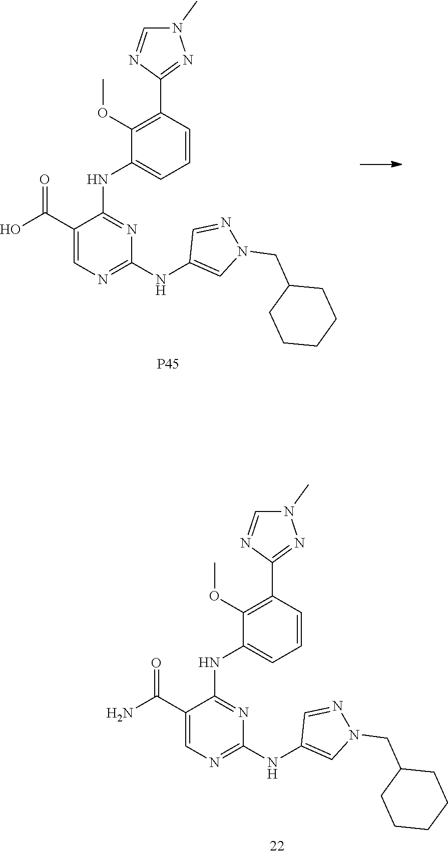

A solution of NH₃ in MeOH (1 mL, 7 mmol), TBTU (193 mg, 0.595 mmol) and DIPEA (109 mg, 0.794 mmol) were added to the solution of P45 (200 mg, 0.397 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 22 (151 mg, 76% yield) as a white solid. ¹H NMR (400 MHz, DMSO-d₆), δ: 11.87 (s, 1H), 9.84 (s, 1H), 8.66 (s, 1H), 8.56 (s, 1H), 8.21-7.85 (m, 2H), 7.73-7.34 (m, 4H), 7.25-7.13 (m, 1H), 3.95 (s, 3H), 3.84-3.68 (m, 6H), 1.89-1.28 (m, 5H), 1.25-0.72 (m, 5H). LCMS (ESI), m/z: 503.4 [M+H]⁺; Rt=5.14 min.

### Example 17. 2-(1-(Adamantylmethyl)-1H-pyrazol-4-ylamino)-4-(2-methoxy-3-(1-methyl-1H-1,2,4-triazol-3-yl)phenylamino)pyrimidine-5-carboxamide (Compound 23)

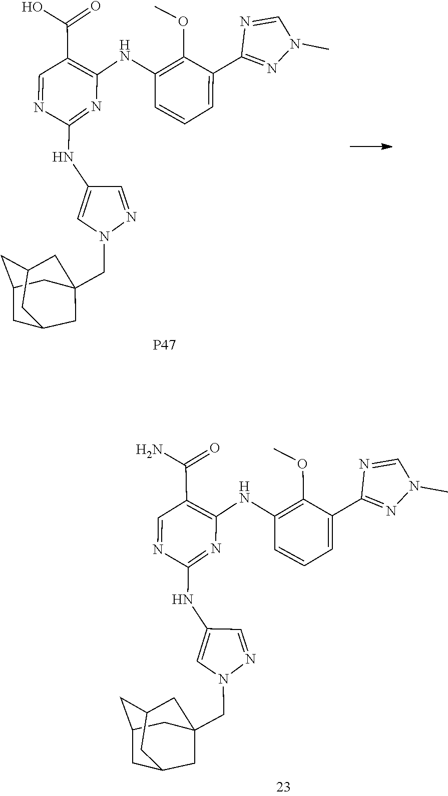 [SMILES: "P47" ; COc1c(Nc2nc(Nc3cnn(CC45CC6CC(CC(C6)C4)C5)c3)ncc2C(=O)O)cccc1-c1ncn(C)n1 ; COc1c(Nc2nc(Nc3cnn(CC45CC6CC(CC(C6)C4)C5)c3)ncc2C(N)=O)cccc1-c1ncn(C)n1]

A solution of NH₃ in MeOH (1 mL, 7 mmol), TBTU (215 mg, 0.57 mmol) and DIPEA (98 mg, 0.76 mmol) were added to the solution of P47 (210 mg, 0.38 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 23 (38 mg, 20% yield) as a white solid. ¹H NMR (400 MHz, DMSO-d₆), δ: 11.79 (s, 1H), 9.81 (s, 1H), 8.66 (s, 1H), 8.56 (s, 1H), 8.24-7.63 (m, 3H), 7.63-7.32 (m, 3H), 7.21 (t, J=8.0 Hz, 1H), 3.94 (s, 3H), 3.73 (s, 3H), 3.60 (s, 2H), 1.98-1.72 (m, 3H), 1.68-1.38 (m, 9H), 1.38-1.20 (m, 3H). LCMS (ESI), m/z: 555.3 [M+H]⁺; Rt=5.12 min.

### Example 18. 2-(1-Isopentyl-1H-pyrazol-4-ylamino)-4-(2-methoxy-3-(1-methyl-1H-1,2,4-triazol-3-yl)phenylamino)pyrimidine-5-carboxamide (Compound 24)

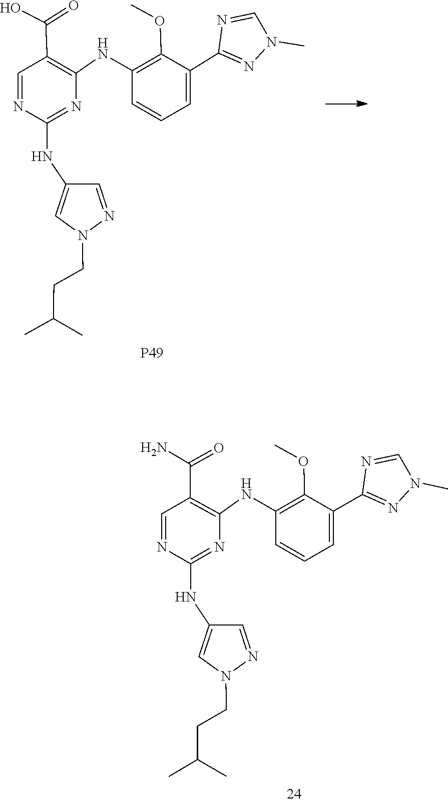 [SMILES: "P49" ; COc1c(Nc2nc(Nc3cnn(CCC(C)C)c3)ncc2C(=O)O)cccc1-c1ncn(C)n1 ; COc1c(Nc2nc(Nc3cnn(CCC(C)C)c3)ncc2C(N)=O)cccc1-c1ncn(C)n1]

A solution of NH₃ in MeOH (1 mL, 7 mmol), TBTU (105 mg, 0.324 mmol) and DIPEA (103 mg, 0.816 mmol) were added to the solution of P49 (103 mg, 0.216 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give 24 (60 mg, 59% yield) as a white solid. ¹H NMR (400 MHz, DMSO-d₆), δ: 11.97 (s, 1H), 9.84 (s, 1H), 8.66 (s, 1H), 8.56 (s, 1H), 8.31-7.82 (m, 3H), 7.82-7.33 (m, 3H), 7.33-7.02 (m, 1H), 4.15-3.98 (m, 2H), 3.95 (s, 3H), 3.76 (s, 3H), 1.79-1.36 (m, 3H), 0.86 (d, J=8.1 Hz, 6H). LCMS (ESI), m/z: 477.4 [M+H]⁺; Rt=4.18 min.

### Example 19. 4-((2-Methoxy-3-(1-methyl-1H-1,2,4-triazol-3-yl)phenyl)amino)-2-((1-octyl-1H-pyrazol-4-yl)amino)pyrimidine-5-carboxamide (Compound 25)

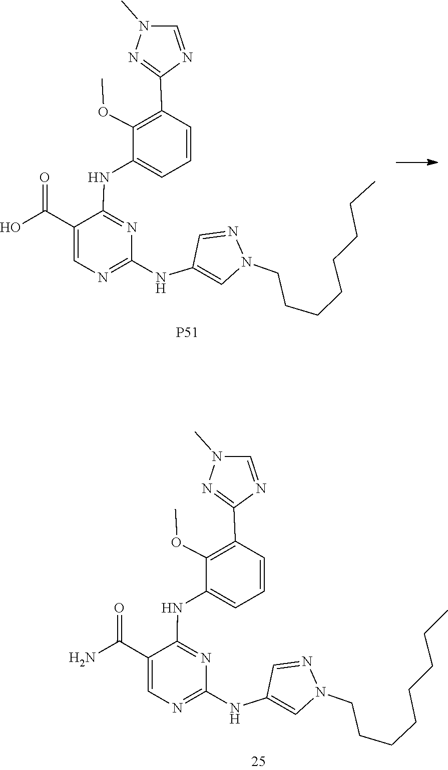

To a solution of P51 (75 mg, 0.14 mmol) in DMF (2 mL), TBTU (75 mg, 0.19 mmol) and DIPEA (0.14 ml, 101 mg, 0.8 mmol) were added. The mixture was stirred at rt for 2 h and then ammonia solution (7N in MeOH, 0.5 mL, 3.5 mmol) was added. After stirring overnight, the mixture was directly purified by C18 reverse phase column (5-100% MeOH in water) to give 25 (50 mg, 66% yield) as a white solid. ¹H NMR (400 MHz, DMSO-d₆), δ: 12.02 (s, 1H), 9.91 (s, 1H), 8.65 (s, 1H), 8.57 (s, 1H), 8.31-7.87 (m, 3H), 7.77-7.31 (m, 3H), 7.31-7.00 (m, 1H), 4.15-4.01 (m, 2H), 3.95 (s, 3H), 3.76 (s, 3H), 1.88-1.61 (m, 2H), 1.32-1.10 (m, 10H), 0.90-0.75 (m, 3H). LCMS (C18 column 20×2 mm, 2.5 μm, pore size 100 Å, water-acetonitrile+0.1% TFA, gradient 5 to 87% for 10 min, retention time 5.84 min). LCMS (ESI), m/z: 519.8 [M+H]⁺.

### Example 20. 2-((1-Hexyl-1H-pyrazol-4-yl)amino(-4-((2-methoxy-3-(1-methyl-1H-1,2,4-triazol-3-yl)phenyl]amino)pyrimidine-5-carboxamide (Compound 26)

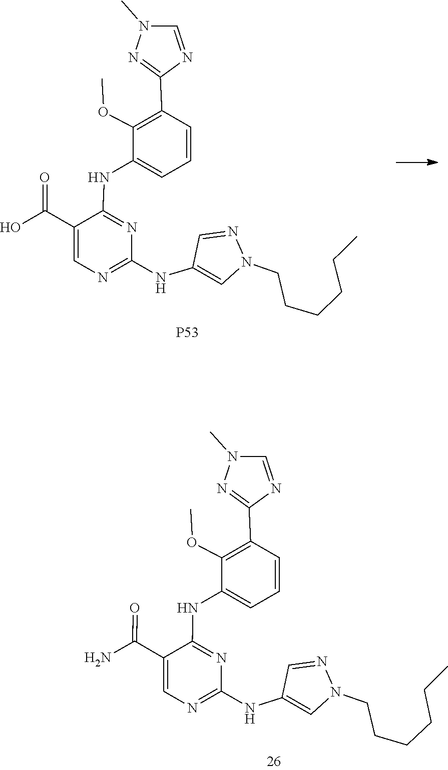 [SMILES: "P53" ; CCCCCCn1cc(Nc2ncc(C(=O)O)c(Nc3cccc(-c4ncn(C)n4)c3OC)n2)cn1 ; CCCCCCn1cc(Nc2ncc(C(N)=O)c(Nc3cccc(-c4ncn(C)n4)c3OC)n2)cn1]

To a solution of P53 (83 mg, 0.16 mmol) in DMF (2 mL), TBTU (83 mg, 0.22 mmol) and DIPEA (0.14 ml, 101 mg, 0.8 mmol) were added. The mixture was stirred at rt for 2 h and then ammonia solution (7N in MeOH, 0.5 mL, 3.5 mmol) was added. After stirring overnight, the mixture was directly purified by C18 reverse phase column (5-100% MeOH in water) to give compound 26 (26 mg, 31% yield) as a white solid. ¹H NMR (400 MHz, DMSO-d₆), δ: 12.02 (s, 1H), 9.91 (s, 1H), 8.65 (s, 1H), 8.57 (s, 1H), 8.31-7.87 (m, 3H), 7.77-7.31 (m, 3H), 7.31-7.00 (m, 1H), 4.15-4.01 (m, 2H), 3.95 (s, 3H), 3.76 (s, 3H), 1.88-1.61 (m, 2H), 1.42-1.14 (m, 6H), 0.90-0.75 (m, 3H). LCMS (C18 column 20×2 mm, 2.5 μm, pore size 100 Å, water-acetonitrile+0.1% TFA, gradient 5 to 87% for 10 min, retention time 5.21 min). LCMS (ESI), m/z: 491.5 [M+H]⁺.

### Example 21. 4-(2-methoxy-3-(1-methyl-1H-1,2,4-triazol-3-yl)phenylamino)-2-(1-(2-(1-methylpiperidin-2-yl)ethyl)-1H-pyrazol-4-ylamino)pyrimidine-5-carboxamide (Compound 27)

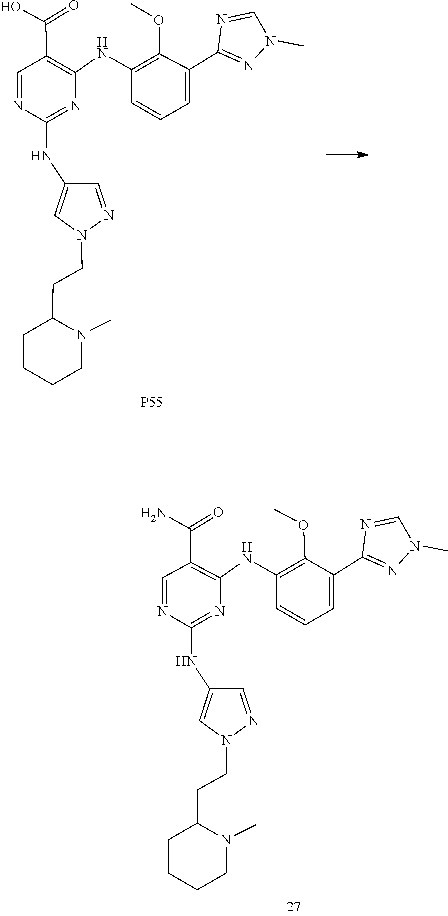 [SMILES: "P55" ; COc1c(Nc2nc(Nc3cnn(CCC4CCCCN4C)c3)ncc2C(=O)O)cccc1-c1ncn(C)n1 ; COc1c(Nc2nc(Nc3cnn(CCC4CCCCN4C)c3)ncc2C(N)=O)cccc1-c1ncn(C)n1]

A solution of NH₃ in MeOH (1 mL, 7 mmol), TBTU (147 mg, 0.45 mmol) and DIPEA (116 mg, 0.9 mmol) were added to the solution of P55 (160 mg, 0.3 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 27 (58 mg, 36% yield) as a white solid. ¹H NMR (400 MHz, DMSO-d₆), δ: 11.88 (s, 1H), 9.67 (s, 1H), 9.18 (s, 1H), 8.69 (s, 1H), 8.56 (s, 1H), 8.37-7.67 (m, 3H), 7.67-7.29 (m, 3H), 7.20 (t, J=7.9 Hz, 1H), 4.36-4.06 (m, 1H), 3.95 (s, 3H), 3.77 (s, 6H), 3.26-2.88 (m, 1H), 2.88-2.60 (m, 3H), 2.18-1.23 (m, 8H). LCMS (ESI), m/z: 532.7 [M+H]⁺; Rt=3.91 min.

### Example 22. 2-(1-(3-(dimethylamino) propyl)-1H-pyrazol-4-ylamino)-4-(2-methoxy-3-(1-methyl-1H-1,2,4-triazol-3-yl)phenylamino)pyrimidine-5-carboxamide (Compound 28)

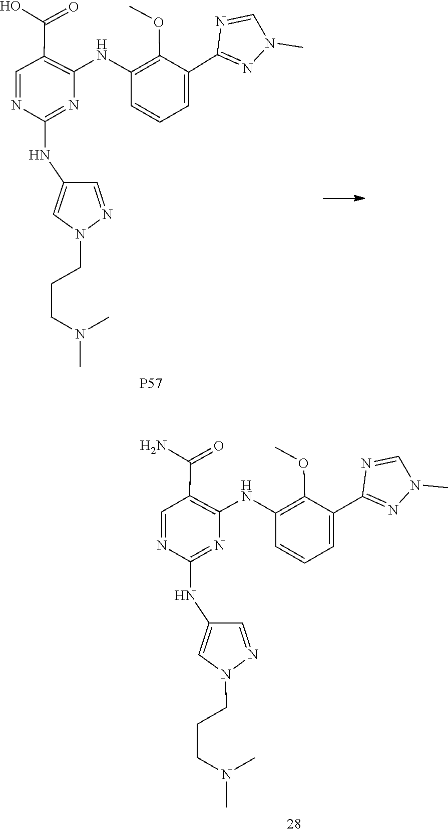 [SMILES: "P57" ; COc1c(Nc2nc(Nc3cnn(CCCN(C)C)c3)ncc2C(=O)O)cccc1-c1ncn(C)n1 ; COc1c(Nc2nc(Nc3cnn(CCCN(C)C)c3)ncc2C(N)=O)cccc1-c1ncn(C)n1]

A solution of NH₃ in MeOH (1 mL, 7 mmol), TBTU (171 mg, 0.53 mmol) and DIPEA (180 mg, 1.4 mmol) were added to the solution of P57 (113 mg, 0.23 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 28 (80 mg, 71% yield) as a white solid. ¹H NMR (400 MHz, DMSO-d₆), δ: 12.00 (s, 1H), 9.76 (s, 1H), 9.41 (s, 1H), 8.69 (s, 1H), 8.56 (s, 1H), 8.41-7.81 (m, 2H), 7.81-7.34 (m, 3H), 7.24-7.14 (m, 1H), 4.30-4.03 (m, 2H), 3.95 (s, 3H), 3.77 (s, 3H), 3.17-2.90 (m, 2H), 2.76 (s, 6H), 2.19-1.99 (m, 3H). LCMS (ESI), m/z: 492.8 [M+H]⁺; Rt=3.57 min.

### Example 23. 2-(1-(2-(Diisobutylamino)ethyl)-1H-pyrazol-4-ylamino)-4-(2-methoxy-3-(1-methyl-1H-1,2,4-triazol-3-yl)phenylamino)pyrimidine-5-carboxamide (Compound 29)

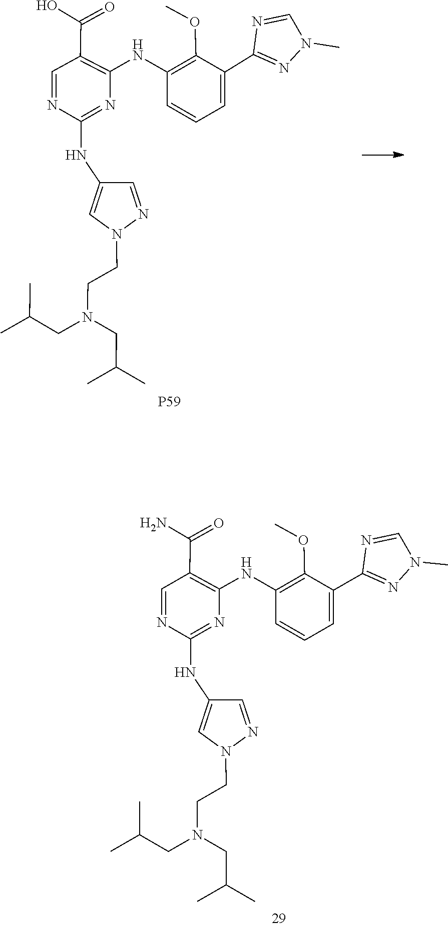

A solution of NH₃ in MeOH (2 mL, 14 mmol), TBTU (173 mg, 0.53 mmol) and DIPEA (180 mg, 1.4 mmol) were added to the solution of P59 (200 mg, 0.35 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 29 (117 mg, 58% yield) as a white solid. ¹H NMR (400 MHz, DMSO-d₆), δ: 12.00 (s, 1H), 9.80 (s, 1H), 8.70 (s, 1H), 8.56 (s, 1H), 8.39-7.79 (m, 3H), 7.73-7.31 (m, 3H), 7.20 (t, J=8.0 Hz, 1H), 4.67-4.47 (m, 2H), 3.95 (s, 3H), 3.77 (s, 3H), 3.63 (s, 2H), 3.03 (s, 4H), 2.07 (s, 2H), 1.07-0.79 (m, 12H). LCMS (ESI), m/z: 562.9 [M+H]⁺; Rt=4.07 min.

### Example 24. 2-[(1-Isopropyl-1H-pyrazol-4-yl)amino]-4-{[2-methoxy-3-(1-methyl-1H-1,2,4-triazol-3-yl)phenyl]amino}-N,N-dimethylpyrimidine-5-carboxamide (Compound 31)

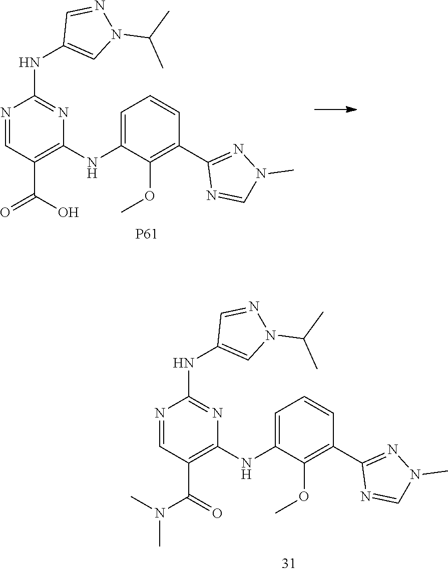

Dimethylamine hydrochloride (0.186 g, 2.28 mmol), TBTU (0.732 g, 2.28 mmol) and DIPEA (0.88 ml, 5 mmol) were added to a solution of P61 (0.75 g, 1.668 mmol) in DMF (25 mL) at rt. The reaction mixture was stirred at rt overnight, the mixture was concentrated under reduced pressure, the residue was purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 31 (CF₃COOH salt) (122 mg, 12% yield) as a white solid. ¹H NMR (400 MHz, DMSO-d₆), δ: 10.05 (s, 1H), 9.90 (s, 1H), 8.56 (s, 1H), 8.22 (s, 1H), 8.03-7.30 (m, 4H), 7.22 (t, J=8.0 Hz, 1H), 4.58-4.22 (m, 1H), 3.94 (s, 3H), 3.74 (s, 3H), 3.08 (s, 6H), 1.52-1.16 (m, 6H). LCMS (ESI) (C18 column 100×4.6 mm, 5.0 μm, pore size 100 Å, water-acetonitrile+0.1% TFA, gradient 5 to 87% for 10 min), m/z: 477.1 [M+H]⁺; Rt=4.32 min.

### Example 25. 4-((2-Methoxy-3-(1-methyl-1H-1,2,4-triazol-3-yl)phenyl)amino)-2-((1-methyl-1H-pyrazol-4-yl)amino)pyrimidine-5-carboxamide (Compound 1)

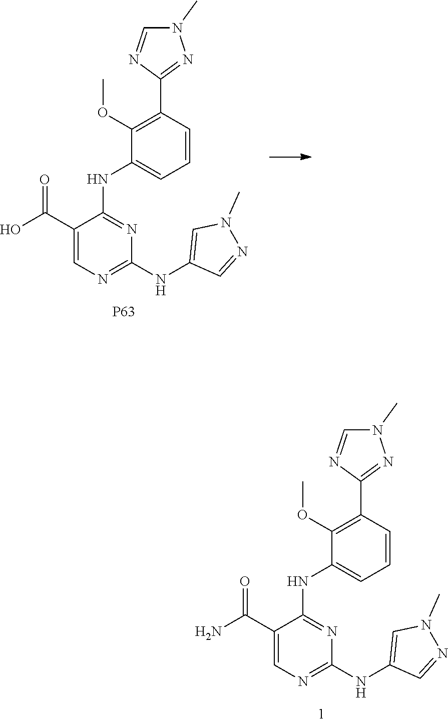 [SMILES: "P63" ; COc1c(Nc2nc(Nc3cnn(C)c3)ncc2C(=O)O)cccc1-c1ncn(C)n1 ; COc1c(Nc2nc(Nc3cnn(C)c3)ncc2C(N)=O)cccc1-c1ncn(C)n1]

A solution of 8M NH₃ in MeOH (1 mL, 8 mmol), TBTU (68 mg, 0.213 mmol) and DIPEA (55 mg, 0.426 mmol) were added to the solution of P63 (60 mg, 0.142 mmol) in DMF (4 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 1 (10 mg, 16% yield) as a white solid. ¹H NMR (300 MHz, DMSO-d₆), δ: 12.27 (s, 1H), 10.61 (s, 1H), 8.80 (s, 1H), 8.69 (s, 1H), 8.56 (s, 1H), 8.16 (s, 1H), 7.67 (d, J=2.2 Hz, 1H), 7.61 (s, 1H), 7.58 (d, J=7.9 Hz, 1H), 7.17 (t, J=8.0 Hz, 1H), 6.34 (d, J=2.3 Hz, 1H), 3.95 (s, 3H), 3.82 (s, 3H), 3.78 (s, 3H). LCMS (ESI), m/z: 421.4 [M+H]⁺; Rt=4.10 min.

### Example 26. 2-((1-Isopropyl-1H-pyrazol-4-yl)amino)-4-((2-methoxy-3-(1-methyl-1H-1,2,4-triazol-3-yl)phenyl)amino)pyrimidine-5-carboxamide (Compound 4)

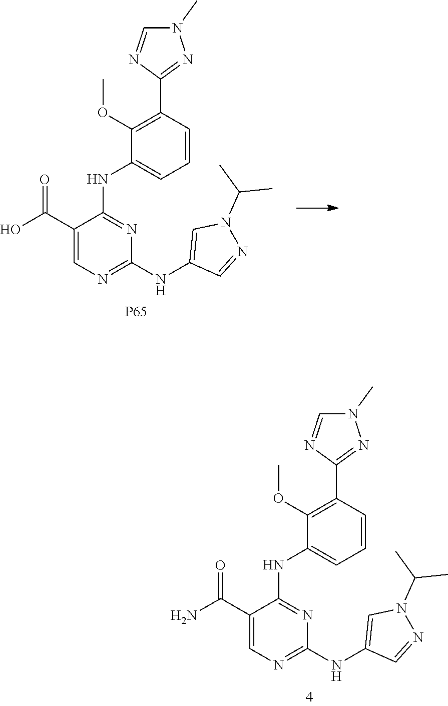 [SMILES: "P65" ; COc1c(Nc2nc(Nc3cnn(C(C)C)c3)ncc2C(=O)O)cccc1-c1ncn(C)n1 ; COc1c(Nc2nc(Nc3cnn(C(C)C)c3)ncc2C(N)=O)cccc1-c1ncn(C)n1]

A solution of 8M NH₃ in MeOH (1 mL, 8 mmol), TBTU (94 mg, 0.293 mmol) and DIPEA (126 mg, 0.975 mmol) were added to the solution of P65 (87 mg, 0.195 mmol) in DMF (10 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 4 (30 mg, 34% yield) as a white solid. ¹H NMR (400 MHz, DMSO-d₆), δ: 12.10 (s, 1H), 9.79 (s, 1H), 8.92 (s, 1H), 8.67 (s, 1H), 8.56 (s, 1H), 8.32-7.87 (m, 2H), 7.70-7.41 (m, 3H), 7.15 (s, 1H), 4.53-4.32 (m, 1H), 3.95 (s, 3H), 3.77 (s, 3H), 1.45-1.32 (m, 6H). LCMS (ESI), m/z: 449.4 [M+H]⁺; Rt=4.07 min.

### Example 27. 2-((1-(2-(dimethylamino)ethyl)-1H-pyrazol-4-yl)amino)-4-((2-methoxy-3-(1-methyl-1H-1,2,4-triazol-3-yl)phenyl)amino)pyrimidine-5-carboxamide (Compound 5)

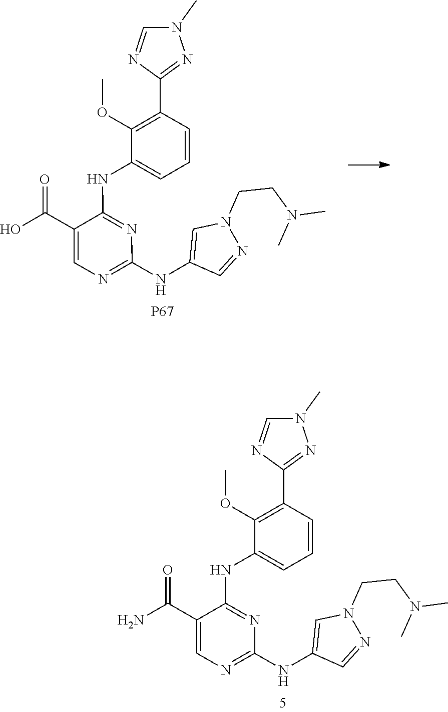 [SMILES: "P67" ; COc1c(Nc2nc(Nc3cnn(CCN(C)C)c3)ncc2C(=O)O)cccc1-c1ncn(C)n1 ; COc1c(Nc2nc(Nc3cnn(CCN(C)C)c3)ncc2C(N)=O)cccc1-c1ncn(C)n1]

A solution of 8M NH₃ in MeOH (1 mL, 8 mmol), TBTU (35 mg, 0.110 mmol) and DIPEA (57 mg, 0.438 mmol) were added to the solution of P67 (35 mg, 0.073 mmol) in DMF (10 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 5 (22 mg, 63% yield) as a white solid. ¹H NMR (400 MHz, DMSO-d₆), δ: 12.01 (s, 1H), 9.80 (s, 1H), 9.44 (s, 1H), 8.70 (s, 1H), 8.56 (s, 1H), 8.28 (s, OH), 8.20-7.75 (m, 2H), 7.71-7.34 (m, 3H), 7.22 (t, J=8.1 Hz, 1H), 4.60-4.32 (m, 2H), 3.95 (s, 3H), 3.77 (s, 3H), 3.55 (s, 2H), 2.81 (s, 6H). LCMS (ESI), m/z: 478.6 [M+H]⁺; Rt=3.45 min.

### Example 28. 4-((2-Methoxy-3-(1-methyl-1H-1,2,4-triazol-3-yl)phenyl)amino)-2-((1-phenyl-1H-pyrazol-4-yl)amino)pyrimidine-5-carboxamide (Compound 6)

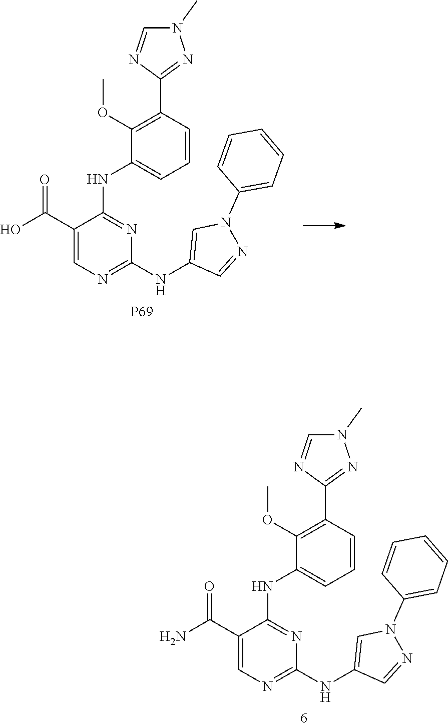 [SMILES: "P69" ; COc1c(Nc2nc(Nc3cnn(-c4ccccc4)c3)ncc2C(=O)O)cccc1-c1ncn(C)n1 ; COc1c(Nc2nc(Nc3cnn(-c4ccccc4)c3)ncc2C(N)=O)cccc1-c1ncn(C)n1]

A solution of 8M NH₃ in MeOH (1 mL, 8 mmol), TBTU (90 mg, 0.279 mmol) and DIPEA (120 mg, 0.930 mmol) were added to the solution of P69 (90 mg, 0.186 mmol) in DMF (10 mL) at rt. The solution was stirred at rt overnight and evaporated in vacuo. The mixture was directly purified by C18 reverse phase column (YMC-Pack ODS-AQ 250×20 mm, S-10 μm, 12 nm, gradient A solution-B solution (A: 1000 ml H₂O—226 μl TFA, B: 1000 ml CH₃CN) to give compound 6 (14 mg, 16% yield) as a white solid. ¹H NMR (400 MHz, DMSO-d₆), δ: 11.93 (s, 1H), 9.93 (s, 1H), 8.73 (s, 1H), 8.57 (s, 1H), 8.41 (s, 1H), 8.29-8.03 (m, 2H), 7.95-7.39 (m, 7H), 7.29 (t, J=7.4 Hz, 1H), 7.02 (s, 1H), 3.95 (s, 3H), 3.78 (s, 3H). LCMS (ESI), m/z: 483.8 [M+H]⁺; Rt=4.98 min.

### Example 29. 4-((2-Methoxy-3-(1-methyl-1H-1,2,4-triazol-3-yl)phenyl)amino)-2-((1-(piperidin-4-yl)-1H-pyrazol-4-yl)amino)pyrimidine-5-carboxamide 2,2,2-trifluoroacetate (Compound 7)

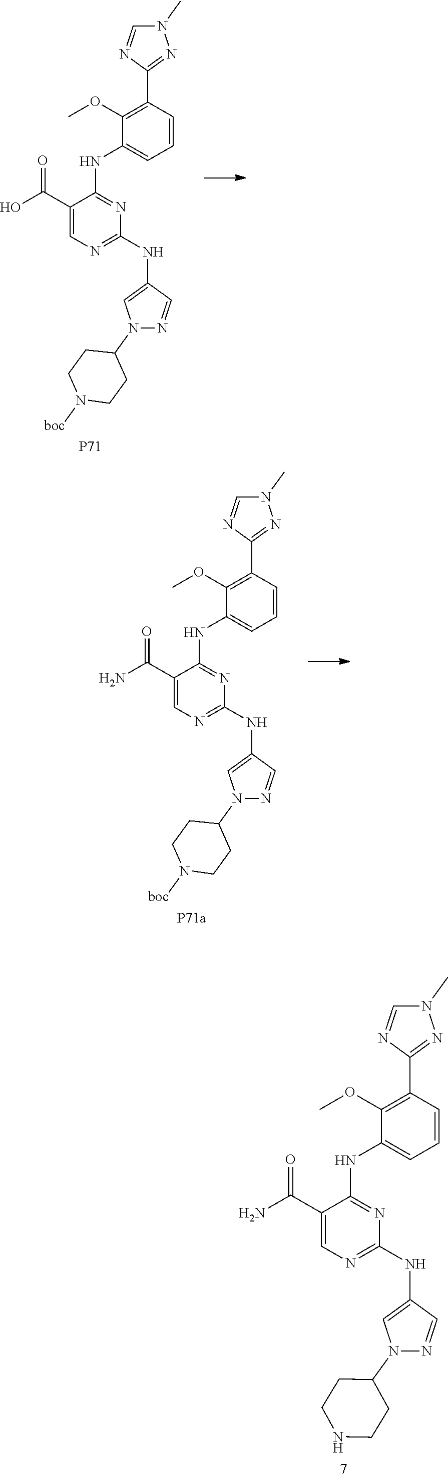

To a solution of P71 (125 mg, 0.212 mmol) in DMF (15 mL) 8M NH₃ in MeOH (1 mL, 8 mmol), TBTU (102 mg, 0.318 mmol) and DIPEA (164 mg, 1.27 mmol) were added at rt. The solution was stirred at rt for 12 h. The solvent was stripped off and the crude product P71a was used further without additional purification. LCMS (C18 column 20×2 mm, 2.5 μm, pore size 100 Å, water-acetonitrile+0.1% TFA, gradient 5 to 87% for 10 min, retention time 4.78 min). MS (ESI), m/z: 590.7 [MH]⁺. A solution of compound P71a (0.120 g, 0.204 mmol) in a mixture isopropanol-DCM (10%, 5 ml) was treated with 3M solution of HCl in dioxane (0.5 ml). The reaction was evaporated in vacuo and treated with concentrated aqueous solution of NaHCO₃ (1 ml). The product 7 was extracted with DCM, concentrated and purified by HPLC in acidic conditions (YMC-Pack ODS-AQ 250×20 mm, S-10 μm, 12 nm, gradient A solution-B solution (A: 1000 ml H₂O—226 μl TFA, B: 1000 ml CH₃CN). Yield 0.065 g (65%). ¹H NMR (400 MHz, DMSO-d₆), δ: 11.93 (s, 1H), 9.79 (s, 1H), 8.92 (s, 1H), 8.68 (s, 1H), 8.56 (s, 1H), 8.39 (s, 1H), 8.21 (s, 1H), 8.00 (s, 1H), 7.76-7.43 (m, 3H), 7.19 (s, 1H), 4.37-4.29 (m, 1H), 3.95 (s, 3H), 3.76 (s, 3H), 3.47-3.34 (m, 2H), 3.07 (q, J=11.9 Hz, 2H), 2.22-2.00 (m, 4H). LCMS (C18 column 20×2 mm, 2.5 μm, pore size 100 Å, water-acetonitrile+0.1% TFA, gradient 5 to 87% for 10 min, retention time 3.77 min). LCMS (ESI), m/z: 490.6 [MH]⁺.

### Example 30. 2-((1-(1-Adamantyl)-1H-pyrazol-4-yl)amino)-4-((2-methoxy-3-(1-methyl-1H-1,2,4-triazol-3-yl)phenyl)amino)pyrimidine-5-carboxamide (Compound 30)

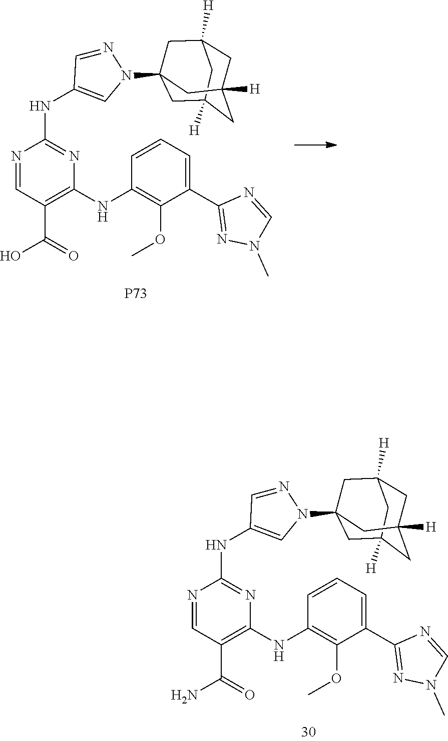 [SMILES: "P73" ; COc1c(Nc2nc(Nc3cnn([C@]45C[C@H]6C[C@H](C[C@H](C6)C4)C5)c3)ncc2C(=O)O)cccc1-c1ncn(C)n1 ; COc1c(Nc2nc(Nc3cnn([C@]45C[C@H]6C[C@H](C[C@H](C6)C4)C5)c3)ncc2C(N)=O)cccc1-c1ncn(C)n1]

To a solution of P73 (143 mg, 0.26 mmol) in DMF (2 mL), TBTU (150 mg, 0.39 mmol) and DIPEA (0.14 ml, 101 mg, 0.8 mmol) were added. The mixture was stirred at rt for 2 h and then ammonia solution (7N in MeOH, 0.5 mL, 3.5 mmol) was added. After stirring overnight, the mixture was directly purified by C18 reverse phase column (5-100% MeOH in water) to give compound 30 (85 mg, 59% yield) as a white solid. ¹H NMR (400 MHz, DMSO-d₆), δ: 9.84 (s, 1H), 12.05 (s, 1H), 8.66 (s, 1H), 8.56 (s, 1H), 8.25-7.70 (m, 3H), 7.70-7.32 (m, 3H), 7.31-6.98 (m, 1H), 3.94 (s, 3H), 3.76 (s, 3H), 2.23-2.04 (m, 6H), 2.04-1.90 (m, 4H), 1.79-1.60 (m, 5H). LCMS (C18 column 20×2 mm, 2.5 μm, pore size 100 Å, water-acetonitrile+0.1% TFA, gradient 5 to 87% for 10 min, retention time 5.09 min). LCMS (ESI), m/z: 541.4 [M+H]⁺.

### Biological Assays

### Example A. JAK2 Assay Enzymatic Protocol

Materials:
Dilution plate: Diamond Well Plate (Axygen, Cat #P-384-120 SQ-C-S);
Compounds plate: Diamond Well Plate (Axygen, Cat #P-384-120 SQ-C-S);
Reaction plate: 384w, NBS, white (Corning, Cat #4513).

Reagents and concentration in reaction:
DMSO: 1%;
Kinase: JAK2 0.1 ng/μL (SignalChem, Cat #J02-11G);
Substrate: PolyE4Y1 0.2 ng/μL (Sigma-Aldrich, Cat #P0275);
ATP: 5 μM (Promega, ADP-Glo™ Kinase Assay, Cat #V9102).

Assay Buffer 1×:
40 mM Tris-HCl pH 7.4-7.6.
20 mM MgCl₂
0.05 mM DTT.
0.1 mg/ml BSA

Procedure:
1. Prepare 100× solutions of compounds in DMSO into Compounds plate;
2. Prepare 2×JAK2-PolyE4Y1 mix in 1× Assay buffer and negative control solution (PolyE4Y1 only). Add 4 μL per well of 2×JAK2-PolyE4Y1 mix and negative control into Reaction plate;
3. Spin off plate 1 min at 200 g;
4. Prepare Dilution plate with 49 μL per well 2×ATP in assay buffer;
5. Add Compounds to Reaction plate using Biomek workstation performing following steps: transfer 1 μL of 100× Compounds from Compounds plate into Dilution plate (with 49 μL of 2×ATP solution), mix thoroughly and then transfer 4 μL from Dilution plate into Reaction plate with 4 μL of 2×JAK2-PolyE4Y1 mix;
6. Spin off plate 1 min at 200 g;
7. Incubate at RT for 1 h;
8. Add 4 μL of ADP-Glo reagent (Promega, ADP-Glo™ Kinase Assay, Cat #V9102) per well;
9. Spin off plate 1 min at 200 g;
10. Incubate at rt for 30 min;
11. Add 8 μL of Kinase detection reagent (Promega, ADP-Glo™ Kinase Assay, Cat #V9102) per well;
12. Spin off plate 1 min at 200 g;
13. Measure luminescence using Microplate Reader (BMG ClarioStar Plus).
   The results of this assay are shown in the Table A. The values of IC₅₀ shown as a letters A-D, where: A≤0.1 μM; 0.1 μM<B≤0.5 μM; 0.5 μM<C≤1 μM; D>1.

### Example B. JAK2 JH2 TR-FRET Assay Protocol

Assay Buffer 1×:
20 mM HEPES pH 7.2-7.4;
10 mM MgCl₂;
0.015% Brij-35;
2 mM DTT;
0.05 mg/ml BSA.

Final assay concentrations:
DMSO: 0.25%;
Streptavidin Tb-cryptate (Cisbio, Cat #610SATLB) 0.2 nM;
Enzyme: JAK2 JH2, His-Avi-Tag, Biotin-Labeled (BPS Bioscience, Cat #79074) 1 nM;
Probe: Tracer ZE84-0003 (ChemRar) 10 nM.

Materials:
Reaction plate: ProxiPlate-384 Plus White Plate (PerkinElmer);
Dilution plate: Diamond Well Plate (Axygen, Cat #P-384-120 SQ-C-S);
Compounds plate: Diamond Well Plate (Axygen, Cat #P-384-120 SQ-C-S).

Assay protocol:
1. Prepare 400× solutions of compounds in DMSO into Compounds plate;
2. Prepare Dilution plate: add 39 μL of 1× Assay buffer per well of Dilution plate;
3. Prepare 2×Tb-cryptate mix in 1× Assay buffer. Add 10 μL of 2×Tb-cryptate mix to K-(Tb-cryptate and Probe without Enzyme) wells and blank (Tb-cryptate without Probe and Enzyme) wells;
4. Add JAK2 JH2 into the rest of 2×Tb-cryptate mix to make 2×JAK2 JH2+Tb-cryptate mix. Add 10 μL per well into Reaction plate;
5. Centrifuge plate at 200 g for 1 min;
6. Dilute 1 μL of 400× compounds in 39 μL 1× Assay buffer in Dilution plate;
7. Add 2 μL of 10× compounds from Dilution plate to Reaction plate with 10 μL of JAK2 JH2+Tb-cryptate mix;
8. Centrifuge plate at 200 g for 1 min;
9. Pre-incubate Reaction plate with compounds 15 min;
10. Prepare 2.5× Tracer mix;
11. Add 8 μL of 2.5× Tracer mix into appropriate wells of Reaction plate;
12. Centrifuge plate at 800 g for 3 min;
13. Incubate at rt for 15 min;
14. Measure Fluorescence using Microplate Reader (Ex: TR, Em1/Em2: 482/520).
   The results of this assay are shown in the Table B. The values of EC₅₀ shown as a letters A-D, where: A≤0.01 μM; 0.01 μM<B≤0.05 μM; 0.05 μM<C≤0.1 μM; D>0.1.

TABLE B
JAK2 JH2 TR-FRET Assay

| Compound | |
|---|---|
| Number | EC₅₀, * μM |
| 1 | B |
| 2 | C |
| 3 | B |
| 4 | A |
| 5 | B |
| 6 | B |
| 7 | B |
| 8 | A |
| 11 | A |
| 12 | B |
| 13 | D |
| 14 | A |
| 16 | B |
| 17 | D |
| 18 | D |
| 19 | D |
| 20 | D |
| 22 | B |
| 23 | D |
| 24 | B |
| 25 | D |
| 26 | B |
| 27 | D |
| 28 | D |
| 29 | D |
| 30 | B |
| 31 | D |
| 32 | B |
| 33 | B |
| 34 | D |
| 35 | B |
| 36 | B |
| 37 | B |
| 38 | B |
| 39 | C |
| 40 | B |
| 41 | D |
| 42 | D |
| 44 | B |
| 45 | D |
| 46 | C |
| 47 | D |
| 48 | C |
| 49 | D |
| 50 | B |
| 51 | B |
| 52 | B |
| 53 | B |
| 54 | B |
| 55 | B |
| 56 | D |
| 57 | D |

* EC₅₀: Half maximal effective concentration: the concentration of a compound which induces a response halfway between the baseline and maximum after a specific exposure time; EC₅₀: A ≤ 0.01 μM; 0.01 μM < B ≤ 0.05 μM; 0.05 μM < C ≤ 0.1 μM; D > 0.1

### Example C. Assessment of STAT5 Phosphorylation by Immunoblotting

Ba/F3 Jak2 wild type cells (ABM, T3076) and Ba/F3 Jak2 V617F cells (WuXiAppTec) were grown in RPMI-1640 (Paneco, cat. #C330) supplemented with 10% FBS (Hyclone Laboratories Inc, Cat #SV30160.03), 1× Antibiotic-antimycotic (Gibco, Cat #15240-062), 2× Sodium pyruvate (Paneco, cat #F023), 2× Essential amino acids (Paneco, cat #F115/100), 1× Non-Essential amino acids (Paneco, cat #F116) at 37° C. in 5% CO₂ atmosphere.

Ba/F3 Jak2 wild type cells or Ba/F3 Jak2 V617F cells were seeded into 24-well CELLSTAR® Cell Culture Polystyrene Sterile Plates (Greiner, cat. #662160) at a density of 2,500,000 cells per well in complete culture medium, with or without 10 ng/ml rmIL-3 (RD systems, USA, Cat #403-ML-025), 1 ml of cell suspension per well.

1000× compounds stocks in DMSO (Sigma Cat #D2650) were prepared and added to complete culture medium in a 96-well V-bottom plate (Thermo Scientific, cat. #249946) to achieve 25× compounds solution. Next, 40 μl of 25× compounds solution was added to 24-well plates with cell suspension to achieve final DMSO concentration 0.1%. Treated cells were incubated for 3 h at 37° C. in 5% CO₂ atmosphere.

After the incubation, cells were harvested by centrifugation at 500 g for 5 min, washed once with PBS (Thermo Scientific, cat. #28372), and lysed using RIPA buffer (ProteinSimple, cat. #040-483) with 1× Halt™ Protease and Phosphatase Inhibitor Cocktail (Thermo Scientific, cat. #78440), 30 μl per well. Then cell lysates were incubated on ice for 20 min and centrifuged at 4° C., 20000 g for 20 min, followed by aspiration of supernatant used for subsequent protein analysis.

Protein concentration in the prepared cell lysates was measured with Pierce™ Coomassie (Bradford) Protein Assay Kit (Thermo Scientific, cat. #23200) using 96-well Flat-Bottom Polystyrene Plates (Greiner, cat. #655061) and Microplate Reader (CLARIOStar). Cell lysates were diluted to the concentration of 1.25 μg/μl with 0.1× Sample buffer (ProteinSimple, cat. #042-195). 10 μl of diluted lysate were mixed with 2.5 μl of Master Mix (ProteinSimple, cat. #PS-ST01EZ-8) and heated at 95° C. for 5 min. Phospho-Stat5 (Tyr694) Rabbit Antibody (1:400) (Cell Signaling, cat #9351S) and beta-Actin Mouse Antibody (1:200) (R&D Systems, cat. #MAB8929) were diluted with Milk-free diluent (ProteinSimple, cat. #043-524). 20× Anti-Rabbit HRP Antibody ((ProteinSimple, cat. #043-426) was diluted to 1× with Anti-Mouse Secondary HRP Antibody (ProteinSimple, cat. #042-205). Jess plate (ProteinSimple, cat. #SM-W004) was filled according to manufacturer's instructions and phospho-Stat5 levels in lysates were measured using Jess (ProteinSimple).

### Example D. Proliferation Inhibition Assay

Ba/F3 (JAK2 wt) (ABM, T3076) cells and Ba/F3 (JAK2 V617F) (WuXi AppTec) cells were grown in RPMI-1640 (Gibco, cat. number 21870) supplemented with 10% FBS (Hyclone Laboratories Inc, Cat #SV30160.03), 1× Antibiotic-antimycotic (Gibco, Cat #15240-062), 2× Sodium pyruvate (Paneco, cat #F023), 2× Essential amino acids (Paneco, cat #F115/100), 1× Non-Essential amino acids (Paneco, cat #F116) at 37° C. in 5% CO₂ atmosphere.

For the proliferation inhibition assay Ba/F3 (JAK2 wt) and Ba/F3 (JAK2 V617F) cells were seeded into a CellBIND® 384-well Flat Clear Bottom Black Polystyrene Microplates (Corning, USA, Cat #3770) at a density of 4000 cells per well in complete culture medium with or without 10 ng/ml rmIL-3 (RD systems, USA, Cat #403-ML-025) using Biomek FX/NX (384) liquid handler, 45 μl of cell suspension in complete culture medium per well. 500× compound's stocks in DMSO (Sigma Cat #D2650) were prepared in compounds plate (Diamond Well Plate, Axigen, Cat #P-384-120 SQ-C-S) applying Biomek 2000. 1 μl of 500× compounds stock was added to 49 μl of complete culture medium into Dilution plate (Diamond Well Plate, Axigen, Cat #P-384-120 SQ-C-S), mixed and then 5 μl of 10× compounds solution was transferred to cells using Biomek FX/NX (384) liquid handler. The centrifugation at 100 g for 1 min at Eppendorf 5810 followed. Final DMSO concentration in assay was 0.2%. All treatments were performed in technical and biological duplicates. Treated cells were incubated for 72 h at 37° C. with 5% CO₂.

After 3 days of incubation, CellTiter-Glo Luminescent Cell Viability Assay (Promega) was performed. 10 μl of CellTiter-Glo (Promega, CAT #G7572) was added to the cells using Biomek FX/NX (384) liquid handler. The assay plate was centrifuged at 100 g for 1 min and luminescence signal was measured using Microplate Reader (CLARIOStar).

TABLE C
JAK2 JH2 TR-FRET Assay

| | Ba/F3 | Ba/F3 | Ba/F3 JAK2 | Ba/F3 JAK2 |
|---|---|---|---|---|
| | JAK2 wt, | JAK2 wt + IL3, | V617F, | V617F + IL3, |
| | CC₅₀ | CC₅₀ | CC₅₀ | CC₅₀ |
| # | 3 days, μM | 3 days, μM | 3 days, μM | 3 days, μM |
| 1 | E | E | C | D |
| 2 | E | E | A | C |
| 3 | E | E | D | E |
| 4 | D | D | A | C |
| 6 | E | E | A | A |
| 7 | E | E | C | E |
| 8 | E | E | C | D |
| 11 | C | C | A | A |
| 12 | E | E | A | C |
| 14 | E | E | A | D |
| 16 | E | D | A | B |
| 17 | E | E | E | E |
| 18 | E | E | D | E |
| 22 | D | D | A | C |
| 24 | E | D | A | B |
| 26 | D | D | A | B |
| 28 | E | E | D | E |
| 29 | C | D | A | C |
| 30 | C | C | A | B |
| 32 | — | C | B | — |
| 33 | — | C | A | — |
| 34 | — | D | C | — |
| 35 | — | E | C | — |
| 36 | — | E | E | — |
| 37 | — | D | D | — |
| 38 | — | D | A | — |
| 39 | — | D | A | — |
| 40 | — | D | A | — |
| 41 | — | D | C | — |
| 42 | — | D | C | — |
| 44 | — | C | A | — |
| 45 | — | D | B | — |
| 46 | — | C | A | — |
| 47 | — | D | C | — |
| 48 | — | C | A | — |
| 49 | — | D | D | — |
| 50 | — | E | A | — |
| 51 | — | E | C | — |
| 52 | — | C | A | — |
| 53 | — | E | C | — |
| 54 | — | C | A | — |
| 55 | — | E | C | — |
| 56 | — | D | B | — |
| 57 | — | E | E | — |
| 58 | — | C | B | — |
| 59 | — | D | C | — |

*CC₅₀: The effective concentration of a cytotoxic compound, which produces 50% of the maximum possible cell death for that compound; CC₅₀: A ≤ 0.5 μM; 0.5 μM < B ≤ 1.0 μM; 1.0 μM < C ≤ 5.0 μM; 5.0 μM < D ≤ 10.0 μM; E > 10.0

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound of Formula (I): 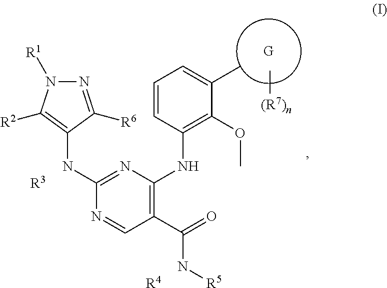
or a pharmaceutically acceptable salt, prodrug, stereoisomer, solvate, or tautomer thereof, wherein
Ring G is 5-10 membered monocyclic or bicyclic heteroaryl, comprising 1-3 heteroatoms selected from N, O, S;
R¹ is selected from C₁-C₆ alkyl, C₂-C₆ alkenyl, C₂-C₆ alkynyl, C₃-C₁₀ cycloalkyl, heterocycle, aryl, and heteroaryl, wherein the alkyl, alkenyl, alkynyl, cycloalkyl, heterocycle, aryl, or heteroaryl is optionally substituted with one or more substituents independently selected from halogen, CN, NO₂, OH, N(R⁸)₂, C₁-C₆ alkyl, C₁-C₆ alkoxy, C₃-C₁₀ cycloalkyl, C₂-C₆ alkenyl, C₂-C₆ alkynyl, heterocyclyl, aryl, heteroaryl, wherein the C₃-C₁₀ cycloalkyl, heterocyclyl, aryl, or heteroaryl is optionally substituted with one or more substituents independently selected from C₁-C₆ alkyl, halogen, OH, CN;
R² is selected from H, C₁-C₆ alkyl;
R³ is selected from hydrogen, C₁-C₆ alkyl, C₃-C₁₀ cycloalkyl;
R⁴ is selected from H, C₁-C₆ alkyl;
R⁵ is selected from H, C₁-C₆ alkyl;
or R⁴ and R⁵ together with the atoms to which they are attached and any intervening atoms, form a 3-14 membered heterocycle optionally substituted with one or more substituents independently selected from halogen, C₁-C₆ alkyl, and C₁-C₆ alkoxy;
R⁶ is selected from H, C₁-C₆ alkyl;
each R⁷ is independently selected from oxo, C₁-C₆ alkyl, C₃-C₁₀ cycloalkyl, heterocyclyl, —NH-heteroaryl, —(CH₂)ₘ—NH—C(O)—R⁸, —(CH₂)ₘ—C(O)NH—R⁸, —(CH₂)ₘ—C(O)NH-heteroaryl, —(CH₂)ₘ—S(O)₂N—R⁸, —(CH₂)ₘ—S(O)₂N-heterocyclyl, wherein the alkyl, cycloalkyl, heterocyclyl or heteroaryl is optionally substituted with one or more substituents independently selected from halogen, —CN, —OH, N(R⁸)₂, C₁-C₆ alkyl, C₁-C₆ alkoxy, cycloalkyl;
each R⁸ is independently selected from H, C₁-C₆ alkyl, C₃-C₁₀ cycloalkyl;
n is an integer selected from 0, 1, 2 and 3;
m is an integer selected from 0, 1, and 2;
wherein,
cycloalkyl is a mono or polycyclic saturated carbon rings containing 3-18 carbon atoms;
aryl is a cyclic, aromatic hydrocarbon groups that have 1 to 3 aromatic rings;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C.

2. The compound of claim 1, wherein the compound is of Formula (I-A): 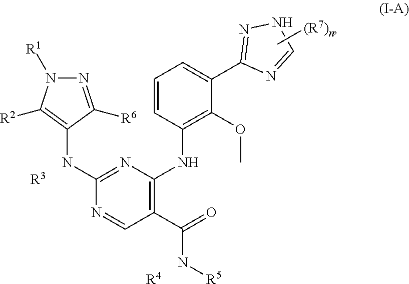
or a pharmaceutically acceptable salt, stereoisomer, solvate, or tautomer thereof.

3. The compound of claim 1, wherein the compound is of Formula (I-B): 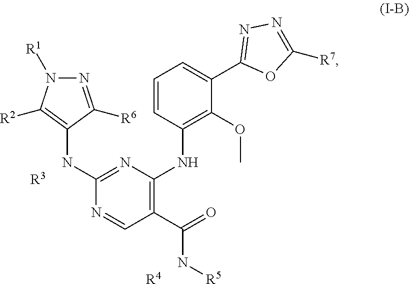
or a pharmaceutically acceptable salt, stereoisomer, solvate, or tautomer thereof.

4. The compound of claim 1, wherein the compound is of Formula (I-C): 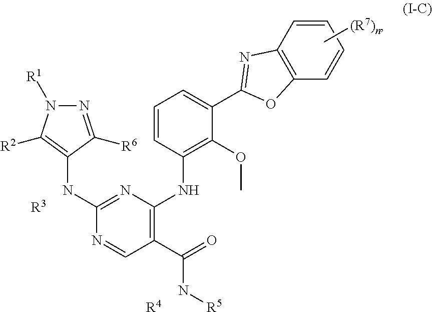
or a pharmaceutically acceptable salt, stereoisomer, solvate, or tautomer thereof.

5. The compound of claim 1, wherein the compound is of Formula (I-D): 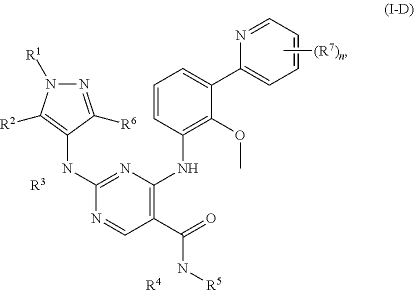
or a pharmaceutically acceptable salt, stereoisomer, solvate, or tautomer thereof.

6. The compound of claim 1, wherein the compound is of Formula (I-E): 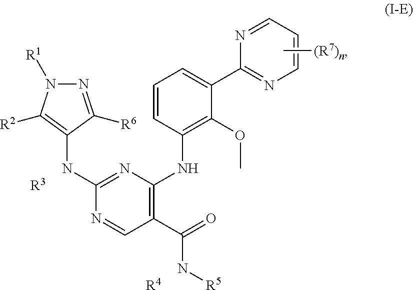
or a pharmaceutically acceptable salt, stereoisomer, solvate, or tautomer thereof.

7. The compound of claim 1, wherein the compound is of Formula (I-ABC): 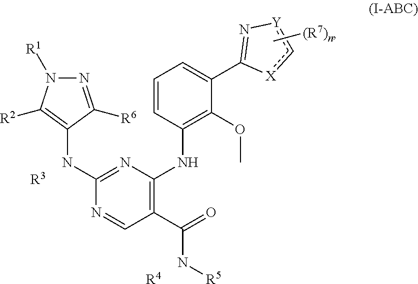
or a pharmaceutically acceptable salt, stereoisomer, solvate, or tautomer thereof, wherein X is selected from N and O, Y is selected from N, O and C, the bond X [symbol] is a single or double bond, the bond [symbol] Y is a single or double bond; provided that when X is N the bond X [symbol] is a double bond and the bond [symbol] Y is a single bond and when X is O the bond X [symbol] is a single bond and the bond [symbol] Y is a double bond; optionally two of R⁷ together with the atoms to which they are attached and any intervening atoms, form an aryl optionally substituted with 1-3 R⁷ and all other variables are as defined herein.

8. The compound of claim 1, wherein the compound is of Formula (I-DE): 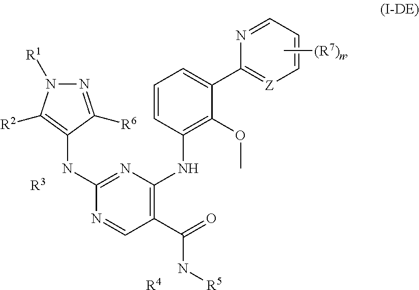
or a pharmaceutically acceptable salt, stereoisomer, solvate, or tautomer thereof, wherein Z is selected from N and C, optionally two of R⁷ together with the atoms to which they are attached and any intervening atoms, form an aryl and all other variables are as defined herein.

9. A compound selected from:

| # | Structure | IUPAC Name |
|---|---|---|
| 1 | 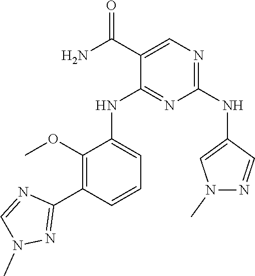 [SMILES: COc1c(Nc2nc(Nc3cnn(C)c3)ncc2C(N)=O)cccc1-c1ncn(C)n1] | 4-[2-methoxy-3-(1-methyl-1,2,4- triazol-3-yl)anilino]-2-[(1- methylpyrazol-4-yl)amino]pyrimidine- 5-carboxamide |
| 2 | 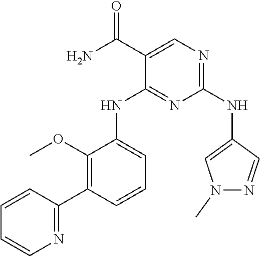 [SMILES: COc1c(Nc2nc(Nc3cnn(C)c3)ncc2C(N)=O)cccc1-c1ccccn1] | 4-[2-methoxy-3-(2-pyridyl)anilino]-2- [(1-methylpyrazol-4- yl)amino]pyrimidine-5-carboxamide |
| 3 | 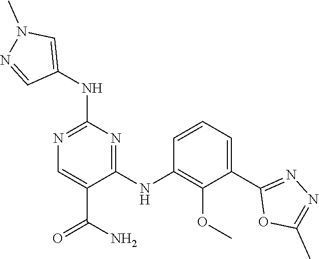 [SMILES: COc1c(Nc2nc(Nc3cnn(C)c3)ncc2C(N)=O)cccc1-c1nnc(C)o1] | 4-[2-methoxy-3-(5-methyl-1,3,4- oxadiazol-2-yl)anilino]-2-[(1- methylpyrazol-4-yl)amino]pyrimidine- 5-carboxamide |
| 4 | 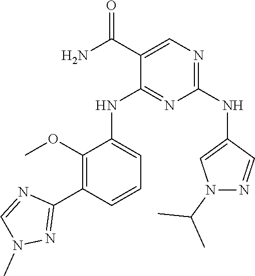 [SMILES: COc1c(Nc2nc(Nc3cnn(C(C)C)c3)ncc2C(N)=O)cccc1-c1ncn(C)n1] | 2-[(1-isopropylpyrazol-4-yl)amino]-4- [2-methoxy-3-(1-methyl-1,2,4-triazol- 3-yl)anilino]pyrimidine-5-carboxamide |
| 5 | 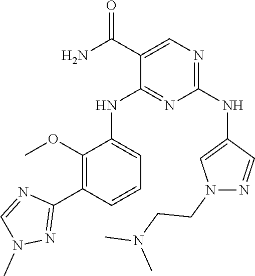 [SMILES: COc1c(Nc2nc(Nc3cnn(CCN(C)C)c3)ncc2C(N)=O)cccc1-c1ncn(C)n1] | 2-[[1-[2-(dimethylamino)ethyl]pyrazol- 4-yl]amino]-4-[2-methoxy-3-(1- methyl-1,2,4-triazol-3- yl)anilino]pyrimidine-5-carboxamide |
| 6 | 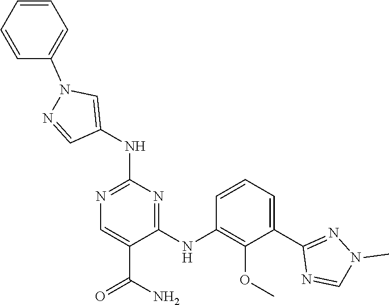 [SMILES: COc1c(Nc2nc(Nc3cnn(-c4ccccc4)c3)ncc2C(N)=O)cccc1-c1ncn(C)n1] | 4-[2-methoxy-3-(1-methyl-1,2,4- triazol-3-yl)anilino]-2-[(1- phenylpyrazol-4-yl)amino]pyrimidine- 5-carboxamide |
| 7 | 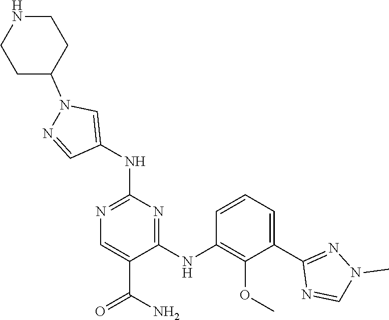 [SMILES: COc1c(Nc2nc(Nc3cnn(C4CCNCC4)c3)ncc2C(N)=O)cccc1-c1ncn(C)n1] | 4-[2-methoxy-3-(1-methyl-1,2,4- triazol-3-yl)anilino]-2-[[1-(4- piperidyl)pyrazol-4- yl]amino]pyrimidine-5-carboxamide |
| 8 | 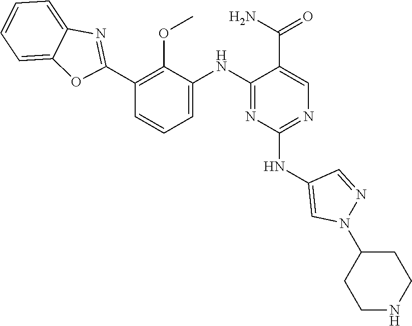 [SMILES: COc1c(Nc2nc(Nc3cnn(C4CCNCC4)c3)ncc2C(N)=O)cccc1-c1nc2ccccc2o1] | 4-[3-(1,3-benzoxazol-2-yl)-2-methoxy- anilino]-2-[[1-(4-piperidyl)pyrazol-4- yl]amino]pyrimidine-5-carboxamide |
| 9 | 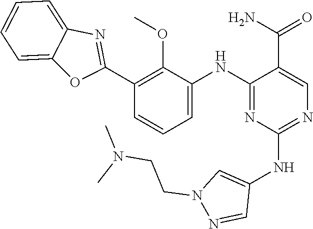 [SMILES: COc1c(Nc2nc(Nc3cnn(CCN(C)C)c3)ncc2C(N)=O)cccc1-c1nc2ccccc2o1] | 4-[3-(1,3-benzoxazol-2-yl)-2-methoxy- anilino]-2-[[1-[2- (dimethylamino)ethyl]pyrazol-4- yl]amino]pyrimidine-5-carboxamide |
| 10 | 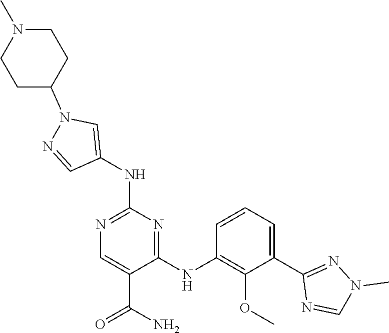 [SMILES: COc1c(Nc2nc(Nc3cnn(C4CCN(C)CC4)c3)ncc2C(N)=O)cccc1-c1ncn(C)n1] | 4-[2-methoxy-3-(1-methyl-1,2,4- triazol-3-yl)anilino]-2-[[1-(1-methyl-4- piperidyl)pyrazol-4- yl]amino]pyrimidine-5-carboxamide |
| 11 | 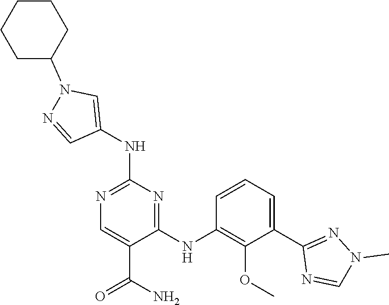 [SMILES: COc1c(Nc2nc(Nc3cnn(C4CCCCC4)c3)ncc2C(N)=O)cccc1-c1ncn(C)n1] | 2-[(1-cyclohexylpyrazol-4-yl)amino]- 4-[2-methoxy-3-(1-methyl-1,2,4- triazol-3-yl)anilino]pyrimidine-5- carboxamide |
| 12 | 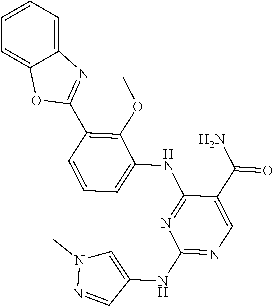 [SMILES: COc1c(Nc2nc(Nc3cnn(C)c3)ncc2C(N)=O)cccc1-c1nc2ccccc2o1] | 4-[3-(1,3-benzoxazol-2-yl)-2-methoxy- anilino]-2-[(1-methylpyrazol-4- yl)amino]pyrimidine-5-carboxamide |
| 13 | 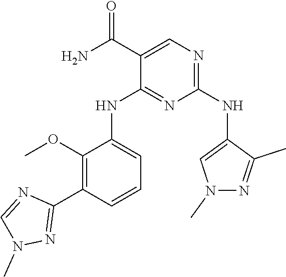 [SMILES: COc1c(Nc2nc(Nc3cn(C)nc3C)ncc2C(N)=O)cccc1-c1ncn(C)n1] | 2-[(1,3-dimethylpyrazol-4-yl)amino]-4- [2-methoxy-3-(1-methyl-1,2,4-triazol- 3-yl)anilino]pyrimidine-5-carboxamide |
| 14 | 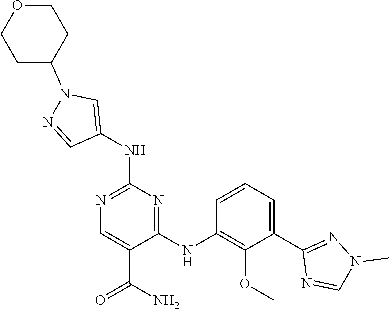 [SMILES: COc1c(Nc2nc(Nc3cnn(C4CCOCC4)c3)ncc2C(N)=O)cccc1-c1ncn(C)n1] | 4-[2-methoxy-3-(1-methyl-1,2,4- triazol-3-yl)anilino]-2-[(1- tetrahydropyran-4-ylpyrazol-4- yl)amino]pyrimidine-5-carboxamide |
| 15 | 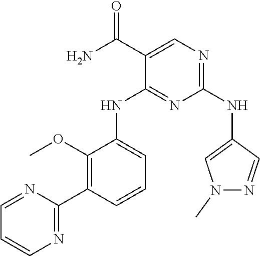 [SMILES: COc1c(Nc2nc(Nc3cnn(C)c3)ncc2C(N)=O)cccc1-c1ncccn1] | 4-(2-methoxy-3-pyrimidin-2-yl- anilino)-2-[(1-methylpyrazol-4- yl)amino]pyrimidine-5-carboxamide |
| 16 | 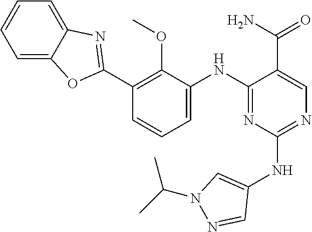 [SMILES: COc1c(Nc2nc(Nc3cnn(C(C)C)c3)ncc2C(N)=O)cccc1-c1nc2ccccc2o1] | 4-[3-(1,3-benzoxazol-2-yl)-2-methoxy- anilino]-2-[(1-isopropylpyrazol-4- yl)amino]pyrimidine-5-carboxamide |
| 17 | 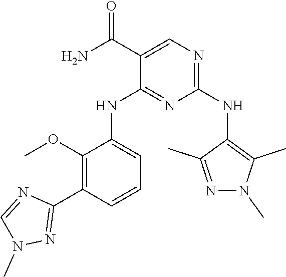 [SMILES: COc1c(Nc2nc(Nc3c(C)nn(C)c3C)ncc2C(N)=O)cccc1-c1ncn(C)n1] | 4-[2-methoxy-3-(1-methyl-1,2,4- triazol-3-yl)anilino]-2-[(1,3,5- trimethylpyrazol-4- yl)amino]pyrimidine-5-carboxamide |
| 18 | 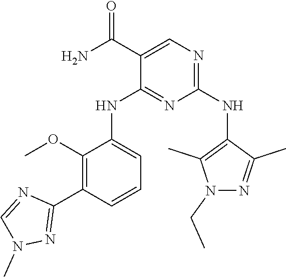 [SMILES: CCn1nc(C)c(Nc2ncc(C(N)=O)c(Nc3cccc(-c4ncn(C)n4)c3OC)n2)c1C] | 2-[(1-ethyl-3,5-dimethyl-pyrazol-4- yl)amino]-4-[2-methoxy-3-(1-methyl- 1,2,4-triazol-3-yl)anilino]pyrimidine-5- carboxamide |
| 19 | 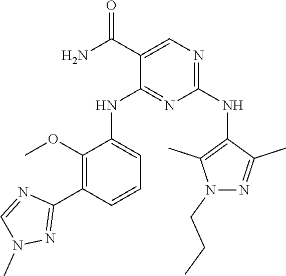 [SMILES: CCCn1nc(C)c(Nc2ncc(C(N)=O)c(Nc3cccc(-c4ncn(C)n4)c3OC)n2)c1C] | 2-[(3,5-dimethyl-1-propyl-pyrazol-4- yl)amino]-4-[2-methoxy-3-(1-methyl- 1,2,4-triazol-3-yl)anilino]pyrimidine-5- carboxamide |
| 20 | 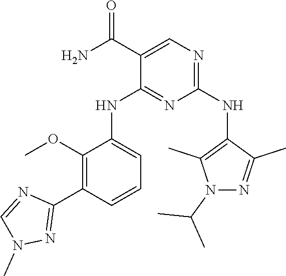 [SMILES: COc1c(Nc2nc(Nc3c(C)nn(C(C)C)c3C)ncc2C(N)=O)cccc1-c1ncn(C)n1] | 2-[(1-isopropyl-3,5-dimethyl-pyrazol- 4-yl)amino]-4-[2-methoxy-3-(1- methyl-1,2,4-triazol-3- yl)anilino]pyrimidine-5-carboxamide |
| 21 | 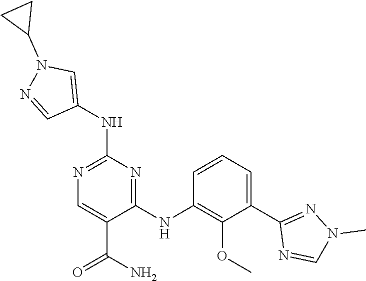 [SMILES: COc1c(Nc2nc(Nc3cnn(C4CC4)c3)ncc2C(N)=O)cccc1-c1ncn(C)n1] | 2-[(1-cyclopropylpyrazol-4-yl)amino]- 4-[2-methoxy-3-(1-methyl-1,2,4- triazol-3-yl)anilino]pyrimidine-5- carboxamide |
| 22 | 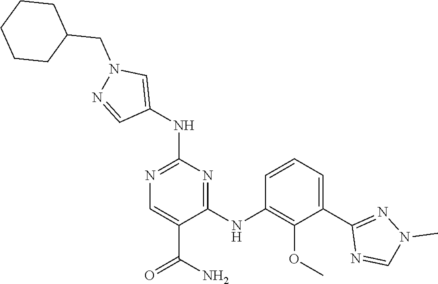 [SMILES: COc1c(Nc2nc(Nc3cnn(CC4CCCCC4)c3)ncc2C(N)=O)cccc1-c1ncn(C)n1] | 2-[[1-(cyclohexylmethyl)pyrazol-4- yl]amino]-4-[2-methoxy-3-(1-methyl- 1,2,4-triazol-3-yl)anilino]pyrimidine-5- carboxamide |
| 23 | 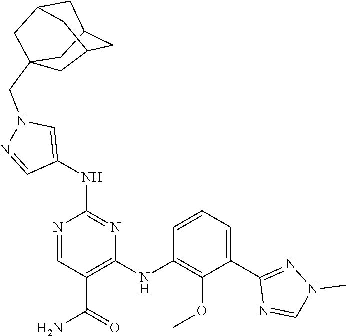 [SMILES: COc1c(Nc2nc(Nc3cnn(CC45CC6CC(CC(C6)C4)C5)c3)ncc2C(N)=O)cccc1-c1ncn(C)n1] | 2-[[1-(1-adamantylmethyl)pyrazol-4- yl]amino]-4-[2-methoxy-3-(1-methyl- 1,2,4-triazol-3-yl)anilino]pyrimidine-5- carboxamide |
| 24 | 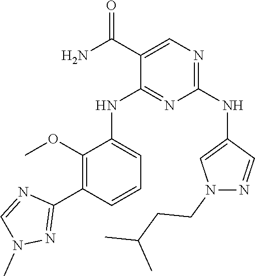 [SMILES: COc1c(Nc2nc(Nc3cnn(CCC(C)C)c3)ncc2C(N)=O)cccc1-c1ncn(C)n1] | 2-[(1-isopentylpyrazol-4-yl)amino]-4- [2-methoxy-3-(1-methyl-1,2,4-triazol- 3-yl)anilino]pyrimidine-5-carboxamide |
| 25 | 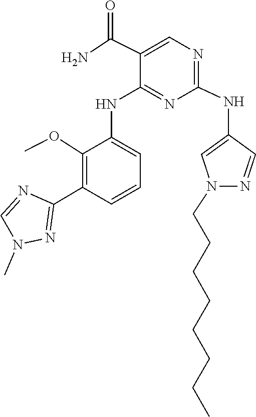 [SMILES: CCCCCCCCn1cc(Nc2ncc(C(N)=O)c(Nc3cccc(-c4ncn(C)n4)c3OC)n2)cn1] | 4-[2-methoxy-3-(1-methyl-1,2,4- triazol-3-yl)anilino]-2-[(1-octylpyrazol- 4-yl)amino]pyrimidine-5-carboxamide |
| 26 | 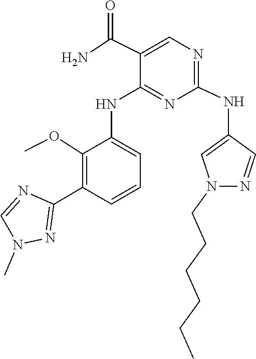 [SMILES: CCCCCCn1cc(Nc2ncc(C(N)=O)c(Nc3cccc(-c4ncn(C)n4)c3OC)n2)cn1] | 2-[(1-hexylpyrazol-4-yl)amino]-4-[2- methoxy-3-(1-methyl-1,2,4-triazol-3- yl)anilino]pyrimidine-5-carboxamide |
| 27 | 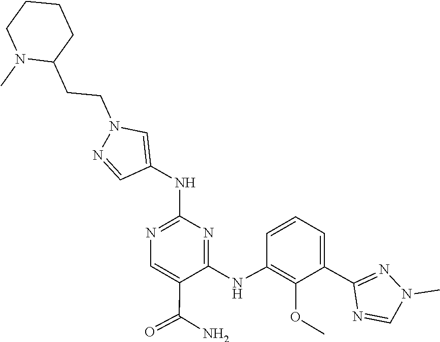 | 4-[2-methoxy-3-(1-methyl-1,2,4- triazol-3-yl)anilino]-2-[[1-[2-(1- methyl-2-piperidyl)ethyl]pyrazol-4- yl]amino]pyrimidine-5-carboxamide |
| 28 | 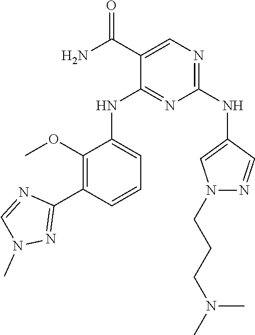 [SMILES: COc1c(Nc2nc(Nc3cnn(CCCN(C)C)c3)ncc2C(N)=O)cccc1-c1ncn(C)n1] | 2-[[1-[3- (dimethylamino)propyl]pyrazol-4- yl]amino]-4-[2-methoxy-3-(1-methyl- 1,2,4-triazol-3-yl)anilino]pyrimidine-5- carboxamide |
| 29 | 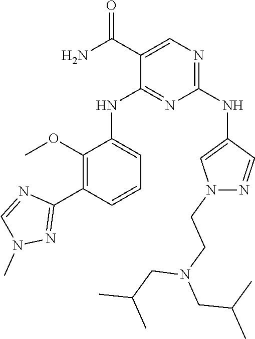 [SMILES: COc1c(Nc2nc(Nc3cnn(CCN(CC(C)C)CC(C)C)c3)ncc2C(N)=O)cccc1-c1ncn(C)n1] | 2-[[1-[2- (diisobutylamino)ethyl]pyrazol-4- yl]amino]-4-[2-methoxy-3-(1-methyl- 1,2,4-triazol-3-yl)anilino]pyrimidine-5- carboxamide |
| 30 | 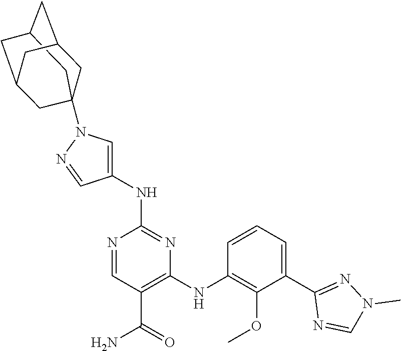 [SMILES: COc1c(Nc2nc(Nc3cnn(C45CC6CC(CC(C6)C4)C5)c3)ncc2C(N)=O)cccc1-c1ncn(C)n1] | 2-[[1-(1-adamantyl)pyrazol-4- yl]amino]-4-[2-methoxy-3-(1-methyl- 1,2,4-triazol-3-yl)anilino]pyrimidine-5- carboxamide |
| 31 | 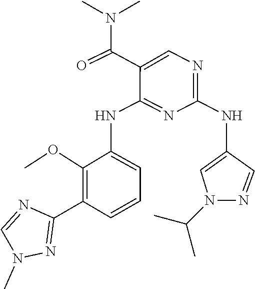 [SMILES: COc1c(Nc2nc(Nc3cnn(C(C)C)c3)ncc2C(=O)N(C)C)cccc1-c1ncn(C)n1] | 2-[(1-isopropylpyrazol-4-yl)amino]-4- [2-methoxy-3-(1-methyl-1,2,4-triazol- 3-yl)anilino]-N,N-dimethyl- pyrimidine-5-carboxamide |
| 32 | 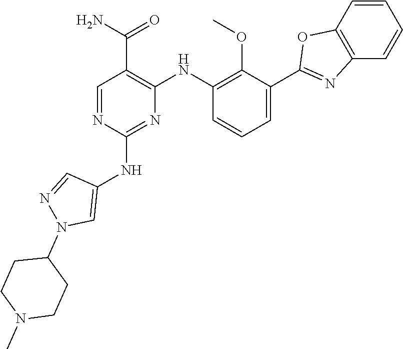 [SMILES: COc1c(Nc2nc(Nc3cnn(C4CCN(C)CC4)c3)ncc2C(N)=O)cccc1-c1nc2ccccc2o1] | 4-[3-(1,3-benzoxazol-2-yl)-2-methoxy- anilino]-2-[[1-(1-methyl-4- piperidyl)pyrazol-4- yl]amino]pyrimidine-5-carboxamide |
| 33 | 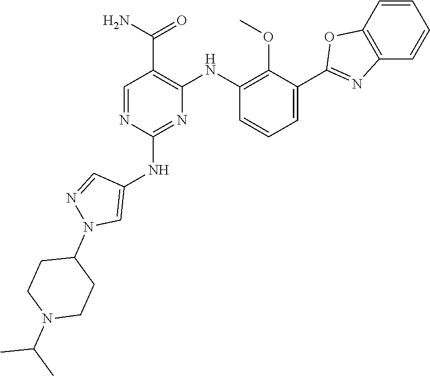 [SMILES: COc1c(Nc2nc(Nc3cnn(C4CCN(C(C)C)CC4)c3)ncc2C(N)=O)cccc1-c1nc2ccccc2o1] | 4-[3-(1,3-benzoxazol-2-yl)-2-methoxy- anilino]-2-[[1-(1-isopropyl-4- piperidyl)pyrazol-4- yl]amino]pyrimidine-5-carboxamide |
| 34 | 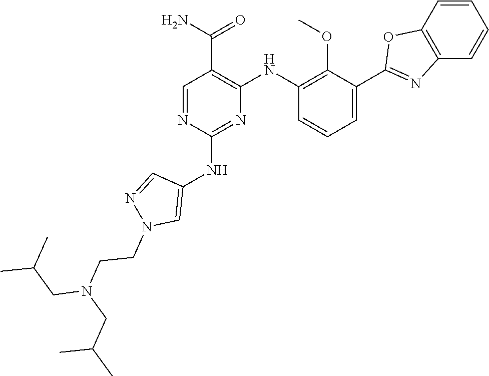 [SMILES: COc1c(Nc2nc(Nc3cnn(CCN(CC(C)C)CC(C)C)c3)ncc2C(N)=O)cccc1-c1nc2ccccc2o1] | 4-[3-(1,3-benzoxazol-2-yl)-2-methoxy- anilino]-2-[[1-[2- (diisobutylamino)ethyl]pyrazol-4- yl]amino]pyrimidine-5-carboxamide |
| 35 | 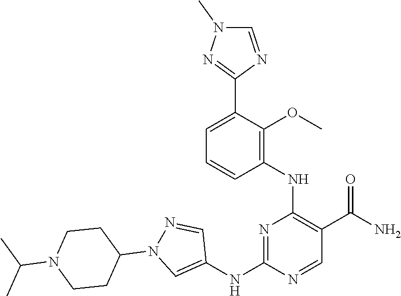 [SMILES: COc1c(Nc2nc(Nc3cnn(C4CCN(C(C)C)CC4)c3)ncc2C(N)=O)cccc1-c1ncn(C)n1] | 2-[[1-(1-isopropyl-4-piperidyl)pyrazol- 4-yl]amino]-4-[2-methoxy-3-(1- methyl-1,2,4-triazol-3- yl)anilino]pyrimidine-5-carboxamide |
| 36 | 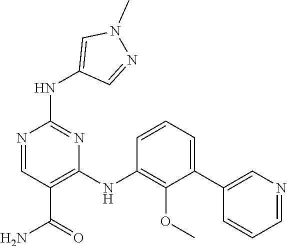 [SMILES: COc1c(Nc2nc(Nc3cnn(C)c3)ncc2C(N)=O)cccc1-c1cccnc1] | 4-[2-methoxy-3-(3-pyridyl)anilino]-2- [(1-methylpyrazol-4- yl)amino]pyrimidine-5-carboxamide |
| 37 | 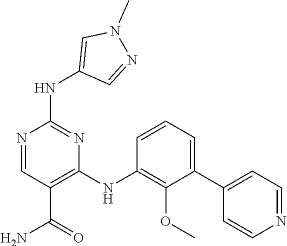 [SMILES: COc1c(Nc2nc(Nc3cnn(C)c3)ncc2C(N)=O)cccc1-c1ccncc1] | 4-[2-methoxy-3-(4-pyridyl)anilino]-2- [(1-methylpyrazol-4- yl)amino]pyrimidine-5-carboxamide |
| 38 | 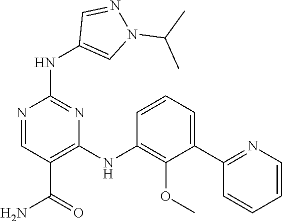 [SMILES: COc1c(Nc2nc(Nc3cnn(C(C)C)c3)ncc2C(N)=O)cccc1-c1ccccn1] | 2-[(1-isopropylpyrazol-4-yl)amino]-4- [2-methoxy-3-(2- pyridyl)anilino]pyrimidine-5- carboxamide |
| 39 | 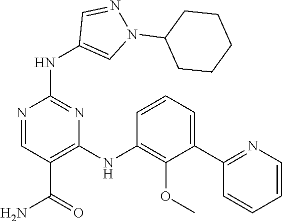 [SMILES: COc1c(Nc2nc(Nc3cnn(C4CCCCC4)c3)ncc2C(N)=O)cccc1-c1ccccn1] | 2-[(1-cyclohexylpyrazol-4-yl)amino]- 4-[2-methoxy-3-(2- pyridyl)anilino]pyrimidine-5- carboxamide |
| 40 | 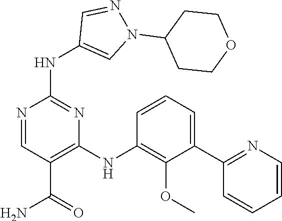 [SMILES: COc1c(Nc2nc(Nc3cnn(C4CCOCC4)c3)ncc2C(N)=O)cccc1-c1ccccn1] | 4-[2-methoxy-3-(2-pyridyl)anilino]-2- [(1-tetrahydropyran-4-ylpyrazol-4- yl)amino]pyrimidine-5-carboxamide |
| 41 | 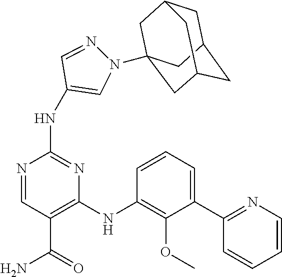 [SMILES: COc1c(Nc2nc(Nc3cnn(C45CC6CC(CC(C6)C4)C5)c3)ncc2C(N)=O)cccc1-c1ccccn1] | 2-[[1-(1-adamantyl)pyrazol-4- yl]amino]-4-[2-methoxy-3-(2- pyridyl)anilino]pyrimidine-5- carboxamide |
| 42 | 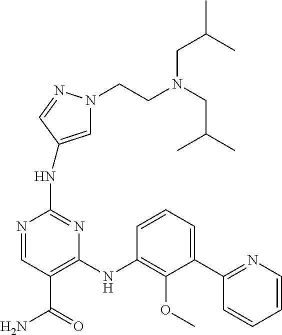 [SMILES: COc1c(Nc2nc(Nc3cnn(CCN(CC(C)C)CC(C)C)c3)ncc2C(N)=O)cccc1-c1ccccn1] | 2-[[1-[2- (diisobutylamino)ethyl]pyrazol-4- yl]amino]-4-[2-methoxy-3-(2- pyridyl)anilino]pyrimidine-5- carboxamide |
| 43 | 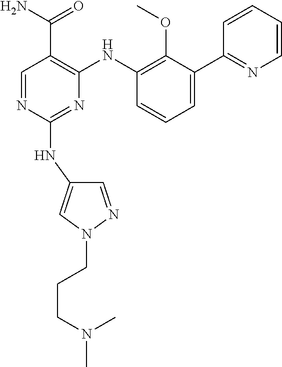 [SMILES: COc1c(Nc2nc(Nc3cnn(CCCN(C)C)c3)ncc2C(N)=O)cccc1-c1ccccn1] | 2-[[1-[3- (dimethylamino)propyl]pyrazol-4- yl]amino]-4-[2-methoxy-3-(2- pyridyl)anilino]pyrimidine-5- carboxamide |
| 44 | 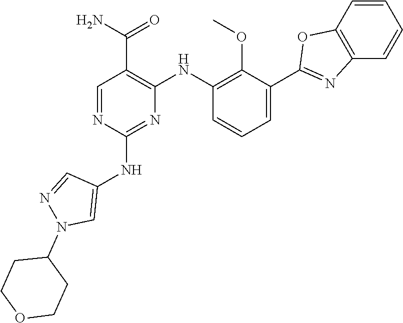 [SMILES: COc1c(Nc2nc(Nc3cnn(C4CCOCC4)c3)ncc2C(N)=O)cccc1-c1nc2ccccc2o1] | 4-[3-(1,3-benzoxazol-2-yl)-2-methoxy- anilino]-2-[(1-tetrahydropyran-4- ylpyrazol-4-yl)amino]pyrimidine-5- carboxamide |
| 45 | 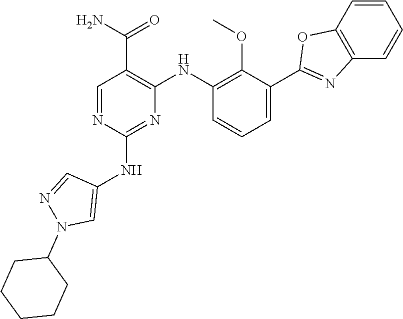 [SMILES: COc1c(Nc2nc(Nc3cnn(C4CCCCC4)c3)ncc2C(N)=O)cccc1-c1nc2ccccc2o1] | 4-[3-(1,3-benzoxazol-2-yl)-2-methoxy- anilino]-2-[(1-cyclohexylpyrazol-4- yl)amino]pyrimidine-5-carboxamide |
| 46 | 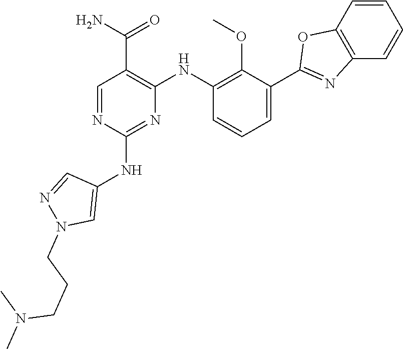 [SMILES: COc1c(Nc2nc(Nc3cnn(CCCN(C)C)c3)ncc2C(N)=O)cccc1-c1nc2ccccc2o1] | 4-[3-(1,3-benzoxazol-2-yl)-2-methoxy- anilino]-2-[[1-[3- (dimethylamino)propyl]pyrazol-4- yl]amino]pyrimidine-5-carboxamide |
| 47 | 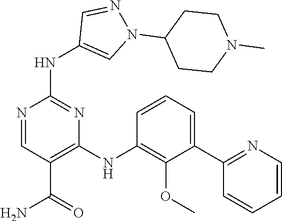 [SMILES: COc1c(Nc2nc(Nc3cnn(C4CCN(C)CC4)c3)ncc2C(N)=O)cccc1-c1ccccn1] | 4-[2-methoxy-3-(2-pyridyl)anilino]-2- [[1-(1-methyl-4-piperidyl)pyrazol-4- yl]amino]pyrimidine-5-carboxamide |
| 48 | 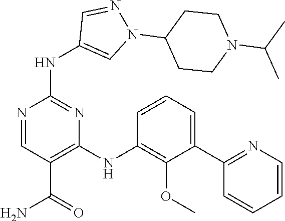 [SMILES: COc1c(Nc2nc(Nc3cnn(C4CCN(C(C)C)CC4)c3)ncc2C(N)=O)cccc1-c1ccccn1] | 2-[[1-(1-isopropyl-4-piperidyl)pyrazol- 4-yl]amino]-4-[2-methoxy-3-(2- pyridyl)anilino]pyrimidine-5- carboxamide |
| 49 | 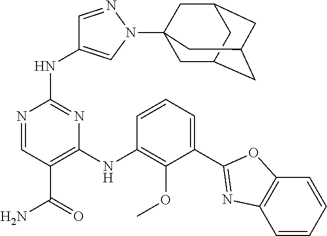 [SMILES: COc1c(Nc2nc(Nc3cnn(C45CC6CC(CC(C6)C4)C5)c3)ncc2C(N)=O)cccc1-c1nc2ccccc2o1] | 2-[[1-(1-adamantyl)pyrazol-4- yl]amino]-4-[3-(1,3-benzoxazol-2-yl)- 2-methoxy-anilino]pyrimidine-5- carboxamide |
| 50 | 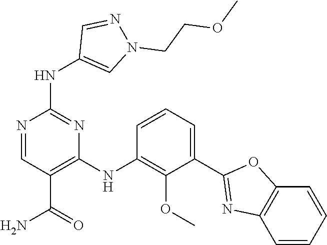 [SMILES: COCCn1cc(Nc2ncc(C(N)=O)c(Nc3cccc(-c4nc5ccccc5o4)c3OC)n2)cn1] | 4-[3-(1,3-benzoxazol-2-yl)-2-methoxy- anilino]-2-[[1-(2- methoxyethyl)pyrazol-4- yl]amino]pyrimidine-5-carboxamide |
| 51 | 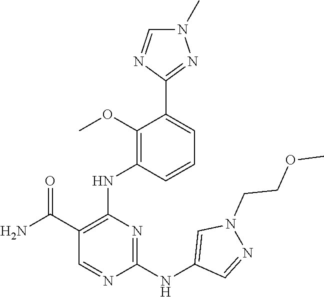 [SMILES: COCCn1cc(Nc2ncc(C(N)=O)c(Nc3cccc(-c4ncn(C)n4)c3OC)n2)cn1] | 2-[[1-(2-methoxyethyl)pyrazol-4- yl]amino]-4-[2-methoxy-3-(1-methyl- 1,2,4-triazol-3-yl)anilino]pyrimidine-5- carboxamide |
| 52 | 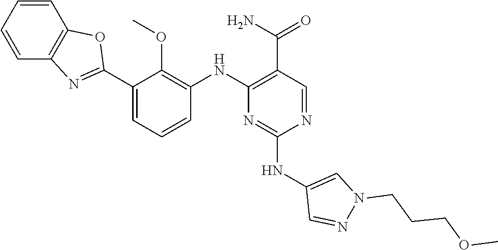 [SMILES: COCCCn1cc(Nc2ncc(C(N)=O)c(Nc3cccc(-c4nc5ccccc5o4)c3OC)n2)cn1] | 4-[3-(1,3-benzoxazol-2-yl)-2-methoxy- anilino]-2-[[1-(3- methoxypropyl)pyrazol-4- yl]amino]pyrimidine-5-carboxamide |
| 53 | 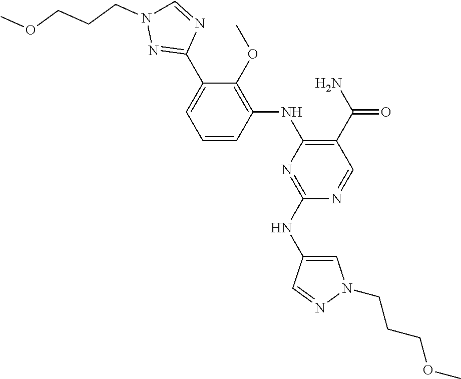 | 4-[2-methoxy-3-[1-(3-methoxypropyl)- 1,2,4-triazol-3-yl]anilino]-2-[[1-(3- methoxypropyl)pyrazol-4- yl]amino]pyrimidine-5-carboxamide |
| 54 | 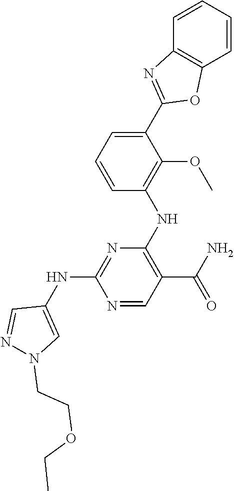 [SMILES: CCOCCn1cc(Nc2ncc(C(N)=O)c(Nc3cccc(-c4nc5ccccc5o4)c3OC)n2)cn1] | 4-[3-(1,3-benzoxazol-2-yl)-2-methoxy- anilino]-2-[[1-(2-ethoxyethyl)pyrazol- 4-yl]amino]pyrimidine-5-carboxamide |
| 55 | 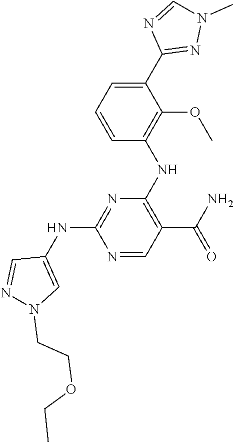 [SMILES: CCOCCn1cc(Nc2ncc(C(N)=O)c(Nc3cccc(-c4ncn(C)n4)c3OC)n2)cn1] | 2-[[1-(2-ethoxyethyl)pyrazol-4- yl]amino]-4-[2-methoxy-3-(1-methyl- 1,2,4-triazol-3-yl)anilino]pyrimidine-5- carboxamide |
| 56 | 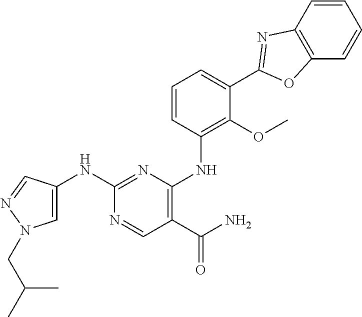 [SMILES: COc1c(Nc2nc(Nc3cnn(CC(C)C)c3)ncc2C(N)=O)cccc1-c1nc2ccccc2o1] | 4-[3-(1,3-benzoxazol-2-yl)-2-methoxy- anilino]-2-[(1-isobutylpyrazol-4- yl)amino]pyrimidine-5-carboxamide |
| 57 | 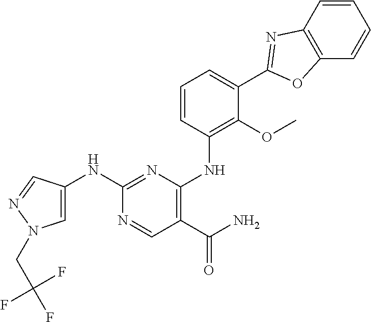 [SMILES: COc1c(Nc2nc(Nc3cnn(CC(F)(F)F)c3)ncc2C(N)=O)cccc1-c1nc2ccccc2o1] | 4-[3-(1,3-benzoxazol-2-yl)-2-methoxy- anilino]-2-[[1-(2,2,2- trifluoroethyl)pyrazol-4- yl]amino]pyrimidine-5-carboxamide |
| 58 | 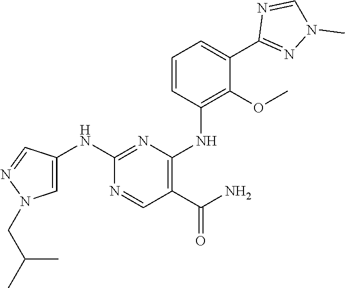 [SMILES: COc1c(Nc2nc(Nc3cnn(CC(C)C)c3)ncc2C(N)=O)cccc1-c1ncn(C)n1] | 2-[(1-isobutylpyrazol-4-yl)amino]-4- [2-methoxy-3-(1-methyl-1,2,4-triazol- 3-yl)anilino]pyrimidine-5-carboxamide |
| 59 | 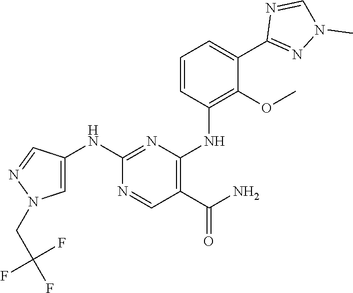 [SMILES: COc1c(Nc2nc(Nc3cnn(CC(F)(F)F)c3)ncc2C(N)=O)cccc1-c1ncn(C)n1] | 4-[2-methoxy-3-(1-methyl-1,2,4- triazol-3-yl)anilino]-2-[[1-(2,2,2- trifluoroethyl)pyrazol-4- yl]amino]pyrimidine-5-carboxamide |
| 60 | 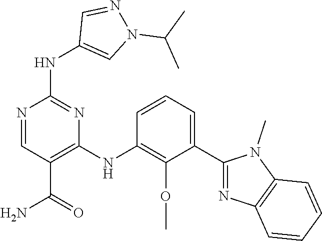 [SMILES: COc1c(Nc2nc(Nc3cnn(C(C)C)c3)ncc2C(N)=O)cccc1-c1nc2ccccc2n1C] | 2-[(1-isopropylpyrazol-4-yl)amino]-4- [2-methoxy-3-(1-methylbenzimidazol- 2-yl)anilino]pyrimidine-5-carboxamide |
| 61 | 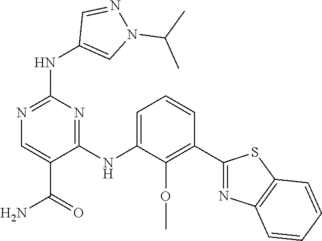 [SMILES: COc1c(Nc2nc(Nc3cnn(C(C)C)c3)ncc2C(N)=O)cccc1-c1nc2ccccc2s1] | 4-[3-(1,3-benzothiazol-2-yl)-2- methoxy-anilino]-2-[(1- isopropylpyrazol-4- yl)amino]pyrimidine-5-carboxamide |
| 62 | 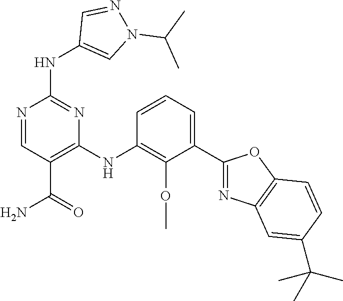 [SMILES: COc1c(Nc2nc(Nc3cnn(C(C)C)c3)ncc2C(N)=O)cccc1-c1nc2cc(C(C)(C)C)ccc2o1] | 4-[3-(5-tert-butyl-1,3-benzoxazol-2-yl)- 2-methoxy-anilino]-2-[(1- isopropylpyrazol-4- yl)amino]pyrimidine-5-carboxamide |
| 63 | 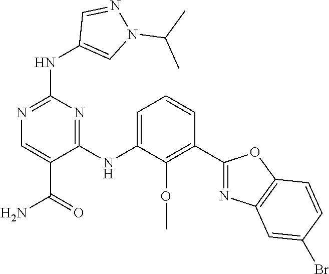 [SMILES: COc1c(Nc2nc(Nc3cnn(C(C)C)c3)ncc2C(N)=O)cccc1-c1nc2cc(Br)ccc2o1] | 4-[3-(5-bromo-1,3-benzoxazol-2-yl)-2- methoxy-anilino]-2-[(1- isopropylpyrazol-4- yl)amino]pyrimidine-5-carboxamide |
| 64 | 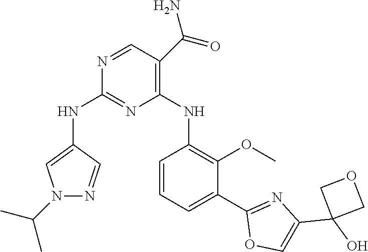 [SMILES: COc1c(Nc2nc(Nc3cnn(C(C)C)c3)ncc2C(N)=O)cccc1-c1nc(C2(O)COC2)co1] | 4-[3-[4-(3-hydroxyoxetan-3-yl)oxazol- 2-yl]-2-methoxy-anilino]-2-[(1- isopropylpyrazol-4- yl)amino]pyrimidine-5-carboxamide |
| 65 | 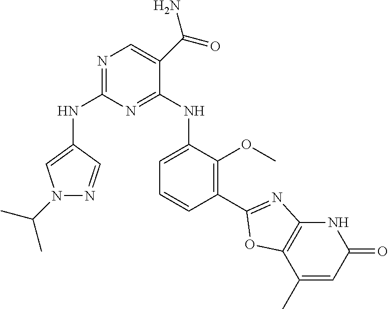 [SMILES: COc1c(Nc2nc(Nc3cnn(C(C)C)c3)ncc2C(N)=O)cccc1-c1nc2[nH]c(=O)cc(C)c2o1] | 2-[(1-isopropylpyrazol-4-yl)amino]-4- [2-methoxy-3-(7-methyl-5-oxo-4H- oxazolo[4,5-b]pyridin-2- yl)anilino]pyrimidine-5-carboxamide |
| 66 | 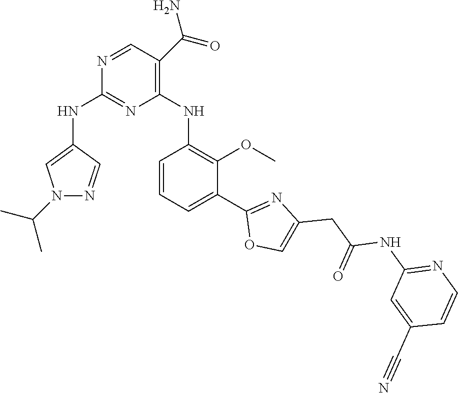 [SMILES: COc1c(Nc2nc(Nc3cnn(C(C)C)c3)ncc2C(N)=O)cccc1-c1nc(CC(=O)Nc2cc(C#N)ccn2)co1] | 4-[3-[4-[2-[(4-cyano-2-pyridyl)amino]- 2-oxo-ethyl]oxazol-2-yl]-2-methoxy- anilino]-2-[(1-isopropylpyrazol-4- yl)amino]pyrimidine-5-carboxamide |
| 67 | 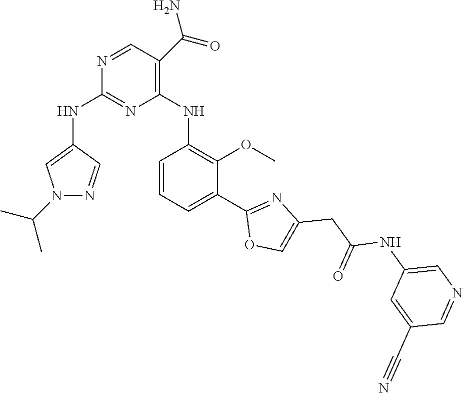 [SMILES: COc1c(Nc2nc(Nc3cnn(C(C)C)c3)ncc2C(N)=O)cccc1-c1nc(CC(=O)Nc2cncc(C#N)c2)co1] | 4-[3-[4-[2-[(5-cyano-3-pyridyl)amino]- 2-oxo-ethyl]oxazol-2-yl]-2-methoxy- anilino]-2-[(1-isopropylpyrazol-4- yl)amino]pyrimidine-5-carboxamide |
| 68 | 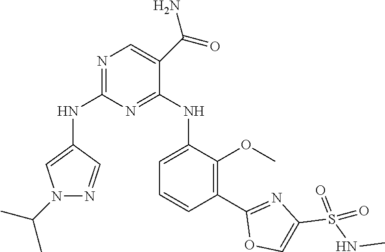 [SMILES: CNS(=O)(=O)c1coc(-c2cccc(Nc3nc(Nc4cnn(C(C)C)c4)ncc3C(N)=O)c2OC)n1] | 2-[(1-isopropylpyrazol-4-yl)amino]-4- [2-methoxy-3-[4- (methylsulfamoyl)oxazol-2- yl]anilino]pyrimidine-5-carboxamide |
| 69 | 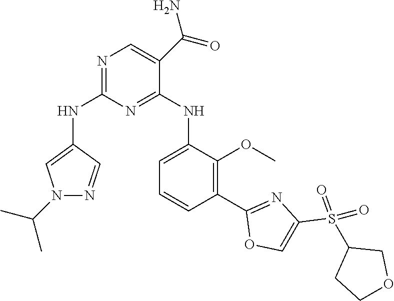 [SMILES: COc1c(Nc2nc(Nc3cnn(C(C)C)c3)ncc2C(N)=O)cccc1-c1nc(S(=O)(=O)C2CCOC2)co1] | 2-[(1-isopropylpyrazol-4-yl)amino]-4- [2-methoxy-3-(4-tetrahydrofuran-3- ylsulfonyloxazol-2- yl)anilino]pyrimidine-5-carboxamide |
| 70 | 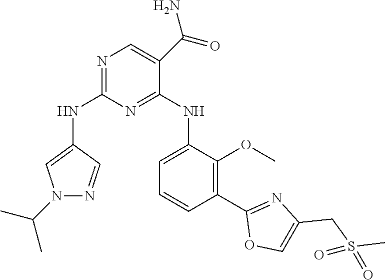 [SMILES: COc1c(Nc2nc(Nc3cnn(C(C)C)c3)ncc2C(N)=O)cccc1-c1nc(CS(C)(=O)=O)co1] | 2-[(1-isopropylpyrazol-4-yl)amino]-4- [2-methoxy-3-[4- (methylsulfonylmethyl)oxazol-2- yl]anilino]pyrimidine-5-carboxamide |
| 71 | 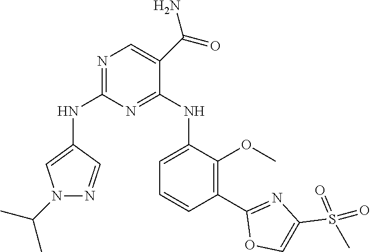 [SMILES: COc1c(Nc2nc(Nc3cnn(C(C)C)c3)ncc2C(N)=O)cccc1-c1nc(S(C)(=O)=O)co1] | 2-[(1-isopropylpyrazol-4-yl)amino]-4- [2-methoxy-3-(4- methylsulfonyloxazol-2- yl)anilino]pyrimidine-5-carboxamide |
| 72 | 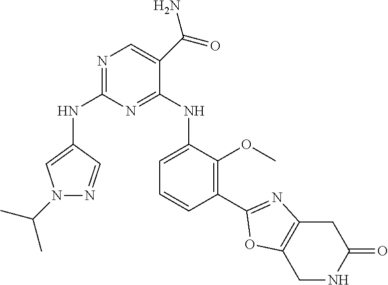 [SMILES: COc1c(Nc2nc(Nc3cnn(C(C)C)c3)ncc2C(N)=O)cccc1-c1nc2c(o1)CNC(=O)C2] | 2-[(1-isopropylpyrazol-4-yl)amino]-4- [2-methoxy-3-(6-oxo-5,7-dihydro-4H- oxazolo[5,4-c]pyridin-2- yl)anilino]pyrimidine-5-carboxamide |
| 73 | 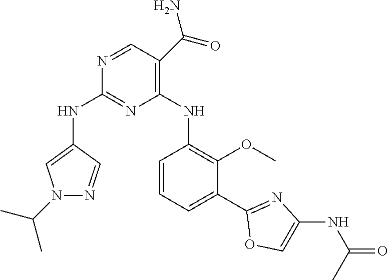 [SMILES: COc1c(Nc2nc(Nc3cnn(C(C)C)c3)ncc2C(N)=O)cccc1-c1nc(NC(C)=O)co1] | 4-[3-(4-acetamidooxazol-2-yl)-2- methoxy-anilino]-2-[(1- isopropylpyrazol-4- yl)amino]pyrimidine-5-carboxamide |
| 74 | 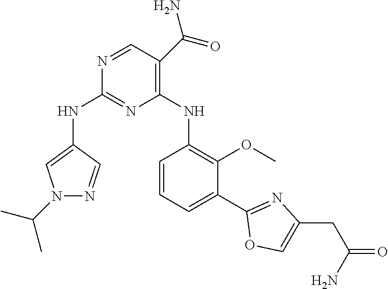 [SMILES: COc1c(Nc2nc(Nc3cnn(C(C)C)c3)ncc2C(N)=O)cccc1-c1nc(CC(N)=O)co1] | 4-[3-[4-(2-amino-2-oxo-ethyl)oxazol- 2-yl]-2-methoxy-anilino]-2-[(1- isopropylpyrazol-4- yl)amino]pyrimidine-5-carboxamide |
| 75 | 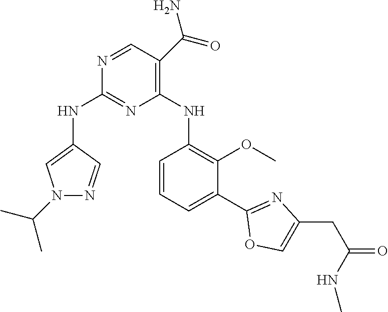 [SMILES: CNC(=O)Cc1coc(-c2cccc(Nc3nc(Nc4cnn(C(C)C)c4)ncc3C(N)=O)c2OC)n1] | 2-[(1-isopropylpyrazol-4-yl)amino]-4- [2-methoxy-3-[4-[2-(methylamino)-2- oxo-ethyl]oxazol-2- yl]anilino]pyrimidine-5-carboxamide |
| 76 | 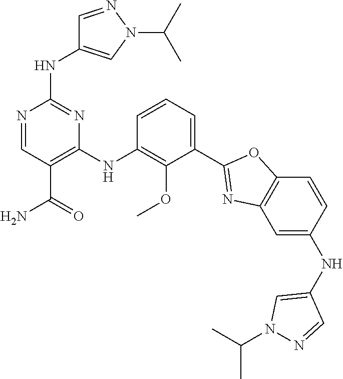 [SMILES: COc1c(Nc2nc(Nc3cnn(C(C)C)c3)ncc2C(N)=O)cccc1-c1nc2cc(Nc3cnn(C(C)C)c3)ccc2o1] | 2-[(1-isopropylpyrazol-4-yl)amino]-4- [3-[5-[(1-isopropylpyrazol-4- yl)amino]-1,3-benzoxazol-2-yl]-2- methoxy-anilino]pyrimidine-5- carboxamide |

or a pharmaceutically acceptable salt, stereoisomer, solvate, or tautomer thereof.

10. A pharmaceutical composition comprising the compound of claim 1 or a pharmaceutically acceptable salt, stereoisomer, solvate, or tautomer thereof, and a pharmaceutically acceptable carrier.

11. The pharmaceutical composition of claim 10, further comprising one or more additional pharmaceutically active agents.

12. A method of inhibiting JAK2 in a cell, comprising contacting the cell with a compound of claim 1.

13. The method of claim 12 or 13, wherein the contacting is in vitro or in vivo.

14. A method for the treatment or prevention of a disease or disorder associated with JAK2 comprising administering to a subject in need thereof a compound of claim 1.

15. The method of claim 14, wherein the disease or disorder is selected from the group consisting of polycythemia vera; essential thrombocytosis; thrombocythemia 3; primary myelofibrosis; chronic myelomonocytic leukemia; acute myeloid leukemia; myelodysplasia; Budd-Chiari syndrome; erythrocytosis, familial, 1 (ECYT1); myeloproliferative neoplasm (MPD); polycythemia; lymphoblastic leukemia, acute, with lymphomatous features (LALL); essential thrombocythemia (ET); premature menopause; thrombocytosis; hypereosinophilic syndrome (HES); splenomegaly; acute leukemia; thrombosis; portal hypertension; papilledema; acquired polycythemia; systemic mastocytosis (SMCD); chronic myelomonocytic leukemia (CMML); primary polycythemia; neutrophilia, hereditary (NEUTROPHILIA); thrombophilia; leukemia; hematologic cancer; myelophthisic anemia; erythroleukemia; myeloid leukemia; antithrombin III Deficiency (AT3D); severe congenital neutropenia; leukemia, chronic myeloid (CML); fibrosarcoma; mastocytosis; myelodysplastic syndrome (MDS); chronic eosinophilic leukemia; bone marrow cancer; behcet syndrome (BD); pancreatic adenocarcinoma; adenocarcinoma; stress polycythemia; B-cell lymphoma; myelodysplastic/myeloproliferative neoplasm; hemangioblastoma; atypical chronic myeloid leukemia, Bcr-Abl1 negative (ACML); chronic neutrophilic leukemia (CNL); gastrointestinal stromal tumor (GIST); beta-thalassemia (B-THAL); acquired von Willebrand syndrome (AVWS); splenic infarction; B-lymphoblastic leukemia/lymphoma, Bcr-Abl1-like; lymphoma, Hodgkin, classic (CHL); Down syndrome; Wernicke encephalopathy; hepatic vascular disease; primary mediastinal B-Cell lymphoma; hyperglycemia; chronic leukemia (CLL); portal vein thrombosis; Diamond-Blackfan anemia (BDA); leptin deficiency or dysfunction (LEPD); deficiency anemia; hepatocellular carcinoma (HCC); Sm-Ahnmd; leukemia, acute lymphoblastic (ALL); sagittal sinus thrombosis; blood coagulation disease; acute erythroid leukemia; immunodeficiency 35 (IMD35); thrombocytopenia; colorectal cancer (CRC); blood platelet disease; temporal arteritis (GCA); amegakaryocytic thrombocytopenia, congenital (CAMT); inflammatory bowel disease; vein disease; erythromelalgia; erythrocytosis, familial, 2 (ECYT2); prostate cancer (PC); erythrocytosis, familial, 6 (ECYT6); aggressive systemic mastocytosis (ASM); myeloid and lymphoid neoplasms associated with pdgfra rearrangement; pancreatic cancer (PNCA); ovarian cancer (OC); juvenile myelomonocytic leukemia (JMML); breast cancer (BC); gastric cancer (GASC); medulloblastoma (MDB); lymphoma, non-Hodgkin, familial (NHL); myocardial infarction (MCI1); body mass index quantitative trait locus 11 (BMIQ11); acute salpingo-oophoritis; autism spectrum disorder (ASD); esophageal cancer (ESCR); leukemia, chronic lymphocytic (CLL); myeloma, multiple (MM); hypertension, essential (EHT); type 2 diabetes mellitus (T2D); skin disease; Rasopathy; connective tissue disease; peripheral nervous system disease; nervous system disease.

16. The method of claim 14, wherein the disease or disorder is leukemia.

17. The method of claim 16, wherein leukemia is selected from: chronic myelomonocytic leukemia; acute myeloid leukemia; lymphoblastic leukemia, acute, with lymphomatous features (LALL); acute leukemia; chronic myelomonocytic leukemia (CMML); myeloid leukemia; leukemia, chronic myeloid (CML); chronic eosinophilic leukemia; atypical chronic myeloid leukemia, Bcr-Abl1 negative (ACML); chronic neutrophilic leukemia (CNL); B-lymphoblastic leukemia/lymphoma, Bcr-Abl1-like; chronic leukemia (CLL); deficiency anemia; hepatocellular carcinoma (HCC); Sm-Ahnmd; leukemia, acute lymphoblastic (ALL); acute erythroid leukemia; juvenile myelomonocytic leukemia (JMML); leukemia, chronic lymphocytic (CLL).

18. The method of any one of claims 12-17, wherein the subject is a mammal.

19. The method of claim 18, wherein the subject is a human.
